(19) United States
(12) Patent Application Publication
(10) Pub. No.: US 2026/0159474 A1
(43) Pub. Date: Jun. 11, 2026

(54) Aminoindane Compounds and Compositions and Methods of Their Therapeutic Use

(71) Applicant: Solvonis Therapeutics Ireland Holdings Limited, Dublin (IE)

(72) Inventors: David Nutt, London (GB); Alan Borthwick, London (GB)

(21) Appl. No.: 19/325,448

(22) Filed: Sep. 10, 2025

Related U.S. Application Data
(62) Division of application No. 18/560,543, now abandoned.
(60) Provisional application No. 63/187,389, filed on May 11, 2021.
(60) Provisional application No. 63/187,397, filed on May 11, 2021.

(51) Int. Cl. C07C 229/46; A61K 9/00; A61K 9/10; A61K 9/20; A61K 9/48; A61K 45/06; A61K 47/10; A61K 47/12; A61K 47/26; A61K 47/32; A61K 47/36; A61K 47/38; A61K 47/44; A61P 25/00; A61P 25/30; A61P 25/32; C07C 229/50; C07D 305/14; C07D 307/77; C07D 311/78; C07D 313/06; C07D 313/20

(52) U.S. Cl. CPC C07C 229/46; A61K 9/0014; A61K 9/0019; A61K 9/006; A61K 9/10; A61K 9/2013; A61K 9/2027; A61K 9/2054; A61K 9/2059; A61K 9/4858; A61K 9/4866; A61K 45/06; A61K 47/10; A61K 47/12; A61K 47/26; A61K 47/32; A61K 47/36; A61K 47/38; A61K 47/44; A61P 25/00; A61P 25/30; A61P 25/32; C07C 229/50; C07D 305/14; C07D 307/77; C07D 311/78; C07D 313/06; C07D 313/20

(57) ABSTRACT

The present invention discloses therapeutic compounds of the aminoindane class, including certain short-acting ester and lactone derivatives, and pharmaceutical compositions made therewith, as well as methods of their use in the treatment of alcohol and other substance use disorders, behavioral addictions, and CNS and mental health disorders, and for the improvement of mental health and psychological functioning.

### CROSS-REFERENCE

This is a divisional application under 35 U.S.C. § 121 of U.S. application Ser. No. 18/560,543, filed Nov. 13, 2023 and published Jun. 13, 2024 as U.S. Pub. No. 2024/0190809A1, which is a U.S. National Stage Entry under 35 U.S.C. § 371 of International Application No. PCT/EP2022/062836, filed May 11, 2022 and published Nov. 17, 2022 as PCT Pub. No. WO2022/238507A1, which claims priority under PCT Article 8(1) and Rule 4.10 to U.S. Provisional Application Nos. 63/187,389 and 63/187,397 both filed May 11, 2021; each are incorporated by reference for all purposes as if fully set forth herein.

### TECHNICAL FIELD

Described are therapeutic aminoindane compounds, including short-acting ester and lactone derivatives, and pharmaceutical compositions made therewith, useful in the treatment of alcohol and other substance use disorders, behavioral addictions, and mental health disorders, and for the improvement of mental health and psychological functioning.

### BACKGROUND OF THE INVENTION

Addiction is one of the world's biggest unmet medical needs. Substance and behavioral addictions affect over 1.3 billion people globally. Between 15-20% of adults suffer from substance addictions, and a great many from behavioral addictions of various types.

All current treatments have high failure rates. For example, of the alcohol use disorder sufferers who seek treatment, up to 75% relapse within 12 months. Behavioral addictions are in fact so poorly served there are few data even available on treatment or success rates.

Accordingly, there is a vast need for effective treatments. Provided herein are therapeutic compounds, pharmaceutical compositions comprising such compounds, and methods of treatment using such compounds and compositions, to meet this compelling need.

### INCORPORATION BY REFERENCE

Each patent, publication, and non-patent literature cited in the application is hereby incorporated by reference in its entirety as if each was incorporated by reference individually. Unless specifically stated otherwise, reference to any document herein is not to be construed as an admission that the document referred to or any underlying information in the document is prior art in any jurisdiction, or forms part of the common general knowledge in the art.

### BRIEF SUMMARY OF THE INVENTION

The following presents a simplified summary of some embodiments of the invention in order to provide a basic understanding thereof. This summary is not an extensive overview, nor is it intended to identify every key or critical element of the invention or to delineate the complete scope of the invention. Its sole purpose is to present some exemplary embodiments of the invention in a simplified form, as a prelude to the more detailed description below.

In some aspects are disclosed compounds having a structure of Formula (I):

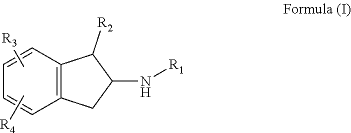

wherein any one of R₁, R₂, or R₃ is:

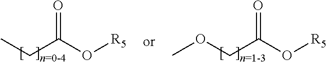

with the others of R₁, R₂, and R₃ both being hydrogen (H);
and wherein R₄ is either H or —OR₅;
   wherein each R₅ is independently any of H, alkyl, aryl, arylalkyl, —(CH₂)ₘZ(CH₂)ₘCH₃, or —(CH₂)ₘR^{cyc}, and each m is independently between 0-4;
      wherein each Z is independently, and in either orientation, —O—, —C═C—, —NH—, —NHNH—, —ONH—, —CONH, —CH(NH₂)—, —S—, —S(O)—, —SO₂—, —CHF—, and —CF₂—;
      wherein each R^{cyc} is independently an aryl or heteroaryl, said aryl or heteroaryl selected from the class consisting of phenyl, naphthyl, naphthalenyl, indolyl, thiophenyl, pyridyl, pyridinyl, pyrimidinyl, furyl, furanyl, tetrahydrofuranyl, pyrrolyl, oxazolyl, thiazolyl, pyrazolyl, isoxazolyl, imidazolyl, isothiazolyl, oxadiazolyl, triazolyl, thiadiazolyl, pyridazinyl, pyrazinyl, triazinyl, indolyzinyl, isoindolyl, benzofuranyl, dihydro benzofuranyl, benzoyl, benzoxazolyl, methylenedioxyphenyl, isobenzofuranyl, benzothiophenyl, benzoxazolyl, indazolyl, benzthiazolyl, benzimidazolyl, quinolinyl, isoquinolinyl, purinyl, quinolizinyl, cinnolinyl, phthalyl, quinoxalinyl, napthyridinyl, pteridinyl, and quinazolinyl;
         and wherein one or more hydrogen atoms of said aryl or heteroaryl are optionally and independently replaced with any of: (a) halogen, (b) C₁₋₇ alkyl, (c) hydroxy, (d) methoxy, (e) isopropoxy, (f) ethoxy,
         (g) hydroxymethyl, (h) hydroxypropyl, (i) tert-butyl, (j) n-butyl, (k) see-butyl, (l) isobutyl, (in) acetamide, (n) carbamoyl, (o) —CN, (p) —COOH, (q) —NO₂, (r) —S(O)₂OH, (s) —S(O)CH₃, (t) —S(O)₂CH₃, (u) —OR₆, or (v) —SR₆;
         wherein R₆ is hydrogen or C₁₋₇ alkyl;
or alternatively, where R₃ is

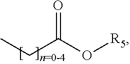

R₅ is joined to the benzene ring at an R₄ position adjacent to the R₃ to form a lactone ring.

In some embodiments, the compound has a structure of Formulas (Ia)-(Ir):

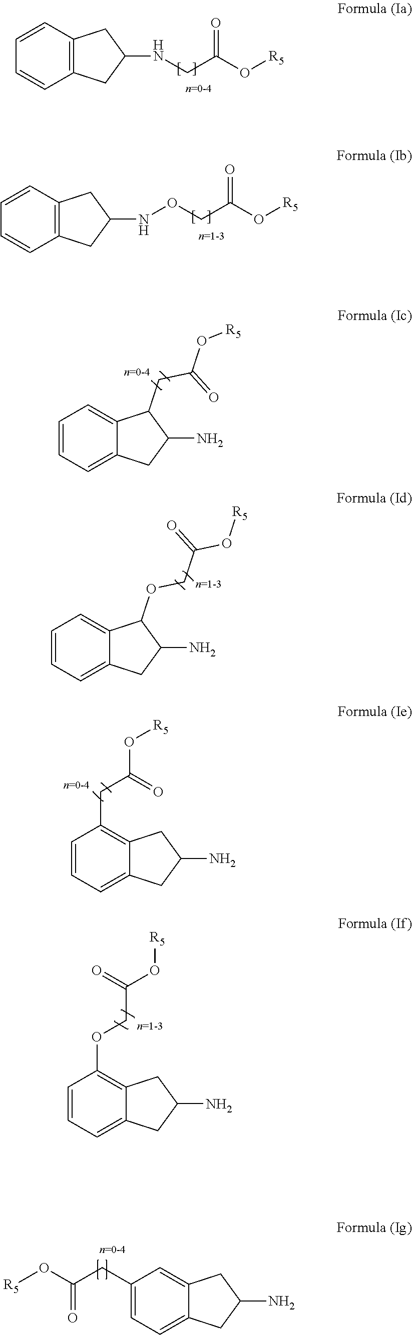

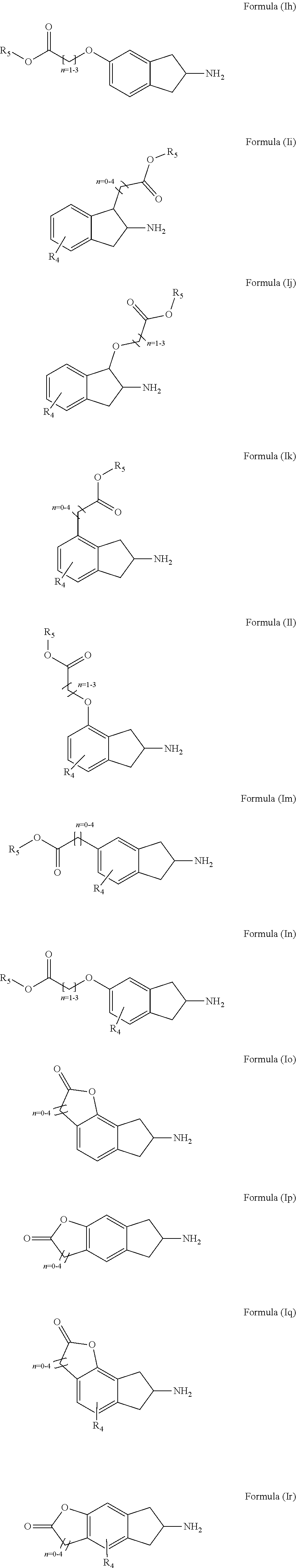

In some embodiments ofOthe compounds, each R₅ is H, or —CH₂—CH₃.

In some embodiments, the compound has any one of the structures below:

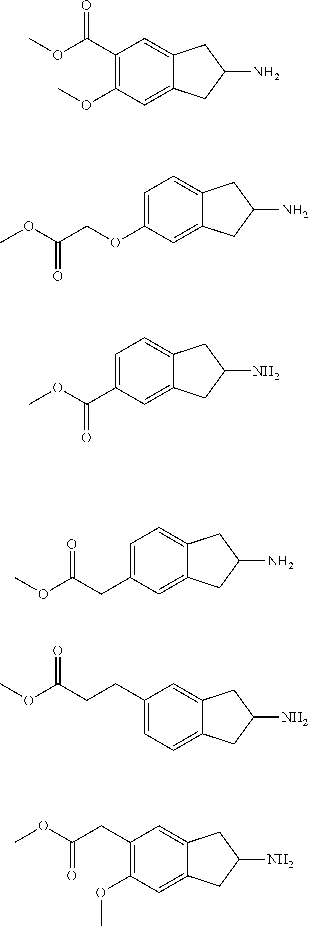 [SMILES: COC(=O)c1cc2c(cc1OC)CC(N)C2 ; COC(=O)COc1ccc2c(c1)CC(N)C2 ; COC(=O)c1ccc2c(c1)CC(N)C2 ; COC(=O)Cc1ccc2c(c1)CC(N)C2 ; COC(=O)CCc1ccc2c(c1)CC(N)C2 ; COC(=O)Cc1cc2c(cc1OC)CC(N)C2]

In some embodiments, the compound is classified as highly soluble in PBS pH 7.4. In some embodiments, the compound is classified as highly permeable, as determined by a MDR1-MDCKII permeability assay. In some embodiments, the compound is not determined to be a substrate for P-glycoprotein, as determined in the presence of a P-glycoprotein inhibitor or in mock MDCKII cells.

In some embodiments, the clearance rate of the compound is greater than the clearance rate of MDMA, as determined in human liver microsomes. In some embodiments, the half-life of the compound is shorter than the half-life of MDMA, as determined by a pharmacokinetic study in vitro or in vivo.

In some embodiments, the compound stimulates release of a monoamine neurotransmitter and inhibits the function of a monoamine transporter. In some embodiments, the compound stimulates release of a monoamine neurotransmitter or inhibits the function of a monoamine transporter. In some embodiments, the monoamine neurotransmitter is any of serotonin (5-HT), dopamine (DA), and norepinephrine (NE), and/or the monoamine transporter is any of a serotonin transporter (SERT), a dopamine transporter (DAT), and a norepinephrine transporter (NET).

In some aspects, the disclosed compounds are for use in modulating neurotransmission. In some embodiments, modulating neurotransmission treats a substance use disorder, a behavioral addiction, or a mental health disorder. In some aspects, the disclosed compounds are for use in the treatment of a substance use disorder, a behavioral addiction, or a mental health disorder. In some embodiments, the substance use disorder, the behavioral addiction, or the mental health disorder is any of alcohol use disorder, opioid use disorder, nicotine dependence, stimulant use disorder, tobacco use disorder, gambling addiction, gambling disorder, sexual addiction, gaming addiction, shopping addiction, internet addiction, binge eating disorder, internet gaming addiction, an anxiety disorder, a depressive disorder, or a trauma or stressor related disorder.

In some aspects are disclosed pharmaceutical compositions comprising a therapeutically effective amount of a disclosed compound for use in treating a substance use disorder, a behavioral addiction, or a mental health disorder, and a pharmaceutically acceptable carrier, diluent, or excipient. In some embodiments, the composition is suitable for oral, buccal, sublingual, injectable, subcutaneous, intravenous, or transdermal administration. In some embodiments, the composition is suitable for oral administration. In some embodiments, the composition is suitable for intravenous administration. In some embodiments, the composition is in a unit dosage form.

In some embodiments, the composition further comprises a therapeutically effective amount of an additional active agent. In some embodiments, the additional active agent is selected from the group consisting of: amino acids, antioxidants, anti-inflammatory agents, analgesics, antineuropathic and antinociceptive agents, antimigraine agents, anxiolytics, antidepressants, antipsychotics, anti-PTSD agents, cannabinoids, immunostimulants, anti-cancer agents, antiemetics, orexigenics, antiulcer agents, antihistamines, antihypertensives, anticonvulsants, antiepileptics, bronchodilators, neuroprotectants, entactogens and empathogens, entheogens, psychedelics, tryptamines, terpenes, beta-carbolines, phenethylamines, monoamine oxidase inhibitors, sedatives, stimulants, serotonergic agents, and vitamins. In some embodiments, the additional active agent acts to increase a therapeutic effect, provide an additional therapeutic effect, decrease an unwanted effect, increase stability or shelf-life, improve bioavailability, induce synergy, or alter pharmacokinetics or pharmacodynamics. In some embodiments, the additional therapeutic effect is an antioxidant, anti-inflammatory, analgesic, antineuropathic, antinociceptive, antimigraine, anxiolytic, antidepressant, antipsychotic, anti-PTSD, immunostimulant, anti-cancer, antiemetic, orexigenic, antiulcer, antihistamine, antihypertensive, anticonvulsant, antiepileptic, bronchodilator, neuroprotective, entactogenic, empathogenic, entheogenic, psychedelic, sedative, or stimulant effect.

In some embodiments, the additional active agent is selected from the group consisting of: opioid antagonists (e.g., nalmefene, naltrexone), CB-1 antagonists (e.g., rimonabant), CRH1 antagonists (e.g., verucerfont, pexacerfont), NK1R antagonists (e.g., tradipitant), OTR agonists (e.g., oxytocin), GABA agents (e.g., topiramate, baclofen, benzodiazepines, such as alprazolam, diazepam or lorazepam), voltage-gated sodium channel inhibitors (e.g., oxacarbazepine, valproic acid, zonisamide), voltage-dependent calcium channel agonists (e.g., gabapentin, pregabalin), a7 nicotinic acetylcholine receptor agonists (e.g., varenicline), 5-HT3 antagonists (e.g., ondansetron), 5-HT1A receptor partial agonists (e.g., aripiprazole), 5-HT2A receptor antagonists (e.g., quetiapine, olanzapine, mirtazapine), 5-HT reuptake inhibitors (e.g., trazodone), SERT inhibitors (e.g., duloxetine), α1 adrenoreceptor antagonists (e.g., doxazosin, prazosin), glucocorticoid receptor antagonists (e.g., mifepristone), α1 adrenoreceptor agonists (e.g., guanfacine), AChE inhibitors (e.g., citicoline), dopamine D2 receptor antagonists (e.g., tiapride), α2 adrenoreceptor agonists (e.g., clonidine), NMDA receptor antagonists (e.g., acamprosate), and aldehyde dehydrogenase inhibitors (e.g., disulfiram), including pharmaceutically acceptable salts.

In some aspects are disclosed unit dosage forms comprising a therapeutically effective amount of a disclosed compound. In some embodiments, the unit dosage form is formulated for oral administration. In some embodiments, the unit dosage form is formulated for immediate release, controlled release, sustained release, extended release, or modified release. In some embodiments, the unit dosage form is formulated for intravenous administration. In some embodiments, the unit dosage form comprises the compound in a total amount of between 1 and 100 mg. In some embodiments, the unit dosage form comprises the compound in a total amount of between 5 and 50 mg.

In some aspects are disclosed methods for modulating neurotransmission in a mammal, comprising administering to the mammal a therapeutically effective amount of a disclosed compound, composition, or unit dosage form. In some embodiments, the neurotransmission is serotonergic neurotransmission. In some embodiments, the neurotransmission is dopaminergic neurotransmission. In some embodiments, modulating neurotransmission comprises one or more of: (a) stimulating release of serotonin, and/or reducing serotonin uptake, by inhibiting the function of a serotonin transporter (SERT); and (b) stimulating release of dopamine, and/or reducing dopamine uptake, by inhibiting the function of a dopamine transporter (DAT). In some embodiments, the neurotransmission comprises reduced peak norepinephrine levels relative to peak serotonin levels and/or peak dopamine levels. In some embodiments, the neurotransmission is noradrenergic neurotransmission. In some embodiments, noradrenaline levels are increased from baseline following administration of the compound in a microdialysis assay. In some embodiments, the increase in noradrenaline levels from baseline is reduced relative to an increase in noradrenaline levels produced by MDMA or MEAI in a microdialysis assay. In some embodiments, modulating neurotransmission treats a substance use disorder, a behavioral addiction, or a mental health disorder in the mammal.

In some embodiments, the compound or the composition is orally administered to a mammal or a subject. In some embodiments, the compound or the composition is intravenously administered to the mammal or the subject.

In some aspects are disclosed methods of treating a medical condition in a mammal in need of such treatment, comprising administering a disclosed compound, composition, or unit dosage form. In some embodiments, the medical condition is a disorder linked to dysregulation or inadequate functioning of neurotransmission. In some embodiments, the disorder is linked to dysregulation or inadequate functioning of neurotransmission is that of serotonergic neurotransmission. In some embodiments, the medical condition is a substance abuse disorder, a behavioral addiction, or a mental health disorder. In some embodiments, the mammal is a human. In some aspects are disclosed methods of reducing the symptoms of a substance use disorder, a behavioral addiction, or a mental health disorder in a human, comprising identifying a human in need of said reducing, and administering to the human a disclosed compound, composition, or unit dosage form. In some aspects are disclosed methods of treating a substance use disorder patient, a behavioral addiction patient, or a mental health patient in need thereof, comprising administering to the patient a therapeutically effective amount of a disclosed compound, composition, or unit dosage form.

In some embodiments, the substance abuse disorder, behavioral addiction, or mental health disorder is selected from the group consisting of: alcohol use disorder, nicotine dependency, opioid use disorder, stimulant use disorder, sedative, hypnotic, or anxiolytic use disorder, tobacco use disorder, gambling disorder, compulsive sexual behavior, sexual addiction, gaming addiction, shopping addiction, internet addiction, kleptomania, pyromania, compulsive buying, pornography addiction, binge eating disorder, internet gaming addiction, exercise addiction or overtraining syndrome, love addiction, work addiction or workaholism, and technological addictions, post-traumatic stress disorder (PTSD), an anxiety disorder, a depressive disorder, adjustment disorder, affective disorder, depression, atypical depression, postpartum depression, catatonic depression, a depressive disorder due to a medical condition, premenstrual dysphoric disorder, seasonal affective disorder, dysthymia, anxiety, phobia disorders, binge disorders, body dysmorphic disorder, alcohol or drug abuse or dependence disorders, substance-related disorders, substance-induced mood disorder, a mood disorder related to another health condition, disruptive behavior disorders, eating disorders, impulse control disorders, obsessive compulsive disorder (OCD), attention deficit hyperactivity disorder (ADHD), personality disorders, attachment disorders, and dissociative disorders. In some embodiments, the substance abuse disorder is any of alcohol use disorder, nicotine dependence, sedative, hypnotic, or anxiolytic use disorder, opioid abuse disorder, stimulant use disorder, and tobacco use disorder. In some embodiments, the behavioral addiction is any of gambling disorder, compulsive sexual behavior, sexual addiction, gaming addiction, shopping addiction, internet addiction, kleptomania, pyromania, compulsive buying, pornography addiction, binge eating disorder, internet gaming addiction, exercise addiction or overtraining syndrome, love addiction, work addiction or workaholism, and technology addiction. In some embodiments, the mental health disorder is any of an anxiety disorder, a depressive disorder, OCD, or PTSD. In some embodiments, the anxiety disorder is generalized anxiety disorder (GAD). In some embodiments, the depressive disorder is major depressive disorder (MDD) or treatment-resistant depression (TRD).

In some aspects are disclosed methods of improving psychological functioning in a human, comprising identifying a human in need of said improving, and administering to the human a disclosed compound, composition, or unit dosage form. In some embodiments, the improvement in psychological functioning is a reduction of neuroticism or psychological defensiveness, an increase in creativity or openness to experience, an increase in decision-making ability, an increase in feelings of wellness or satisfaction, or an increase in ability to fall or stay asleep.

In some embodiments, the compound is administered in combination with one or more psychotherapy sessions. In some embodiments, the patient has successfully completed a group therapy preparation course prior to the one or more psychotherapy sessions. In some embodiments, the patient has met all predefined inclusion criteria, and the patient has not met any predefined exclusion criteria, prior to the one or more psychotherapy sessions. In some embodiments, the one or more psychotherapy sessions is between 5 and 20 psychotherapy sessions, during which at least two of said psychotherapy sessions the patient is administered the compound. In some embodiments, the method of the invention results in one or more of: (a) a reduction of substance use, (b) a reduction of substance cravings, (c) a promotion of substance abstinence, (d) a prevention of relapse, or (e) an improvement of at least one symptom of the substance use disorder. In some embodiments, the method of the invention results in one or more of: (a) an increase in quality of life, (b) an increase in psychosocial functioning, (c) a decrease in use or frequency of a prescription medication, (d) a decrease in use or frequency of a recreational drug, (e) a decrease in obsessive compulsive thoughts, (f) a decrease in suicidality, (g) an increase in feelings of empathy, or (h) an increase in self-compassion. In some embodiments, the method of the invention results in an improvement of at least one symptom of a comorbid psychiatric disorder, such as antisocial personality disorder, borderline personality disorder, depression, anxiety, schizophrenia, attention deficit hyperactivity disorder, bipolar disorder, obsessive compulsive disorder, binge eating disorder, or PTSD. In some embodiments, the results are measured at least 1 month, at least 3 months, at least 6 months, at least 9 months, or at least 1 year from baseline, or from the first psychotherapy session.

In some embodiments, the patient is also administered a therapeutically effective amount of an additional active agent selected from the group consisting of: an opioid antagonist (e.g., nalmefene, naltrexone), a CB-1 antagonist (e.g., rimonabant), a CRH1 receptor antagonist (e.g., verucerfont, pexacerfont), a NK1R antagonist (e.g., tradipitant), an OTR agonist (e.g., oxytocin), a GABA agent (e.g., topiramate, baclofen, a benzodiazepine, such as alprazolam, diazepam or lorazepam), a voltage-gated sodium channel inhibitor (e.g., oxacarbazepine, valproic acid, zonisamide), a voltage-dependent calcium channel agonist (e.g., gabapentin, pregabalin), an 07 nicotinic acetylcholine receptor agonist (e.g., varenicline), a 5-HT3 antagonist (e.g., ondansetron), a 5-HT1A receptor partial agonist (e.g., aripiprazole), a 5-HT2A receptor antagonist (e.g., quetiapine, olanzapine, mirtazapine), a 5-HT reuptake inhibitor (e.g., trazodone), a SERT inhibitor (e.g., duloxetine), an α1 adrenoreceptor antagonist (e.g., doxazosin, prazosin), a glucocorticoid receptor antagonist (e.g., mifepristone), an α1 adrenoreceptor agonist (e.g., guanfacine), an AChE inhibitor (e.g., citicoline), a dopamine D2 receptor antagonist (e.g., tiapride), an α2 adrenoreceptor agonist (e.g., clonidine), an NMDA receptor antagonist (e.g., acamprosate), an aldehyde dehydrogenase inhibitor (e.g., disulfiram), and pharmaceutically acceptable salts thereof.

In some embodiments, the patient has a genetic variation associated with a mental health disorder, SUD, AUD, trauma or stressor related disorder, depression, or anxiety, and including a genetic variation in mGluR5 or FKBP5. In some embodiments, the patient has a genetic variation associated with the metabolism of ester bonds, including metabolism in the plasma, gut, liver, or other tissues, such as a polymorphism relating to an endogenous esterase. In some embodiments, the patient has AUD, nicotine dependency, opioid use disorder, sedative, hypnotic, or anxiolytic use disorder, stimulant use disorder, or tobacco use disorder. In some embodiments, the patient has gambling disorder, compulsive sexual behavior, sexual addiction, gaming addiction, shopping addiction, internet addiction, kleptomania, pyromania, compulsive buying, pornography addiction, binge eating disorder, internet gaming addiction, exercise addiction or overtraining syndrome, love addiction, work addiction or workaholism, or technology addiction. In some embodiments, the patient has a depressive disorder or an anxiety disorder.

The foregoing has outlined broadly some pertinent features of certain exemplary embodiments of the present disclosure so that the detailed description of the invention that follows may be better understood and so that the present contribution to the art can be more fully appreciated. Additional features of the invention will be described hereinafter which form the subject of the claims of the invention. It should be appreciated by those skilled in the art that the conception and the disclosed specific formulations and methods may be readily utilized as a basis for modifying or designing other formulations and methods for carrying out the same purposes of the disclosure. It should be also realized that such equivalent formulations and methods do not depart from the spirit and scope of the invention as set forth in the claims. Hence, this summary is made with the understanding that it will be considered as a brief and general synopsis of only some of the objects and embodiments herein, is provided solely for the benefit and convenience of the reader, and is not intended to limit in any manner the scope, or range of equivalents, to which the claims are lawfully entitled.

### BRIEF SUMMARY OF THE DRAWINGS

To further clarify various aspects of the invention, a more particular description is rendered by reference to certain exemplary embodiments illustrated in the figures. It will be appreciated that these figures depict only illustrated embodiments of the invention and should not be considered limiting of its scope. They are merely provided as exemplary illustrations of certain concepts of some embodiments of the invention. Certain aspects of the invention are therefore further described and explained with additional specificity and detail, but still by way of example only, with reference to the accompanying figures in which:

FIG. 1: Effects of 7 mg/kg of MDMA (n=11), MEAI (n=11), and exemplary compound CMP-405 (n=10) on serotonin (5-HT) levels (% from baseline) in an in vivo microdialysis experiment following intraperitoneal (IP) administration.

FIG. 2: Effects of 7 mg/kg of MDMA (n=12), MEAI (n=11), and exemplary compound CMP-405 (n=12) on dopamine (DA) levels (% from baseline) in an in vivo microdialysis experiment following IP administration.

FIG. 3: Effects of 7 mg/kg of MDMA (n=12), MEAI (n=10), and exemplary compound CMP-405 (n=12) on norepinephrine (NE) levels (% from baseline) in an in vivo microdialysis experiment following IP administration.

FIG. 4: A comparison of rat body temperature (° C. rectal) following administration of MDMA, MEAI, and exemplary compound CMP-405.

FIG. 5: Exemplary drug-assisted psychotherapy protocol.

### DETAILED DESCRIPTION

Among the various aspects of the present invention are novel therapeutic aminoindane compounds, pharmaceutical compositions thereof, and methods of their use. Methods of use may include methods of treatment, such as for substance use disorders, behavioral addictions, and CNS and other mental health disorders, as well as for the improvement of mental health and psychological functioning. In some embodiments, the compounds and compositions may be used in conjunction with psychotherapy, such as drug-assisted or psychedelic-assisted therapies. In some embodiments, the disclosed compounds and compositions, including when used in conjunction with psychotherapy, such as drug-assisted or psychedelic-assisted therapies, will provide improvements over currently-known compounds.

While various aspects and features of certain embodiments are summarized above, the following detailed description illustrates several exemplary embodiments in further detail to enable one having ordinary skill in the art to which the invention belongs (“one of skill”) to practice such embodiments, and to make and use the full scope of the invention claimed.

It will be understood that many modifications, substitutions, changes, and variations in the described examples, embodiments, applications, and details of the invention illustrated herein can be made by those skilled in the art without departing from the spirit of the invention, or the scope of the invention as described in the appended claims, and the general principles defined herein may be applied to a wide range of aspects. Thus, the invention is not intended to be limited to the aspects presented, but is to be accorded the widest scope consistent with the principles and novel features disclosed. The description below is designed to make such embodiments apparent to a person of ordinary skill, in that the embodiments shall be both readily cognizable and readily creatable without undue experimentation, solely using the teachings herein together with general knowledge of the art.

The terminology used herein is for describing embodiments and is not intended to be limiting. As used herein, the singular forms “a,” “an,” “and,” “the,” and “said” are intended to mean that there are one or more of the elements, and hence include plural referents, unless the content and context clearly dictate otherwise. Thus, for example, a reference to “an excipient” may include a single such excipient, or a combination of two or more excipients.

The terms “comprising,” “including,” “such as,” and “having” are intended to be inclusive and not exclusive (i.e., there may be other elements in addition to the recited elements). Thus, the term “including” as used herein means, and is used interchangeably with, the phrase “including but not limited to.” The term “or” is used herein to mean, and is used interchangeably with, the term “and/or,” unless context clearly indicates otherwise.

The recitation of ranges of values serves as a shorthand for referring individually to each separate value falling within the range. Unless otherwise indicated, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention. Examples are provided for illustrative purposes, not to limit the scope of the invention or its applications.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment.

In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Unless defined otherwise, all technical and scientific terms have the meaning as commonly understood by one of skill. Further definitions to assist the reader in understanding the embodiments are as follows; however, it will be appreciated that they are not intended to limit the scope of the invention, which shall be properly interpreted and understood by reference to the full specification (as well as any plain meaning known to one of skill) in view of the language used in the appended claims. The terminology used herein is for the purpose of describing particular embodiments only and is not intended to be limiting.

Generally, the nomenclature used and procedures performed herein are those known in fields relating to that of one or more aspects of the invention, such as those of biology, pharmacology, neuroscience, psychology, organic chemistry, synthetic chemistry, medicinal chemistry, natural products chemistry, medicine, statistics, and the like, and are those that will be well-known and commonly employed in such fields. Standard techniques and procedures will be those generally performed according to conventional methods in the art.

It will be appreciated that the headings in this document are utilized only to expedite its review by a reader. They should not be construed as limiting the invention in any manner.

### A. MDMA-ASSISTED PSYCHOTHERAPY AND A NEED FOR IMPROVED ENTACTOGENS

Drug-assisted psychotherapies with “classic” psychedelics, such as psilocybin and LSD, utilize the subjective mystical effects of the psychedelic experience and have found the depth of this experience is strongly associated with maintained abstinence from substance use (Bogenschutz, J. Psychopharmacol., 2015; 29(3):289-99; Johnson et al., J Psychopharmacol., 2014; 28(11):983-92; Krupitsky & Grinenko, J. Psychoactive Drugs, 1997; 29(2):165-183).

However, not all patients may be able to tolerate a psychedelic experience. “Entactogens” or “empathogens” such as MDMA, by contrast, are generally more easily tolerated than classic psychedelics, with minimal perceptually disturbing effects and alterations in consciousness, and a reduced likelihood of a challenging or so-called “bad” trip. But whether this in fact offers an alternative opportunity for enhanced psychotherapy has not been well demonstrated. Indeed, the depth of the mystical experience is strongly associated with maintained abstinence from substance use (Roseman et al., Front Pharmacol. 2017; 8: 974). Thus, it would be logical to presume that MDMA-assisted therapy for substance use disorders, behavioral addictions, and mental health disorders would have less success in treating patients than LSD- or psilocybin-assisted therapy, as would drug-assisted therapy using other non-psychedelic or more “MDMA-like” analogs, such as other entactogens generally, or compounds of the aminoindane class.

MDMA produces elevated mood and feelings of closeness to others, slight alterations in perception that facilitate imagination and memory (Harris et al., Psychopharmacol. (Berl), 2002; 162(4): 396-405), greater compassion, feelings of sociability, closeness and increased empathy for others and themselves (Hysek et al., Soc. Cogn. Affect Neurosci., 2013; 9(11): 1645-52). Such entactogenic effects may facilitate powerful introspective and reflective states that can yield therapeutic benefit (Nichols, Front. Psychiatry, 2022; 13). The effects of MDMA at reducing activity in the amygdala (Gamma et al., Neuropsychopharmacology, 2000; 23: 388-95) results in a reduced fear response, and greater activity in the reward pathways in the Ventral Tegmental Area (VTA), in comparison to placebo. Together, these effects can beneficially influence the outcome of psychotherapeutic treatments for psychological disorders. However, an MDMA-assisted psychotherapy session may span 6-8 hours (Mithoefer, MAPS, A Manual for MDMA-Assisted Psychotherapy in the Treatment of PTSD, 2015). Thus, adoption and use of MDMA- or other entactogen-assisted psychotherapies may be limited unless the therapeutic characteristics of the entactogen can be optimized to provide, e.g., a more rapid onset of activity and/or a shorter duration of action.

Additionally, MDMA and its metabolites can pose safety risks associated with toxicity. MDMA-induced elevation of serotonin levels has been implicated in neurotoxicity, and co-administration with additional serotonergic drugs may result in serotonin syndrome (Parrott, Pharmacol Biochem Behav., 2002; 71(4):837-44; Parrott, Neurosci Biobehav Rev., 2013; 37(8):1466-8; Makunts et al., Front Psychiatry. 2021; 12: 824288). Side effects range in severity and include but are not limited to elevation in heart rate and blood pressure, hyperthermia, and hepatic toxicity (Kalant, CMAJ: Can. Med. Assoc. J., 2001; 165(7): 917-928). Major metabolites of MDMA include 3,4-methylenedioxyamphetamine (MDA), 4-hydroxy-3-methoxy-methamphetamine (HMMA) and 4-hydroxy-3-methoxy-amphetamine (HMA). MDMA is mainly metabolized in the liver, where several different enzymes play a role in its metabolism, including CYP2D6, but such enzymes may be saturated at relatively low levels of the drug (Tucker et al., Biochemical Pharmacology, 1994; 47(7):1151-1156; de la Torre, Frontiers In Genetics, 2012; 3:235). Non-linearity in MDMA pharmacokinetics has been identified, and a small dose increase has been shown to disproportionately escalate plasma concentration (de la Torre et al., British J. Clin. Pharmacol., 2000; 49(2):104-109). MDMA metabolites, such as MDA, retain pharmacological activity and extend the duration of action, which can increase the likelihood of toxicity. The catechol moieties of MDMA and MDA are postulated to be inherently reactive. Downstream effects of such reactivity include generation of reactive oxygen species, reactive nitrogen species, and other toxic byproducts (Carvalho et al., Curr. Pharm. Biotechnol., 2010; 11(5):476-95).

Some subjects who are administered clinical MDMA may experience an increase in anxiety associated with derealization-type experiences (Mithoefer et al., J. Psychopharmacol., 2010; 25(4): 439-452). The effects of MDMA on the noradrenergic system, such as stimulating release of noradrenaline (norepinephrine), may be responsible for serious adverse effects on the cardiovascular system and amphetamine-like effects, which may potentiate abuse (Kalant, CMAJ. 200; 165(7): 917-928). Jaw tightness and bruxism are also common, as well as reduced appetite, poor concentration and impaired balance (Mithoefer et al., J. Psychopharmacol., 2010; 25(4): 439-452; Oehen et al., J. Psychopharmacol., 2013; 27(1): 40-52). When the recreational drug “ecstasy” is taken outside of the clinical setting, more serious adverse effects have been observed, including hyperthermia, liver disease, and hyponatremia (Rogers et al., Health Technol. Assess, 2009; 13(6): iii-iv, ix-xii, 1-315). An improved alternative to MDMA may avoid these issues, and a compound having an improved side effect profile may lead to better therapeutic outcomes.

### B. IMPROVED STRATEGIES ARE NEEDED TO TREAT SUBSTANCE USE DISORDERS, BEHAVIORAL ADDICTIONS, AND MENTAL HEALTH DISORDERS

In some aspects are provided improved treatment options for substance use disorders (SUDs) such as alcohol use disorder (AUD), behavioral addictions, and CNS and other mental health disorders. Herein, the terms mental health disorders and psychiatric disorders may be used interchangeably. SUDs, behavioral addictions, and mental health disorders pose major obstacles to human health and productivity. Available treatments may lack efficacy and result in toxicity, necessitating improved treatment options, such as those disclosed herein.

a. Substance Use Disorders

Improved strategies are necessary to treat SUDs. In one example, the impact of AUD, both harmful use and dependent use, is widespread, encompassing alcohol related illness and injuries as well as significant social impacts to family, friends, and the community. Patients who have become dependent upon alcohol frequently have a past history of psychological trauma, and there is an association between alcohol dependence and PTSD. People with AUD commonly present with high levels of depression, social anxiety, and social exclusion and may have subsequently developed a dependence upon alcohol as a form of self-medication (Castillo-Carniglia et al., The Lancet Psychiatry, 2019; 6(12):1068-1080).

The outcomes of current treatments are generally considered relatively poor. Licensed pharmacological options for treating AUDs include acamprosate, disulfiram, naltrexone and nalmefene. The FDA has approved only disulfiram, naltrexone and acamprosate for the treatment of AUD, and the EMA has approved, in addition, nalmefene. Acamprosate, a glutamate antagonist, reduces NMDA activity associated with alcohol withdrawal and reduces relapse rates (ROsner et al., Cochrane Database of Systematic Reviews, 2010; (9):CD004332). Disulfiram is used as an agent to deter relapse by preventing the elimination of acetaldehyde. This results in an unpleasant physical reaction if alcohol is used in combination with this medication (Krampe et al., Alcoholism: Clinical and Experimental Research 2006; 30(1):86-95). Naltrexone, a competitive opioid antagonist, decreases cravings from alcohol (Soyka & Rösner, Curr Drug Abuse Rev., 2008; 1(3): 280-91). Nalmefene is an opioid antagonist, like naltrexone but with a longer half life and lower risk of toxicity. This can be given to patients while still drinking in order to reduce cravings (Paille & Martini, Substance Abuse and Rehabilitation 2014; 5(5): 87-94). Benzodiazepines are also prescribed commonly as a time limited course as part of alcohol detoxification program (Lingford-Hughes et al., J. Psychopharmacol. 2012; 26(7):899-952).

The efficacy of each of these medications is only modest. Thus there is still a high medical need to find more effective treatments for AUD. Of all of the pharmacological agents tested, none have shown marked promise, including opioid antagonists (e.g., nalmefene), CB-1 antagonists (e.g., rimonabant), CRH1 antagonists (e.g., verucerfont, pexacerfont), NK1R antagonists (e.g., tradipitant), OTR agonists (e.g., oxytocin), GABA agents (e.g., topiramate, baclofen, benzodiazepines), voltage gated sodium channel inhibitors (e.g., oxacarbazepine, valproic acid, zonisamide), voltage dependent calcium channel agonists (e.g., gabapentin, pregabalin), α7 nicotinic acetylcholine receptor agonists (e.g., varenicline), 5-HT3 antagonists (e.g., ondansetron), 5 HT 1A receptor partial agonists e.g., aripiprazole), 5-HT 2A receptor antagonists e.g., quetiapine, olanzapine, mirtazapine), 5-HT reuptake inhibitors (e.g., trazodone), SERT inhibitors (e.g., duloxetine), al adrenorecept or antagonists (e.g., doxazosin, prazosin), glucocorticoid receptor antagonists (e.g., mifepristone), al adrenoreceptor agonists (e.g., guanfacine), AChE inhibitors e.g., citicoline), dopamine D2 receptor antagonists (e.g., tiapride), and α2 adrenoreceptor agonists (e.g., clonidine).

In terms of psychosocial interventions, the best approaches are those that foster good relationships and are delivered by the most experienced, highly trained therapists. However, the efficacy of current available treatments is far from satisfactory, with high rates of relapse. A systematic review of 361 controlled studies of treatments for alcohol dependence in 2002 identified 46 possible interventions (Miller & Wilbourne, Addiction, 2002; 97:265-277). Interventions were ranked according to rates of abstinence achieved. The lowest ranked approaches were designed to educate, confront, shock or foster insight regarding the nature of alcoholism. From this systematic review psychotherapy produced the best outcomes.

In recent years mindfulness techniques, originally derived from Vipassana meditation, which encourage awareness and acceptance of thoughts, feelings and bodily sensations have been increasingly explored as a potential approach for treating AUD (Marcus & Zgierska, Mindfulness-Based Therapies for SUDs: Part 1, 2009; 30(4), 263; Shapiro et al., J. Clin. Psych., 2006; 62(3):373-386; Hsu et al., Recent Developments in Alcoholism, Vol. 18, 2008; 229-250). While mindfulness shows promise as an adjunctive treatment to help individuals endure craving, evidence of its effectiveness has been limited, with many individuals needing further cognitive and behavioral skills to cope with high risk situations for relapse.

b. Behavioral Addictions

Certain behavioral addictions may respond positively to some of the same pharmacological and psychosocial treatments as SUDs. The 12-step self-help approaches, motivational enhancement, and cognitive behavioral therapies commonly used to treat SUDs have been used in the treatment of gambling disorder, kleptomania, and compulsive buying (Grant et al., Can. J. Psych., 2013; 58(5), 252-259). Psychosocial interventions for both behavioral addictions and SUDs often focus on a relapse prevention model that encourages abstinence by identifying patterns of abuse, avoiding or coping with high-risk situations, and making lifestyle changes that reinforce healthier behaviors.

To date, FDA has not approved any drug to treat any behavioral addiction (Ginley et al., APA Handbook of Psychopharmacol., 2019; 631-646). A number of medications have been proposed as treatments for behavioral addictions (Grant et al., The Canadian Journal of Psychiatry, 2013; 58(5), 252-259). Naltrexone, a mu opioid receptor antagonist, has been in controlled clinical trials to treat problem gambling and kleptomania (Kim et al., Biol. Psychiatry, 2001; 49:914-921). Opiate antagonists are thought to decrease dopamine neurotransmission in the nucleus accumbens and linked motivational neurocircuitry, thus dampening gambling-related excitement and cravings. Medications that alter glutamatergic activity have been used (Olive et al., Pharmacol. Biochem. Behav., 2012; 100(4): 801-810).

It is difficult to find agreement regarding the outcomes of treatments for behavioral addictions due to the diversity of such addictions and their idiosyncrasies, the severity of the addiction, multiple confounding psychosocial factors and the range of treatment approaches used. There is literature exploring the efficacy of treatments for gambling disorder. A 2017 systematic review of psychological interventions for problem gambling reviewed cognitive therapies, cognitive behavioral therapy (CBT), motivational interventions, self-directed cognitive behavioral interventions, and brief feedback interventions. The reviewers analyzed 21 moderate to large studies and noted that the interventions offered no consistent or persistent changes in gambling in relation to any specific treatment (Petry et al., Psych. Addictive Behav., 2017; 31(8), 951).

Pharmacological treatments for gambling disorder are not commonly utilized as a first step in treatment. There are currently no FDA-approved medications for the treatment of gambling disorder, though there are currently a number of pharmacological approaches based on treatments for disorders that share similar characteristics (Lupi et al., BioMed Res. Int'l, 2014). Research has explored the efficacy of pharmacological treatments for gambling disorder. In one double-blind, placebo-controlled study of naltrexone, 52 patients with gambling disorder and co-occurring AUDs were also provided with 7 sessions of CBT (Toneatto et al., Am. J. Addict., 2009; 18(3): 219-225). Both groups responded and there were no significant differences between naltrexone and placebo. The researchers found that the use of naltrexone to treat concurrent alcohol use and gambling problems was not supported. Clinical trials examining the use of SSRI antidepressants (e.g., sertraline, paroxetine, and fluvoxamine) have been mixed. Limitations of existing literature indicate that it is not possible to conclude which antidepressants should be recommended, and which individuals are more likely to respond to these agents (Grant et al., Can. J. Psych., 2013; 58(5), 252-259). Clinical studies of mood stabilizing medications such as lithium, topiramate, valproic acid, and carbamazepine have also delivered mixed results, with some studies failing to prove superiority to placebo in reducing gambling disorder symptoms.

Mindfulness meditation has also recently become increasingly explored as a potential approach for treating behavioral addictions (Shonin et al., J. Addiction Res. & Ther., 2014; 5(1)). Empirical investigation exploring the role of mindfulness in the treatment of behavioral addictions has exclusively focused on addictions to gambling or work. In a 2014 controlled pilot study, mindfulness intervention was integrated with CBT for gamblers (Toneatto et al., Int'l J. Mental Health & Addiction, 2014; 12(2), 197-205). This was shown to reduce the severity of gambling, gambling urges, and psychiatric symptoms at end-of-treatment. However, although mindfulness shows promise as an adjunctive treatment to help individuals endure urges, evidence of its effectiveness has been limited, with many individuals needing further cognitive and behavioral skills to cope with high-risk situations for relapse.

c. Mental Health Disorders

Currently, treatment options for mental health disorders consist generally of psychotherapy, pharmacotherapy, and direct brain intervention either provided alone or in combination (for example, psychotherapy together with one or more daily medications). Such treatment options may only offer limited efficacy and some are associated with serious drawbacks. Psychotherapy takes various forms including CBT, exposure therapy, eye movement desensitization and reprocessing (EMDR) and numerous forms of talk therapy. Psychotherapy frequently fails to provide complete or even partial symptom reduction. For example, CBT is generally believed to be fully effective for only a third of patients with mood disorders, with another third receiving partial benefit, and a last third receiving no benefit at all, even when paired together with a pharmacological agent.

First line pharmacological treatment for most mental health disorders, including depression, panic and anxiety disorders, obsessive compulsive disorder, phobias, bulimia, adjustment disorder, and PTSD, typically involves use of selective serotonin reuptake inhibitors (SSRIs) and other depressants such as serotonin-norepinephrine reuptake inhibitors (SNRIs). SSRIs block the reabsorption, i.e., reuptake of serotonin into neurons, thereby increasing levels of serotonin in the brain. Behavioral and neuropsychological processes modulated by serotonin include mood, perception, reward, anger, aggression, appetite, memory, sexuality, attention, and others. Indeed, it is difficult to find any human behavior not regulated by serotonin (see, e.g., Berger et al., Ann. Rev. Med., 2009; 60:355-66).

The most commonly prescribed antidepressants, however, all have serious drawbacks For example, many patients taking SSRIs develop insomnia, skin rashes, headaches, joint and muscle pain, stomach upset, nausea, or diarrhea. Patients also report insomnia, hypersomnia, fatigue, and weight gain (Papakostas, J. Clin. Psych., 2007; 68, 11-17). Moreover, for many patients, SSRIs result in sexual dysfunction and can diminish sexual interest, desire, performance, satisfaction, or all four. In men, SSRIs can delay or inhibit ejaculation, and in women, delay or prevent orgasm. A more serious potential problem is reduced blood clotting capacity because of a decreased concentration of the neurotransmitter serotonin in platelets.

Besides antidepressants, another broad class of agents for treatment of mental health disorders is monoamine releasers, which induce the release of monoamine neurotransmitters, e.g., dopamine, serotonin, or epinephrine, from neurons. Monoamine releasers rapidly modulate the brain systems that are more gradually affected by SSRIs. However, their stimulant and euphoric effects can result in high abuse liability. For example, although psychostimulants in this class were widely employed in the mid 20th century to treat affective disorders, OCD, and schizophrenia, use of such agents, e.g., amphetamines, is now primarily limited to treating ADHD, and even this use is increasingly restricted.

Another class of medications commonly used to treat mental health disorders, and anxiety disorders in particular are benzodiazepines, which include drugs such as diazepam (Valium) and alprazolam (Xanax) (Bandelow et al., Dialogues Clin. Neurosci., 2017; 19(2), 93-107). Associated adverse effects including fatigue, dizziness, increased reaction time, impaired driving skills, and more (id). Long term treatment with benzodiazepines is associated with dependence (id). The high burden of disability of generalized anxiety disorder coupled with the long latency period and the high number of adverse effects associated with current drug treatments further underscores the need to pursue alternative therapies to reduce symptoms of anxiety and increase the quality of life for affected patients.

As disclosed by Applicant, certain psychoactive agents of the aminoindane class, administered alone or as adjunctive pharmacotherapy together with psychotherapy or other healing modalities, provide superior methods of treating subjects with numerous significant improvements over prior art methods. As described herein, the disclosed compounds, compositions, and methods have significant advantages over standard psychotherapy and other existing treatments for these disorders offering patients novel approaches to many devastating, chronic, and relapsing conditions, and additionally providing improvements in mental health and functioning to both diseased and healthy subjects.

### C. AMINOINDANES

Compounds in the aminoindane class of molecules generally share a common 2-aminoindane (2-AI) core structure, although positional isomers (e.g., 1-aminoindane) exist.

2-AI is a cyclic, rigid analog of amphetamine, whose rigidity is due to the bridge between the α-carbon and aromatic ring. Its similarity to amphetamine led to its original development in the early 20th century as a vasoactive, bronchodilatory, analgesic compound. Analogs were shown to have less toxicity than amphetamine and more effective bronchodilation than ephedrine. Aminoindane molecules were later developed to treat Parkinson's disease. They are capable of inhibiting mitochondrial monoamine oxidase subtypes within minimal side effects, and two analogs were eventually patented as Parkinson's treatments (Pinterova et al., Front. Psychiatry, 2017; 8). Certain 2-AIs also have been disclosed as selective dopamine D3 ligands (U.S. Pat. No. 5,708,018).

Aminoindanes including 5,6-methylenedioxy-2-aminoindane (MDAI), 5-iodo-2-aminoindane (5-IAI), 5-methoxy-aminoindane (5-MeO-AI or MEAI), 5-methoxy-6-methyl-2-aminoindane (MMAI), 5,6-methylenedioxy-2-(methylamino)indane (MDMAI), and N-methyl-2-aminoindane (NM-2-AI) have been studied (Nichols et al., J. Med. Chem., 1990; 33, 703-710; Pinterova, op. cit., 2017; Halberstadt et al., Psychopharmacol., 2019; 236(3), 989-999; Oeri, J. Psychopharmacol., 2020). MEAI (i.e., 5-methoxy-2-aminoindane or 5-MeO-AI) has been described for regulating binge behavior, including excessive alcohol intake (see, e.g., Shimshoni et al., Toxicol. Appl. Pharmacol. 2017; 319, 59-68; PCT Pub. No. WO2016/092546A1). MEAI was evaluated herein alongside MDMA to compare the effects of these compounds on monoamine release and transporter inhibition in vitro and monoamine levels in an in vivo microdialysis experiment, among others. MEAI produced comparable effects to MDMA in the microdialysis experiment, as described further in Example 18, preferentially stimulating release of serotonin to nearly 2500% above baseline.

While certain aminoindanes are thus known, Applicant discloses novel compounds of the class including those to which Applicant's knowledge have not been previously synthesized or studied, nor used for the purposes or in the methods of treatment taught below.

### D. COMPOUNDS

The compounds of the invention, referred to also as “therapeutic compounds,” include certain compounds of the aminoindane class, including ester and lactone derivatives.

In embodiments, the therapeutic compounds have a short duration of action relative to known compounds. In embodiments, the short duration of action is based on a soft drug design approach. In embodiments, compounds may be based on a soft drug design approach, but provide surprising or unexpected results when tested.

In some preferred embodiments are ester aminoindane derivatives that are rapidly hydrolyzed by esterases in the blood and liver. In some embodiments, the half life of a therapeutic compound of the invention is less than 4 hours, less than 3 hours, less than 2 hours, less than 1 hour, and in certain preferred embodiments, about 30 minutes.

The compounds of the invention include those having a structure of Formula (I):

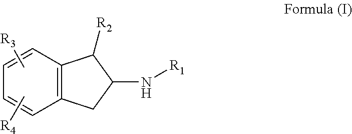

wherein any one of R₁, R₂, or R₃ is:

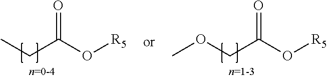

with the others of R₁, R₂, and R₃ both being hydrogen (H);
   and wherein R₄ is either H or —OR₅;
wherein each R₅ is independently any of H, alkyl, aryl, arylalkyl (as the terms are defined), —(CH₂)ₘZ(CH₂)ₘCH₃, or —(CH₂)ₘR^{cyc}, and each m is independently between 0-4;
   wherein each Z is independently, and in either orientation, —O—, —C═C—, —NH—, —NHNH—, —ONH—, —CONH, —CH(NH₂)—, —S—, —S(O)—, —SO₂—, —CHF—, and —CF₂—;
   wherein each R^{cyc} is independently an aryl or heteroaryl, said aryl or heteroaryl selected from the class consisting of phenyl, naphthyl, naphthalenyl, indolyl, thiophenyl, pyridyl, pyridinyl, pyrimidinyl, furyl, furanyl, tetrahydrofuranyl, pyrrolyl, oxazolyl, thiazolyl, pyrazolyl, isoxazolyl, imidazolyl, isothiazolyl, oxadiazolyl, triazolyl, thiadiazolyl, pyndazinyl, pyrazinyl, triazinyl, indolyzinyl, isoindolyl, benzofuranyl, dihydro benzofuranyl, benzoyl, benzoxazolyl, methylenedioxyphenyl, isobenzofuranyl, benzothiophenyl, benzoxazolyl, indazolyl, benzthiazolyl, benzimidazolyl, quinolinyl, isoquinolinyl, purinyl, quinolizinyl, cinnolinyl, phthalyl, quinoxalinyl, napthyridinyl, pteridinyl, and quinazolinyl;
      and wherein one or more hydrogen atoms of said aryl or heteroaryl are optionally and independently replaced with any of: (a) halogen, (b) C₁₋₇ alkyl, (c) hydroxy, (d) methoxy, (e) isopropoxy, (f) ethoxy, (g) hydroxymethyl, (h) hydroxypropyl, (i) tert-butyl, (j) n-butyl, (k) sec-butyl, (l) isobutyl, (m) acetamide, (n) carbamoyl, (o) —CN, (p) —COOH, (q) —NO₂, (r) —S(O)₂OH, (s) —S(O)CH₃, (t) —S(O)₂CH₃, (u) —OR₆, or (v) —SR₆;
         wherein R₆ is hydrogen or C₁₋₇ alkyl;
or alternatively, where R₃ is

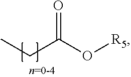

R₅ is joined to the benzene ring at an R₄ position adjacent to the R₃ to form a lactone ring (and both of R₁ and R₂ are hydrogen).

It will be understood that where a number above is defined as a range, such as where n is between 1-3 or between 0-4, and where m is between 0-4, the range is inclusive.

“Alkyl” includes radicals having any degree or level of saturation, i.e., groups having exclusively single C—C bonds, groups having one or more double C═C bonds, groups having one or more triple C═C bonds and groups having any mixtures of single, double, and triple carbon-carbon bonds. Where a specific level of saturation is intended, the expressions “alkanyl,” “alkenyl,” and “alkynyl” also can be used. Preferably, an alkyl group comprises from 1 to 26 carbon atoms, and more preferably, from 1 to 10 carbon atoms.

“Alkanyl” refers to a saturated branched, straight-chain, or cyclic alkyl radical derived by the removal of a hydrogen from a single carbon atom of a parent alkane. Alkanyl groups include methanyl; ethanyl; propanyls such as propan-1-yl, propan- [symbol] -yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like. “Alkenyl” refers to an unsaturated branched, straight-chain, or cyclic alkyl radical having at least one C═C double bond derived by the removal of a hydrogen from a single carbon atom of a parent alkene. The group may be in either the cis or trans conformation about the double bond(s). Alkenyl groups include ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2yl, buta-1,3-dien-1-yl, buta-1,3-dien-2-yl, cyclobut-1-en-1-yl, cyclobut-1-en-3-yl, cyclobuta-1,3-dien-1-yl, etc.; and the like. “Alkynyl” refers to an unsaturated branched, straight-chain, or cyclic alkyl radical having at least one C═C triple bond derived by the removal of a hydrogen from a single carbon atom of a parent alkyne. Alkynyl groups include ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Aryl” refers to a monovalent aromatic hydrocarbon radical derived by the removal of one hydrogen atom from a single carbon atom of a parent aromatic ring system. Aryl groups include groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. Preferably, an aryl group comprises from 6 to 20 carbon atoms, more preferably, between 6 to 12 carbon atoms. “Arylalkyl” refers to an acyclic alkyl radical in which one of the hydrogen atoms bonded to a carbon atom, typically a terminal or sp3 carbon atom, is replaced with an aryl group. Arylalkyl groups include benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. Preferably, an arylalkyl group is (C₆-C₃₀) arylalkyl, e.g., the alkanyl, alkenyl or alkynyl moiety of the arylalkyl group is (C₁-C₁₀) and the aryl moiety is (C₆-C₂₀), more preferably, an arylalkyl group is (C₆-C₂₀) arylalkyl, e.g., the alkanyl, alkenyl or alkynyl moiety of the arylalkyl group is (C₁-C₈) and the aryl moiety is (C₆-C₁₂).

“Cycloalkyl” refers to a saturated or unsaturated cyclic alkyl radical including groups derived from cyclopropane, cyclobutane, cyclopentane, cyclohexane, and the like. “Heteroaryl” refers to a monovalent heteroaromatic radical derived by the removal of one hydrogen atom from a single atom of a parent heteroaromatic ring system. Heteroaryl groups include groups derived from acridine, arsindole, benzothiophene, benzofuran, carbazole, carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. Preferably, the heteroaryl group is between 5-20 membered heteroaryl, with 5-10 membered particularly preferred.

In certain embodiments, the therapeutic compounds are those having the structure of Formula (I) above, wherein each R₅ is independently H, —CH₃, or —CH₂CH₃.

In certain embodiments, wherein n may be from 0-4 inclusive, the lactone ring may be a four-, five-, six-, seven-, eight-membered lactone ring.

In some embodiments, the therapeutic compounds are those having a structure of Formula (Ia), and in exemplary such embodiments may be any one of the below:

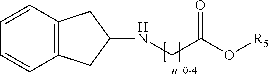

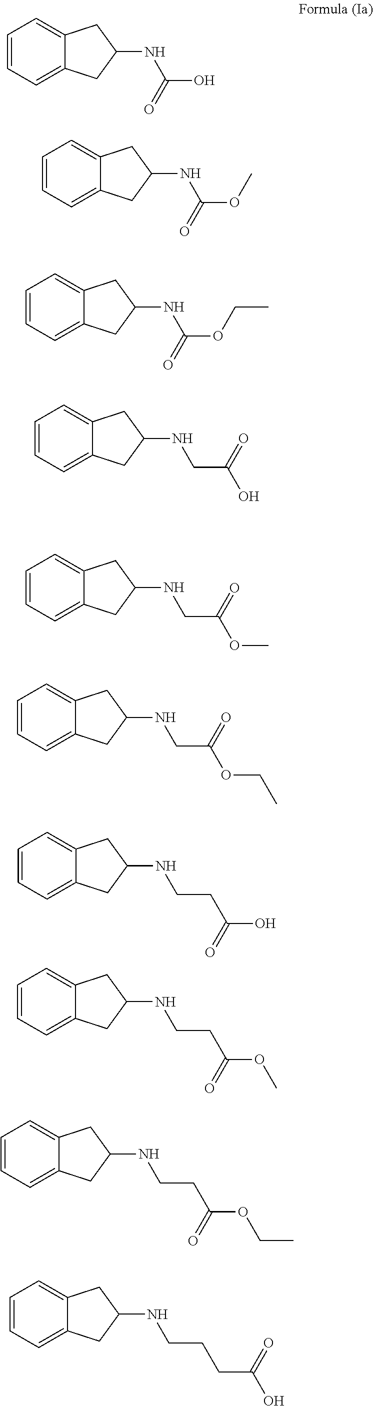

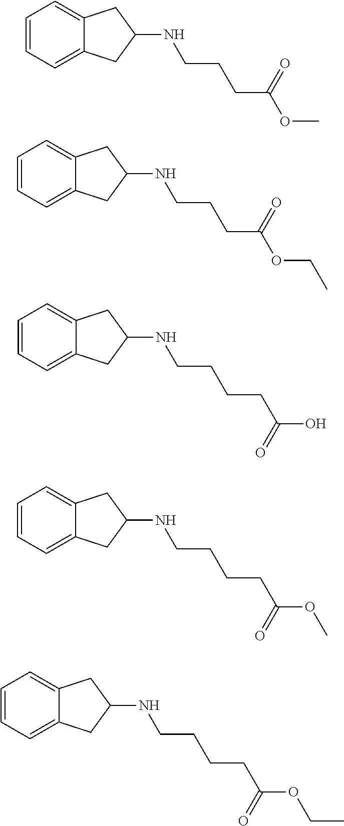 [SMILES: COC(=O)CCCNC1Cc2ccccc2C1 ; CCOC(=O)CCCNC1Cc2ccccc2C1 ; O=C(O)CCCCNC1Cc2ccccc2C1 ; COC(=O)CCCCNC1Cc2ccccc2C1 ; CCOC(=O)CCCCNC1Cc2ccccc2C1]

In some embodiments, the therapeutic compounds are those having a structure of Formula (Ib), and in exemplary such embodiments may be any one of the below:

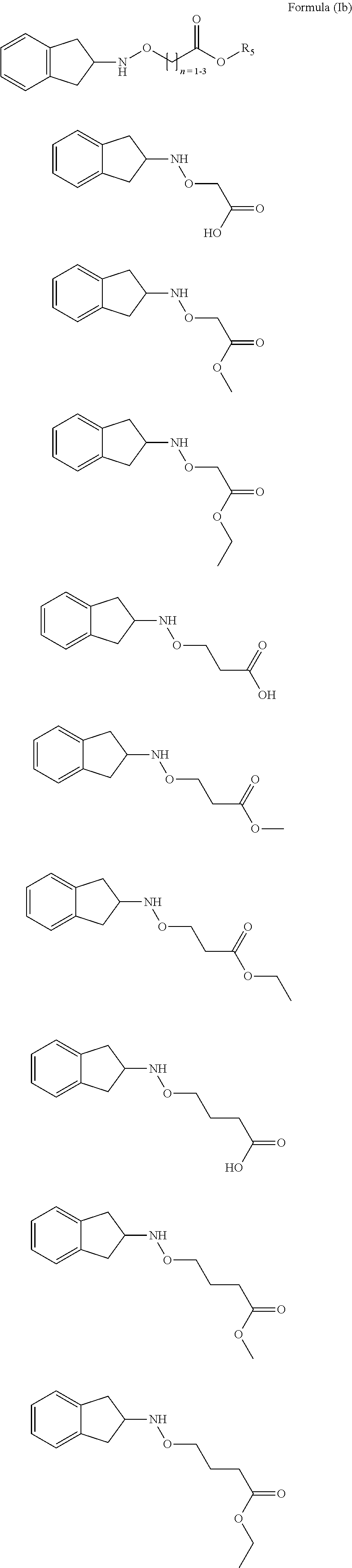

In some embodiments, the therapeutic compounds are those having a structure of Formula (Ic), and in exemplary such embodiments may be any one of the below:

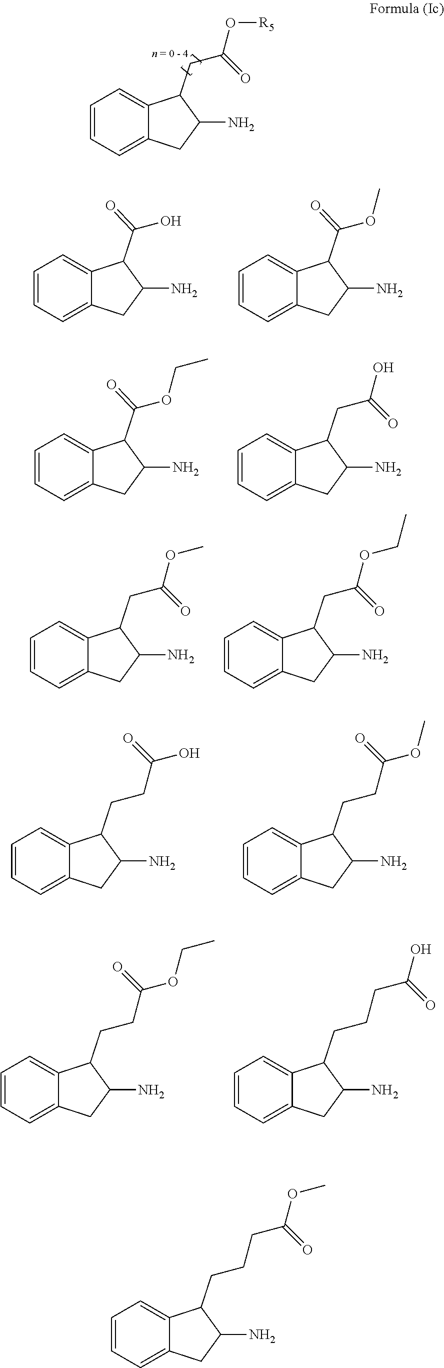

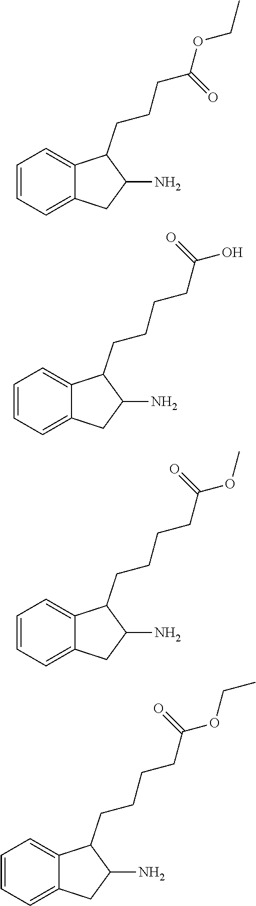 [SMILES: CCOC(=O)CCCC1c2ccccc2CC1N ; NC1Cc2ccccc2C1CCCCC(=O)O ; COC(=O)CCCCC1c2ccccc2CC1N ; CCOC(=O)CCCCC1c2ccccc2CC1N]

In some embodiments, the therapeutic compounds are those having a structure of Formula (Id), and in exemplary such embodiments may be any one of the below:

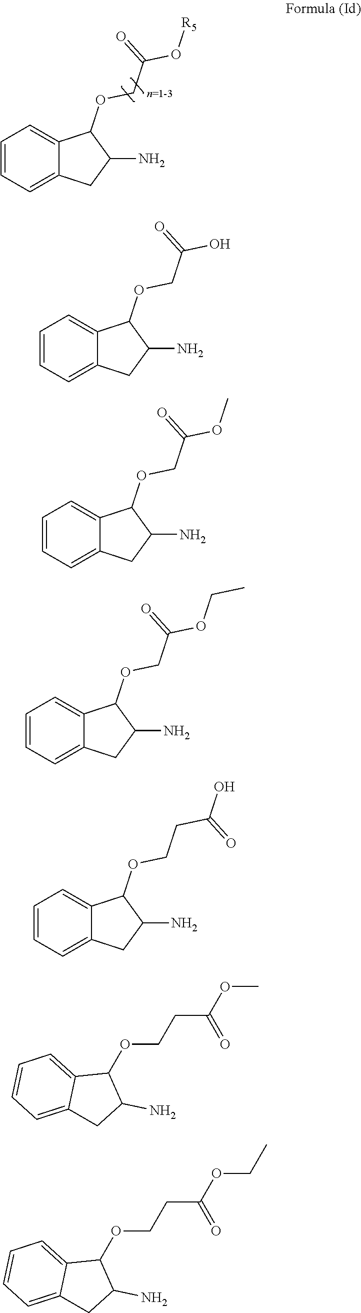

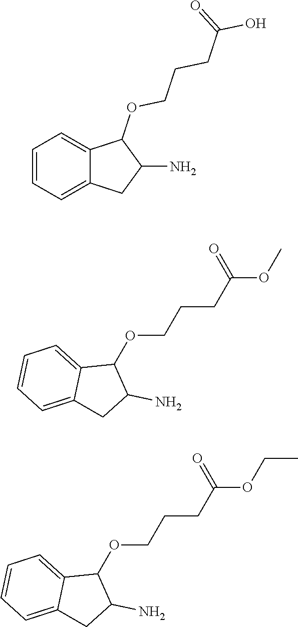 [SMILES: NC1Cc2ccccc2C1OCCCC(=O)O ; COC(=O)CCCOC1c2ccccc2CC1N ; CCOC(=O)CCCOC1c2ccccc2CC1N]

In some embodiments, the therapeutic compounds are those having a structure of Formula (Ie), and in exemplary such embodiments may be any one of the below:

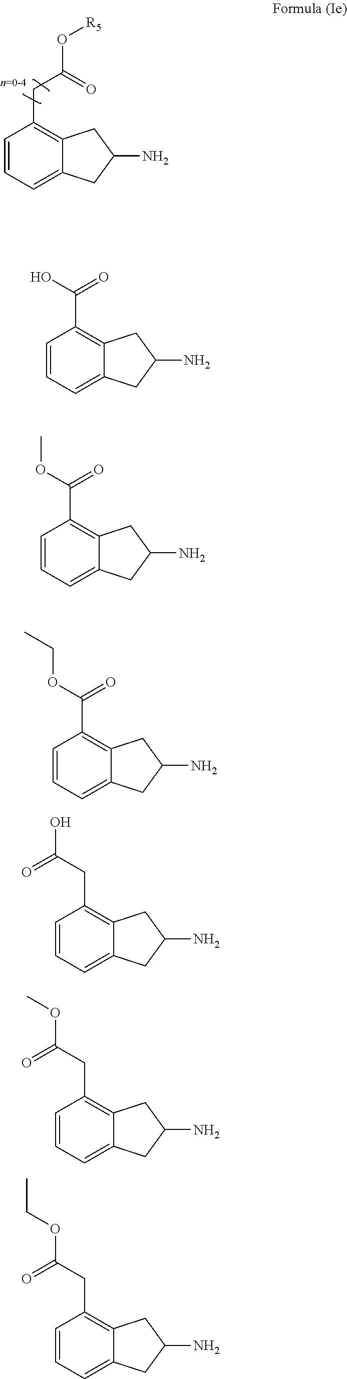

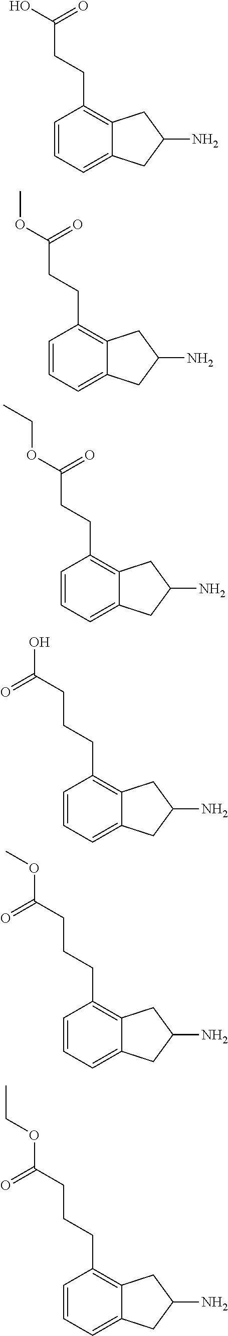

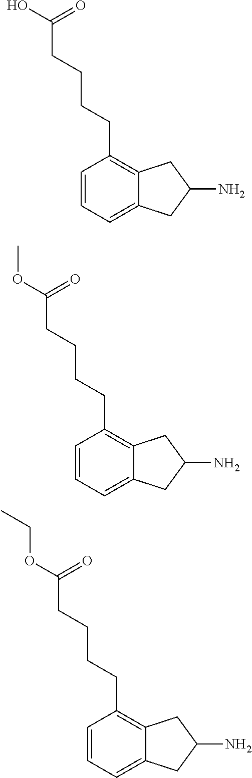 [SMILES: NC1Cc2cccc(CCCCC(=O)O)c2C1 ; COC(=O)CCCCc1cccc2c1CC(N)C2 ; CCOC(=O)CCCCc1cccc2c1CC(N)C2]

In some embodiments, the therapeutic compounds are those having a structure of Formula (If), and in exemplary such embodiments may be any one of the below:

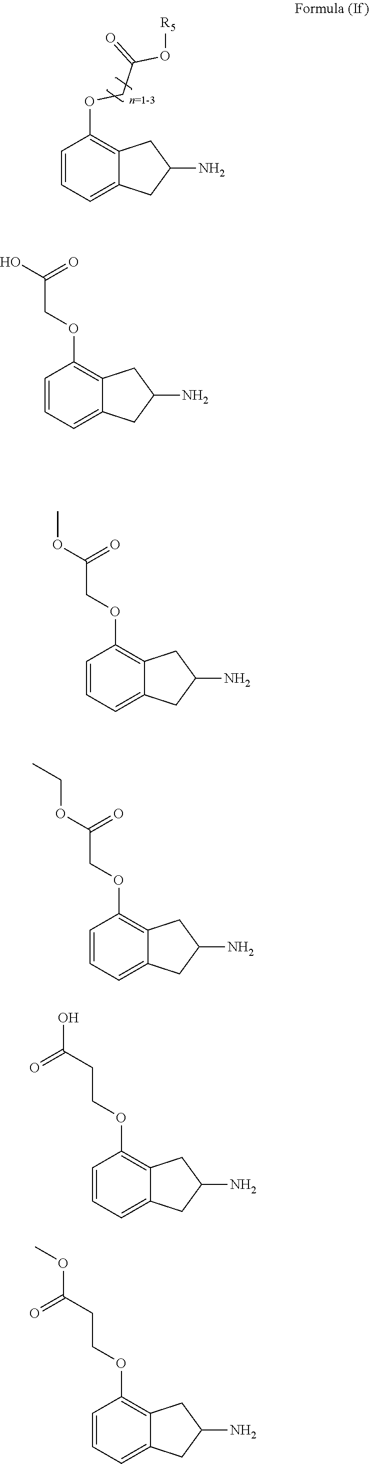

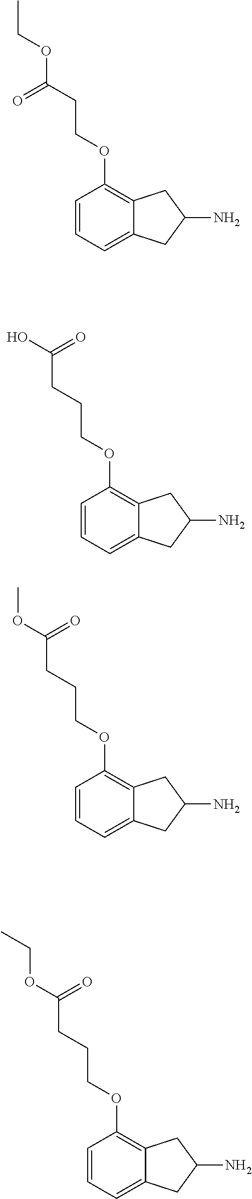 [SMILES: CCOC(=O)CCOc1cccc2c1CC(N)C2 ; NC1Cc2cccc(OCCCC(=O)O)c2C1 ; COC(=O)CCCOc1cccc2c1CC(N)C2 ; CCOC(=O)CCCOc1cccc2c1CC(N)C2]

In some embodiments, the therapeutic compounds are those having a structure of Formula (Ig), and in exemplary such embodiments may be any one of the below:

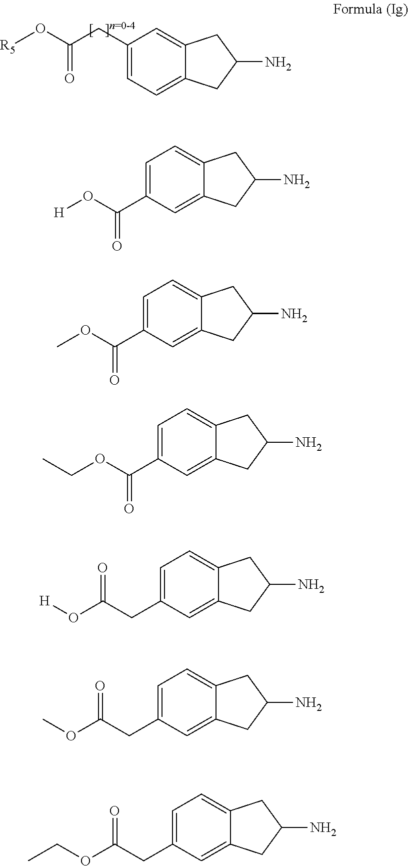

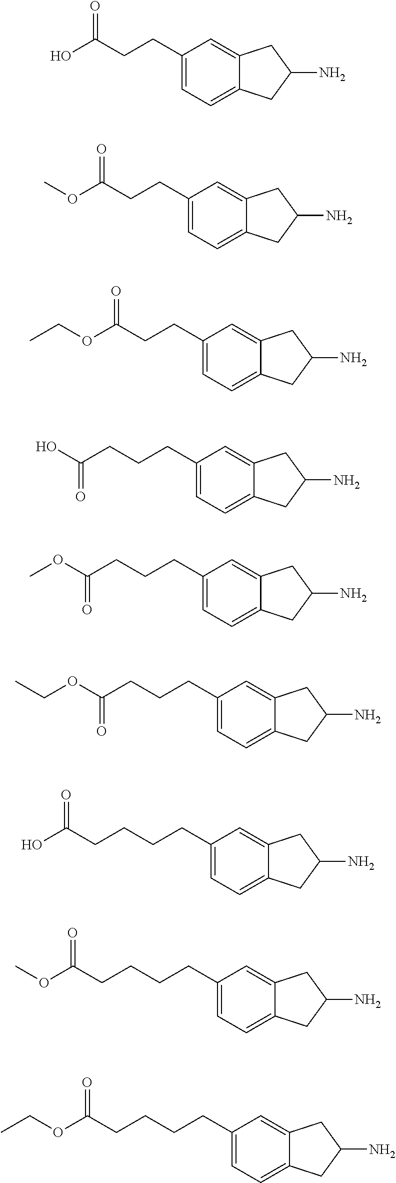

In some embodiments, the therapeutic compounds are those having a structure of Formula (Ih), and in exemplary such embodiments may be any one of the below:

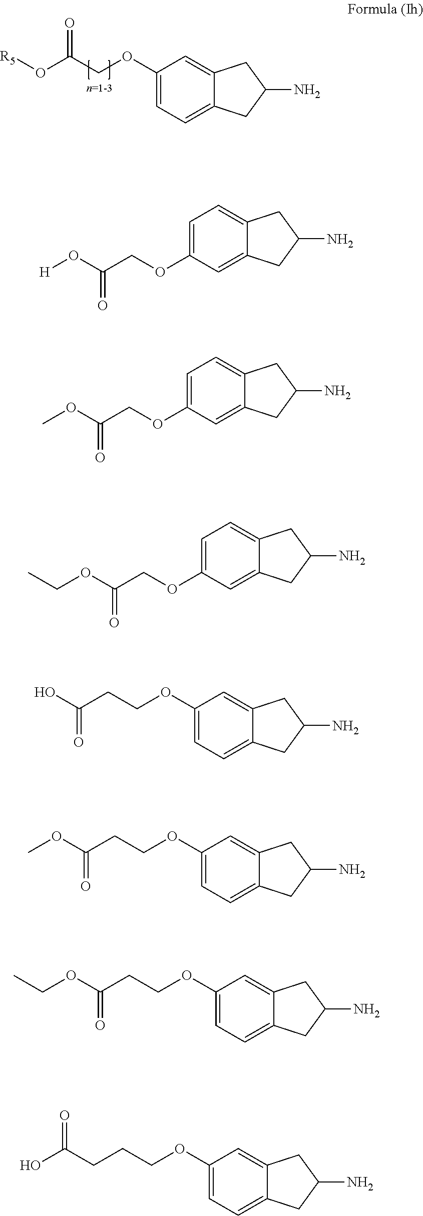

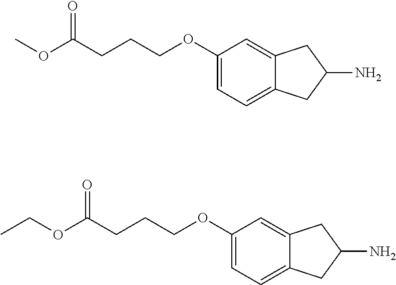 [SMILES: COC(=O)CCCOc1ccc2c(c1)CC(N)C2 ; CCOC(=O)CCCOc1ccc2c(c1)CC(N)C2]

In some embodiments, the therapeutic compounds are those having a structure of Formula (Ii), and in exemplary such embodiments may be any one of the below:

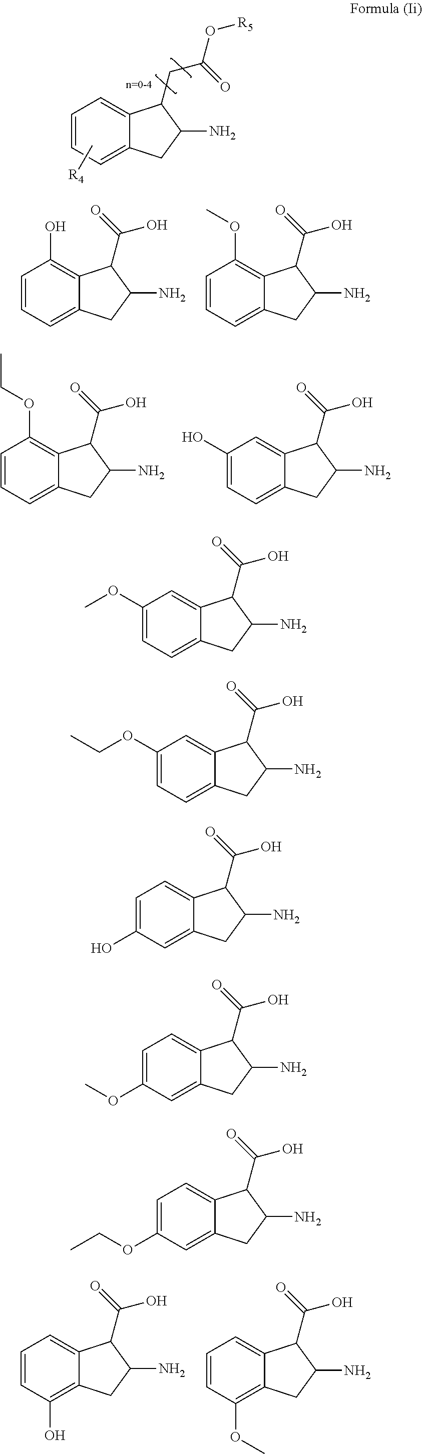

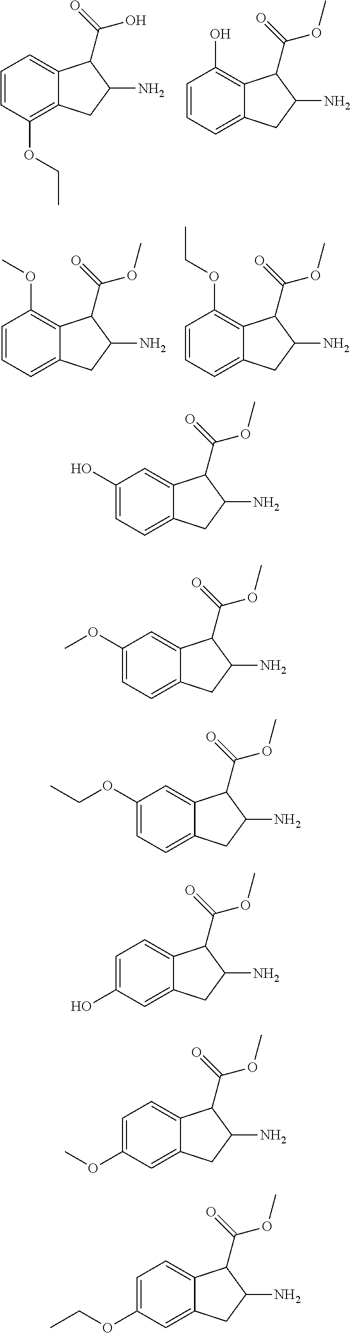

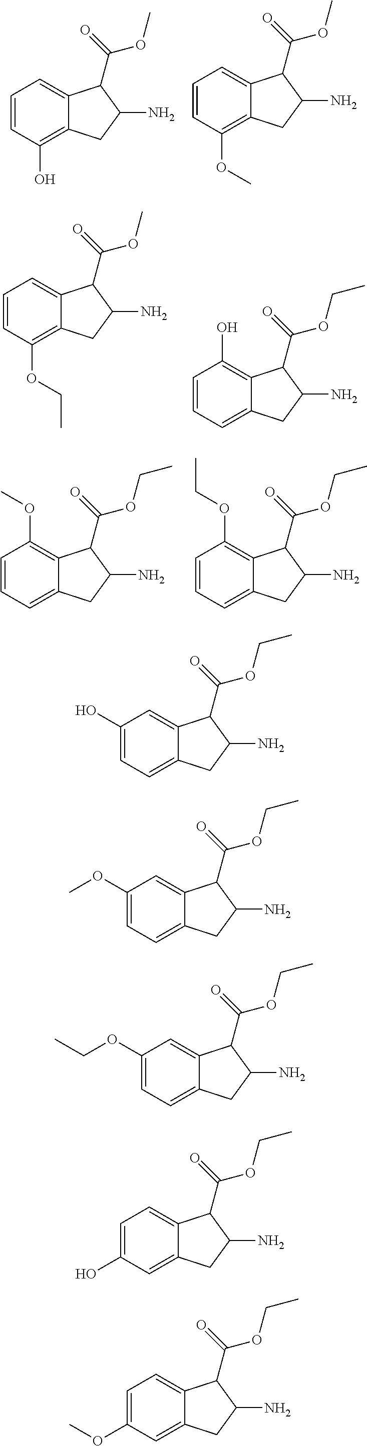

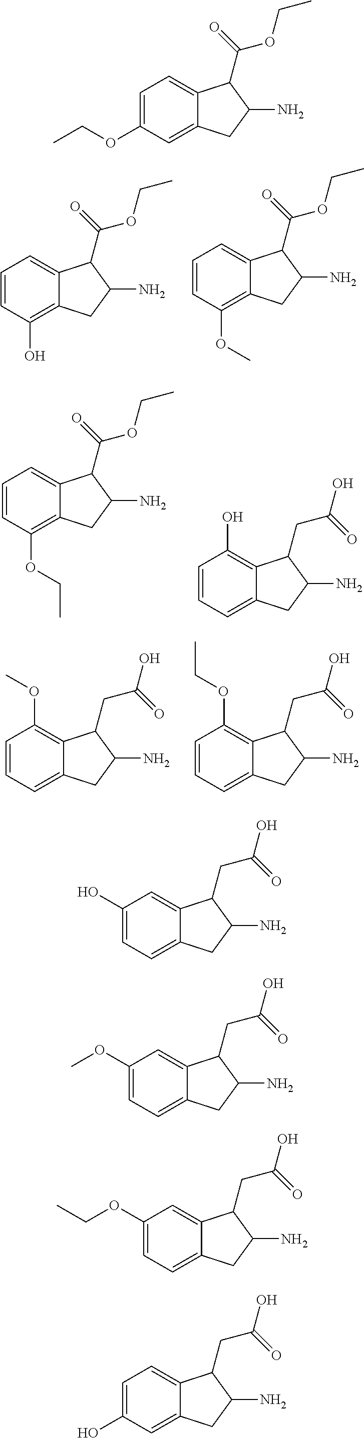

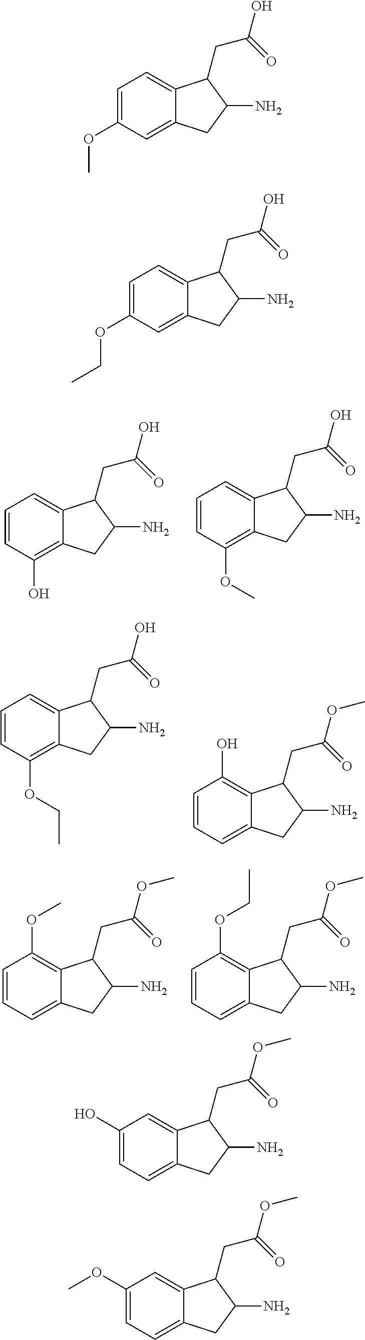

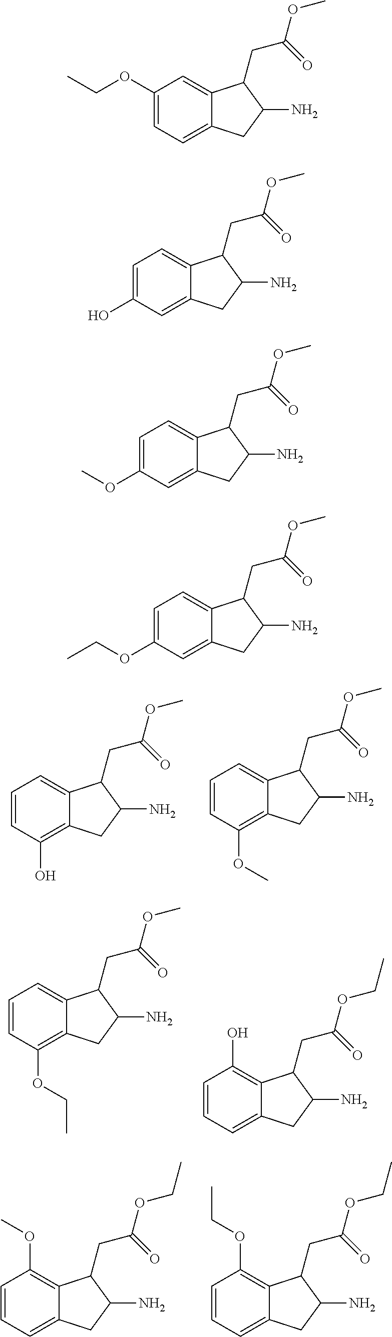

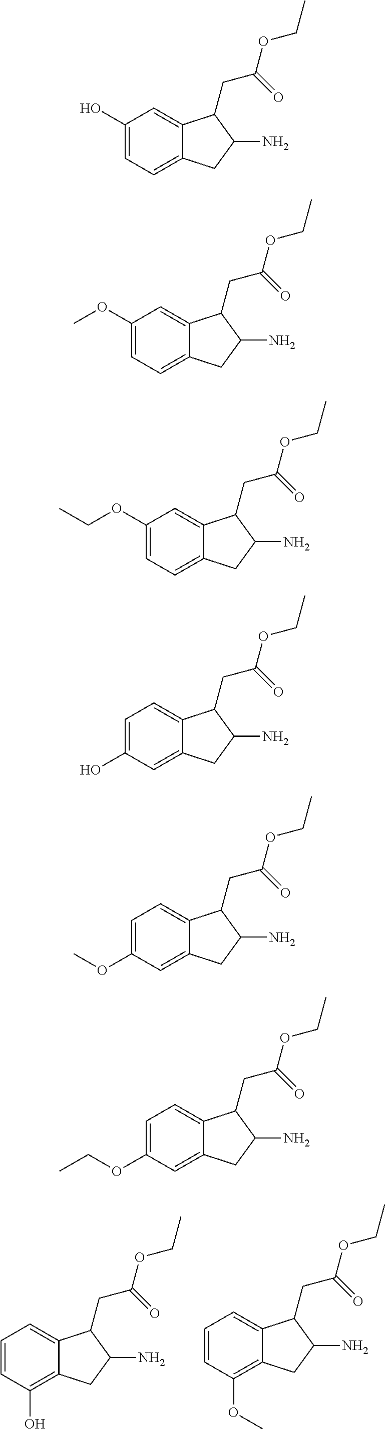

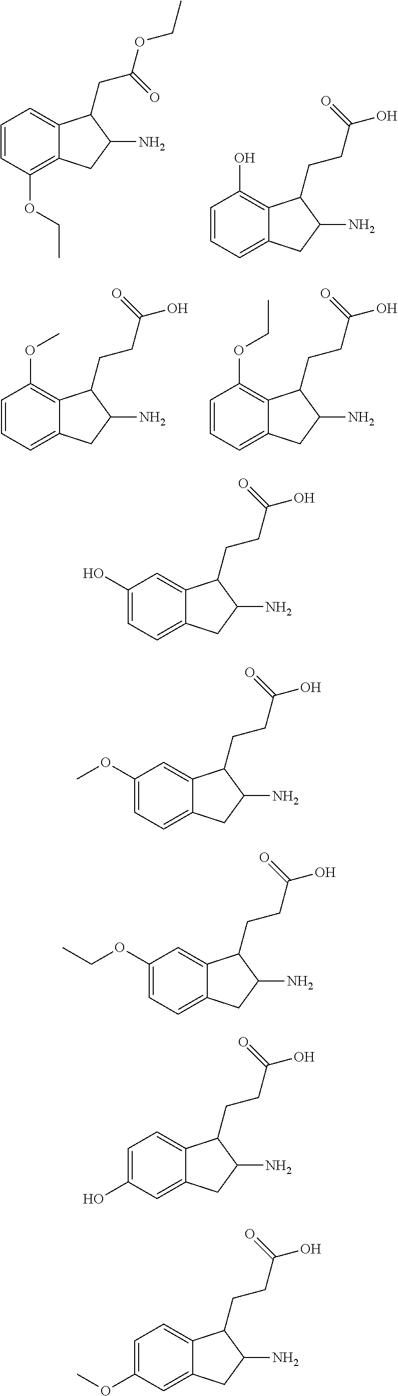

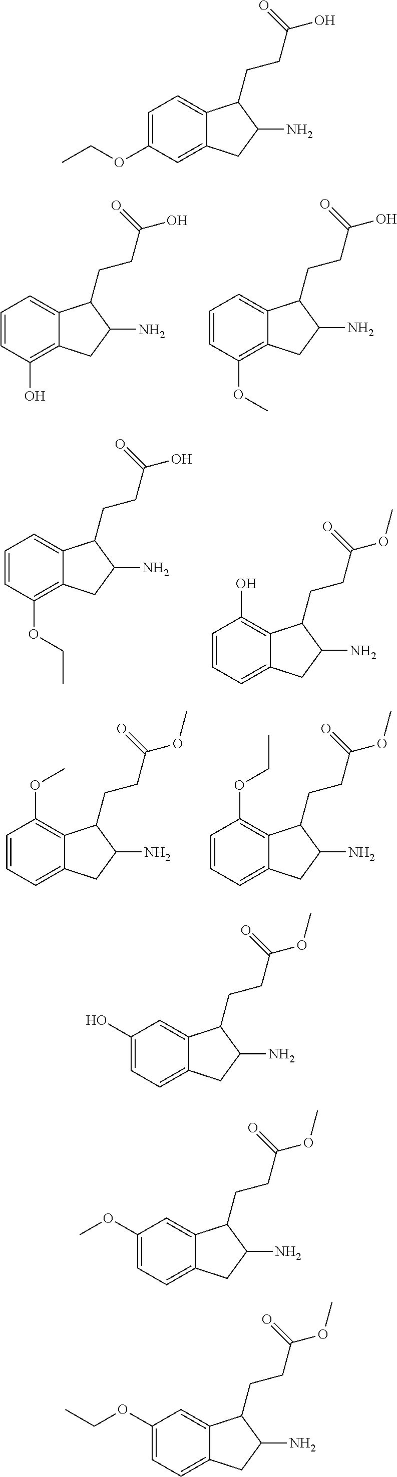

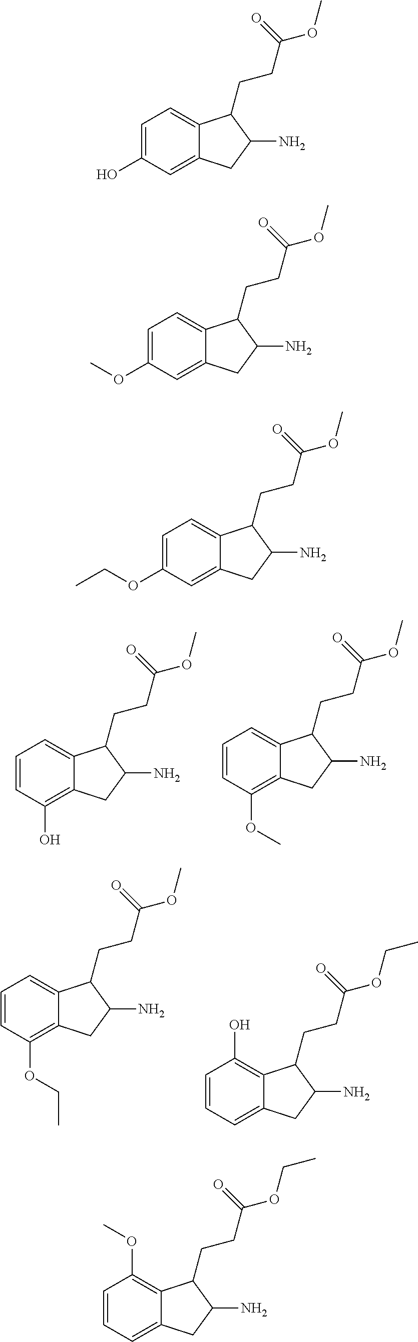

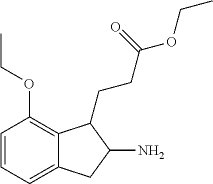 [SMILES: CCOC(=O)CCC1c2c(cccc2OCC)CC1N]

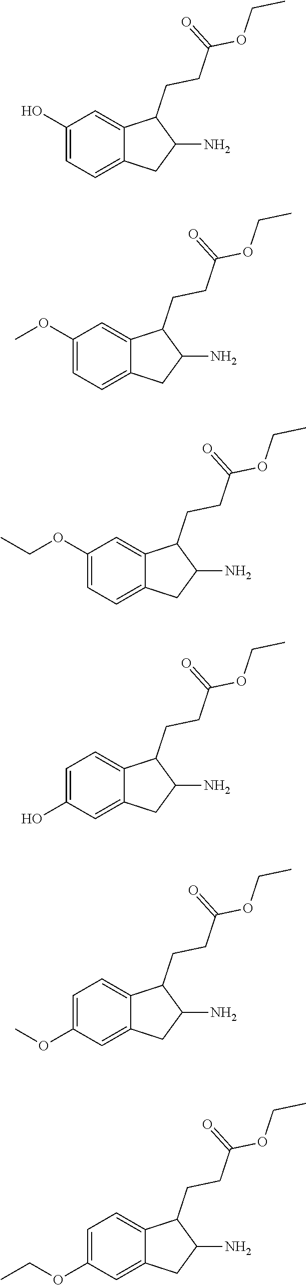

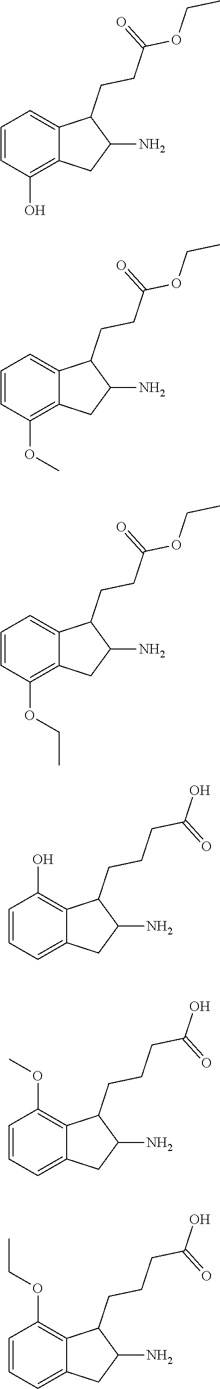 [SMILES: CCOC(=O)CCC1c2cccc(O)c2CC1N ; CCOC(=O)CCC1c2cccc(OC)c2CC1N ; CCOC(=O)CCC1c2cccc(OCC)c2CC1N ; NC1Cc2cccc(O)c2C1CCCC(=O)O ; COc1cccc2c1C(CCCC(=O)O)C(N)C2 ; CCOc1cccc2c1C(CCCC(=O)O)C(N)C2]

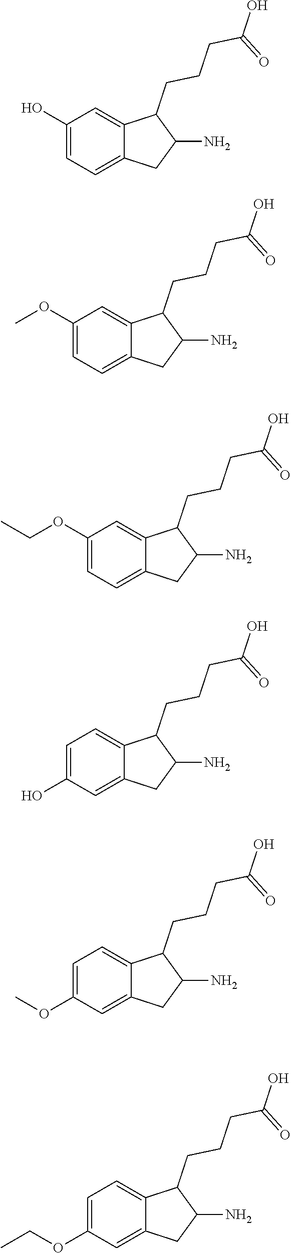 [SMILES: NC1Cc2ccc(O)cc2C1CCCC(=O)O ; COc1ccc2c(c1)C(CCCC(=O)O)C(N)C2 ; CCOc1ccc2c(c1)C(CCCC(=O)O)C(N)C2 ; NC1Cc2cc(O)ccc2C1CCCC(=O)O ; COc1ccc2c(c1)CC(N)C2CCCC(=O)O ; CCOc1ccc2c(c1)CC(N)C2CCCC(=O)O]

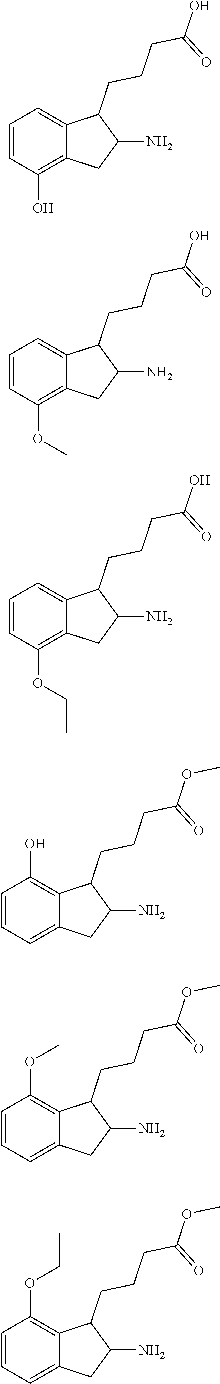 [SMILES: NC1Cc2c(O)cccc2C1CCCC(=O)O ; COc1cccc2c1CC(N)C2CCCC(=O)O ; CCOc1cccc2c1CC(N)C2CCCC(=O)O ; COC(=O)CCCC1c2c(O)cccc2CC1N ; COC(=O)CCCC1c2c(cccc2OC)CC1N ; CCOc1cccc2c1C(CCCC(=O)OC)C(N)C2]

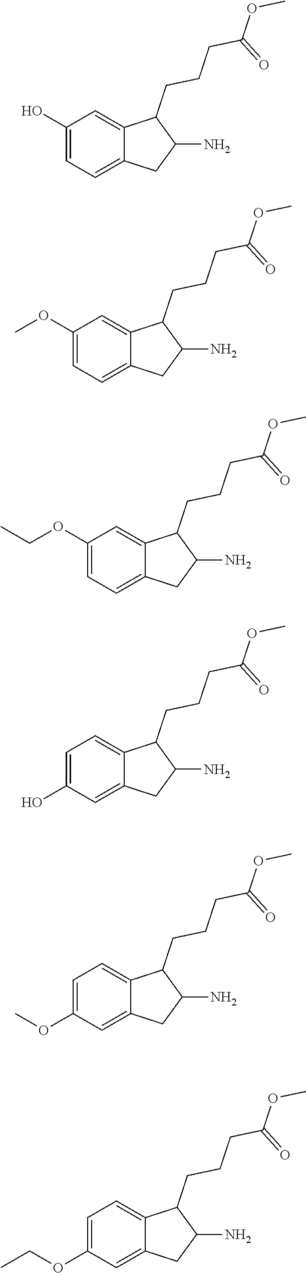

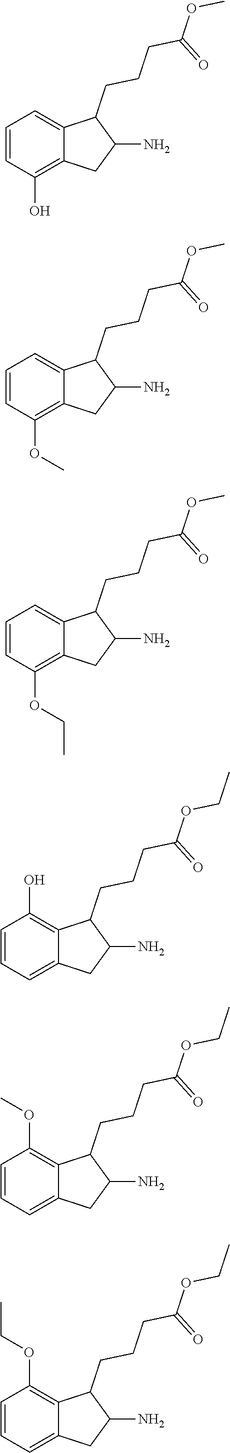 [SMILES: COC(=O)CCCC1c2cccc(O)c2CC1N ; COC(=O)CCCC1c2cccc(OC)c2CC1N ; CCOc1cccc2c1CC(N)C2CCCC(=O)OC ; CCOC(=O)CCCC1c2c(O)cccc2CC1N ; CCOC(=O)CCCC1c2c(cccc2OC)CC1N ; CCOC(=O)CCCC1c2c(cccc2OCC)CC1N]

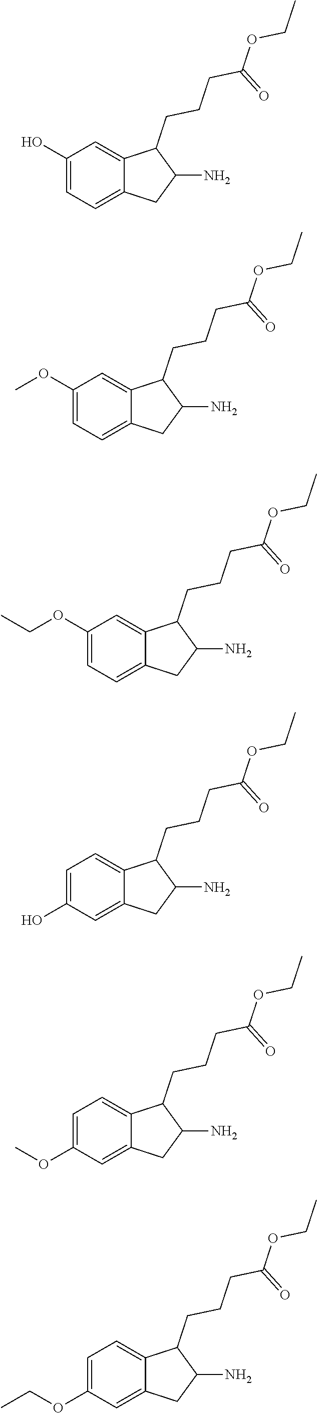

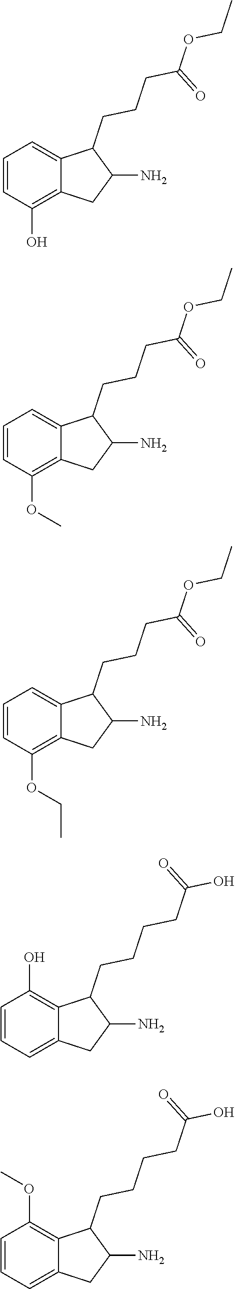 [SMILES: CCOC(=O)CCCC1c2cccc(O)c2CC1N ; CCOC(=O)CCCC1c2cccc(OC)c2CC1N ; CCOC(=O)CCCC1c2cccc(OCC)c2CC1N ; NC1Cc2cccc(O)c2C1CCCCC(=O)O ; COc1cccc2c1C(CCCCC(=O)O)C(N)C2]

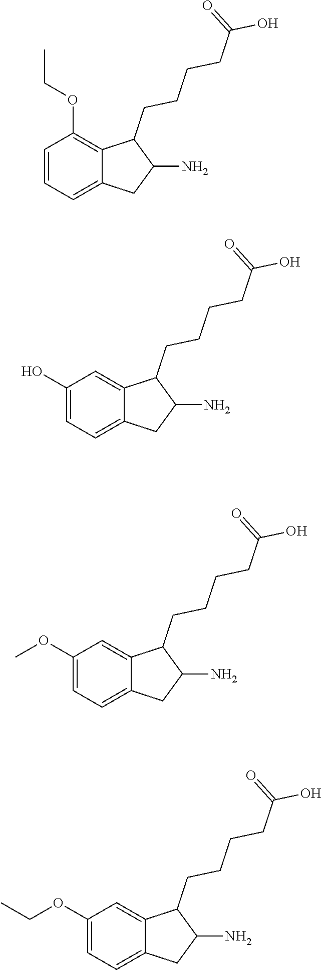 [SMILES: CCOc1cccc2c1C(CCCCC(=O)O)C(N)C2 ; NC1Cc2ccc(O)cc2C1CCCCC(=O)O ; COc1ccc2c(c1)C(CCCCC(=O)O)C(N)C2 ; CCOc1ccc2c(c1)C(CCCCC(=O)O)C(N)C2]

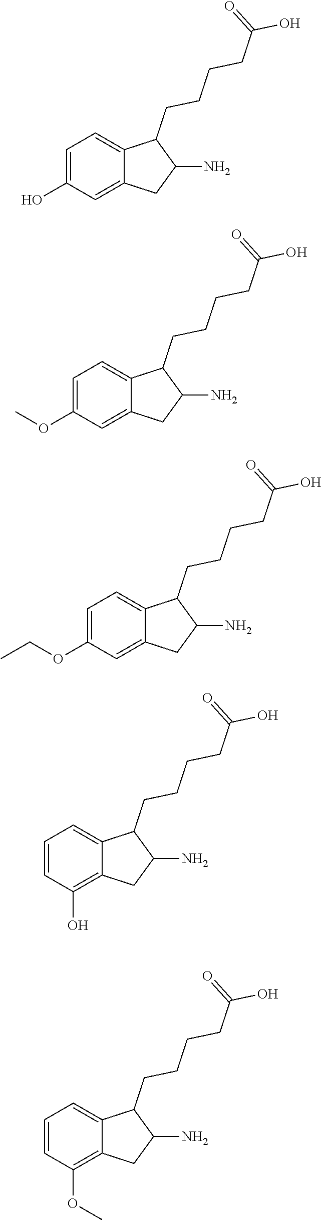 [SMILES: NC1Cc2cc(O)ccc2C1CCCCC(=O)O ; COc1ccc2c(c1)CC(N)C2CCCCC(=O)O ; CCOc1ccc2c(c1)CC(N)C2CCCCC(=O)O ; NC1Cc2c(O)cccc2C1CCCCC(=O)O ; COc1cccc2c1CC(N)C2CCCCC(=O)O]

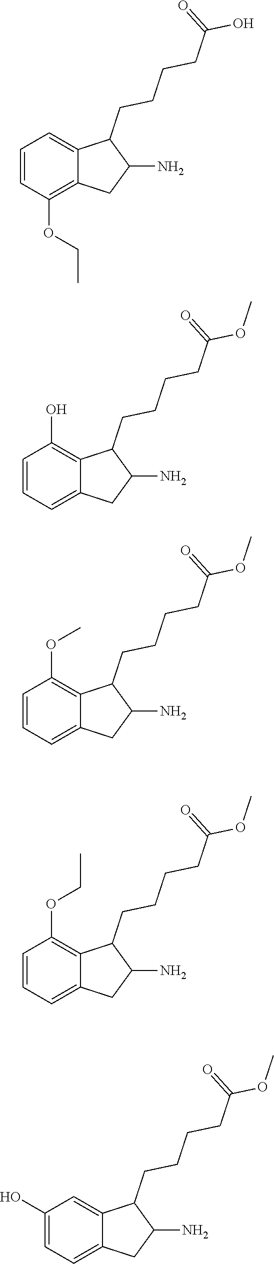 [SMILES: CCOc1cccc2c1CC(N)C2CCCCC(=O)O ; COC(=O)CCCCC1c2c(O)cccc2CC1N ; COC(=O)CCCCC1c2c(cccc2OC)CC1N ; CCOc1cccc2c1C(CCCCC(=O)OC)C(N)C2 ; COC(=O)CCCCC1c2cc(O)ccc2CC1N]

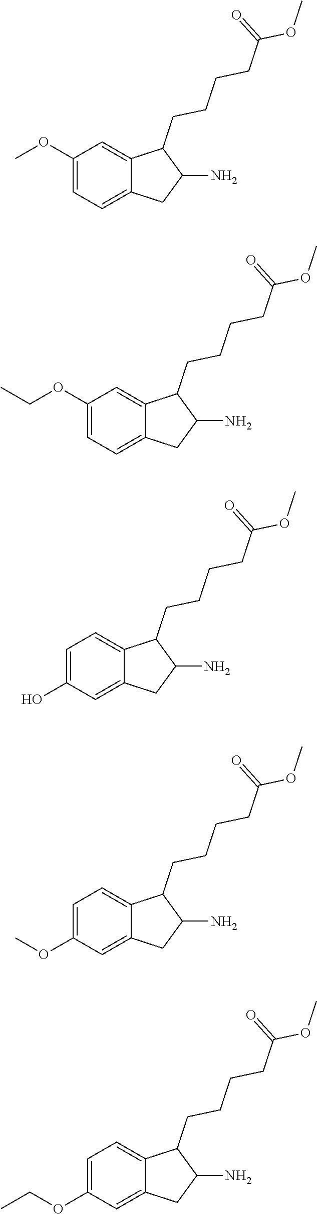

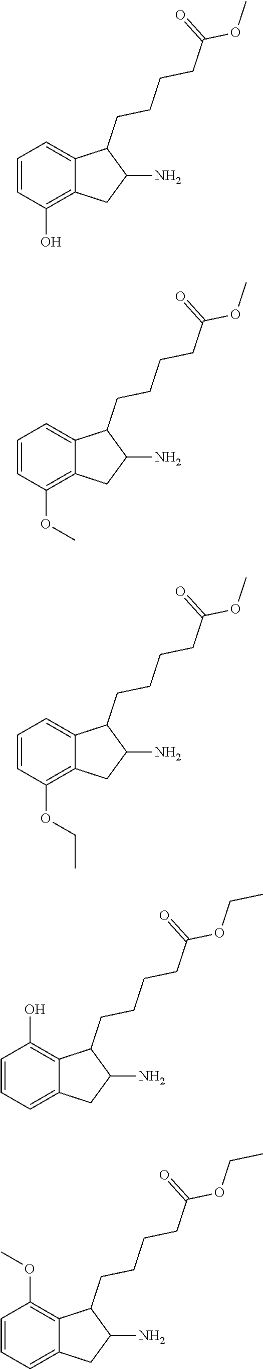 [SMILES: COC(=O)CCCCC1c2cccc(O)c2CC1N ; COC(=O)CCCCC1c2cccc(OC)c2CC1N ; CCOc1cccc2c1CC(N)C2CCCCC(=O)OC ; CCOC(=O)CCCCC1c2c(O)cccc2CC1N ; CCOC(=O)CCCCC1c2c(cccc2OC)CC1N]

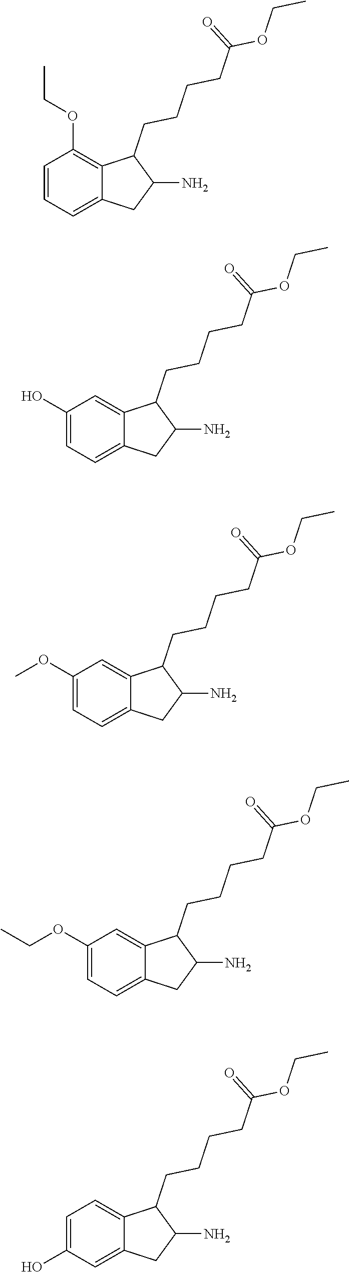

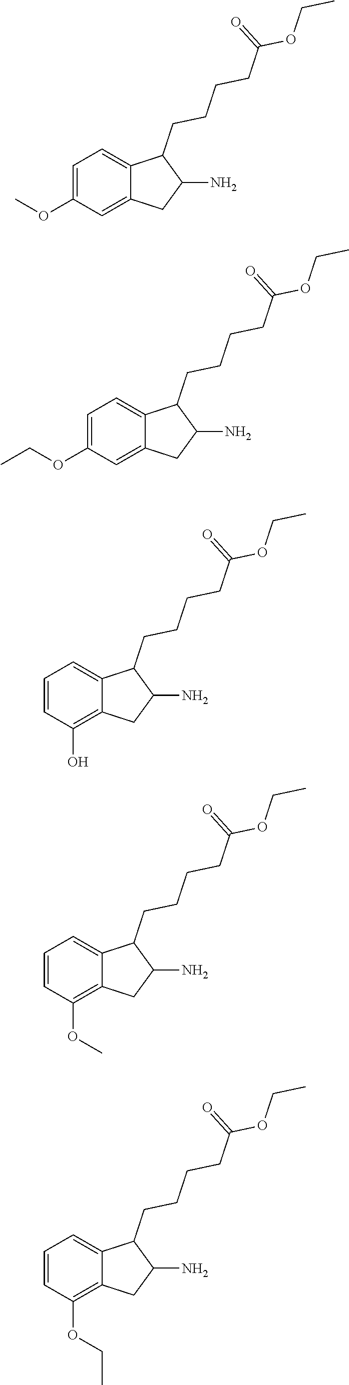

In some embodiments, the therapeutic compounds are those having a structure of Formula (Ij), and in exemplary such embodiments may be any one of the below:

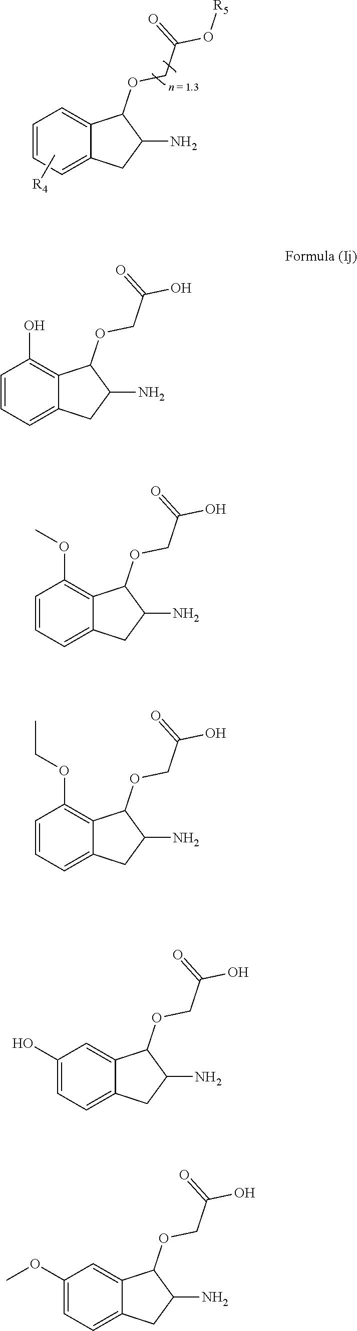

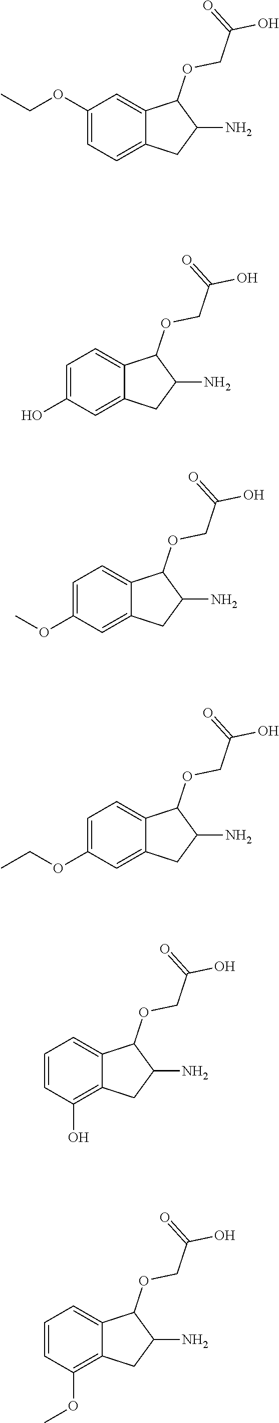 [SMILES: CCOc1ccc2c(c1)C(OCC(=O)O)C(N)C2 ; NC1Cc2cc(O)ccc2C1OCC(=O)O ; COc1ccc2c(c1)CC(N)C2OCC(=O)O ; CCOc1ccc2c(c1)CC(N)C2OCC(=O)O ; NC1Cc2c(O)cccc2C1OCC(=O)O ; COc1cccc2c1CC(N)C2OCC(=O)O]

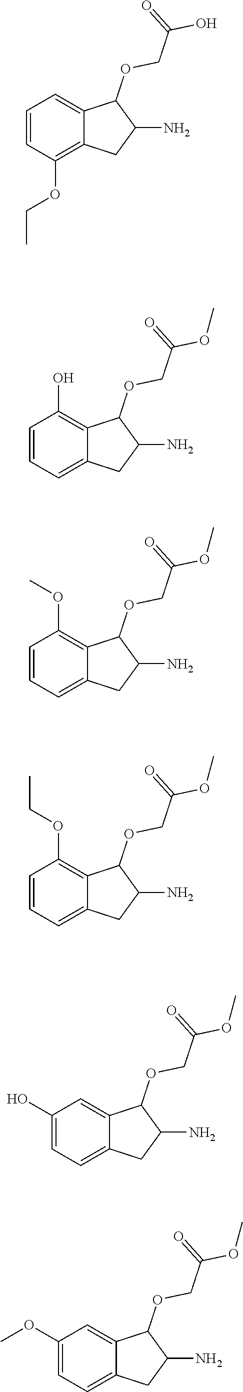

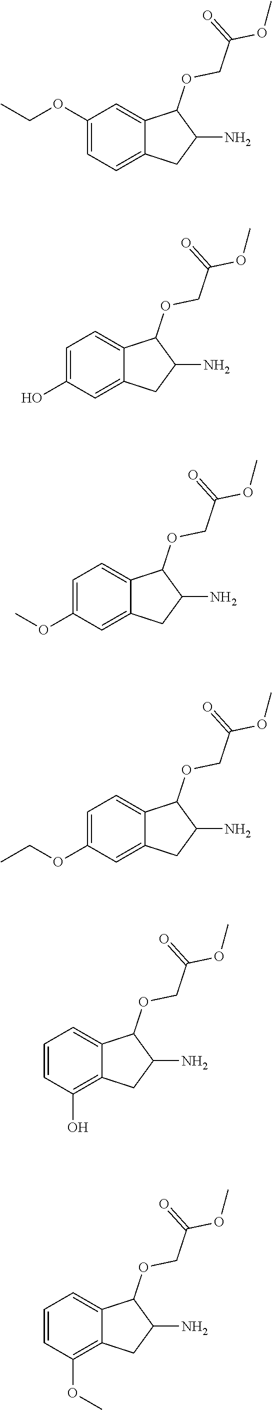

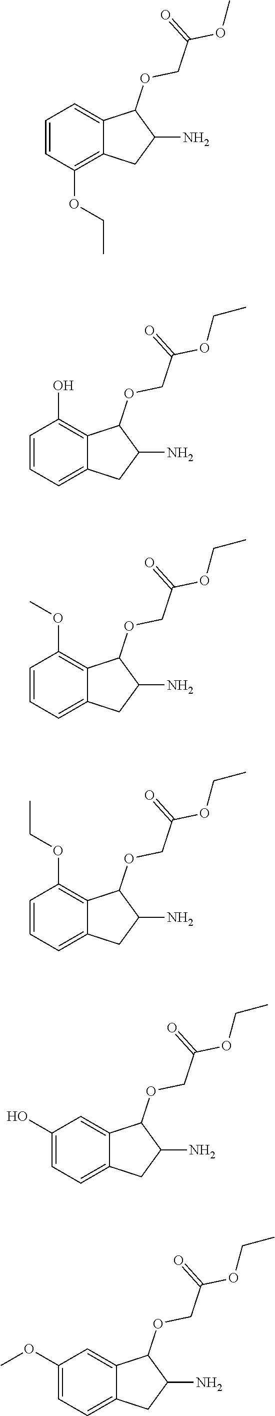

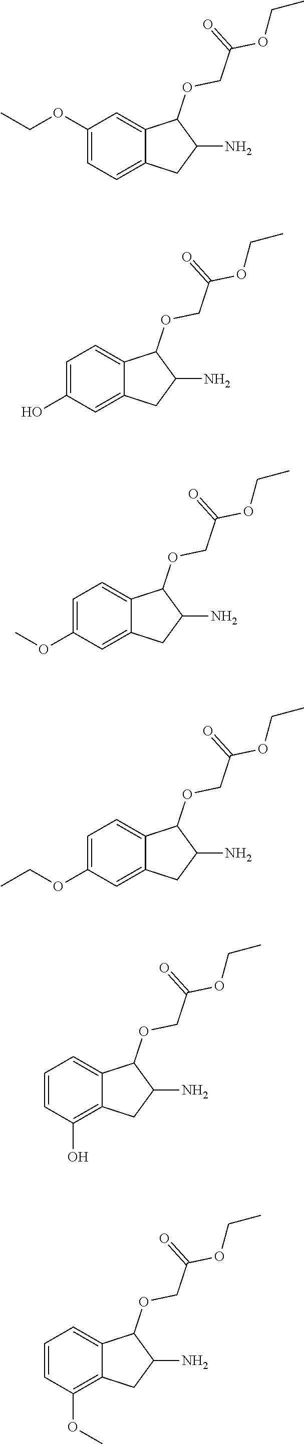

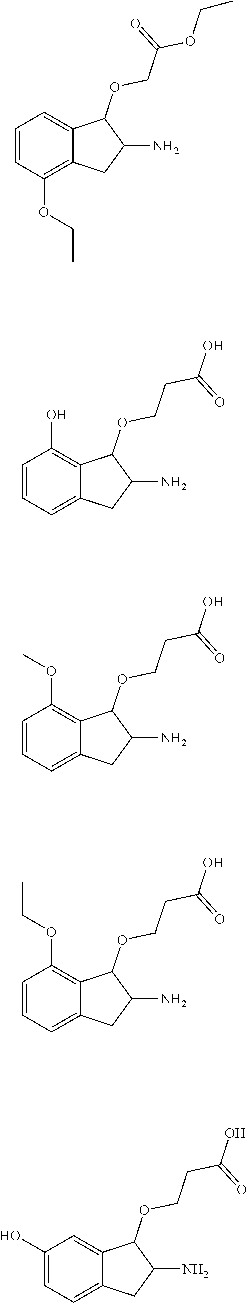 [SMILES: CCOC(=O)COC1c2cccc(OCC)c2CC1N ; NC1Cc2cccc(O)c2C1OCCC(=O)O ; COc1cccc2c1C(OCCC(=O)O)C(N)C2 ; CCOc1cccc2c1C(OCCC(=O)O)C(N)C2 ; NC1Cc2ccc(O)cc2C1OCCC(=O)O]

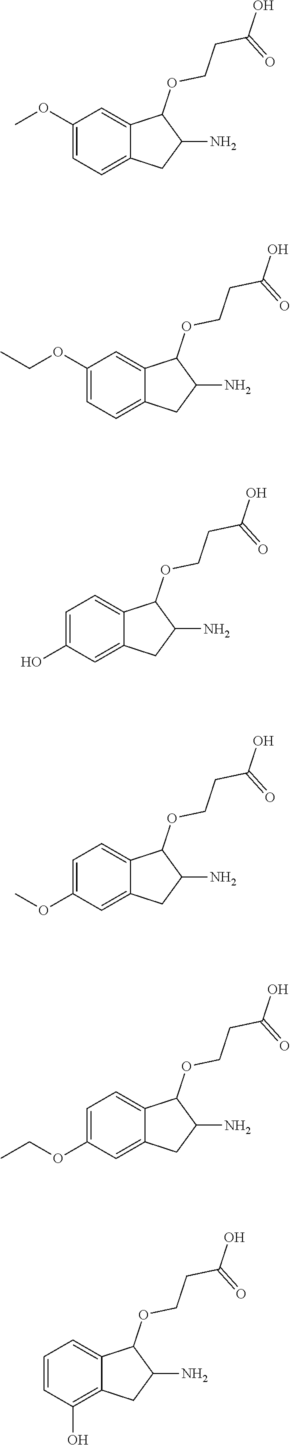 [SMILES: COc1ccc2c(c1)C(OCCC(=O)O)C(N)C2 ; CCOc1ccc2c(c1)C(OCCC(=O)O)C(N)C2 ; NC1Cc2cc(O)ccc2C1OCCC(=O)O ; COc1ccc2c(c1)CC(N)C2OCCC(=O)O ; CCOc1ccc2c(c1)CC(N)C2OCCC(=O)O ; NC1Cc2c(O)cccc2C1OCCC(=O)O]

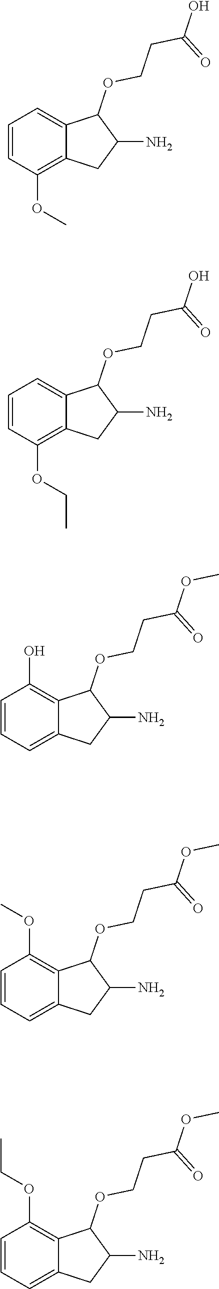 [SMILES: COc1cccc2c1CC(N)C2OCCC(=O)O ; CCOc1cccc2c1CC(N)C2OCCC(=O)O ; COC(=O)CCOC1c2c(O)cccc2CC1N ; COC(=O)CCOC1c2c(cccc2OC)CC1N ; CCOc1cccc2c1C(OCCC(=O)OC)C(N)C2]

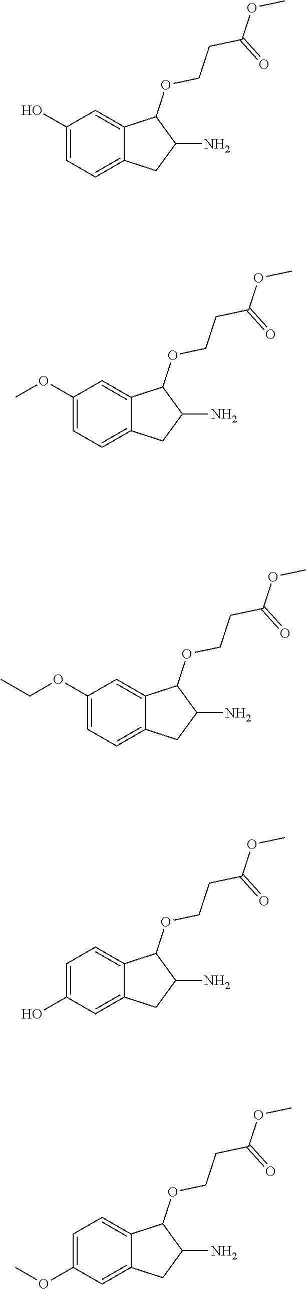

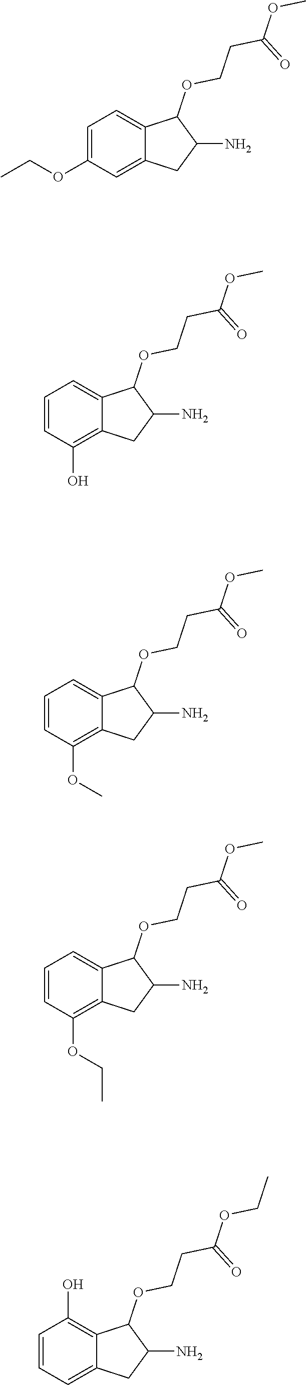 [SMILES: CCOc1ccc2c(c1)CC(N)C2OCCC(=O)OC ; COC(=O)CCOC1c2cccc(O)c2CC1N ; COC(=O)CCOC1c2cccc(OC)c2CC1N ; CCOc1cccc2c1CC(N)C2OCCC(=O)OC ; CCOC(=O)CCOC1c2c(O)cccc2CC1N]

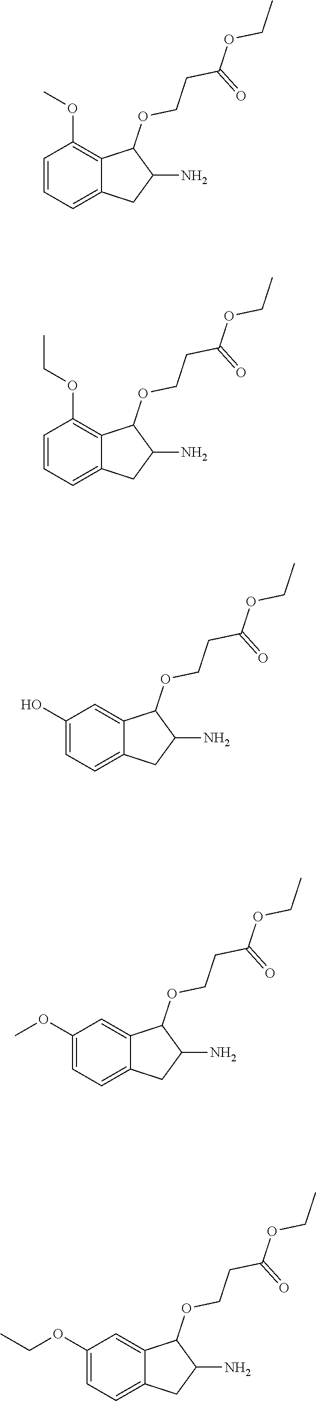

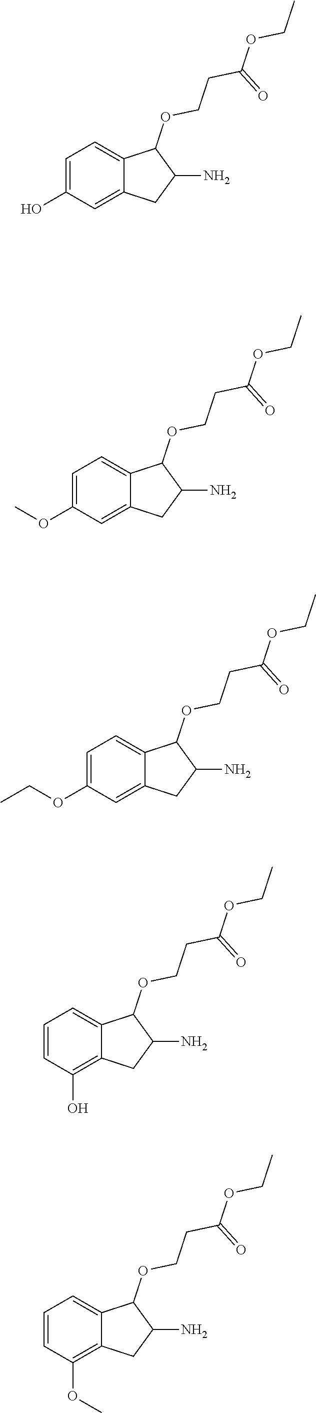

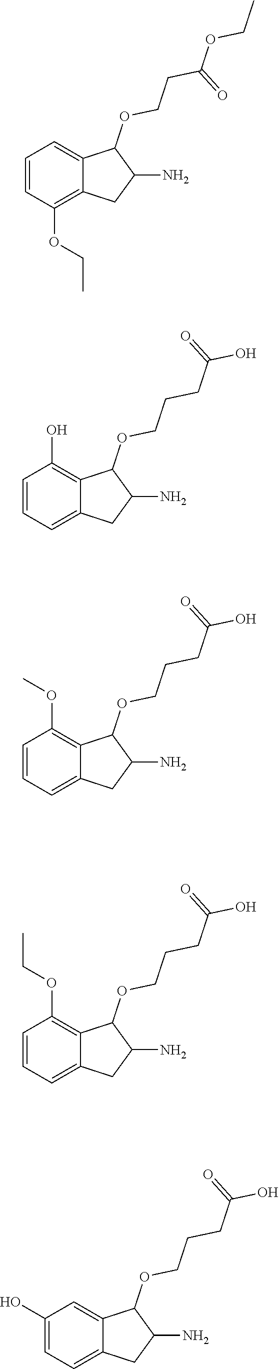 [SMILES: CCOC(=O)CCOC1c2cccc(OCC)c2CC1N ; NC1Cc2cccc(O)c2C1OCCCC(=O)O ; COc1cccc2c1C(OCCCC(=O)O)C(N)C2 ; CCOc1cccc2c1C(OCCCC(=O)O)C(N)C2 ; NC1Cc2ccc(O)cc2C1OCCCC(=O)O]

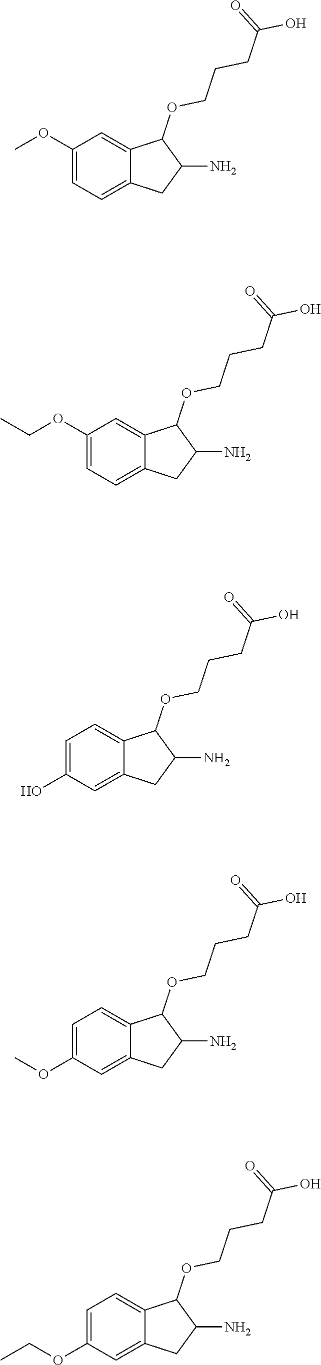 [SMILES: COc1ccc2c(c1)C(OCCCC(=O)O)C(N)C2 ; CCOc1ccc2c(c1)C(OCCCC(=O)O)C(N)C2 ; NC1Cc2cc(O)ccc2C1OCCCC(=O)O ; COc1ccc2c(c1)CC(N)C2OCCCC(=O)O ; CCOc1ccc2c(c1)CC(N)C2OCCCC(=O)O]

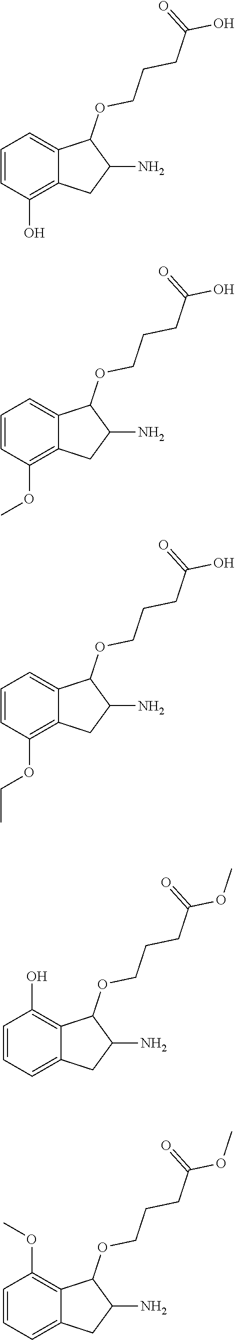

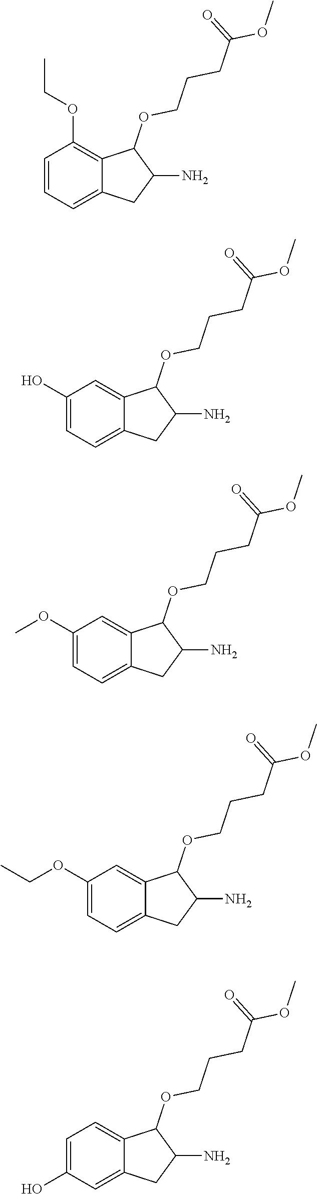

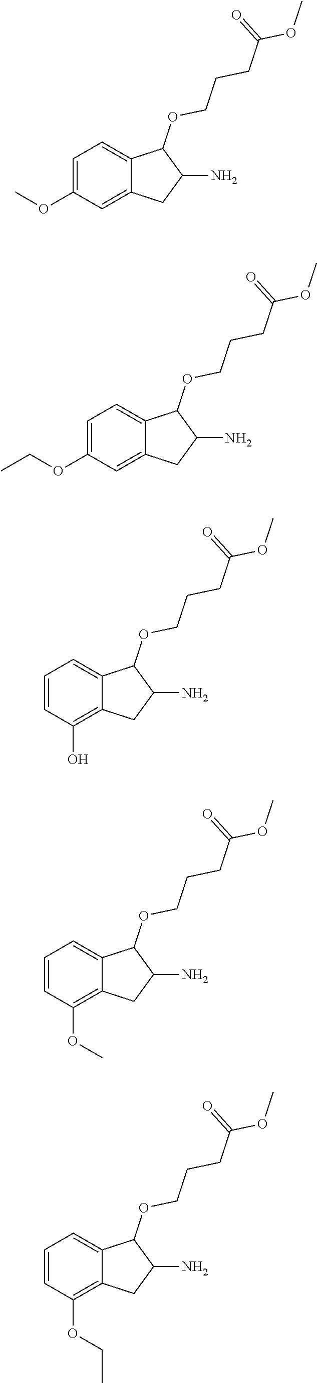

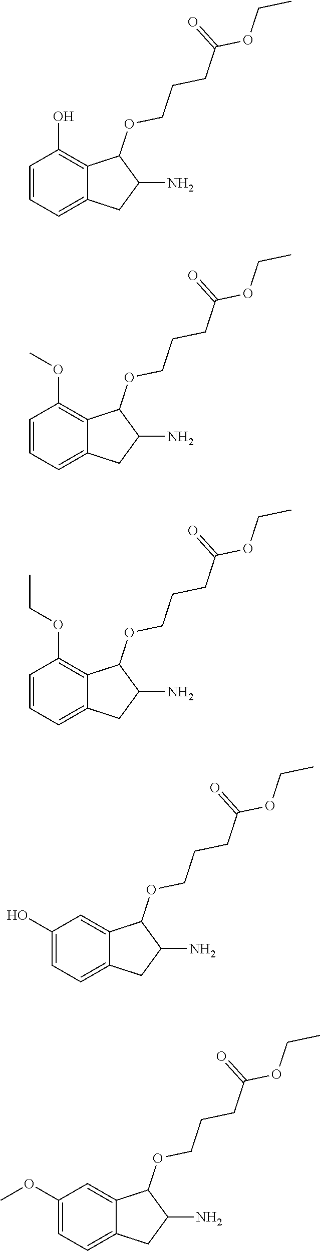

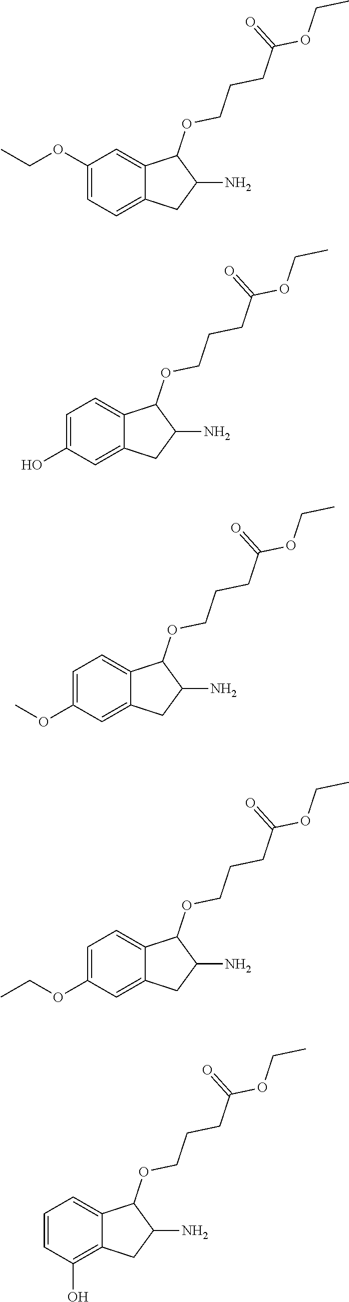

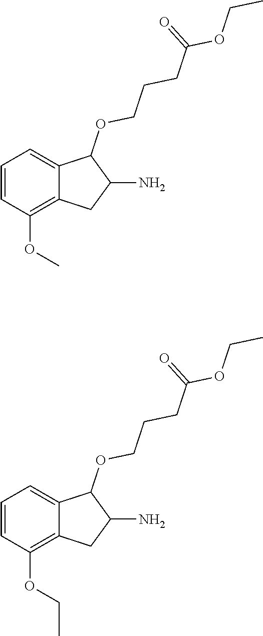 [SMILES: CCOC(=O)CCCOC1c2cccc(OC)c2CC1N ; CCOC(=O)CCCOC1c2cccc(OCC)c2CC1N]

In some embodiments, the therapeutic compounds are those having a structure of Formula (Ik), and in exemplary such embodiments may be any one of the below:

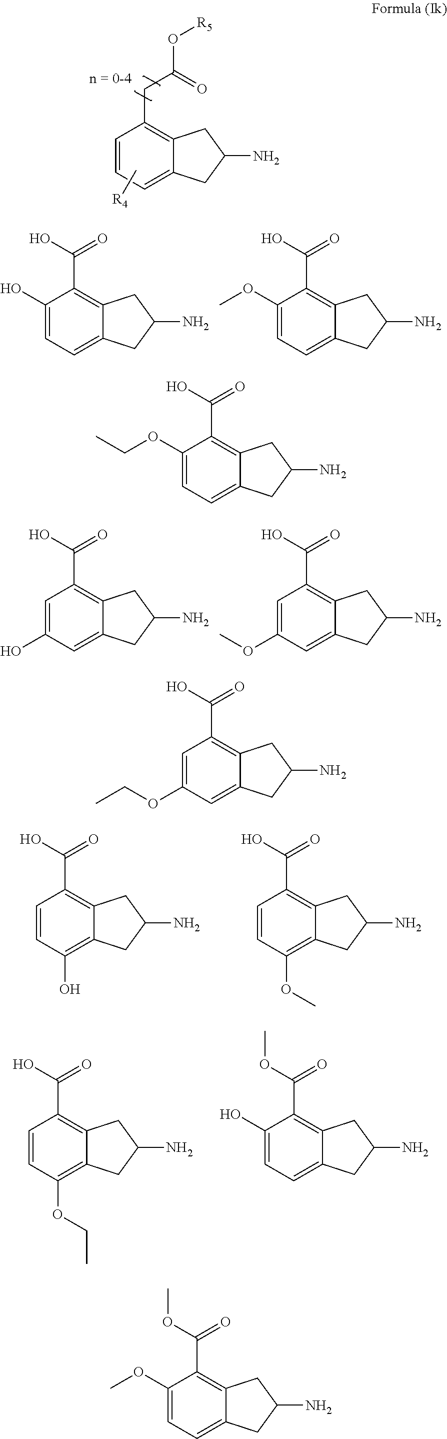

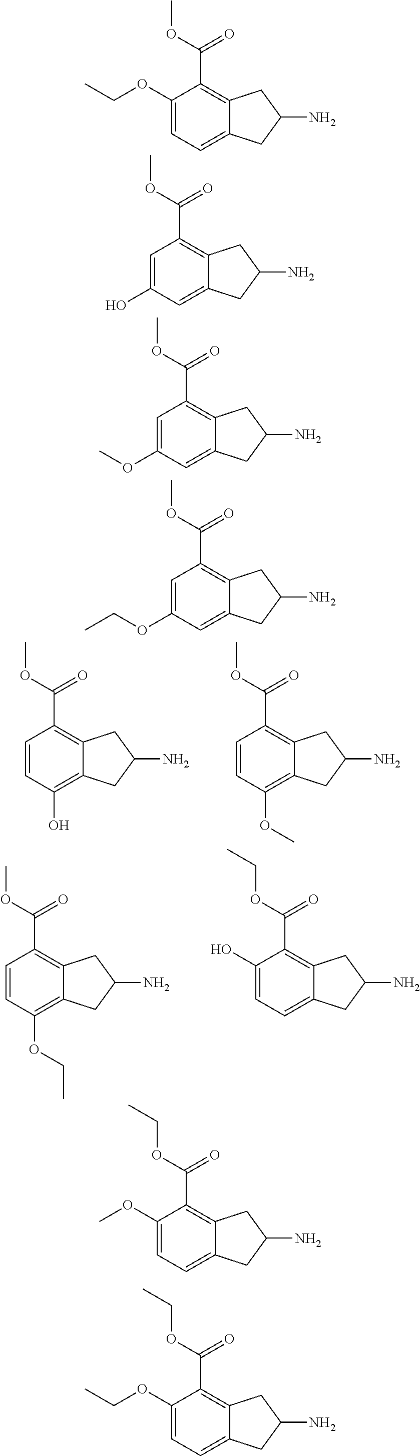

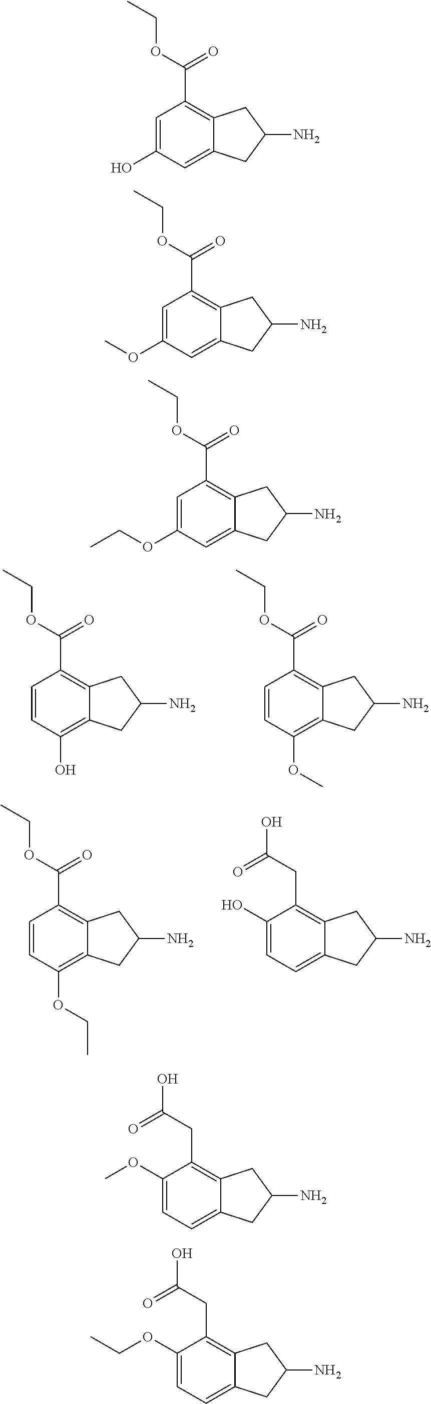

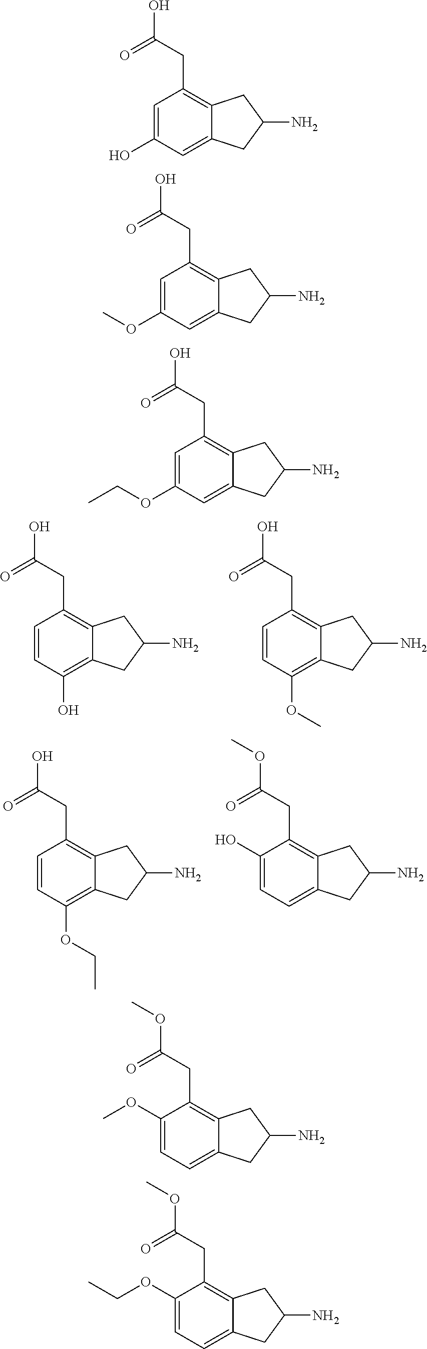

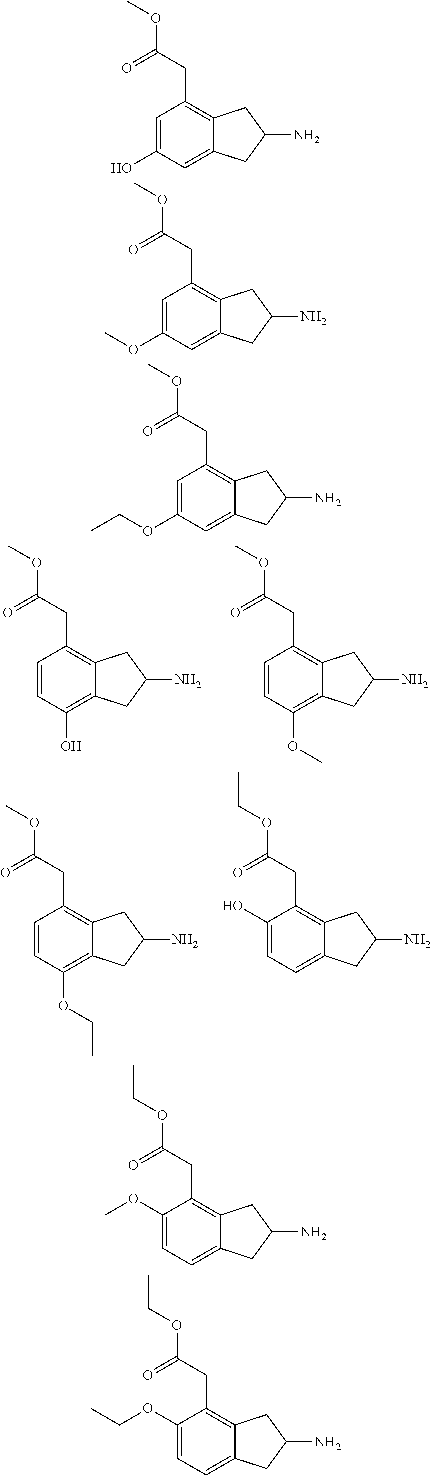

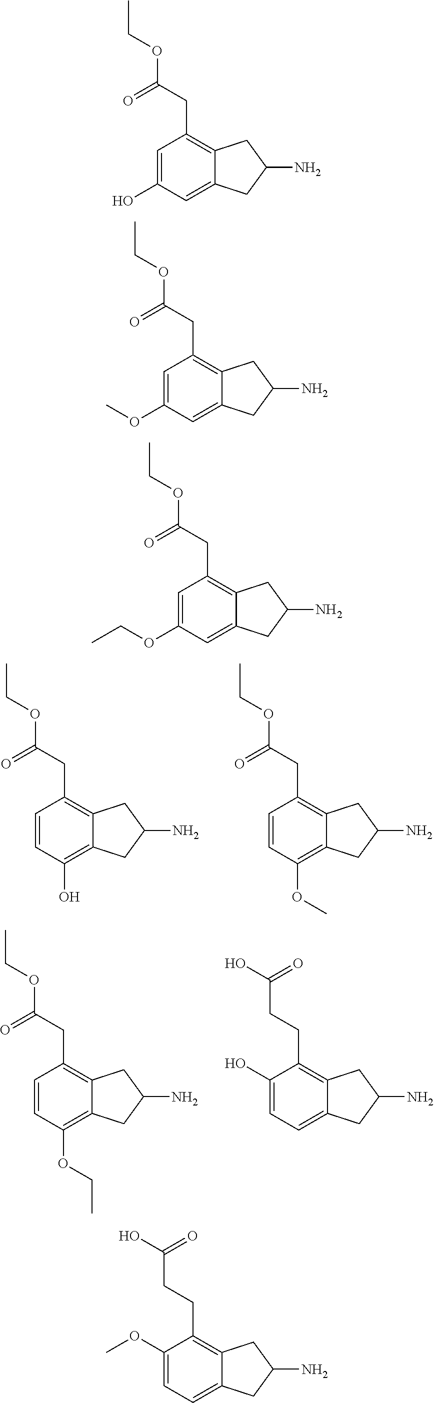

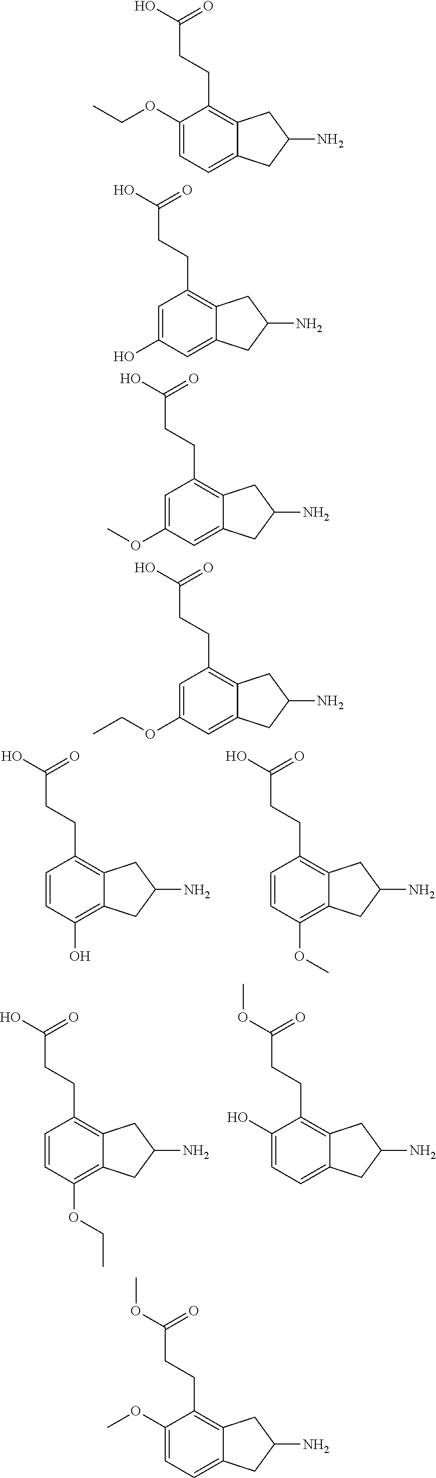

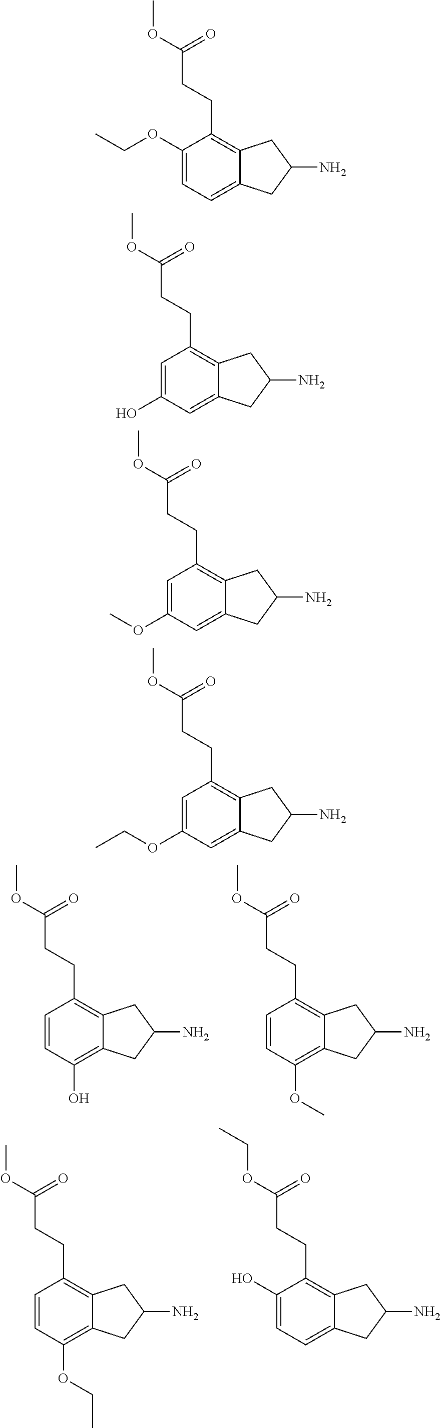

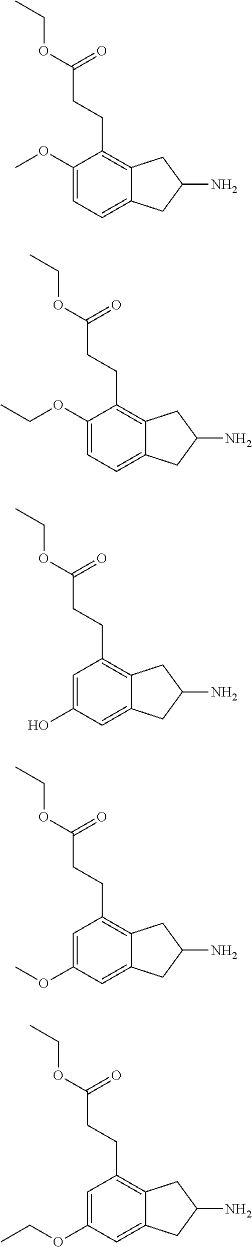

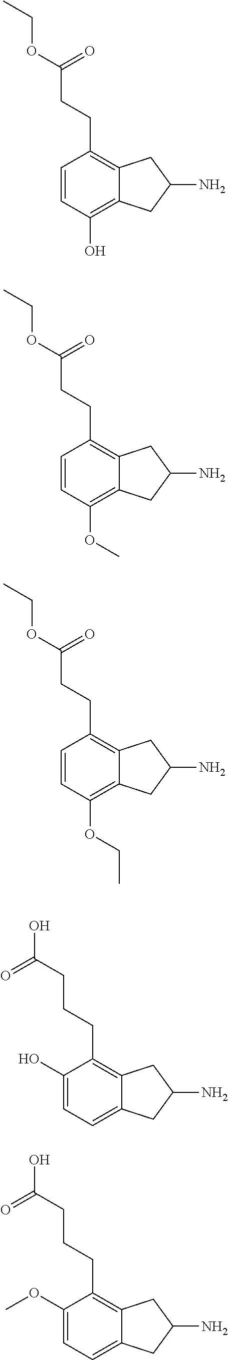 [SMILES: CCOC(=O)CCc1ccc(O)c2c1CC(N)C2 ; CCOC(=O)CCc1ccc(OC)c2c1CC(N)C2 ; CCOC(=O)CCc1ccc(OCC)c2c1CC(N)C2 ; NC1Cc2ccc(O)c(CCCC(=O)O)c2C1 ; COc1ccc2c(c1CCCC(=O)O)CC(N)C2]

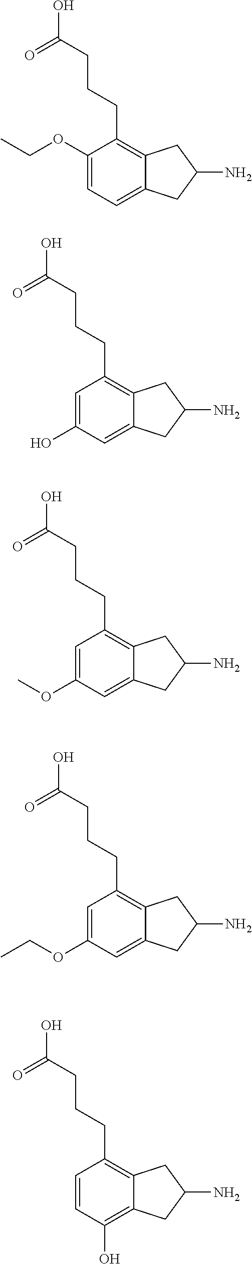 [SMILES: CCOc1ccc2c(c1CCCC(=O)O)CC(N)C2 ; NC1Cc2cc(O)cc(CCCC(=O)O)c2C1 ; COc1cc(CCCC(=O)O)c2c(c1)CC(N)C2 ; CCOc1cc(CCCC(=O)O)c2c(c1)CC(N)C2 ; NC1Cc2c(O)ccc(CCCC(=O)O)c2C1]

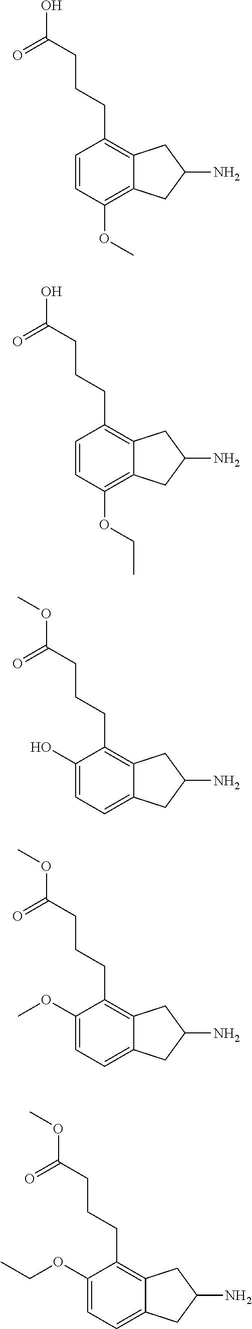 [SMILES: COc1ccc(CCCC(=O)O)c2c1CC(N)C2 ; CCOc1ccc(CCCC(=O)O)c2c1CC(N)C2 ; COC(=O)CCCc1c(O)ccc2c1CC(N)C2 ; COC(=O)CCCc1c(OC)ccc2c1CC(N)C2 ; CCOc1ccc2c(c1CCCC(=O)OC)CC(N)C2]

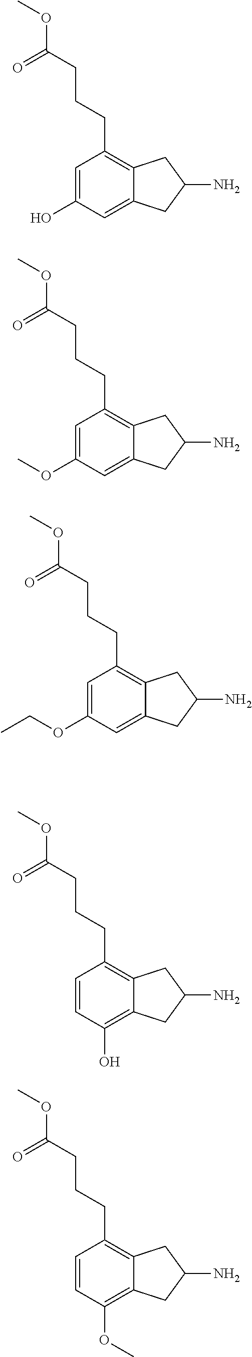

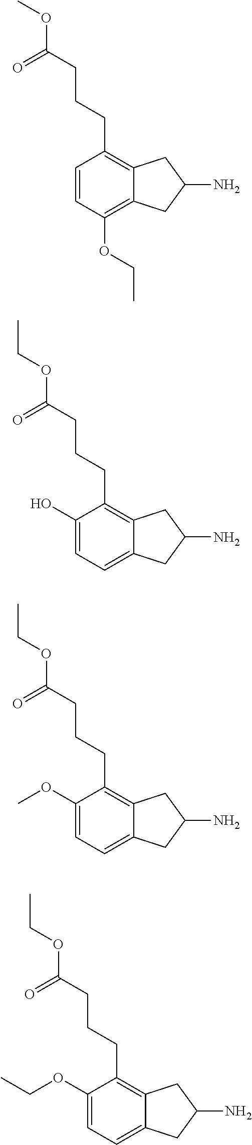 [SMILES: CCOc1ccc(CCCC(=O)OC)c2c1CC(N)C2 ; CCOC(=O)CCCc1c(O)ccc2c1CC(N)C2 ; CCOC(=O)CCCc1c(OC)ccc2c1CC(N)C2 ; CCOC(=O)CCCc1c(OCC)ccc2c1CC(N)C2]

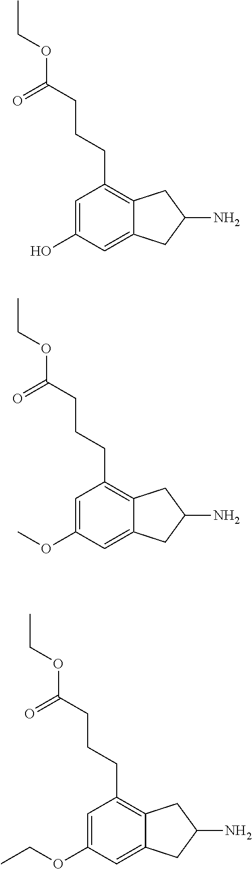 [SMILES: CCOC(=O)CCCc1cc(O)cc2c1CC(N)C2 ; CCOC(=O)CCCc1cc(OC)cc2c1CC(N)C2 ; CCOC(=O)CCCc1cc(OCC)cc2c1CC(N)C2]

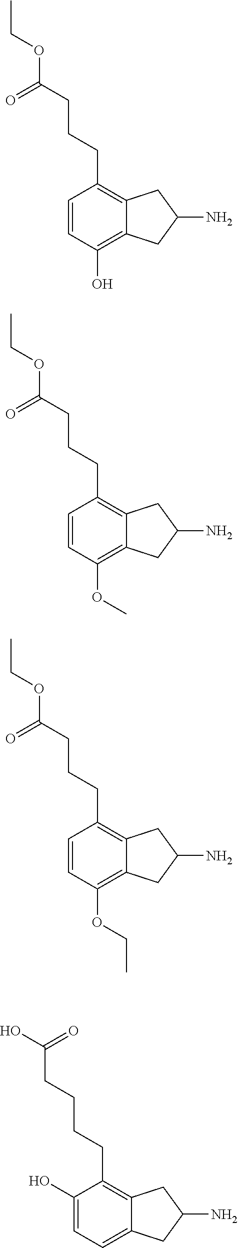 [SMILES: CCOC(=O)CCCc1ccc(O)c2c1CC(N)C2 ; CCOC(=O)CCCc1ccc(OC)c2c1CC(N)C2 ; CCOC(=O)CCCc1ccc(OCC)c2c1CC(N)C2 ; NC1Cc2ccc(O)c(CCCCC(=O)O)c2C1]

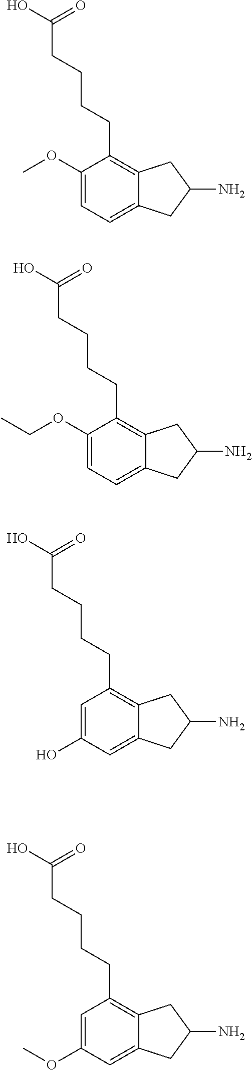 [SMILES: COc1ccc2c(c1CCCCC(=O)O)CC(N)C2 ; CCOc1ccc2c(c1CCCCC(=O)O)CC(N)C2 ; NC1Cc2cc(O)cc(CCCCC(=O)O)c2C1 ; COc1cc(CCCCC(=O)O)c2c(c1)CC(N)C2]

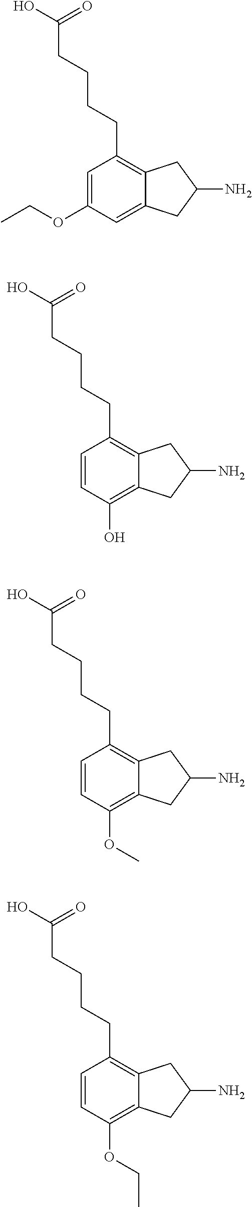 [SMILES: CCOc1cc(CCCCC(=O)O)c2c(c1)CC(N)C2 ; NC1Cc2c(O)ccc(CCCCC(=O)O)c2C1 ; COc1ccc(CCCCC(=O)O)c2c1CC(N)C2 ; CCOc1ccc(CCCCC(=O)O)c2c1CC(N)C2]

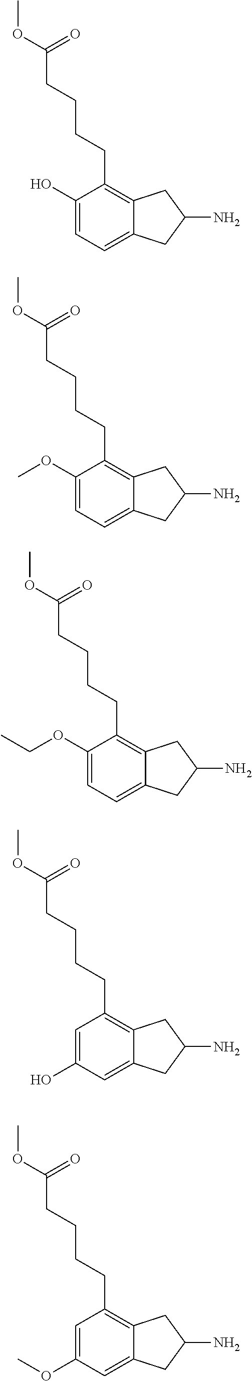 [SMILES: COC(=O)CCCCc1c(O)ccc2c1CC(N)C2 ; COC(=O)CCCCc1c(OC)ccc2c1CC(N)C2 ; CCOc1ccc2c(c1CCCCC(=O)OC)CC(N)C2 ; COC(=O)CCCCc1cc(O)cc2c1CC(N)C2 ; COC(=O)CCCCc1cc(OC)cc2c1CC(N)C2]

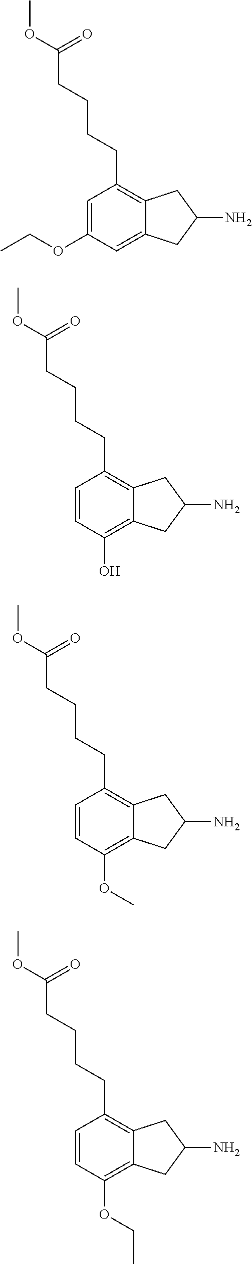

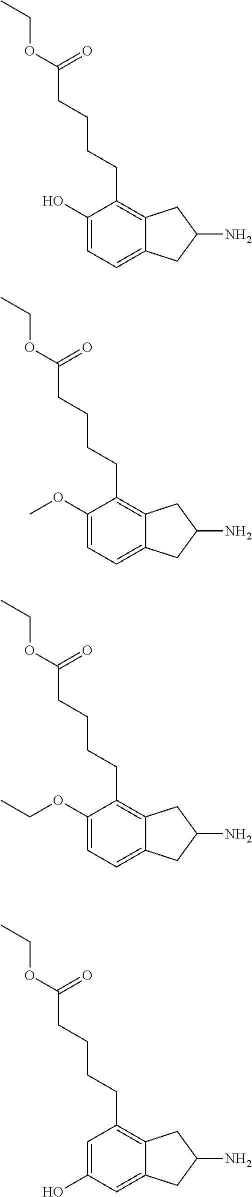 [SMILES: CCOC(=O)CCCCc1c(O)ccc2c1CC(N)C2 ; CCOC(=O)CCCCc1c(OC)ccc2c1CC(N)C2 ; CCOC(=O)CCCCc1c(OCC)ccc2c1CC(N)C2 ; CCOC(=O)CCCCc1cc(O)cc2c1CC(N)C2]

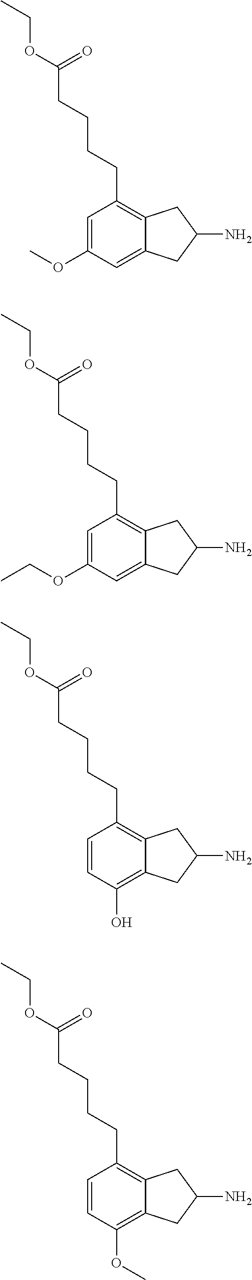

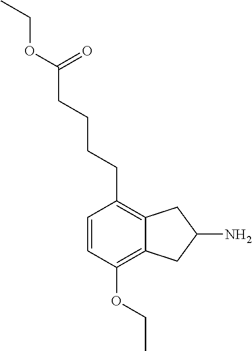 [SMILES: CCOC(=O)CCCCc1ccc(OCC)c2c1CC(N)C2]

In some embodiments, the therapeutic compounds are those having a structure of Formula (Il), and in exemplary such embodiments may be any one of the below:

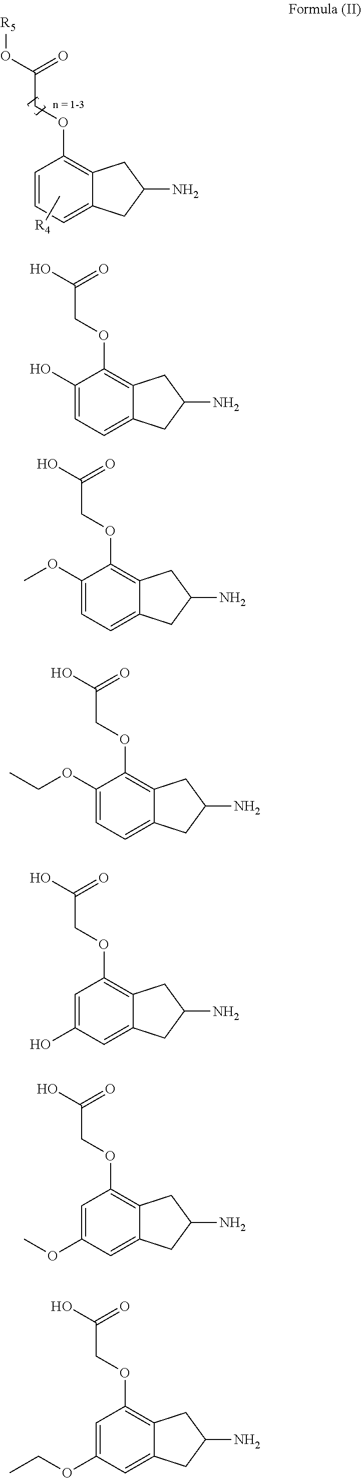

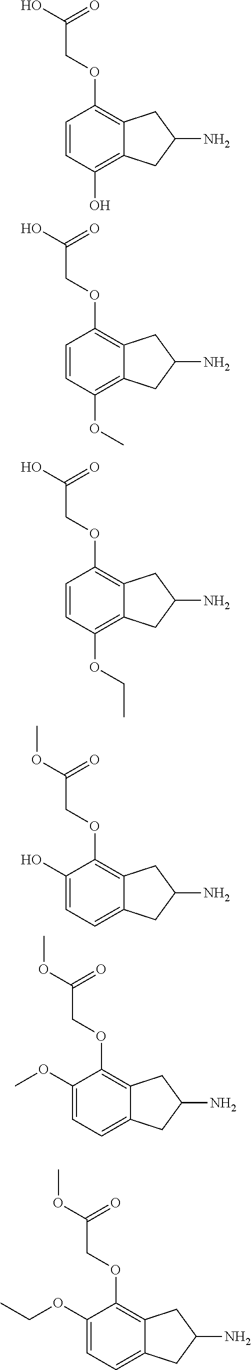

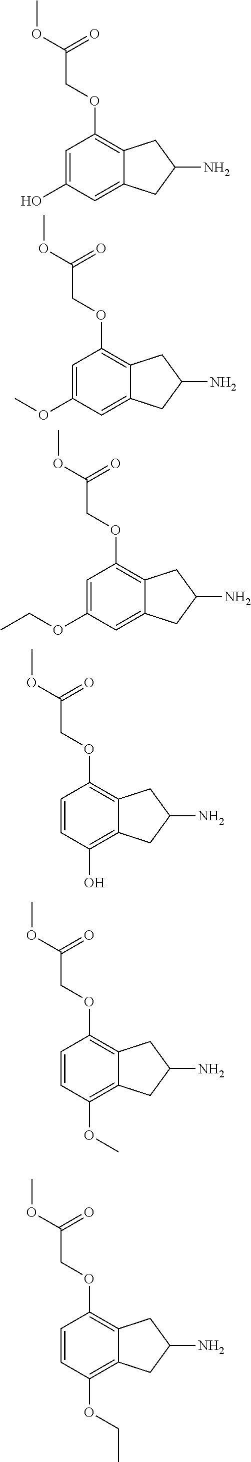

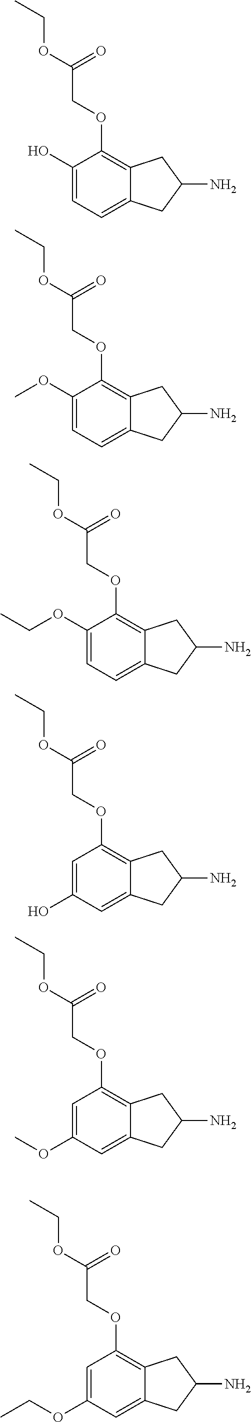

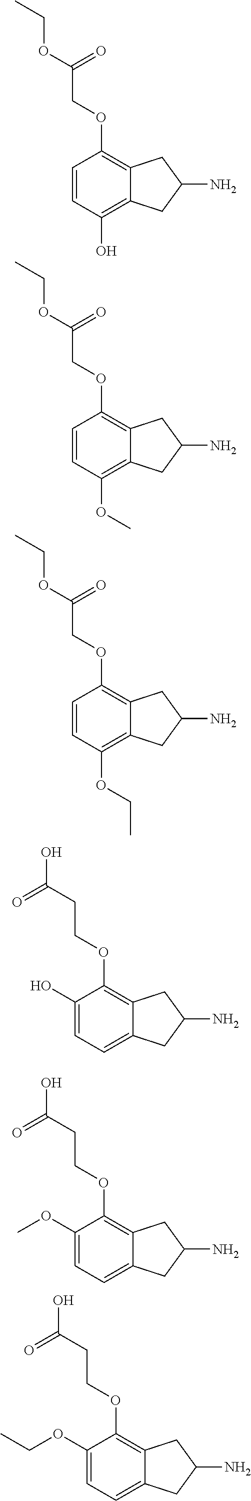

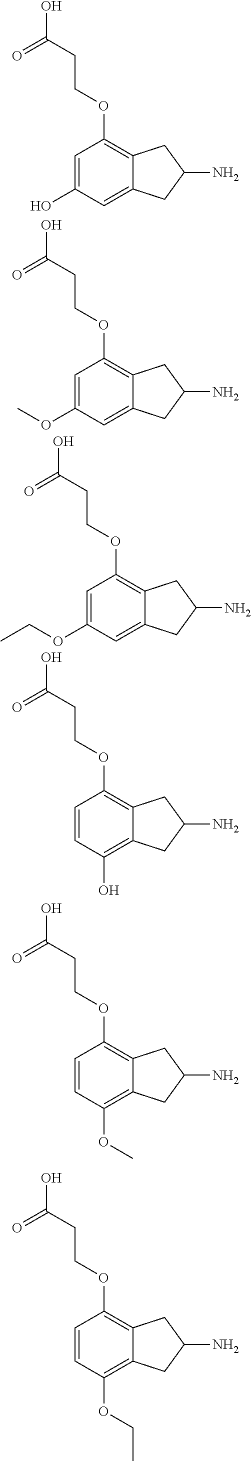

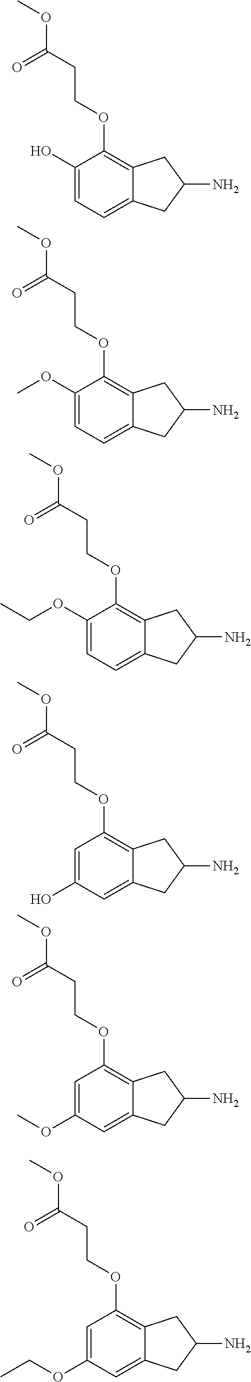

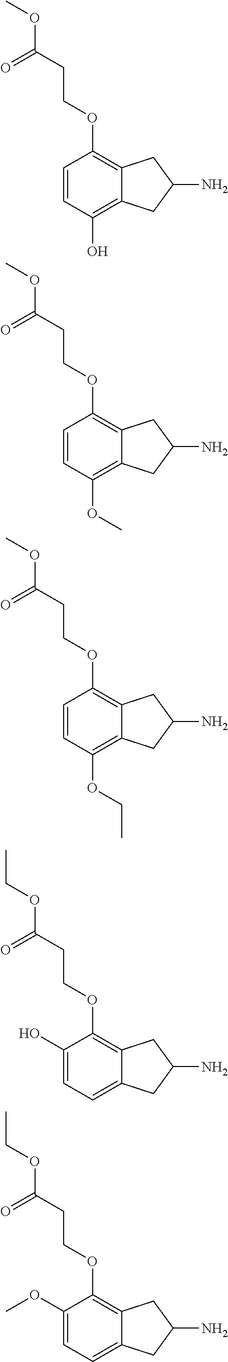 [SMILES: COC(=O)CCOc1ccc(O)c2c1CC(N)C2 ; COC(=O)CCOc1ccc(OC)c2c1CC(N)C2 ; CCOc1ccc(OCCC(=O)OC)c2c1CC(N)C2 ; CCOC(=O)CCOc1c(O)ccc2c1CC(N)C2 ; CCOC(=O)CCOc1c(OC)ccc2c1CC(N)C2]

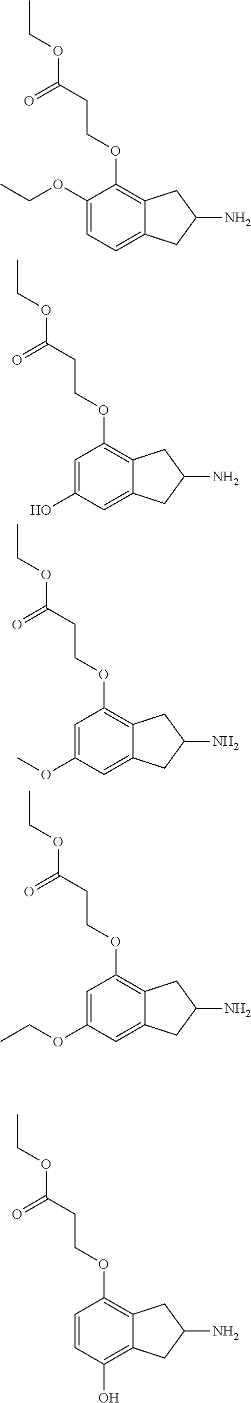

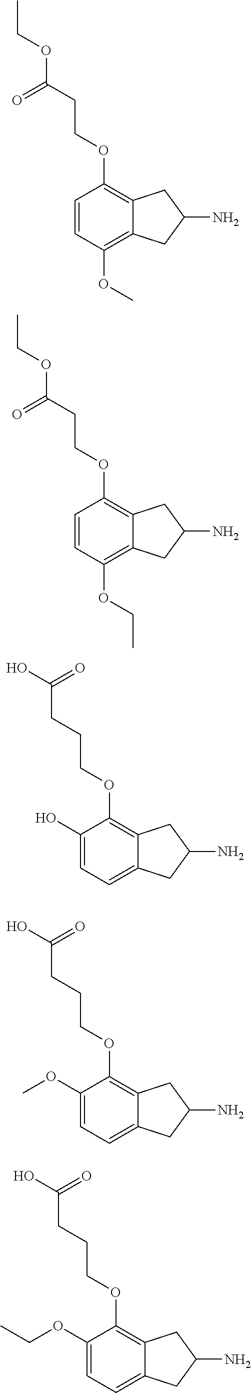

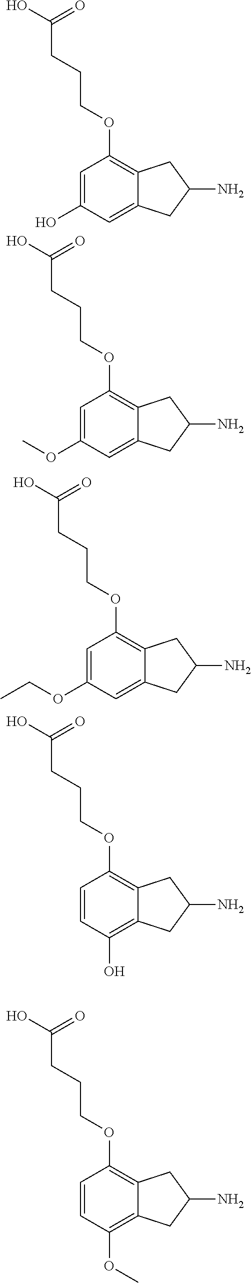

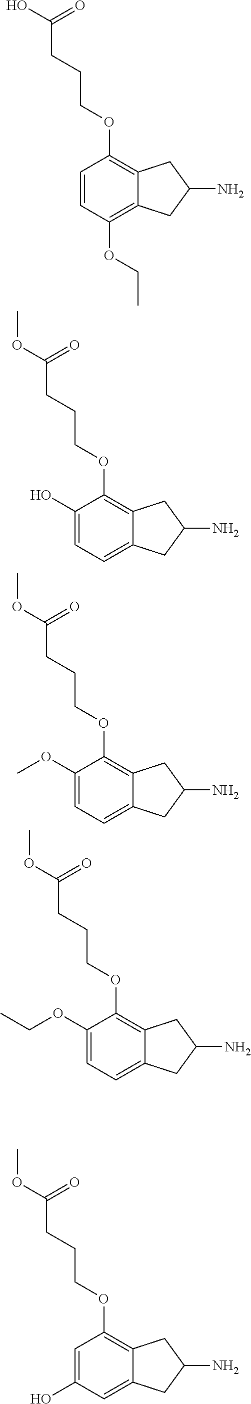

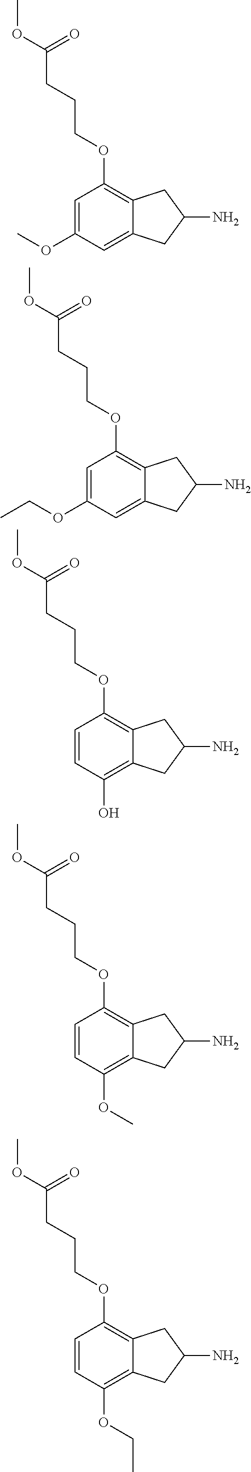

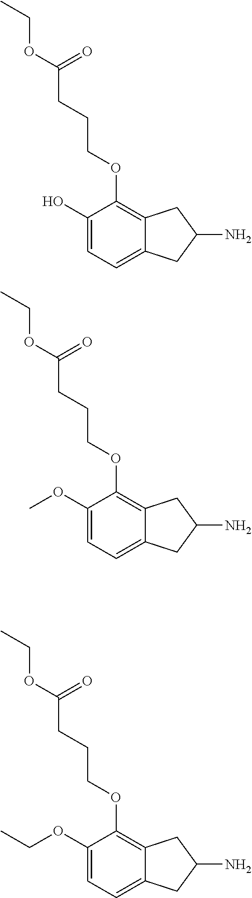 [SMILES: CCOC(=O)CCCOc1c(O)ccc2c1CC(N)C2 ; CCOC(=O)CCCOc1c(OC)ccc2c1CC(N)C2 ; CCOC(=O)CCCOc1c(OCC)ccc2c1CC(N)C2]

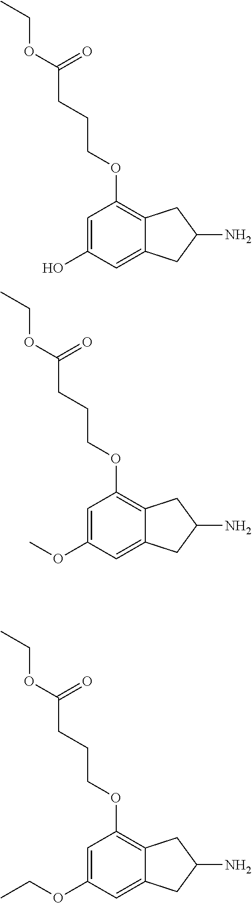 [SMILES: CCOC(=O)CCCOc1cc(O)cc2c1CC(N)C2 ; CCOC(=O)CCCOc1cc(OC)cc2c1CC(N)C2 ; CCOC(=O)CCCOc1cc(OCC)cc2c1CC(N)C2]

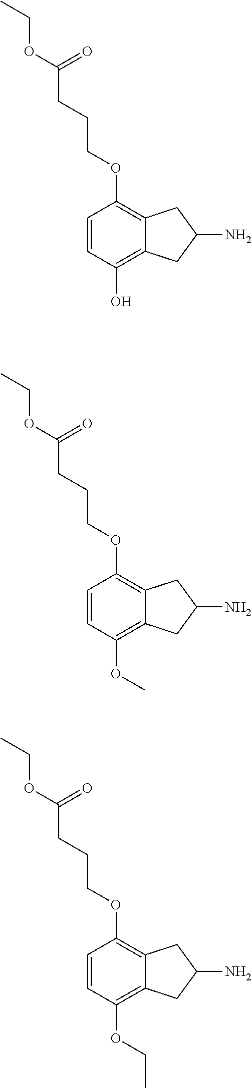 [SMILES: CCOC(=O)CCCOc1ccc(O)c2c1CC(N)C2 ; CCOC(=O)CCCOc1ccc(OC)c2c1CC(N)C2 ; CCOC(=O)CCCOc1ccc(OCC)c2c1CC(N)C2]

In some embodiments, the therapeutic compounds are those having a structure of Formula (Im), and in exemplary such embodiments may be any one of the below:

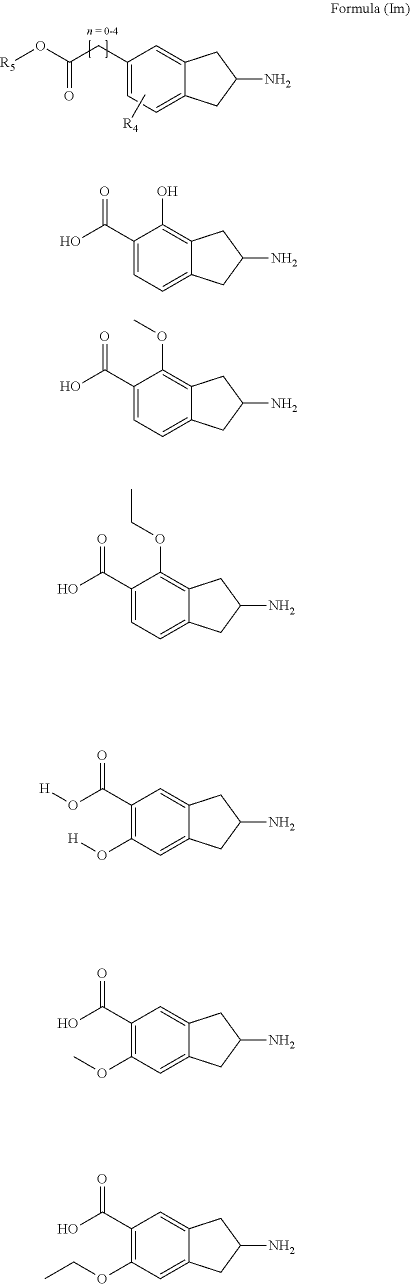

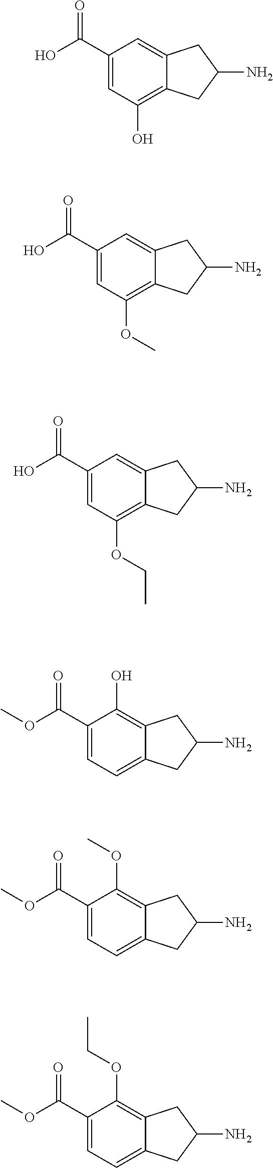 [SMILES: NC1Cc2cc(C(=O)O)cc(O)c2C1 ; COc1cc(C(=O)O)cc2c1CC(N)C2 ; CCOc1cc(C(=O)O)cc2c1CC(N)C2 ; COC(=O)c1ccc2c(c1O)CC(N)C2 ; COC(=O)c1ccc2c(c1OC)CC(N)C2 ; CCOc1c(C(=O)OC)ccc2c1CC(N)C2]

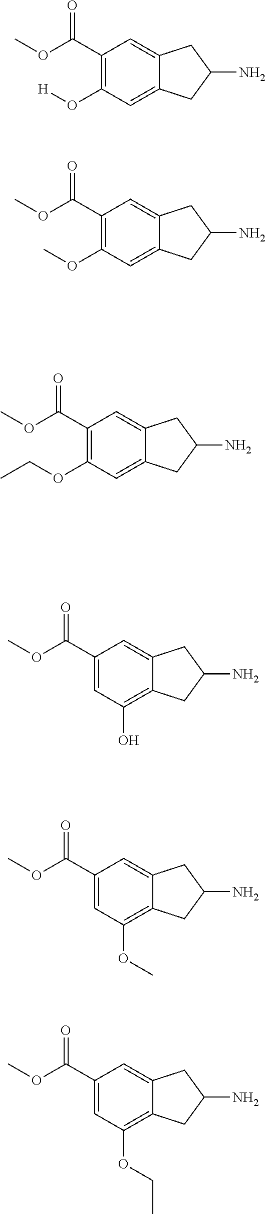 [SMILES: COC(=O)c1cc2c(cc1O)CC(N)C2 ; COC(=O)c1cc2c(cc1OC)CC(N)C2 ; CCOc1cc2c(cc1C(=O)OC)CC(N)C2 ; COC(=O)c1cc(O)c2c(c1)CC(N)C2 ; COC(=O)c1cc2c(c(OC)c1)CC(N)C2 ; CCOc1cc(C(=O)OC)cc2c1CC(N)C2]

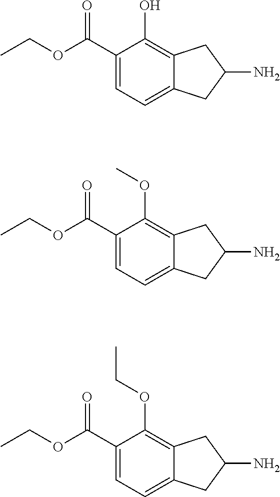 [SMILES: CCOC(=O)c1ccc2c(c1O)CC(N)C2 ; CCOC(=O)c1ccc2c(c1OC)CC(N)C2 ; CCOC(=O)c1ccc2c(c1OCC)CC(N)C2]

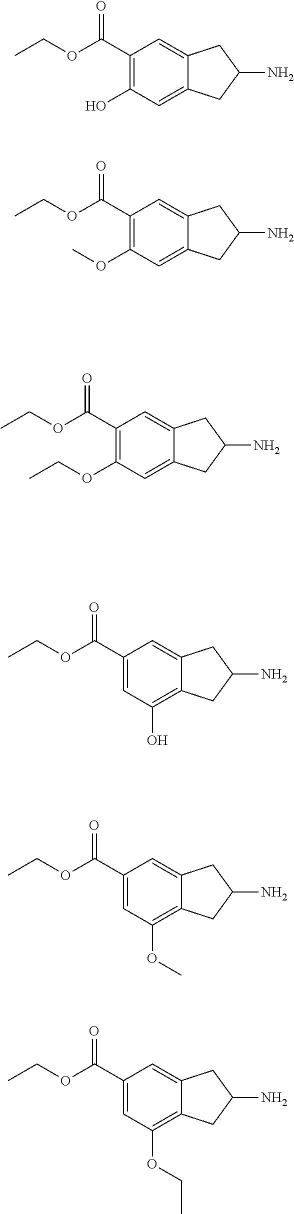 [SMILES: CCOC(=O)c1cc2c(cc1O)CC(N)C2 ; CCOC(=O)c1cc2c(cc1OC)CC(N)C2 ; CCOC(=O)c1cc2c(cc1OCC)CC(N)C2 ; CCOC(=O)c1cc(O)c2c(c1)CC(N)C2 ; CCOC(=O)c1cc2c(c(OC)c1)CC(N)C2 ; CCOC(=O)c1cc2c(c(OCC)c1)CC(N)C2]

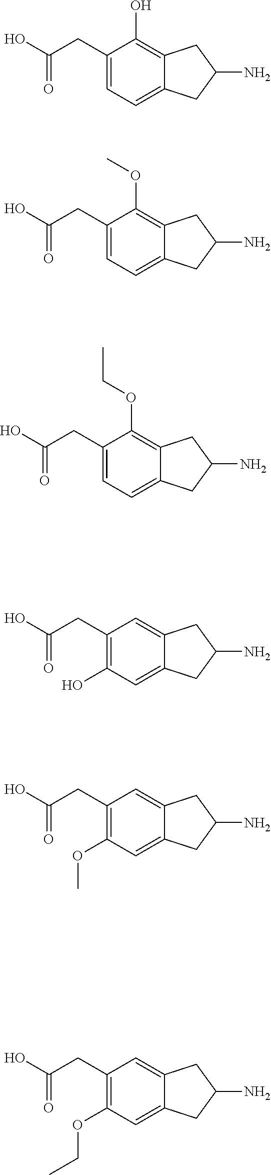 [SMILES: NC1Cc2ccc(CC(=O)O)c(O)c2C1 ; COc1c(CC(=O)O)ccc2c1CC(N)C2 ; CCOc1c(CC(=O)O)ccc2c1CC(N)C2 ; NC1Cc2cc(O)c(CC(=O)O)cc2C1 ; COc1cc2c(cc1CC(=O)O)CC(N)C2 ; CCOc1cc2c(cc1CC(=O)O)CC(N)C2]

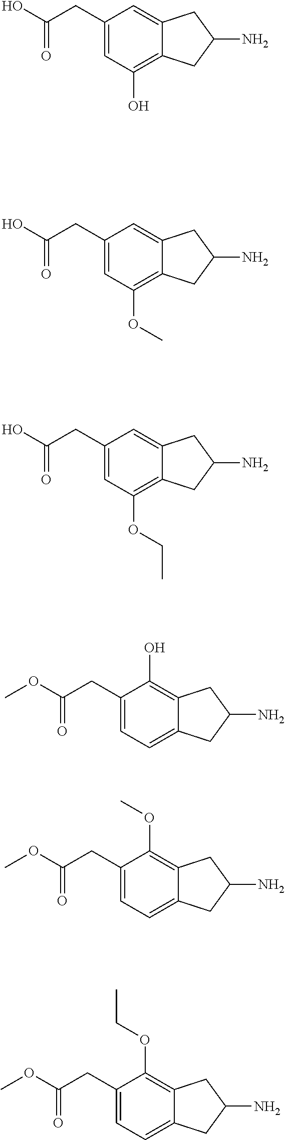 [SMILES: NC1Cc2cc(CC(=O)O)cc(O)c2C1 ; COc1cc(CC(=O)O)cc2c1CC(N)C2 ; CCOc1cc(CC(=O)O)cc2c1CC(N)C2 ; COC(=O)Cc1ccc2c(c1O)CC(N)C2 ; COC(=O)Cc1ccc2c(c1OC)CC(N)C2 ; CCOc1c(CC(=O)OC)ccc2c1CC(N)C2]

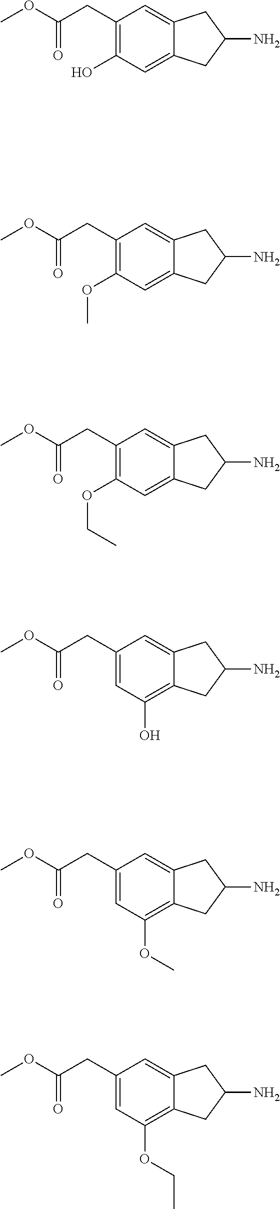 [SMILES: COC(=O)Cc1cc2c(cc1O)CC(N)C2 ; COC(=O)Cc1cc2c(cc1OC)CC(N)C2 ; CCOc1cc2c(cc1CC(=O)OC)CC(N)C2 ; COC(=O)Cc1cc(O)c2c(c1)CC(N)C2 ; COC(=O)Cc1cc2c(c(OC)c1)CC(N)C2 ; CCOc1cc(CC(=O)OC)cc2c1CC(N)C2]

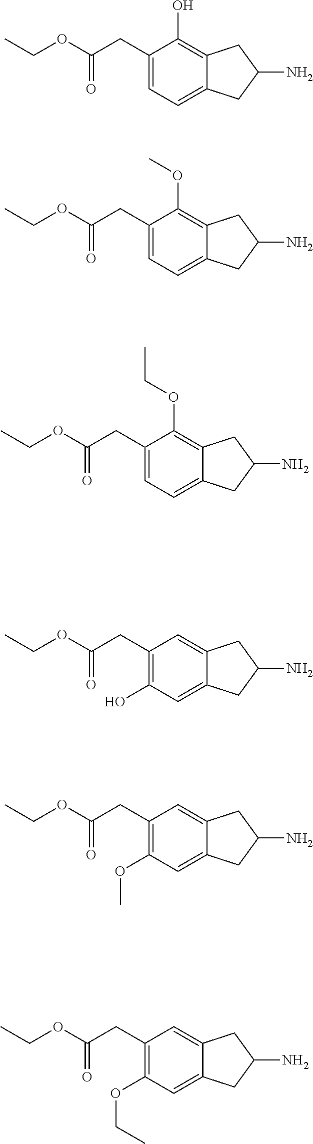 [SMILES: CCOC(=O)Cc1ccc2c(c1O)CC(N)C2 ; CCOC(=O)Cc1ccc2c(c1OC)CC(N)C2 ; CCOC(=O)Cc1ccc2c(c1OCC)CC(N)C2 ; CCOC(=O)Cc1cc2c(cc1O)CC(N)C2 ; CCOC(=O)Cc1cc2c(cc1OC)CC(N)C2 ; CCOC(=O)Cc1cc2c(cc1OCC)CC(N)C2]

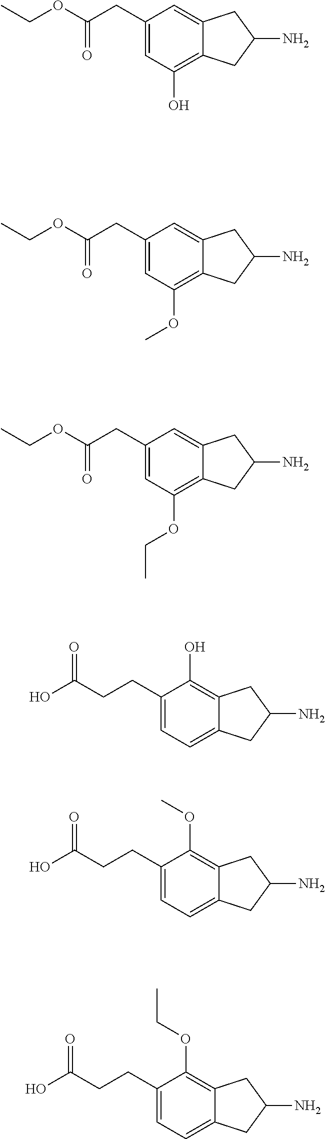 [SMILES: CCOC(=O)Cc1cc(O)c2c(c1)CC(N)C2 ; CCOC(=O)Cc1cc2c(c(OC)c1)CC(N)C2 ; CCOC(=O)Cc1cc2c(c(OCC)c1)CC(N)C2 ; NC1Cc2ccc(CCC(=O)O)c(O)c2C1 ; COc1c(CCC(=O)O)ccc2c1CC(N)C2 ; CCOc1c(CCC(=O)O)ccc2c1CC(N)C2]

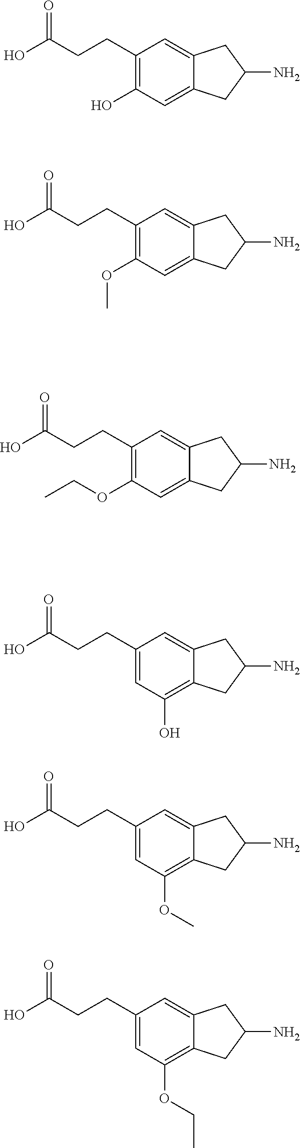 [SMILES: NC1Cc2cc(O)c(CCC(=O)O)cc2C1 ; COc1cc2c(cc1CCC(=O)O)CC(N)C2 ; CCOc1cc2c(cc1CCC(=O)O)CC(N)C2 ; NC1Cc2cc(CCC(=O)O)cc(O)c2C1 ; COc1cc(CCC(=O)O)cc2c1CC(N)C2 ; CCOc1cc(CCC(=O)O)cc2c1CC(N)C2]

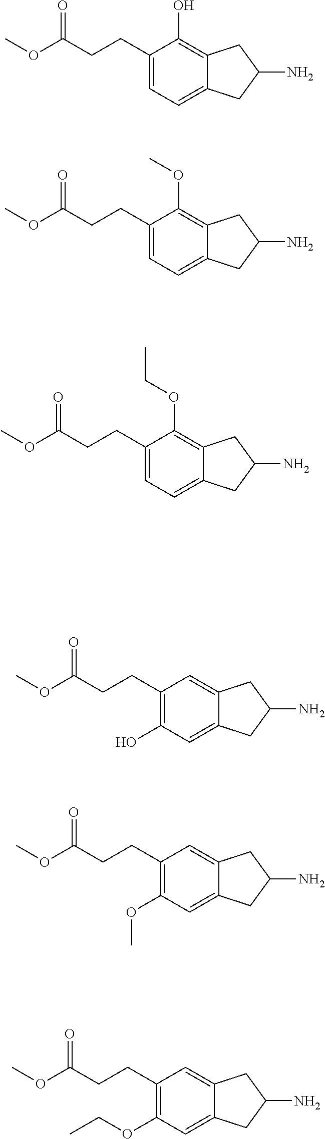 [SMILES: COC(=O)CCc1ccc2c(c1O)CC(N)C2 ; COC(=O)CCc1ccc2c(c1OC)CC(N)C2 ; CCOc1c(CCC(=O)OC)ccc2c1CC(N)C2 ; COC(=O)CCc1cc2c(cc1O)CC(N)C2 ; COC(=O)CCc1cc2c(cc1OC)CC(N)C2 ; CCOc1cc2c(cc1CCC(=O)OC)CC(N)C2]

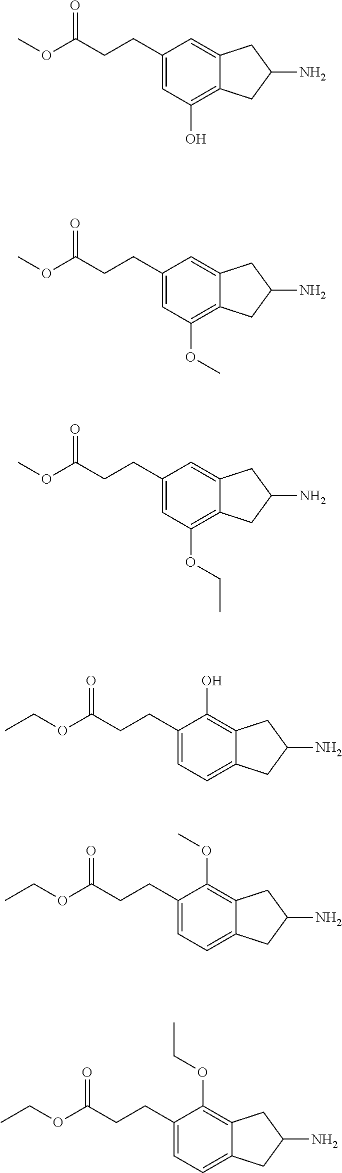 [SMILES: COC(=O)CCc1cc(O)c2c(c1)CC(N)C2 ; COC(=O)CCc1cc2c(c(OC)c1)CC(N)C2 ; CCOc1cc(CCC(=O)OC)cc2c1CC(N)C2 ; CCOC(=O)CCc1ccc2c(c1O)CC(N)C2 ; CCOC(=O)CCc1ccc2c(c1OC)CC(N)C2 ; CCOC(=O)CCc1ccc2c(c1OCC)CC(N)C2]

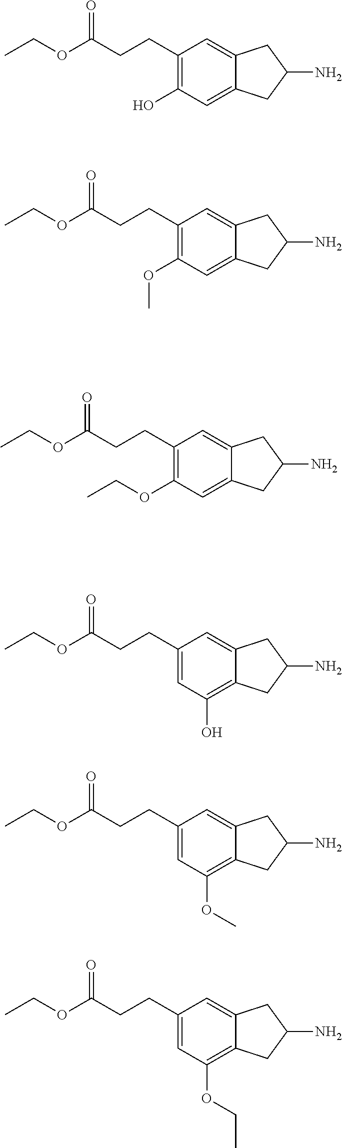 [SMILES: CCOC(=O)CCc1cc2c(cc1O)CC(N)C2 ; CCOC(=O)CCc1cc2c(cc1OC)CC(N)C2 ; CCOC(=O)CCc1cc2c(cc1OCC)CC(N)C2 ; CCOC(=O)CCc1cc(O)c2c(c1)CC(N)C2 ; CCOC(=O)CCc1cc2c(c(OC)c1)CC(N)C2 ; CCOC(=O)CCc1cc2c(c(OCC)c1)CC(N)C2]

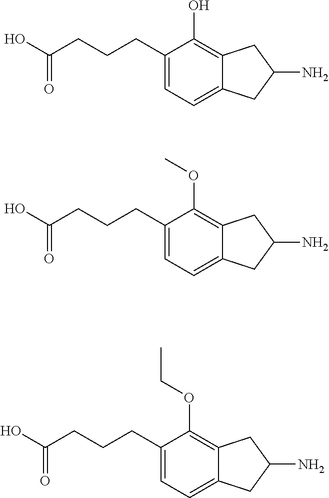 [SMILES: NC1Cc2ccc(CCCC(=O)O)c(O)c2C1 ; COc1c(CCCC(=O)O)ccc2c1CC(N)C2 ; CCOc1c(CCCC(=O)O)ccc2c1CC(N)C2]

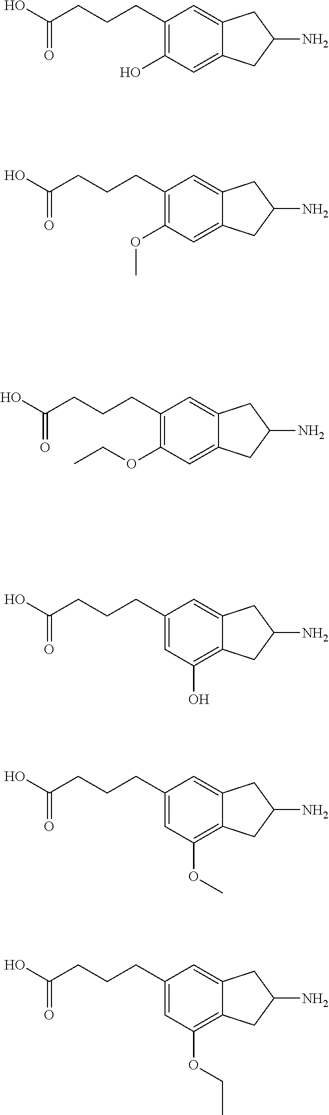 [SMILES: NC1Cc2cc(O)c(CCCC(=O)O)cc2C1 ; COc1cc2c(cc1CCCC(=O)O)CC(N)C2 ; CCOc1cc2c(cc1CCCC(=O)O)CC(N)C2 ; NC1Cc2cc(CCCC(=O)O)cc(O)c2C1 ; COc1cc(CCCC(=O)O)cc2c1CC(N)C2 ; CCOc1cc(CCCC(=O)O)cc2c1CC(N)C2]

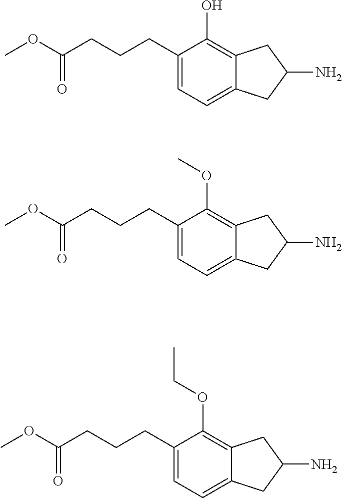 [SMILES: COC(=O)CCCc1ccc2c(c1O)CC(N)C2 ; COC(=O)CCCc1ccc2c(c1OC)CC(N)C2 ; CCOc1c(CCCC(=O)OC)ccc2c1CC(N)C2]

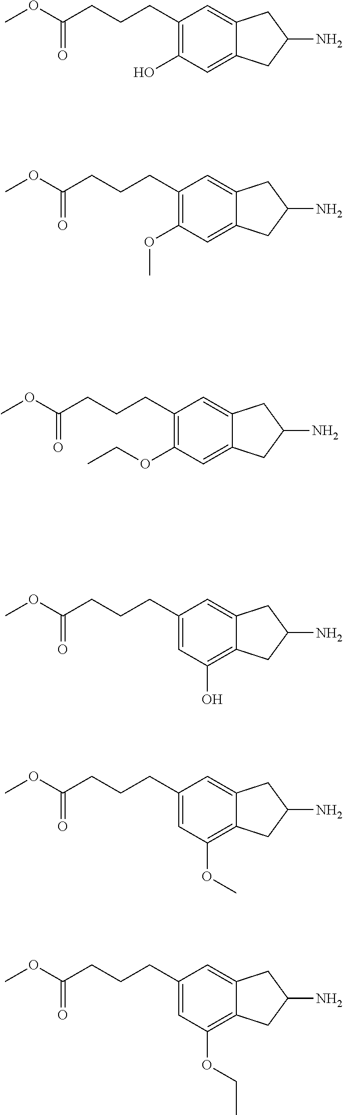 [SMILES: COC(=O)CCCc1cc2c(cc1O)CC(N)C2 ; COC(=O)CCCc1cc2c(cc1OC)CC(N)C2 ; CCOc1cc2c(cc1CCCC(=O)OC)CC(N)C2 ; COC(=O)CCCc1cc(O)c2c(c1)CC(N)C2 ; COC(=O)CCCc1cc2c(c(OC)c1)CC(N)C2 ; CCOc1cc(CCCC(=O)OC)cc2c1CC(N)C2]

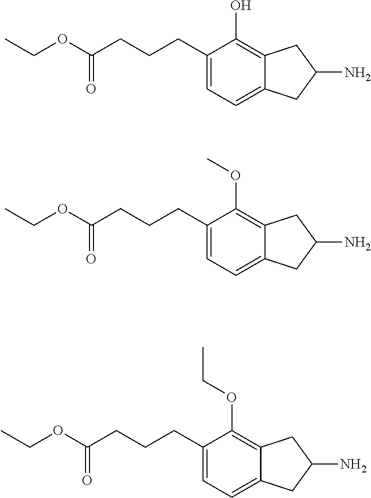 [SMILES: CCOC(=O)CCCc1ccc2c(c1O)CC(N)C2 ; CCOC(=O)CCCc1ccc2c(c1OC)CC(N)C2 ; CCOC(=O)CCCc1ccc2c(c1OCC)CC(N)C2]

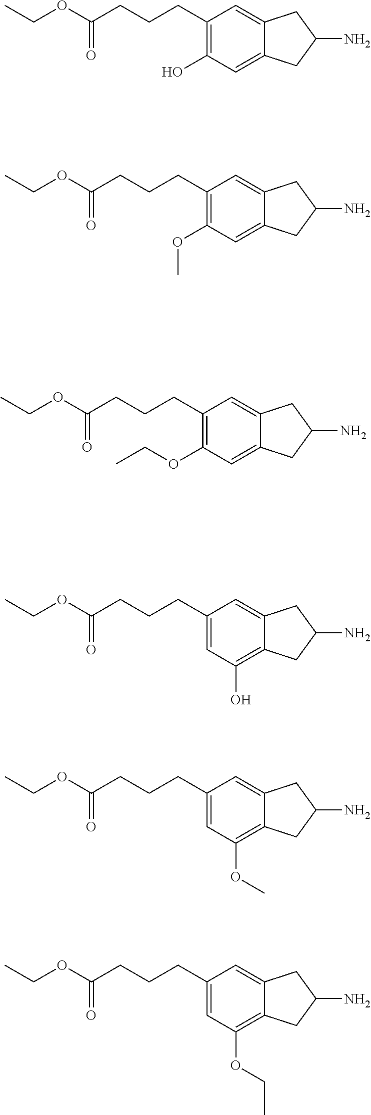 [SMILES: CCOC(=O)CCCc1cc2c(cc1O)CC(N)C2 ; CCOC(=O)CCCc1cc2c(cc1OC)CC(N)C2 ; CCOC(=O)CCCc1cc2c(cc1OCC)CC(N)C2 ; CCOC(=O)CCCc1cc(O)c2c(c1)CC(N)C2 ; CCOC(=O)CCCc1cc2c(c(OC)c1)CC(N)C2 ; CCOC(=O)CCCc1cc2c(c(OCC)c1)CC(N)C2]

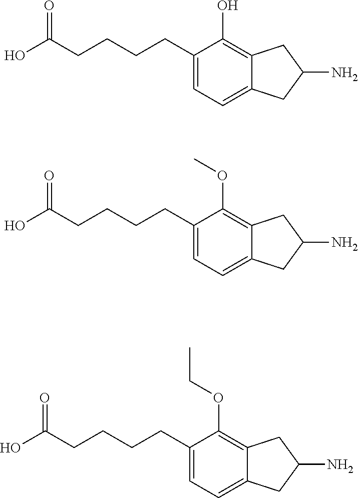 [SMILES: NC1Cc2ccc(CCCCC(=O)O)c(O)c2C1 ; COc1c(CCCCC(=O)O)ccc2c1CC(N)C2 ; CCOc1c(CCCCC(=O)O)ccc2c1CC(N)C2]

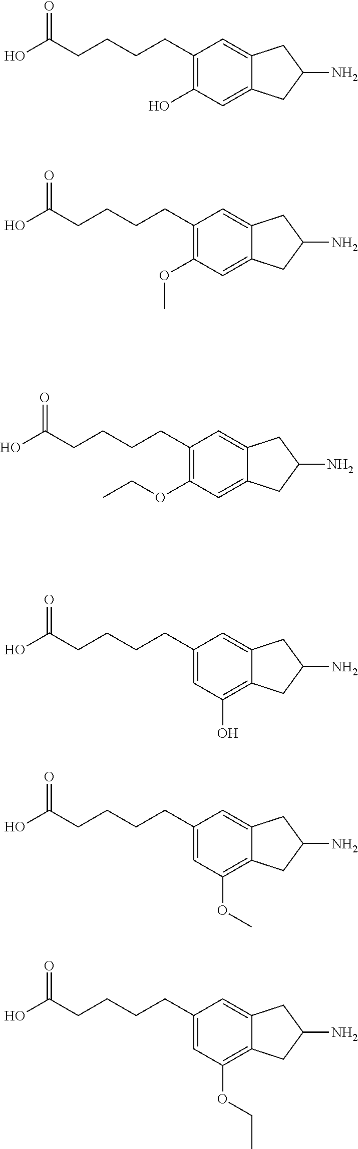 [SMILES: NC1Cc2cc(O)c(CCCCC(=O)O)cc2C1 ; COc1cc2c(cc1CCCCC(=O)O)CC(N)C2 ; CCOc1cc2c(cc1CCCCC(=O)O)CC(N)C2 ; NC1Cc2cc(CCCCC(=O)O)cc(O)c2C1 ; COc1cc(CCCCC(=O)O)cc2c1CC(N)C2 ; CCOc1cc(CCCCC(=O)O)cc2c1CC(N)C2]

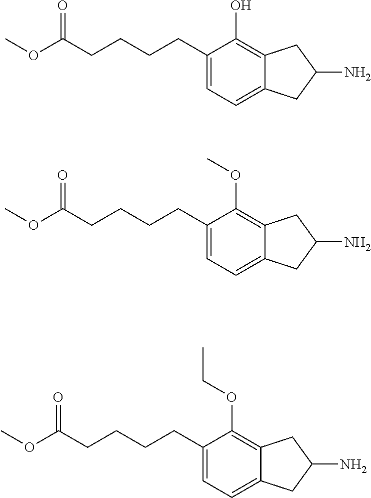 [SMILES: COC(=O)CCCCc1ccc2c(c1O)CC(N)C2 ; COC(=O)CCCCc1ccc2c(c1OC)CC(N)C2 ; CCOc1c(CCCCC(=O)OC)ccc2c1CC(N)C2]

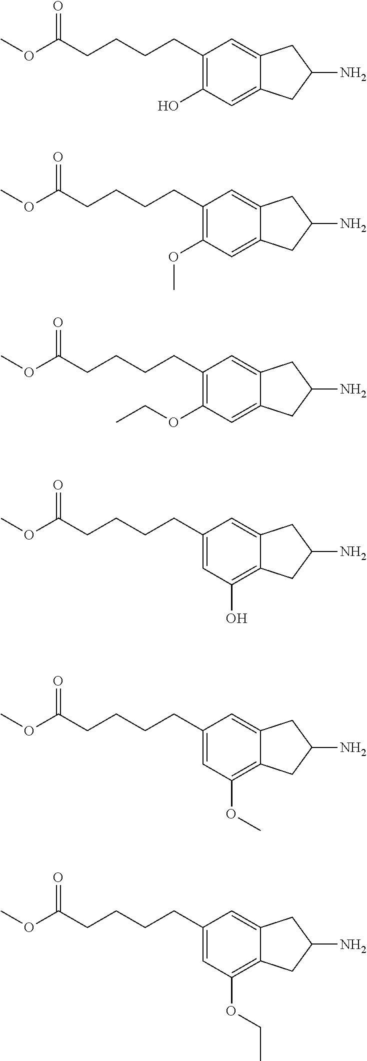 [SMILES: COC(=O)CCCCc1cc2c(cc1O)CC(N)C2 ; COC(=O)CCCCc1cc2c(cc1OC)CC(N)C2 ; CCOc1cc2c(cc1CCCCC(=O)OC)CC(N)C2 ; COC(=O)CCCCc1cc(O)c2c(c1)CC(N)C2 ; COC(=O)CCCCc1cc2c(c(OC)c1)CC(N)C2 ; CCOc1cc(CCCCC(=O)OC)cc2c1CC(N)C2]

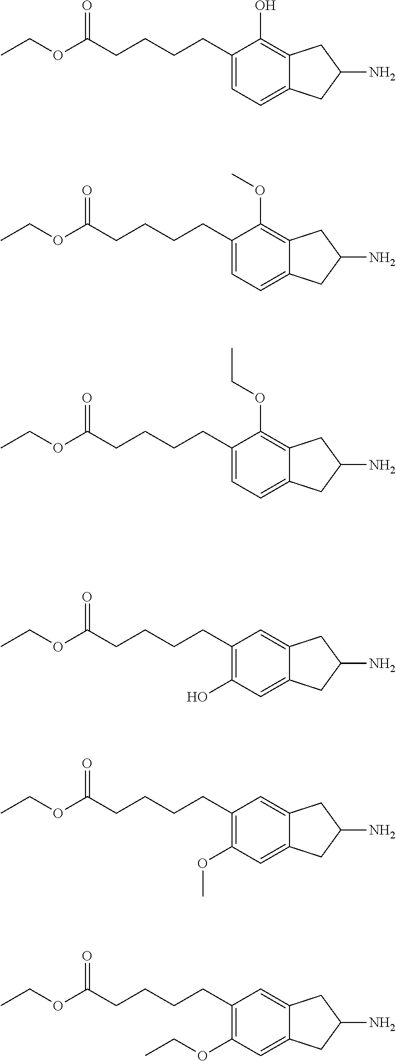 [SMILES: CCOC(=O)CCCCc1ccc2c(c1O)CC(N)C2 ; CCOC(=O)CCCCc1ccc2c(c1OC)CC(N)C2 ; CCOC(=O)CCCCc1ccc2c(c1OCC)CC(N)C2 ; CCOC(=O)CCCCc1cc2c(cc1O)CC(N)C2 ; CCOC(=O)CCCCc1cc2c(cc1OC)CC(N)C2 ; CCOC(=O)CCCCc1cc2c(cc1OCC)CC(N)C2]

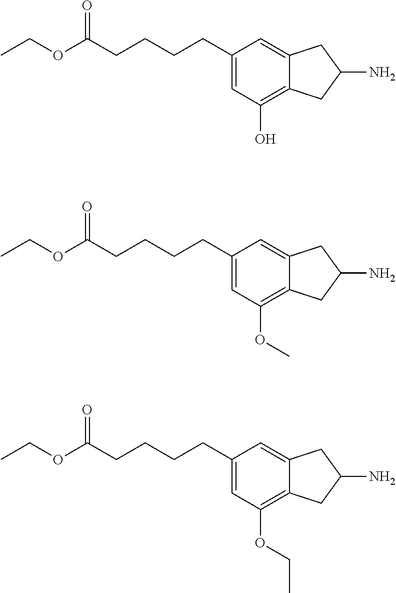 [SMILES: CCOC(=O)CCCCc1cc(O)c2c(c1)CC(N)C2 ; CCOC(=O)CCCCc1cc2c(c(OC)c1)CC(N)C2 ; CCOC(=O)CCCCc1cc2c(c(OCC)c1)CC(N)C2]

In some embodiments, the therapeutic compounds are those having a structure of Formula (In), and in exemplary such embodiments may be any one of the below:

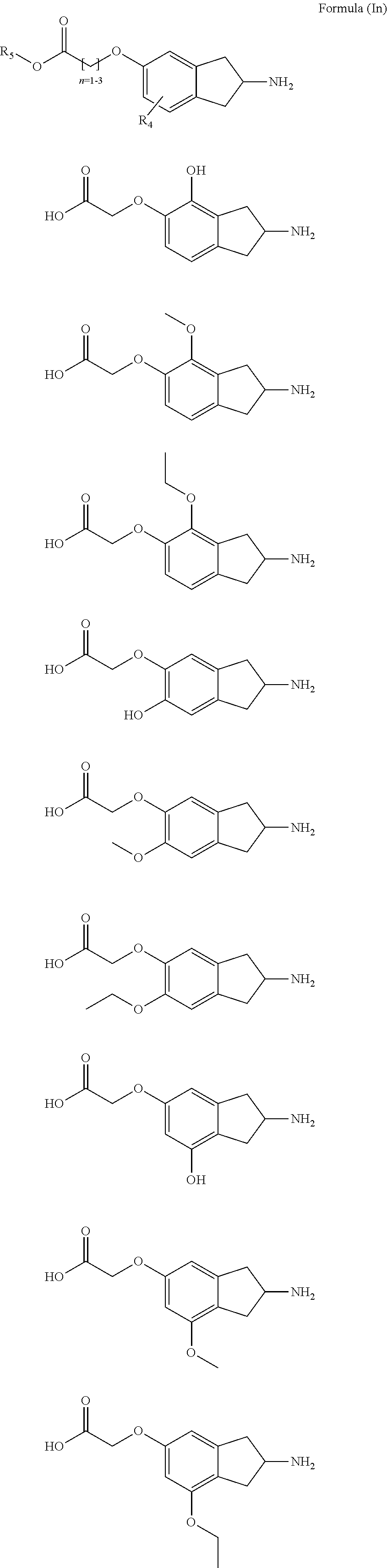

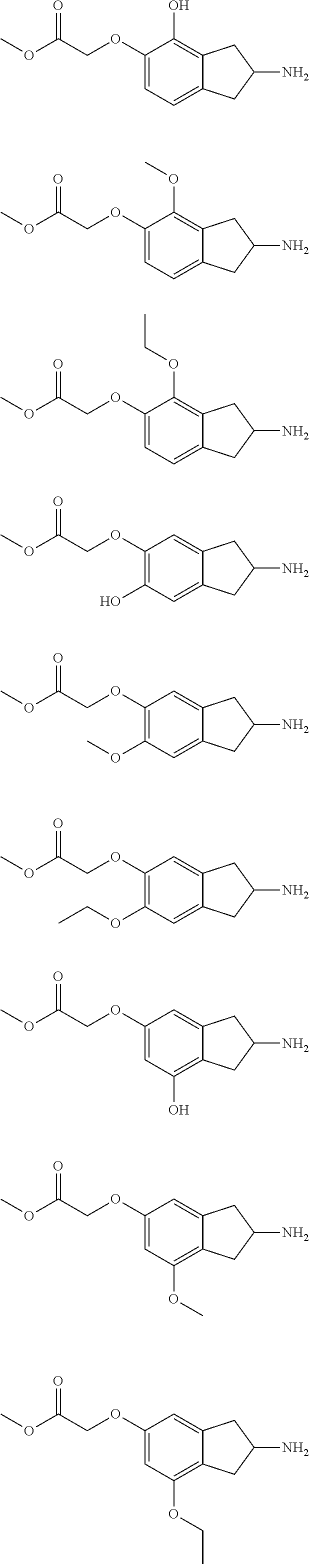

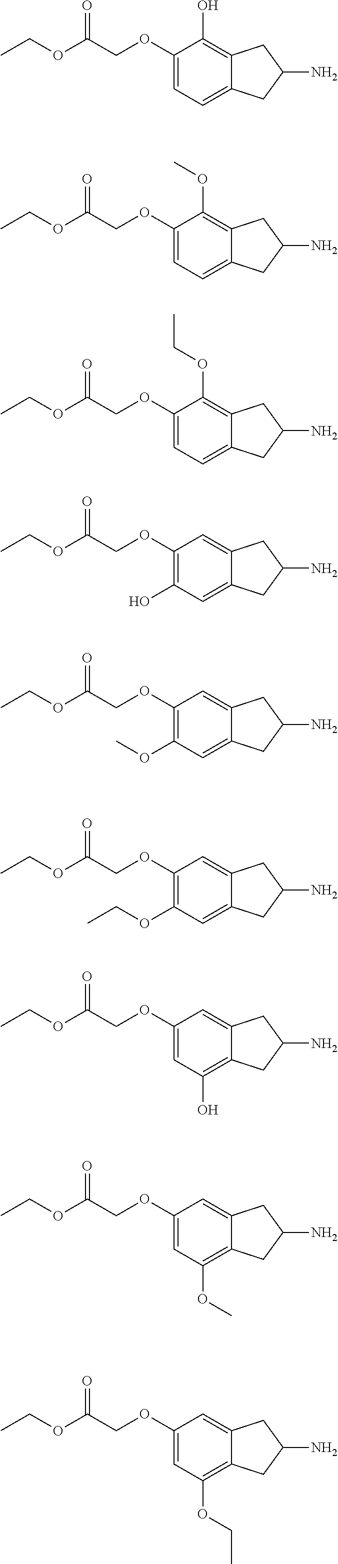

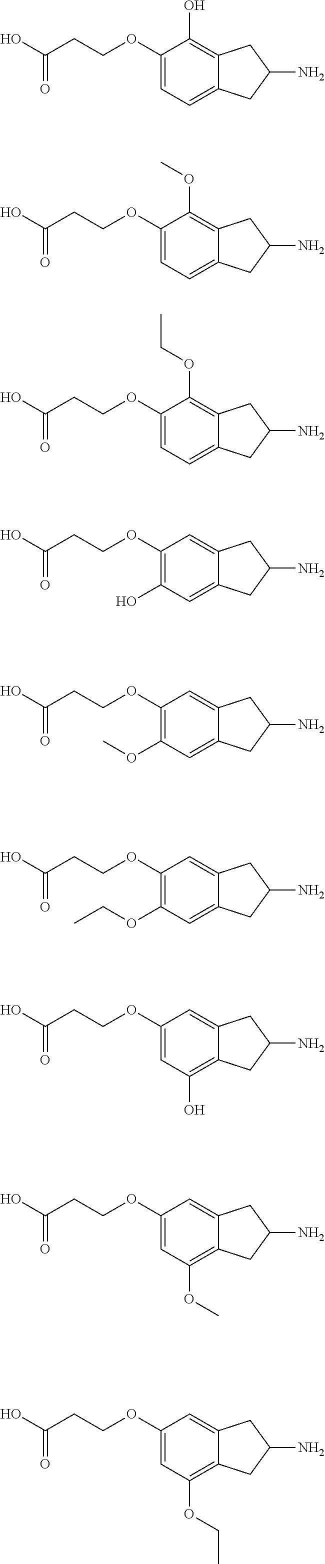

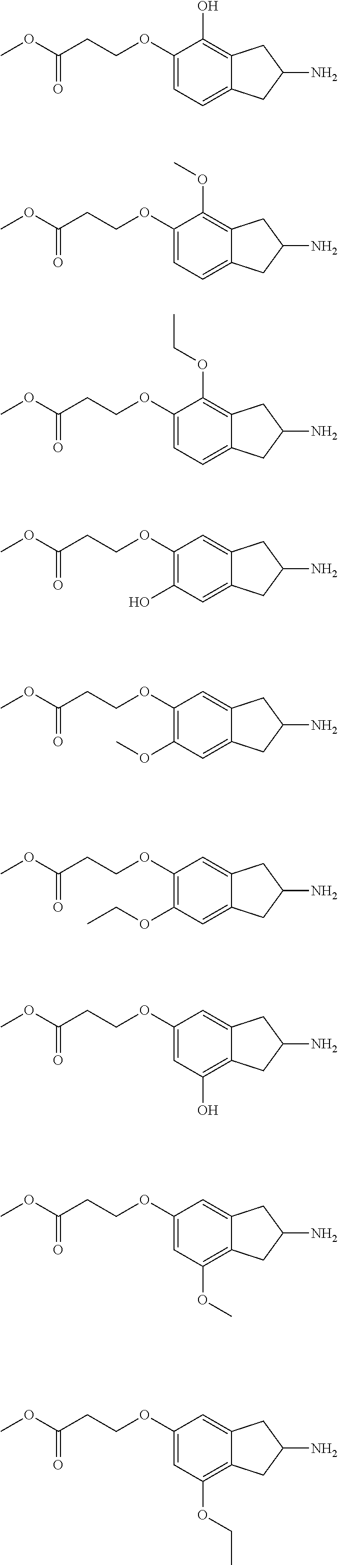

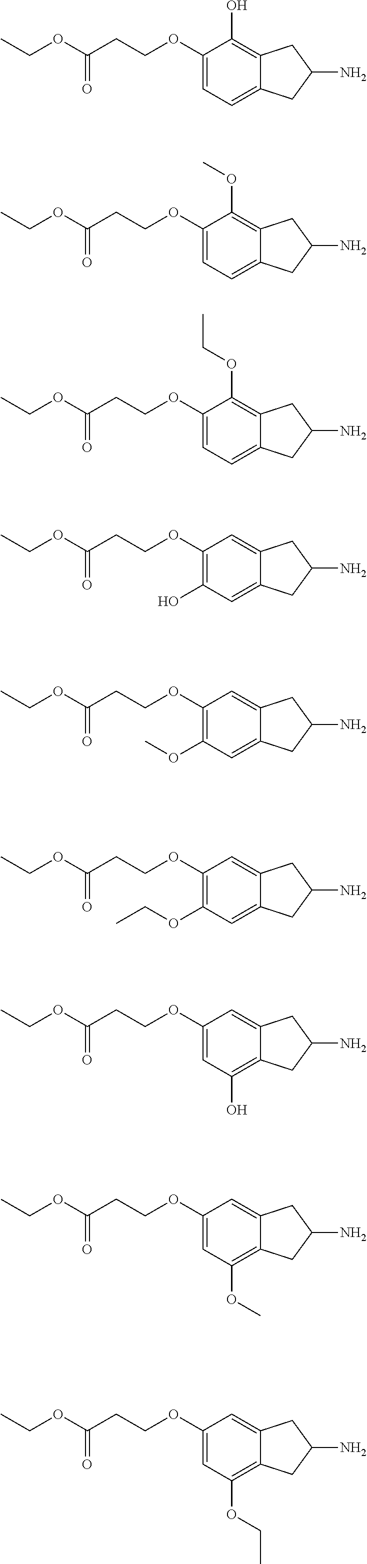

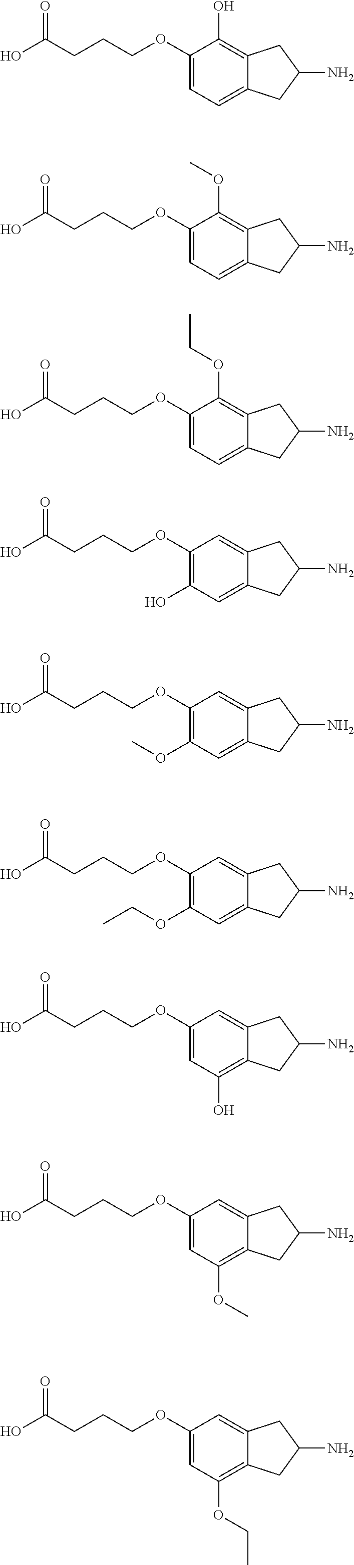

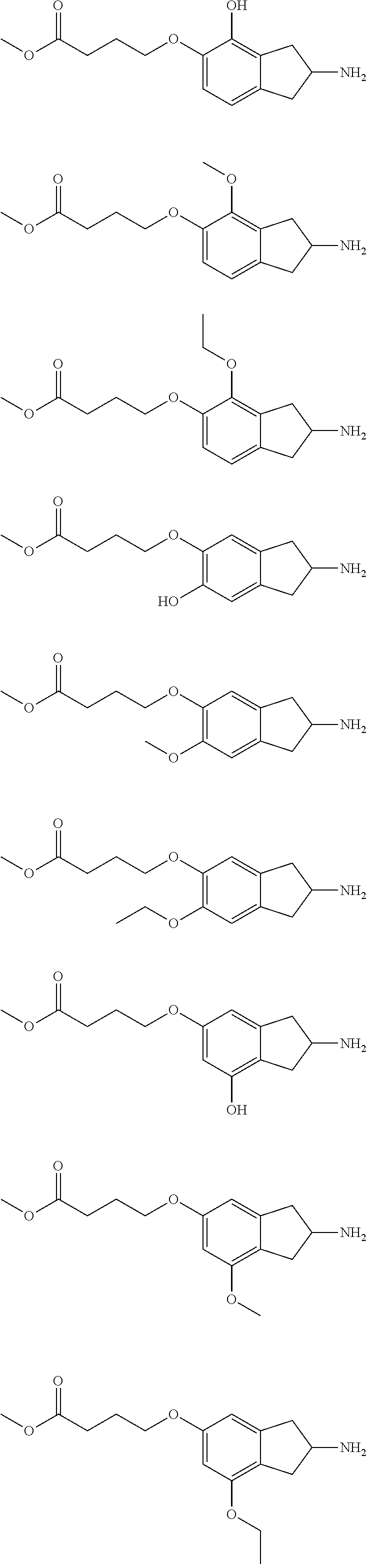

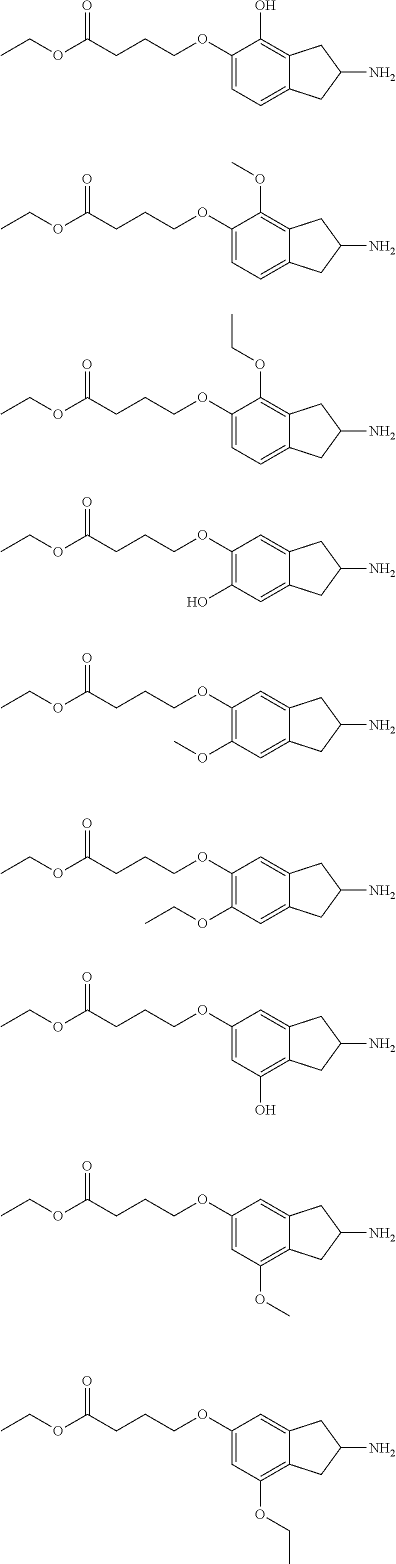

In some embodiments, the therapeutic compounds are those having a structure of Formula (Io), and in exemplary such embodiments may be any one of the below:

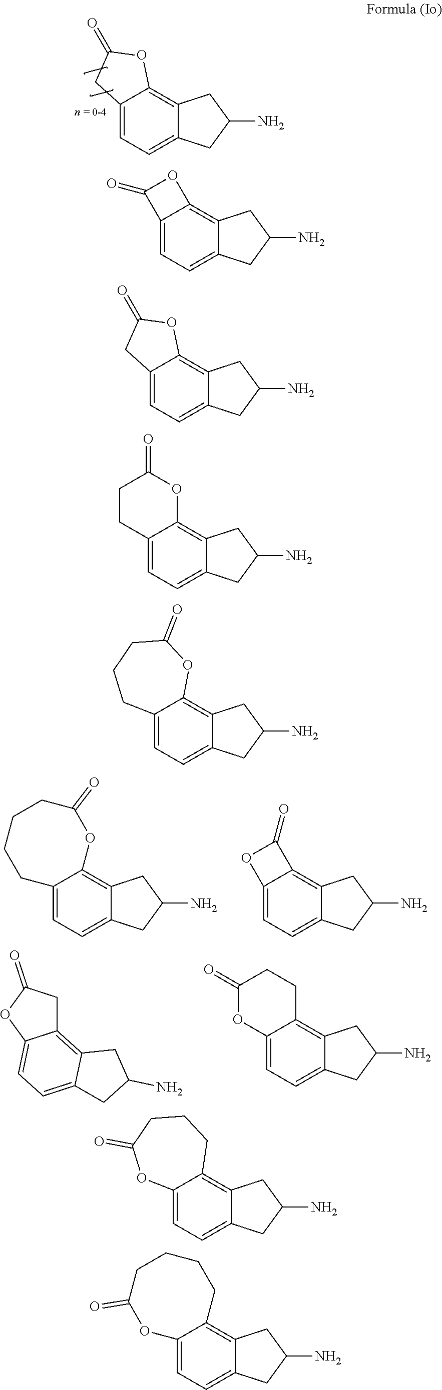

In some embodiments, the therapeutic compounds are those having a structure of Formula (Ip), and in exemplary such embodiments may be any one of the below:

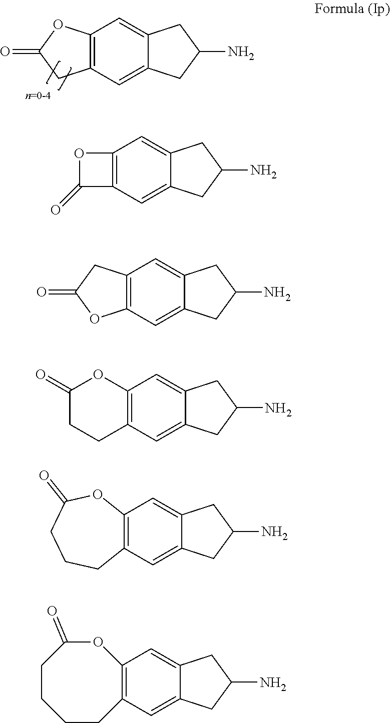

In some embodiments, the therapeutic compounds are those having a structure of Formula (Iq), and in exemplary such embodiments may be any one of the below:

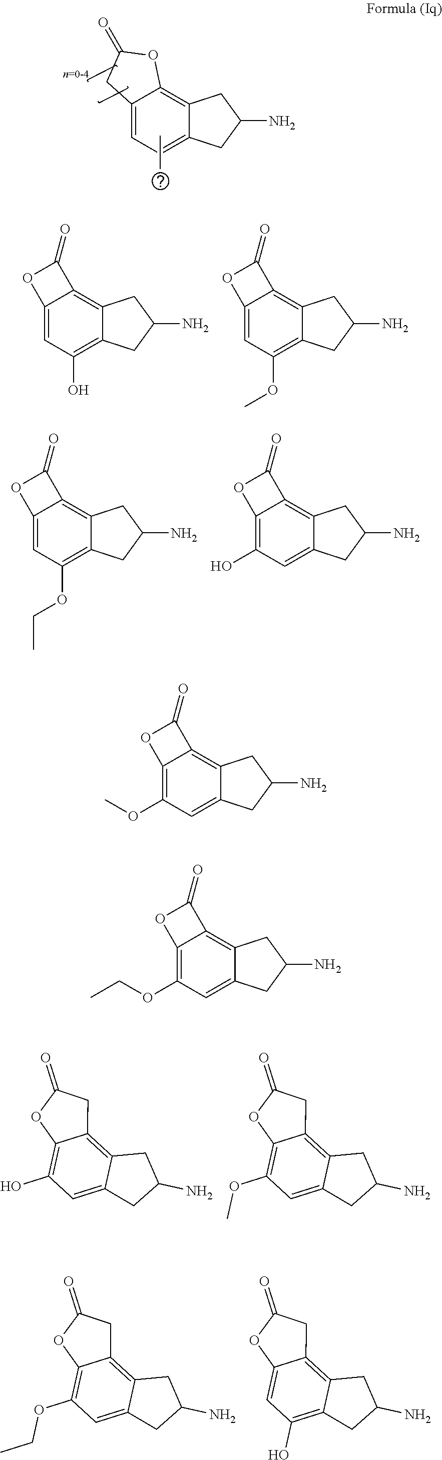

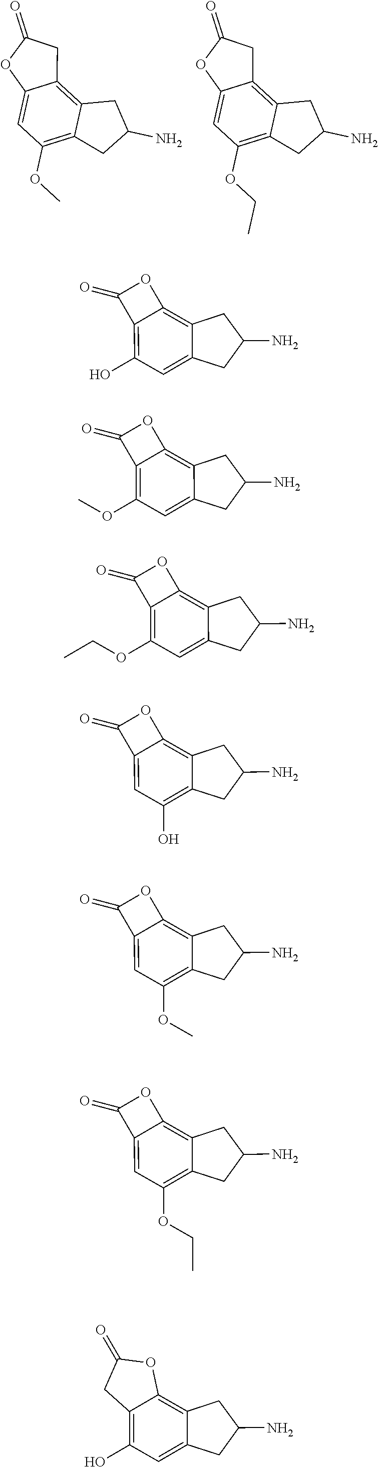

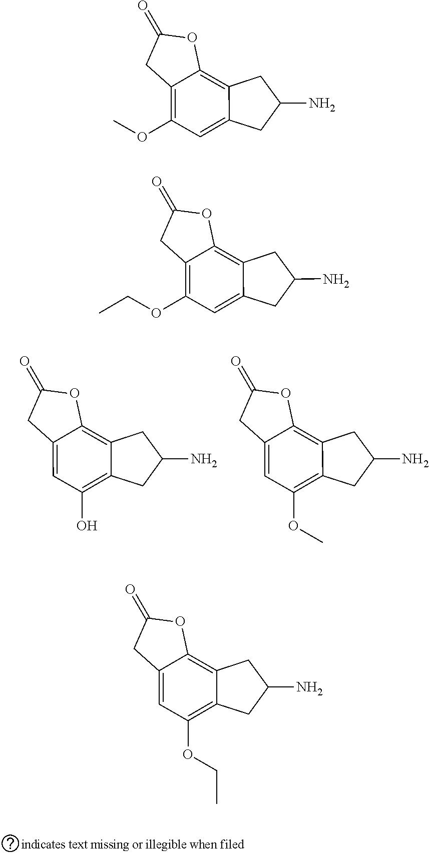

In some embodiments, the therapeutic compounds are those having a structure of Formula (Ir), and in exemplary such embodiments may be any one of the below:

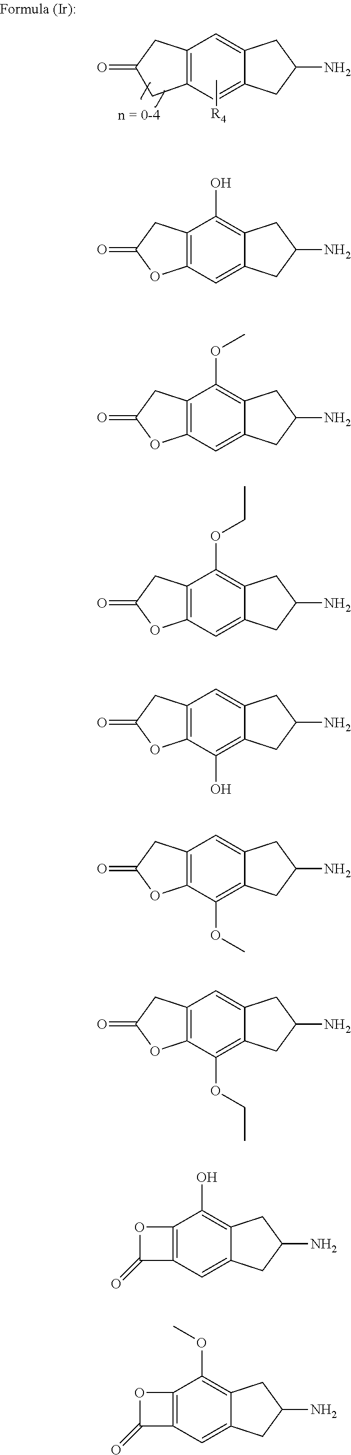

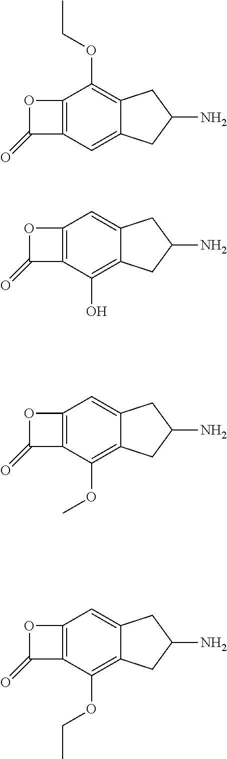 [SMILES: CCOc1c2c(cc3c1OC3=O)CC(N)C2 ; NC1Cc2cc3c(c(O)c2C1)C(=O)O3 ; COc1c2c(cc3c1C(=O)O3)CC(N)C2 ; CCOc1c2c(cc3c1C(=O)O3)CC(N)C2]

In some embodiments, a compound has a structure of any of the Formulas below, wherein R is defined as R₅ above. In some such embodiments, R is H, —CH₃, or —CH₂CH₃.

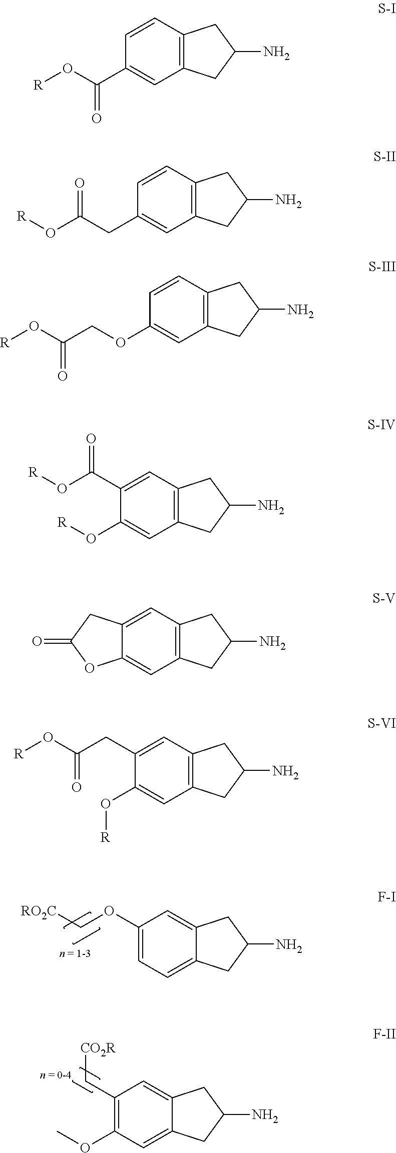

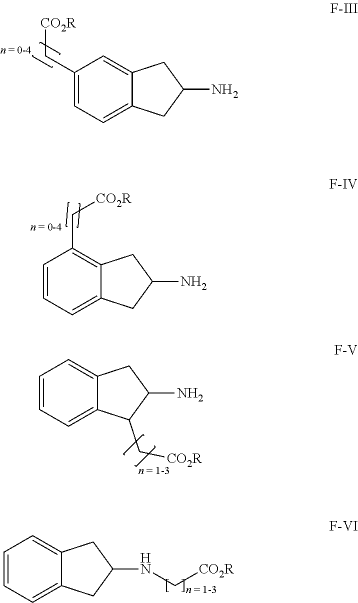

In some embodiments, a compound is one having a structure of Formula (Ig), wherein the structure is any one of:

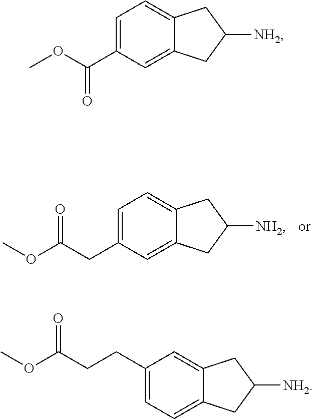 [SMILES: COC(=O)c1ccc2c(c1)CC(N)C2 ; COC(=O)Cc1ccc2c(c1)CC(N)C2 ; COC(=O)CCc1ccc2c(c1)CC(N)C2]

In some preferred embodiments of compounds having a structure of Formula (Ig), the compound is the 2-aminoindane-5-methylcarboxylate having the structure as follows, which herein also will be referred to as “CMP-405”:

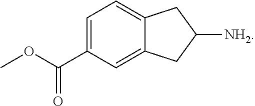

In some embodiments, a compound is one having a structure of Formula (Ih), wherein the structure is

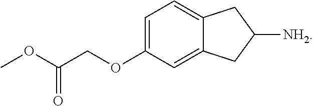 [SMILES: COC(=O)COc1ccc2c(c1)CC(N)C2]

In some embodiments, a compound is one having a structure of Formula (Im), wherein the structure is either

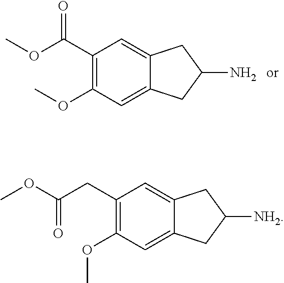 [SMILES: COC(=O)c1cc2c(cc1OC)CC(N)C2 ; "or" ; COC(=O)Cc1cc2c(cc1OC)CC(N)C2]

In some embodiments, a compound is one having a structure of Formula (in), wherein tlie structure is

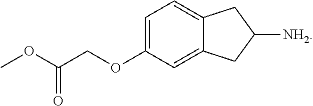 [SMILES: COC(=O)COc1ccc2c(c1)CC(N)C2]

In some embodiments, a compound is one having a structure of Formula (Ip), wherein the structure is

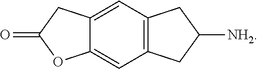 [SMILES: NC1Cc2cc3c(cc2C1)OC(=O)C3]

In some embodiments, a compound is one having the structures and names below:

| | |
|---|---|
| CMP-401: | 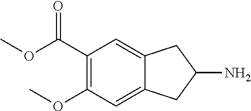 [SMILES: COC(=O)c1cc2c(cc1OC)CC(N)C2] |
| CMP-402: | 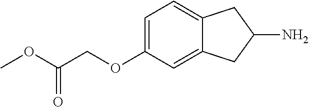 [SMILES: COC(=O)COc1ccc2c(c1)CC(N)C2] |
| CMP-405: | 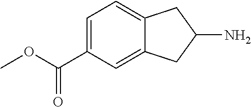 |
| CMP-406: | 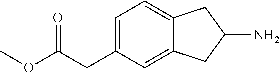 |
| CMP-407: | 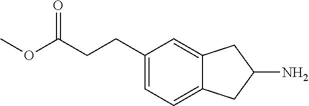 [SMILES: COC(=O)CCc1ccc2c(c1)CC(N)C2] |
| CMP-408: | 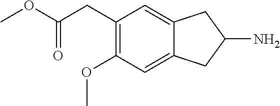 [SMILES: COC(=O)Cc1cc2c(cc1OC)CC(N)C2] |

Methods for synthesis of the therapeutic compounds and/or starting materials therefor are either described in the art or will be readily apparent to the skilled artisan in view of the teachings herein combined with general references well known in the art (see, e.g., Green et al., “Protective Groups in Organic Chemistry,” (Wiley, 2 nd ed. 1991); Harrison et al., “Compendium of Synthetic Organic Methods,” Vols. 1-8 (John Wiley and Sons, 1971-1996); “Beilstein Handbook of Organic Chemistry,” Beilstein Institute of Organic Chemistry, Frankfurt, Germany; Feiser et al, “Reagents for Organic Synthesis,” Volumes 1-17, Wiley Interscience; Trost et al., “Comprehensive Organic Synthesis,” Pergamon Press, 1991; “Theilheimer's Synthetic Methods of Organic Chemistry,” Volumes 1-45, Karger, 1991; March, “Advanced Organic Chemistry,” Wiley Interscience, 1991; Larock “Comprehensive Organic Transformations,” VCH Publishers, 1989; Paquette, “Encyclopedia of Reagents for Organic Synthesis,” John Wiley & Sons, 1995) and may be used to synthesize the therapeutic compounds. In general, the approaches used for similar compounds (Shulgin & Shulgin, PiHKAL, A chemical love story, Transform Press, Berkeley CA, 1992; Glennon et al., Journal of Medicinal Chemistry, 1986; 29(2), 194-199; Nichols et al., Journal of Medicinal Chemistry, 1991; 34(1), 276-281; Kedrowski et al., Organic Letters, 2007; 9(17), 3205-3207; Heravi & Zadsirjan, Current Organic Synthesis, 2016; 13(6), 780-833; Keri et al., European Journal of Medicinal Chemistry, 2017; 138, 1002-1033; PdrezSilanes et al., Journal of Heterocyclic Chemistry, 2001; 38(5), 1025-1030; and references therein), such adaptation being that known and understood to those of ordinary skill.

Certain therapeutic compounds may contain one or more asymmetric centers and give rise to enantiomers, diastereomers, and other stereoisomeric forms. Each chiral center may be defined, in terms of absolute stereochemistry, as (R)- or (S)-. The invention includes all such possible isomers, as well as mixtures thereof, including racemic and optically pure forms. In embodiments, enantiomerically enriched compounds may have an enantiomeric excess of one enantiomer of at least 50%, at least 60%, at least 65%, at least 70%, at least 75%, at least 80%, at least 85%, at least 90%, at least 95%, at least 98%, at least 99%, or at least 99.5%.

Optically active (R)- and (S)-, (−)- and (+)-, or (D)- and (L)-isomers may be prepared using chiral synthons or chiral reagents, or resolved using conventional techniques. When the therapeutic compounds contain olefinic double bonds or other centers of geometric asymmetry, and unless specified otherwise, it is intended that the compounds include both E and Z geometric isomers. Likewise, tautomeric forms are also intended to be included.

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods that are well known to practitioners in the art. Unless otherwise indicated, all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

Stereoisomers may include enantiomers, diastereomers, racemic mixtures, and combinations thereof. Such stereoisomers can be prepared and separated using conventional techniques, either by reacting enantiomeric starting materials, or by separating isomers of compounds disclosed herein.

Isomers may include geometric isomers. Examples of geometric isomers include cis isomers or trans isomers across a double bond. Other isomers are contemplated among the compounds of the present disclosure. The isomers may be used either in pure form or in admixture with other isomers of the therapeutic compounds.

Various methods are known in the art for preparing optically active forms and determining activity. Such methods include standard tests described herein and other similar tests which are well known in the art. Examples of methods that can be used to obtain optical isomers of the compounds according to the present disclosure include the following:
i) physical separation of crystals whereby macroscopic crystals of the individual enantiomers are manually separated. This technique may particularly be used if crystals of the separate enantiomers exist (i.e., the material is a conglomerate), and the crystals are visually distinct;
ii) simultaneous crystallization whereby the individual enantiomers are separately crystallized from a solution of the racemate, possible only if the latter is a conglomerate in the solid state;
iii) enzymatic resolutions whereby partial or complete separation of a racemate by virtue of differing rates of reaction for the enantiomers with an enzyme;
iv) enzymatic asymmetric synthesis, a synthetic technique whereby at least one step of the synthesis uses an enzymatic reaction to obtain an enantiomerically pure or enriched synthetic precursor of the desired enantiomer;
v) chemical asymmetric synthesis whereby the desired enantiomer is synthesized from an achiral precursor under conditions that produce asymmetry (i.e., chirality) in the product, which may be achieved using chiral catalysts or chiral auxiliaries;
vi) diastereomer separations whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomers;
viii) kinetic resolutions comprising partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors whereby the desired enantiomer is obtained from non-chiral starting materials and where the stereochemical integrity is not or is only minimally compromised over the course of the synthesis;
x) chiral liquid chromatography whereby the enantiomers of a racemate are separated in a liquid mobile phase by virtue of their differing interactions with a stationary phase. The stationary phase can be made of chiral material or the mobile phase can contain an additional chiral material to provoke the differing interactions;
xi) chiral gas chromatography whereby the racemate is volatilized and enantiomers are separated by virtue of their differing interactions in the gaseous mobile phase with a column containing a fixed non-racemic chiral adsorbent phase;
xii) extraction with chiral solvents whereby the enantiomers are separated by virtue of preferential dissolution of one enantiomer into a particular chiral solvent; and
xiii) transport across chiral membranes whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane, which allows only one enantiomer of the racemate to pass through.

Where therapeutic compounds have unfilled valencies, then the valencies are to be filled with hydrogens or isotopes thereof, e.g., hydrogen-1 (protium) and hydrogen-2 (deuterium). It also will be understood that the therapeutic compounds can be labeled isotopically. Substitution with isotopes such as deuterium may afford certain therapeutic advantages resulting from greater metabolic stability, such as, for example, increased in vivo half-life or reduced dosage requirements, resulting for example from the “kinetic isotope effect.” Certain deuterated compounds may also produce fewer toxic metabolites. Accordingly, it will be appreciated that each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a therapeutic compound encompasses all potential isotopic forms (i.e., the compound being deuterated at the location of one or more hydrogens) unless the context clearly dictates otherwise.

Where therapeutic compounds contain one or more hydrogens, within the scope of said compounds are those wherein one or more of said hydrogens is replaced with a halogen. “Halogen” means any of fluoro (F), chloro (Cl), bromo (Br), or iodo (I), and any hydrogen in a therapeutic compound may be independently replaced with any such halogen.

It will be understood that therapeutic compounds as referred to herein will include their crystalline forms (or “polymorphs,” which include the different crystal packing arrangements of the same elemental composition of the compound), amorphous phases, salts, solvates, and hydrates. In some embodiments, the therapeutic compounds exist in solvated forms with pharmaceutically acceptable solvents such as water, ethanol, or the like. In other embodiments, the therapeutic compounds exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

The individual compounds of the compositions of the invention will be understood to also encompass pharmaceutically acceptable salts of such compounds. The term “pharmaceutically acceptable salt” refers to salts prepared from pharmaceutically acceptable non-toxic acids or bases, and which may be synthesized by conventional chemical methods. Generally, such salts are prepared by reacting the free acid or base forms of these agents with a stoichiometric amount of the appropriate base or acid in water or in an organic solvent, or in a mixture of the two; generally, nonaqueous media (e.g., ether, ethyl acetate, ethanol, isopropanol, or acetonitrile) are preferred. For therapeutic use, salts of the compounds are those wherein the counter-ion is pharmaceutically acceptable.

Exemplary salts include 2-hydroxyethanesulfonate, 2-naphthalenesulfonate, 3-hydroxy-2-naphthoate, 3-phenylpropionate, acetate, adipate, alginate, amsonate, aspartate, benzenesulfonate, benzoate, besylate, bicarbonate, bisulfate, bitartrate, borate, butyrate, calcium edetate, camphorate, camphorsulfonate, camsylate, carbonate, citrate, clavulariate, cyclopentanepropionate, digluconate, dodecylsulfate, edetate, edisylate, estolate, esylate, ethanesulfonate, fumarate, gluceptate, glucoheptanoate, gluconate, glutamate, glycerophosphate, glycollylarsanilate, hemisulfate, heptanoate, hexafluorophosphate, hexanoate, hexylresorcinate, hydrabamine, hydrobromide, hydrochloride, hydroiodide, hydroxynaphthoate, iodide, isothionate, lactate, lactobionate, laurate, laurylsulphonate, malate, maleate, mandelate, mesylate, methanesulfonate, methylbromide, methylnitrate, methylsulfate, mucate, naphthylate, napsylate, nicotinate, nitrate, N-methylglucamine ammonium salt, oleate, oxalate, palmitate, pamoate, pantothenate, pectinate, persulfate, phosphate, phosphateldiphosphate, picrate, pivalate, polygalacturonate, propionate, p-toluenesulfonate, saccharate, salicylate, stearate, subacetate, succinate, sulfate, sulfosaliculate, suramate, tannate, tartrate, teoclate, thiocyanate, tosylate, triethiodide, undecanoate, and valerate salts, and the like (see Berge et al., “Pharmaceutical Salts,” J. Pharm. Sci. 1977; 66:1-19). Preferred salts are those employing a hydrochloride anion.

The therapeutic compounds are generally administered as part of a pharmaceutical composition or formulation, but may be prepared for inclusion in such composition or formulations as isolated or purified compounds. The terms “isolated,” “purified,” or “substantially pure” as used herein, refer to material that is substantially or essentially free from components that normally accompany the material when the material is synthesized, manufactured, or otherwise produced. An “isolated,” “purified,” or “substantially pure” preparation of a compound is accordingly defined as a preparation having a chromatographic purity (of the desired compound) of greater than 90%, more preferably greater than 95%, more preferably greater than 96%, more preferably greater than 97%, more preferably greater than 98%, more preferably greater than 99% and most preferably greater than 99.5%, as determined by area normalization of an HPLC profile or other similar detection method. Preferably the substantially pure compound used in the invention is substantially free of any other active compounds which are not intended to be administered to a subject. In this context “substantially free” can be taken to mean that no active compound(s) other than the active compound intended to be administered to a subject are detectable by HPLC or other similar detection method, or are below a desired threshold of detection such as defined above.

In some embodiments, the therapeutic compounds provide entactogenic effects with a magnitude or intensity that is comparable to a known entactogen, such as MDMA, or is enhanced relative thereto. Non-limiting examples of determining the magnitude or intensity of an entactogenic experience include measuring blood plasma levels of various monoamines and collecting or rating subjective and/or observer reports of drug effects. Various measures of the entactogenic experience include the evaluating responses to stimuli, for example, as in the Multifaceted Empathy Test or the Face Emotion Recognition Test, and assessing responses to psychometric evaluations, for example, using the Psychometric Evaluation of the Altered States of Consciousness Rating Scale (OAV) (Studerus et al., PloS one, 2010; 5(8), e12412; Hysek et al., Social Cognitive and Affective Neuroscience, 2014; 9(11), 1645-1652). In some embodiments, the fast-acting and/or the short-acting therapeutic compounds provide subjective effects with a magnitude or intensity that is comparable to a known aminoindane, such as MEAI, or that is enhanced relative thereto.

In some embodiments, the compounds provided herein provide a rapid onset of therapeutic effect. In some embodiments, the compounds provided herein provide a short-term duration of action. In some embodiments, the compounds provided herein provide rapid onset of therapeutic effect and a short-term duration of action. As used herein, “fast-acting” is used to describe a compound that produces a therapeutic effect more rapidly than a comparator. As used herein, “short-acting” is used to describe a compound that exhibits a shorter duration of action relative to a comparator. In some embodiments, rapid onset of action and/or short duration of action is determined from subjective and/or objective measures of drug effects. In some embodiments, identifying compounds with rapid onset and/or abbreviated drug action involves comparing pharmacokinetic parameters, for example, between a parent compound and a derivative of the same, modified as described herein. Exemplary pharmacokinetic parameters, such as a relatively short half-life (t1/2), increased rate of clearance (C₁), reduced time to peak concentration (Tmax), and decreased area under the curve (AUC), may be representative of rapid onset and/or short-term duration of action.

Regarding an abbreviated duration of action, which may translate to improved accessibility, disclosed compounds with a greater clearance and a shorter half-life that retain high plasma exposure, relative to MDMA, may be selected. In some embodiments, the half-life of an exemplary compound is reduced relative to MDMA, such as by about 5%-75%, 10%-65%, 15%-60%, 20%-55%, or 25%-50%, wherein each range is inclusive. In some embodiments, the half-life of an exemplary compound is about 50%, 55%, 60%, 65%, 70%, 75%, 80%, 85%, 90%, or 95% that of the half-life of MDMA.

In some embodiments, a fast-acting and/or a short-acting compound as described herein exhibits a half-life (t1/2) that is reduced by at least about 5%, at least about 10%, at least about 15%, at least about 20%, at least about 25%, at least about 30%, at least about 35%, at least about 50%, at least about 75%, or at least about 100% relative to a comparator. In some embodiments, a fast-acting and/or a short-acting compound as described herein exhibits a clearance rate that is at least 5%, at least 10%, at least 15%, at least 20%, at least 25%, at least 30%, at least 35%, at least 50%, at least 75%, or at least 100% faster than a comparator. In some embodiments, a comparator comprises the parent compound of a fast-acting and/or a short-acting derivative, for example, an ester provided herein. In some embodiments a comparator is a known entactogen, for example, MDMA. In some embodiments a comparator is a known aminoindane, for example, MEAI.

In embodiments, a fast-acting and/or short-acting compound reaches peak concentration (Tmax) at least about 5%, at least about 10%, at least about 15%, at least about 20%, at least about 25%, at least about 30%, at least about 35%, at least about 50%, at least about 75%, or at least about 100% faster than a comparator. In some embodiments, the AUC of a fast-acting and/or short-acting compound is at least about 5%, at least about 10%, at least about 15%, at least about 20%, at least about 25%, at least about 30%, at least about 35%, at least about 50%, at least about 75%, or at least about 100% less than a comparator. In some embodiments, a comparator comprises the parent compound of a fast-acting and/or a short-acting derivative, for example, an ester provided herein. In some embodiments a comparator comprises a known entactogen, for example, MDMA

In embodiments, an exemplary compound is less metabolically stable than MDMA. In some embodiments, the clearance of an exemplary compound, as determined either in vitro or in vivo, is increased relative to MDMA. In some embodiments, the clearance of an exemplary compound is increased relative to MDMA, such as by about 5%-75%, 10%-65%, 15%-60%, 20%-55%, 25%-50%, or 30%-45% wherein each range is inclusive. In some embodiments, the clearanace of an exemplary compound is about 30%, 35%, 40%, 45%, 50%, 55%, 60%, 65%, 70%, 75%, 80%, 85%, 90%, or 95% greater than the clearance of MDMA.

Introduction of esters has been described in the pursuit of providing rapid onset anesthetics with a short duration, for example, midazolam and ketamine derivatives. Some have exhibited unpredictable effects on receptor binding and potency relative to the parent compound (Pacofsky et al., Bioorg Med. Chem., 2002; 12:3219-22; Dimitrov et al., Molecules, 2020; 25(12):2950). In some embodiments, the introduced ester does not prevent a compound provided herein from interacting with, such as binding to, a target, for example, a receptor or a transporter. In some embodiments, the acid metabolite of ester degradation displays a lower binding affinity to a target, exhibits reduced efficacy, or is less potent than the ester-containing compound. In some embodiments, the bulk of the introduced ester group may modify the duration of action, for example, the half-life, of a compound provided herein. In some embodiments, increasing the bulk of the introduced ester may extend a compound's duration of action, for example, increase the half-life. An exemplary non-limiting series of increasing ester bulk is provided: CO2Methyl<CO2Ethyl<CO2Propyl<CO2Butyl<CO2Pentyl<CO2Hexyl<CO2Heptyl<CO2Octyl.

In embodiments, rapid onset and/or short-acting properties are provided by introducing an ester into a structure provided herein, e.g., an aminoindane scaffold. In some embodiments, an internal ester may be introduced to provide a fast-acting derivative. In some embodiments, an internal ester may be introduced to provide a short-acting derivative. In some embodiments, an external ester may be introduced to provide a fast-acting derivative. In some embodiments, an external ester may be introduced to provide a short-acting derivative.

In some embodiments, biologically active pharmaceutical esters described herein may be “soft drugs.” Soft drugs may exert therapeutic effects, for example, by interacting with a target receptor or site of action, prior to rapid metabolic conversion into a biologically inactive metabolite. In addition to considering efficacy, but without being bound by theory, an aim of soft drug design is to leverage moieties that will yield inactive and/or non-toxic compounds after metabolism, such as a predictable one-step metabolic conversion (Buchwald & Bodor, Pharmazie. 2014; 69(6):403-13). Quick metabolism of soft drugs may yield various effects, including minimizing duration of action, eliminating exposure to toxic metabolites, and reducing any possible drug-drug interactions. For example, various esters of ketamine have exhibited diverse effects on potency, duration of action, and psychomimetic effects of the drug (Jose et al., Bioorg Med Chem., 2013; 21(17):5098-10; Harvey et al., Anesth Analg., 2015; 121(4):925-933; Dimitrov et al., Bioorg Med Chem. 2019; 27(7):1226-1231). Provided that CYP450 enzymes, the major metabolizers, are subject to saturation and inhibition, fast metabolism may be facilitated by directing metabolism to hydrolytic enzymes, for example, by incorporating a metabolically sensitive “soft spot,” such as an ester (Buchwald, Expert Opinion on Drug Metabolism & Toxicology, 2020; 16:8:645-650).

Several strategies exist for preparing soft drugs. Some major approaches described herein pertain to soft analogs, active metabolite-based soft drugs, inactive metabolite-based soft drugs, controlled release endogenous agents or natural soft drugs, and activated soft compounds. Soft analogs are close structural analogs of a known or lead compound wherein metabolically sensitive moieties have been introduced to modify inactivation and/or excretion properties. The active metabolite approach leverages the biotransformation of a compound. Selecting an active species of biotransformation that exerts a desired pharmacological effect and undergoes a predictable and singular conversion into an inactive metabolite is one example of this approach. In contrast, the inactive metabolite approach relies on chemically modifying an inactive form of a compound to produce a pharmacologically active compound.

A predictable one-step metabolic conversion would then restore the initial inactive compound and eliminate exposure to any toxic intermediates. Endogenous biologically active agents that are efficiently metabolized can be described as natural soft drugs. A pro-soft drug approach can be adopted to modify the metabolism and duration of action of such compounds, for example, when metabolism of a desired compound is so rapid that it interferes with or precludes a desired pharmacological effect. Activated soft compounds result from incorporating a moiety with known pharmacological activity into a non-toxic compound. After exerting therapeutic activity, activated soft compounds are metabolized into their original non-toxic state (Bhardwaj et al., Saudi Pharm J. 2014; 22(4):290-302).

Esterases, hydrolases that split ester bonds, are ubiquitously distributed throughout the body, and can facilitate metabolism in the plasma, gut, liver, and other tissues (Williams, Clin Pharmacokinet., 1985; 10(5):392-403). Hydrolytic degradation can inactivate an ester, for example, by introducing a change in charge and/or shape that diminishes the metabolite's binding affinity for the original target (Buchwald, Expert Opinion on Drug Metabolism & Toxicology, 2020; 16:8:645-650). However, like CYP enzymes, genetic polymorphisms of esterases exist, and the enzymes are subject to induction, inhibition, and altered activity resulting from liver disease (Laizure et al., Pharmacotherapy. 2013; 33(2):210-222). Additionally, the activity of esterases can be unpredictable. The activity of such enzymes varies across tissues and between individuals, and is influenced by a variety of factors, including age (Di, Current Drug Metabolism, 2019; 20(2):91-102).

In some embodiments, the compounds provided herein are quickly metabolized. In some embodiments, aspects of the metabolism of the compounds provided herein, such as the rate of metabolism, can be modified by adjusting the size and bulk of the R group in an ester (—CO₂R). In some embodiments, the compounds provided herein are rapidly inactivated and/or eliminated, such as in and from the body of a subject. In some embodiments, rapid inactivation and/or elimination of the compounds provided herein facilitates a short duration of action. In some embodiments, hydrolysis of esters of the compounds provided herein facilitates a predictable duration of action. In some embodiments, hydrolysis of esters of the compounds provided herein facilitates a short duration of action. In embodiments, the compounds provided herein exhibit a reduced duration of action relative to a comparator compound, such as MDMA.

Pro-soft drugs, which require metabolic transformation for conversion into an active soft drug, have been described (Mukker et al., J Pharm Sci. 2016; 105(9):2509-2514). “Prodrugs” are differentiated from soft drugs in that they must undergo metabolic conversion to become biologically active, whereas metabolism of soft drugs, which are delivered as active agents, promotes inactivation and excretion. Incorporation of an ester moiety is an approach in preparing prodrugs (Rautio et al., Nat Rev Drug Discov. 2008; 7(3):255-70). Examples of prodrugs using ester or phosphoramidate as biologically labile or cleavable (protecting) groups are disclosed in U.S. Pat. Nos. 6,875,751, 7,585,851, and 7,964,580.

Prodrugs of the therapeutic compounds also will be appreciated to be within the scope of the invention. The term “prodrug” refers to a precursor of a biologically active pharmaceutical agent. Prodrugs undergo a chemical or a metabolic conversion to become a biologically active pharmaceutical agent. A prodrug can be converted ex vivo to the biologically active pharmaceutical agent by chemical transformative processes. In vivo, a prodrug is converted to the biologically active pharmaceutical agent by the action of a metabolic process, an enzymatic process or a degradative process that removes the prodrug moiety, such as a glycoside or acetyl group, to form the biologically active pharmaceutical agent. Other examples include addition of hydroxy groups (Tsujikawa et al., Xenobiotica, 2011; 41(7), 578-584; Yamamoto et al., Xenobiotica, 1984; 14(11), 867-875), acyloxyalkoxycarbonyl derivatives, amino acids, or peptides (Vig et al. 2013. Advanced Drug Delivery Reviews, 65(10), 1370-1385), which are generally added to the amine, and can be removed within the body by chemical reactions or enzymes, but other prodrugs and precursors, at the amine and other sites, should be understood to be within the scope of the invention (Simplicio, Clancy, & Gilmer, Molecules, 2008; 13(3), 519-547; Shah, Chauhan, Chauhan, & Mishra (Eds.). 2020. Recent Advancement in Prodrugs. CRC Press).

Among derivatives of a compound are included its “physiologically functional derivatives,” which refers to physiologically tolerated chemical derivatives of the compound having the same physiological function thereof, for example, by being convertible in the body thereto, and which on administration to a mammal such as a human is able to form (directly or indirectly) the compound or an active metabolite thereof (acting therefore, like a prodrug), or by otherwise having the same physiological function, despite one or more structural differences. According to the present invention, examples of physiologically functional derivatives include esters, amides, carbamates, ureas, and heterocycles.

In some embodiments, the compounds provided herein are not converted, such as metabolized, into pharmacologically active metabolites. In some embodiments, the compounds provided herein are not converted, such as metabolized, into toxic metabolites, such as metabolites associated with side effects. In some embodiments, the compounds provided herein are not associated with side effects, such as following administration to a subject. In some embodiments, the compounds provided herein do not cause side effects, such as following administration to a subject.

In some aspects are provided combinations comprising a disclosed compound and an additional active agent. The goal of increasing an existing therapeutic effect, providing an additional therapeutic effect, increasing a desired property such as stability or shelf life, decreasing an unwanted effect or property, altering a property in a desirable way (e.g., pharmacokinetics or pharmacodynamics), modulating a desired system or pathway (e.g., a neurotransmitter system), providing synergistic effects or otherwise inducing synergy, also can be achieved by the inclusion of an additional active compound.

Synergistic effects will be understood as including increases in potency, bioactivity, bioaccessibility, bioavailability, or therapeutic effect (including one or more additional therapeutic effects), that are greater than the additive contributions of the components acting alone, and/or are greater than the contribution of the isolated compounds on their own Numerous methods known to those of skill in the art exist to determine whether there is synergy as to a particular effect, i.e., whether, when two or more components are mixed together, the effect is greater than the sum of the effects of the individual components when applied alone, thereby producing “1+1>2. One such method is the isobologram analysis (or contour method) (Huang et al., Front Pharmacol., 2019; 10:1222).

Additional therapeutic effects that may be provided, or therapeutic effects that may be increased in embodiments of the invention include, but are not limited to, antioxidant, anti inflammatory, analgesic, antineuropathic, antinociceptive, antimigraine, anxiolytic, antidepressant, antipsychotic, anti-PTSD, immunostimulant, anti cancer, antiemetic, orexigenic, anti-ulcer, antihistamine, antihypertensive, anticonvulsant, antiepileptic, bronchodilator, neuroprotective, entactogenic, empathogenic, entheogenic, euphoric, psychedelic, sedative, and stimulant effects. Additional active agents may be selected from the group consisting of: amino acids, antioxidants, anti-inflammatory agents, analgesics, antineuropathic and antinociceptive agents, antimigraine agents, anxiolytics, antidepressants, antipsychotics, anti-PTSD agents, immunostimulants, anti-cancer agents, antiemetics, orexigenics, antiulcer agents, antihistamines, antihypertensives, anticonvulsants, antiepileptics, bronchodilators, neuroprotectants, other entactogens and empathogens, entheogens, psychedelics, monoamine oxidase inhibitors, sedatives, stimulants, and vitamins. These agents may be in ion, freebase, or salt form, include polymorphs, and may be isomers.

In embodiments, the additional active agent is selected from the group consisting of an opioid antagonist (e.g., nalmefene, naltrexone), a CB-1 antagonist (e.g., rimonabant), a CRH1 antagonist (e.g., verucerfont, pexacerfont), a NK1R antagonist (e.g., tradipitant), an OTR agonist (e.g., oxytocin), a GABA agent (e.g., topiramate, baclofen, a benzodiazepine, such as alprazolam, diazepam or lorazepam), a voltage-gated sodium channel inhibitor (e.g., oxacarbazepine, valproic acid, zonisamide), a voltage-dependent calcium channel agonist (e.g., gabapentin, pregabalin), an α7 nicotinic acetylcholine receptor agonist (e.g., varenicline), a 5-HT3 antagonist (e.g., ondansetron), a 5-HT1A, receptor partial agonist (e.g., aripiprazole), a 5-HT2A receptor antagonist (e.g., quetiapine, olanzapine, mirtazapine), a 5-HT reuptake inhibitor (e.g., trazodone), a SERT inhibitor (e.g., duloxetine), an α 1 adrenoreceptor antagonist (e.g., doxazosin, prazosin), a glucocorticoid receptor antagonist (e.g., mifepristone), an al adrenoreceptor agonist (e.g., guanfacine), an AChE inhibitor (e.g., citicoline), a dopamine D2 receptor antagonist (e.g., tiapride), an α2 adrenoreceptor agonist (e.g., clonidine), an NMDA receptor antagonist (e.g., acamprosate) and an aldehyde dehydrogenase inhibitor (e.g., disulfiram), and pharmaceutically acceptable salts thereof.

In embodiments, the additional active agent is a serotonergic agent. A “serotonergic agent” may refer to a compound that binds to, blocks, activates, inhibits, or otherwise influences (e.g., via an allosteric reaction) activity at one or more serotonin receptors, including any one or more serotonin receptor subtypes. In embodiments, a serotonergic agent binds to a serotonin receptor. In embodiments, a serotonergic agent indirectly affects a serotonin receptor, e.g., via interactions affecting the reactivity of other molecules at the serotonin receptor. In embodiments, a serotonergic agent is an agonist, e.g., a compound activating a serotonin receptor. In embodiments, a serotonergic agent is an antagonist, e.g., a compound binding to but not activating a serotonin receptor, e.g., blocking a receptor. In embodiments, a serotonergic agent is an effector molecule, e.g., a compound binding to an enzyme for allosteric regulation. In embodiments, a serotonergic agent acts (either directly or indirectly) at more than one type of receptor, including receptors other than serotonergic or other monoaminergic receptors. In embodiments, a serotonergic agent blocks the serotonin transporter (SERT) and results in an elevation of the synaptic concentration of serotonin, and an increase of neurotransmission. In embodiments, a serotonergic agent acts as a reuptake modulator and inhibits the plasmalemmal transporter-mediated reuptake of serotonin from the synapse into the presynaptic neuron, leading to an increase in extracellular concentrations of serotonin and an increase in neurotransmission. In embodiments, a serotonergic agent inhibits the activity of one or both monoamine oxidase enzymes, resulting in an increase in concentrations of serotonin and an increase in neurotransmission. In embodiments, a serotonergic agent is an antidepressant or anxiolytic, such as an SSRI, serotonin-norepinephrine reuptake inhibitor (SNRI), tricyclic antidepressant (TCA), monoamine oxidase inhibitor (MAOI), or atypical antidepressant. In other embodiments, a serotonergic agent is selected from the group consisting of: (1) serotonin transport inhibitors; (2) serotonin receptor modulators; (3) serotonin reuptake inhibitors; (4) serotonin and norepinephrine reuptake inhibitors; (5) serotonin dopamine antagonists; (6) monoamine reuptake inhibitors; (7) pyridazinone aldose reductase inhibitors; (8) stimulants of serotonin receptors; (9) stimulants of serotonin synthesis; (10) serotonin receptor agonists; (11) serotonin receptor antagonists; and (12) serotonin metabolites.

In embodiments, the additional active agent is a phenethylamine disclosed in Shulgin and Shulgin, PIHKAL: A Chemical Love Story, Transform Press (1994), or a tryptamine disclosed in Shulgin and Shulgin, TIHKAL: The Continuation, Transform Press (1997), both of which are incorporated by reference herein as if fully set forth herein.

In some embodiments, the one or more additional active agents is co-administered with a therapeutic compound, where such co-administration may be separately, simultaneously, or sequentially. In some embodiments, the one or more additional active agents is formulated in a pharmaceutical composition together with a therapeutic compound, such as in a fixed-dose combination (FDC), or other single dosage form.

The therapeutic compounds now generally described will be more readily understood by reference to the following description and examples, which are included for purposes of illustration of certain aspects of embodiments of the invention. The following is not intended to limit the invention, as one of skill would recognize from the teachings and examples herein that other techniques and methods can satisfy the claims and be employed without departing from the scope and spirit of the invention. Indeed, while the invention has been particularly shown and described with reference to certain exemplary embodiments, it will be understood by those skilled in the art that various changes in form and details may be made without departing from the scope or spirit encompassed by the appended claims.

### E. COMPOSITIONS

In some aspects are provided compositions, such as pharmaceutical compositions, comprising the therapeutic compounds. Pharmaceutical compositions are compositions that include the therapeutic compound(s) together in an amount (for example, in a unit dosage form) with a pharmaceutically acceptable carrier, diluent, or excipient. It should be understood that some embodiments do not have a single carrier, diluent, or excipient alone, but include multiple carriers, diluents, and/or excipients.

Compositions can be prepared by standard pharmaceutical formulation techniques such as disclosed in Remington: The Science and Practice of Pharmacy (2005) 21th ed., Mack Publishing Co., Easton, Pa.; The Merck Index (1996) 12th ed., Merck Publishing Group, Whitehouse, N.J.; Pharm. Principles of Solid Dosage Forms (1993), Technomic Publishing Co., Inc., Lancaster, Pa.; and Ansel and Stoklosa, Pharm. Calculations (2001) 11th ed., Lippincott Williams & Wilkins, Baltimore, Md.; and Poznansky et al. Drug Delivery Systems (1980), R. L. Juliano, ed., Oxford, N.Y., pp. 253-315.

Although the compositions and formulations of the invention are herein referred to as “pharmaceuticals,” or for “pharmaceutical” purpose or preparation, it will be readily appreciated that the term simply means that a composition is contemplated or shown to possess therapeutic effects when administered for its intended purpose to a mammal, such as a human. It therefore will be understood that the pharmaceutical compositions described herein are useful regardless of the regulatory regime under which they are ultimately sold (e.g., as prescription pharmaceutical drug products or non-prescription over-the-counter (OTC) drug products), and even if not sold under a specific regulatory regime at all.

“Pharmaceutically acceptable” as used in connection with one or more ingredients means that the ingredients are generally safe and, within the scope of sound medical judgment, suitable for use in contact with the cells of humans and other animals without undue toxicity, irritation, allergic response, or complication, and commensurate with a reasonable risk/benefit ratio.

These compositions can be prepared as formulations suitable for administration by a variety of routes including intramuscular, intravenous, oral, mucosal (e.g., buccal, sublingual), rectal, transdermal, subcutaneous, intraperitoneal, inhaled, and intranasal. In some embodiments, disclosed therapeutic compounds are formulated for parenteral administration. In some embodiments, disclosed therapeutic compounds are formulated for intravenous administration. In making the compositions employed in the present invention the active ingredients are often mixed with an excipient, diluted by an excipient, or enclosed within such a carrier which can be in the form of a capsule, sachet, paper, or other container. When the excipient serves as a diluent, it can be a solid, semi-solid, or liquid material, which acts as a vehicle, carrier, or medium for the active ingredient. Thus, the compositions can be in the form of tablets, pills, lozenges, sachets, cachets, elixirs, suspensions, emulsions, solutions, syrups, aerosols (as a solid or in a liquid medium), ointments, soft or hard gelatin capsules, suppositories, sterile injectable solutions, and sterile packaged powders. Different embodiments include immediate, delayed, extended, and controlled release forms. Many other variations are possible and known to those skilled in the art.

Another embodiment includes multiple routes of administration, which may differ in different patients according to patient preferences, comorbidities, side effect profiles, pharmacokinetic and pharmacodynamic considerations, and other factors. In some embodiments, therapeutic compounds are administered parenterally, i.e., via a parenteral route. In some embodiments, therapeutic compounds are administered intravenously, i.e., via the intravenous route. In some embodiments, therapeutic compounds are administered orally, i.e., via the oral route. Another embodiment includes the presence of other substances with the therapeutic compounds, known to those skilled in the art, such as fillers, carriers, gels, skin patches, lozenges, or other modifications in the preparation to facilitate absorption through various routes (e.g., gastrointestinal, transdermal, etc.), to extend the effect of the drugs, and/or attain higher or more stable serum levels or enhance the therapeutic effect of the therapeutic compounds in the combination.

In preparing a formulation, it may be necessary to mill an active compound to provide the appropriate particle size prior to combining with the other ingredients. If an active compound is substantially insoluble, it may be milled to a particle size of less than 200 mesh. If an active compound is substantially water soluble, the particle size is normally adjusted by milling to provide a substantially uniform distribution in the formulation, e.g., about 40 mesh.

Examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. Formulations can additionally include: lubricating agents such as talc, magnesium stearate, and mineral oil; wetting agents; emulsifying and suspending agents; preserving agents such as methyl- and propyl-hydroxybenzoates; sweetening agents; and flavoring agents. Compositions can be formulated so as to provide quick, sustained or delayed release of the therapeutic compounds after administration by employing procedures known in the art.

Pharmaceutical compositions can be formulated into any suitable dosage form, including aqueous oral dispersions, aqueous oral suspensions, solid dosage forms including oral solid dosage forms, aerosols, controlled release formulations, fast melt formulations, effervescent formulations, self-emulsifying dispersions, solid solutions, liposomal dispersions, lyophilized formulations, tablets, capsules, pills, powders, delayed-release formulations, immediate-release formulations, modified release formulations, extended-release formulations, pulsatile release formulations, multi particulate formulations, and mixed immediate release and controlled release formulations. Generally speaking, one will desire to administer an amount of the therapeutic compound that is effective to achieve a plasma level commensurate with the concentrations found to be effective in vivo for a period of time effective to elicit the desired therapeutic effect(s).

In some embodiments, therapeutic compounds are formulated for administration by injection, such as intravenous administration. Formulations suitable for intramuscular, intravenous, or subcutaneous injection may comprise physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propylene glycol, polyethylene-glycol, glycerol, cremophor and the like), suitable mixtures thereof, vegetable oils (such as olive oil) and injectable organic esters such as ethyl oleate. Additionally, therapeutic compounds can be dissolved at concentrations of >1 mg/mL using water-soluble beta cyclodextrins (e.g., beta-sulfobutyl-cyclodextrin and 2-hydroxypropylbetacyclodextrin). Proper fluidity can be maintained, for example, by the use of a coating such as a lecithin, by the maintenance of the required particle size in the case of dispersions, and by the use of surfactants.

Formulations suitable for injection may also contain additives such as preserving, wetting, emulsifying, and dispensing agents. Prevention of the growth of microorganisms can be ensured by various antibacterial and antifungal agents, such as parabens, benzoic acid, benzyl alcohol, chlorobutanol, phenol, sorbic acid, and the like. It may also be desirable to include isotonic agents, such as sugars, sodium chloride, and the like. Prolonged drug absorption of the injectable pharmaceutical form can be brought about by the use of agents delaying absorption, such as aluminum monostearate and gelatin. Suspension formulations designed for extended-release via subcutaneous or intramuscular injection can avoid first-pass metabolism and lower dosages of the therapeutic compounds will be necessary to maintain plasma levels of about 50 ng/mL. In such formulations, the particle size of the therapeutic compounds particles and the range of the particle sizes of the therapeutic compounds particles can be used to control the release of the drug by controlling the rate of dissolution in fat or muscle.

In some embodiments, the compositions are formulated in a unit dosage form. The term “unit dosage form” refers to a physically discrete unit suited as unitary dosages for the subject to be treated, each unit containing a predetermined quantity of active material calculated to produce the desired therapeutic effect(s), in association with a suitable pharmaceutical carrier, diluent, or excipient. Unit dosage forms are often used for ease of administration and uniformity of dosage. Unit dosage forms can contain a single or individual dose or unit, a sub-dose, or an appropriate fraction thereof (e.g., one half a “full” dose), of the pharmaceutical composition administered.

Unit dosage forms include capsules, troches, cachets, lozenges, tablets, ampules and vials, which may include a composition in a freeze-dried or lyophilized state; a sterile liquid carrier, for example, can be added prior to administration or delivery in vivo. Unit dosage forms also include ampules and vials with liquid compositions disposed therein. Unit dosage forms further include compounds for transdermal administration, such as “patches” that contact the epidermis of a subject for an extended or brief period of time.

In some embodiments, the compositions of the invention are formulated in a pharmaceutically acceptable oral dosage form. Oral dosage forms include oral liquid dosage forms and oral solid dosage forms.

a. Oral Liquid Dosage Forms

Oral liquid dosage forms include tinctures, drops, emulsions, syrups, elixirs, suspensions, and solutions, and the like. These oral liquid dosage forms may be formulated with any pharmaceutically acceptable excipient known to those of skill in the art for the preparation of liquid dosage forms, and with solvents, diluents, carriers, excipients, and the like chosen as appropriate to the solubility and other properties of the therapeutic compounds and other ingredients. Solvents may be, for example, water, glycerin, simple syrup, alcohol, medium chain triglycerides (MCT), and combinations thereof.

Liquid dosage forms for oral administration may be in the form of pharmaceutically acceptable emulsions, syrups, elixirs, suspensions, and solutions, which may contain an inactive diluent, such as water. Pharmaceutical formulations may be prepared as liquid suspensions or solutions using a sterile liquid, such as but not limited to, an oil, water, an alcohol, and combinations of these pharmaceutically suitable surfactants, suspending agents, emulsifying agents, may be added for oral or parenteral administration. Liquid formulations also may be prepared as single dose or multi-dose beverages. Suspensions may include oils. Such oils include peanut oil, sesame oil, cottonseed oil, corn oil, and olive oil. Suitable oils also include carrier oils such as MCT and long chain triglyceride (LCT) oils. Suspension preparation may also contain esters of fatty acids such as ethyl oleate, isopropyl myristate, fatty acid glycerides, and acetylated fatty acid glycerides. Suspension formulations may include alcohols, (such as ethanol, isopropyl alcohol, hexadecyl alcohol), glycerol, and propylene glycol. Ethers, such as poly(ethylene glycol), petroleum hydrocarbons such as mineral oil and petrolatum, and water may also be used in suspension formulations. Suspension can thus include an aqueous liquid or a non-aqueous liquid, an oil-in-water liquid emulsion, or a water-in-oil emulsion.

In some embodiments, formulations are provided comprising the compositions of the invention and at least one dispersing agent or suspending agent for oral administration to a subject. The formulation may be a powder and/or granules for suspension, and upon admixture with water, a substantially uniform suspension is obtained. The aqueous dispersion can comprise amorphous and non-amorphous particles consisting of multiple effective particle sizes such that a drug is absorbed in a controlled manner over time.

Dosage forms for oral administration can be aqueous suspensions selected from the group including pharmaceutically acceptable aqueous oral dispersions, emulsions, solutions, and syrups. See, e.g., Singh et al., Encyclopedia of Pharm. Tech., 2nd Ed., 754-757 (2002). In addition to the therapeutic compounds, the liquid dosage forms may comprise additives, such as one or more (a) disintegrating agents, (b) dispersing agents, (c) wetting agents, (d) preservatives, (e) viscosity enhancing agents, (f) sweetening agents, or (g) flavoring agents.

Examples of disintegrating agents for use in the aqueous suspensions and dispersions include a starch, e.g., a natural starch such as corn starch or potato starch, a pregelatinized starch, or sodium starch glycolate; a cellulose such as a wood product, microcrystalline cellulose, methylcellulose, croscarmellose, or a cross-linked cellulose, such as cross-linked sodium carboxymethylcellulose, cross-linked carboxymethylcellulose, or cross-linked croscarmellose; a cross-linked starch such as sodium starch glycolate; a cross-linked polymer such as crosspovidone; a cross-linked polyvinylpyrrolidone; alginate such as alginic acid or a salt of alginic acid such as sodium alginate; a clay; a gum such as agar, guar, locust bean, Karaya, pectin, or tragacanth; sodium starch glycolate; bentonite; a natural sponge; a surfactant; a resin such as a cation-exchange resin; citrus pulp; and sodium lauryl sulfate.

Examples of dispersing agents suitable for the aqueous suspensions and dispersions include hydrophilic polymers, electrolytes, Tween® 60 or 80, polyethylene glycol (PEG), polyvinylpyrrolidone (PVP), carbohydrate-based dispersing agents, noncrystalline cellulose, magnesium aluminum silicate, triethanolamine, polyvinyl alcohol (PVA), polyvinylpyrrolidone/vinyl acetate copolymer, poloxamers, and poloxamines.

Examples of wetting agents (including surfactants) suitable for the aqueous suspensions and dispersions include acetyl alcohol, glycerol monostearate, polyoxyethylene sorbitan fatty acid esters, PEG, oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, and phosphatidylcholine.

Examples of preservatives suitable for aqueous suspensions or dispersions include potassium sorbate, parabens (e.g., methylparaben and propylparaben) and their salts, benzoic acid and its salts, other esters of para hydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Examples of viscosity enhancing agents suitable for aqueous suspensions or dispersions include methyl cellulose, xanthan gum, carboxymethylcellulose, hydroxypropyl cellulose, hydroxypropylmethyl cellulose, Plasdone® S-630, carbomer, polyvinyl alcohol, alginates, acacia, chitosans, and combinations thereof. The concentration of the viscosity-enhancing agent will depend upon the agent selected and the viscosity desired.

In addition to the additives above, liquid formulations can also comprise inert diluents commonly used in the art, such as water or other solvents, solubilizing agents, emulsifiers, flavoring agents and/or sweeteners. Co-solvents and adjuvants also may be added to a formulation. Non-limiting examples of co-solvents contain hydroxyl groups or other polar groups, for example, alcohols, glycols, glycerol, polyoxyethylene alcohols, and polyoxyethylene fatty acid esters. Adjuvants include surfactants such as soy lecithin and oleic acid, sorbitan esters such as sorbitan trioleate, and PVP.

b. Oral Solid Dosage Forms

Oral solid dosage forms may include but are not limited to, lozenges, troches, tablets, capsules, caplets, powders, pellets, multiparticulates, beads, spheres, and/or any combinations thereof. Oral solid dosage forms may be formulated as immediate release, controlled release, sustained release, extended release, or modified release formulations. Accordingly, in some embodiments, oral solid dosage forms may be in the form of a tablet (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder), a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including a fast-melt tablet. Additionally, pharmaceutical formulations may be administered as a single capsule or in multiple capsule dosage form. In some embodiments, the pharmaceutical formulation is administered in 2, 3, 4, or more capsules or tablets.

Oral solid dosage forms may contain pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof. Oral solid dosage forms also can comprise one or more pharmaceutically acceptable additives such as a compatible carrier, complexing agent, ionic dispersion modulator, disintegrating agent, surfactant, lubricant, colorant, moistening agent, plasticizer, stabilizer, penetration enhancer, wetting agent, anti-foaming agent, alone or in combination, as well as supplementary active compound(s).

Supplementary active compounds include preservatives, antioxidants, antimicrobial agents including biocides and biostats such as antibacterial, antiviral and antifungal agents. Preservatives can be used to inhibit microbial growth or increase stability of the active ingredient thereby prolonging the shelf life of the pharmaceutical formulation. Suitable preservatives are known in the art and include EDTA, EGTA, benzalkonium chloride or benzoic acid or benzoates, such as sodium benzoate. Antioxidants include vitamin A, vitamin C (ascorbic acid), vitamin E, tocopherols, other vitamins or provitamins, and compounds such as alpha lipoic acid.

Using standard coating procedures, a film coating may be provided around the therapeutic compounds (see Remington: The Science and Practice of Pharmacy (2005) 21th ed., Mack Publishing Co., Easton, Pa, supra). In one embodiment, some or all of the therapeutic compounds are coated. In another embodiment, some or all of the therapeutic compounds are microencapsulated. In another embodiment, some or all of the therapeutic compounds is amorphous material coated and/or microencapsulated with inert excipients. In another embodiment, the therapeutic compounds are not microencapsulated and are uncoated.

Suitable carriers for use in oral solid dosage forms include acacia, gelatin, colloidal silicon dioxide, calcium glycerophosphate, calcium lactate, maltodextrin, glycerin, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose (HPMC), hydroxypropylmethylcellulose acetate stearate (HPMCAS), sucrose, microcrystalline cellulose, lactose, and mannitol.

Suitable filling agents for use in oral solid dosage forms include lactose, calcium carbonate, calcium phosphate, dibasic calcium phosphate, calcium sulfate, microcrystalline cellulose, cellulose powder, dextrose, dextrates, dextrose, dextran, starches, pregelatinized starch, HPMC, HPMCAS, hydroxypropylmethylcellulose phthalate, sucrose, xylitol, lactitol, mannitol, sorbitol, sodium chloride, and PEG.

Suitable disintegrants for use in oral solid dosage forms include those disclosed above for aqueous suspensions and dispersions. Suitable binders impart cohesiveness to solid oral dosage form formulations. For powder-filled capsules, they aid in plug formation that can be filled into soft or hard shell capsules. For tablets, they ensure that the tablet remains intact after compression and help assure blend uniformity prior to a compression or fill step. Materials suitable for use as binders in the solid dosage forms described herein include celluloses, microcrystalline dextrose, amylose, magnesium aluminum silicate, polysaccharide acids, bentonites, gelatin, polyvinylpyrrolidone/vinyl acetate copolymer, cross-povidone, povidone, starch, pregelatinized starch, tragacanth, dextrin, a sugar (e.g., sucrose, glucose, dextrose, molasses, mannitol, sorbitol, xylitol, lactose), a natural or synthetic gum (e.g., acacia, tragacanth, ghatti gum, mucilage of isapol husks), starch, PVP, larch arabogalactan, Veegum®, PEG, waxes, and sodium alginate.

In general, binder levels of 20-70% are used in powder-filled gelatin capsule formulations. Binder usage level in tablet formulations is a function of whether direct compression, wet granulation, roller compaction, or usage of other excipients such as fillers which itself can act as moderate binders are used. Formulators skilled in the art can determine binder level for formulations. In some embodiments, a disclosed composition comprises up to 70% of one or more binder(s).

Suitable lubricants or glidants for use in oral solid dosage forms include stearic acid, calcium hydroxide, talc, corn starch, sodium stearyl fumarate, alkali-metal and alkaline earth metal salts, stearic acid, sodium stearates, magnesium stearate, zinc stearate, waxes, Stearowet®, boric acid, sodium benzoate, sodium acetate, sodium chloride, leucine, PEG, methoxypolyethylene glycol, propylene glycol, sodium oleate, glyceryl behenate, glyceryl palmitostearate, glyceryl benzoate, and magnesium or sodium lauryl sulfate.

Suitable diluents for use in oral solid dosage forms include sugars (including lactose, sucrose, and dextrose), polysaccharides (including dextrates and maltodextrin), polyols (including mannitol, xylitol, and sorbitol), and cyclodextrins. Non-water-soluble diluents are compounds typically used in the formulation of pharmaceuticals, such as calcium phosphate, calcium sulfate, starches, modified starches, microcrystalline cellulose, and microcellulose (e.g., having a density of about 0.45 g/cm3, e.g., Avicel, powdered cellulose), and talc.

Suitable wetting agents for use in oral solid dosage forms include oleic acid, triethanolamine oleate, glyceryl monostearate, sorbitan monooleate, sorbitan monolaurate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, and vitamin E TPGS. Wetting agents include surfactants. Suitable surfactants for use in the solid dosage forms described herein include docusate and its pharmaceutically acceptable salts, sodium lauryl sulfate, sorbitan monooleate, polyoxyethylene sorbitan monooleate, polysorbates, poloxamers, bile salts, glyceryl monostearate, copolymers of ethylene oxide and propylene oxide, e.g., Pluronic® (BASF), and the like.

Suitable suspending agents for use in oral solid dosage forms include polyvinylpyrrolidone, PEG (having a molecular weight of about 300 to about 6000, or about 3350 to about 4000, or about 7000 to about 18000), vinylpyrrolidone/vinyl acetate copolymer (S630), sodium alginate, gums (e.g., gum tragacanth and gum acacia, guar gum, xanthans including xanthan gum), sugars, celluloses, polysorbate-80, polyethoxylated sorbitan monolaurate, polyethoxylated sorbitan monolaurate, and povidone.

Suitable antioxidants for use in oral solid dosage forms include butylated hydroxytoluene (BHT), butyl hydroxyanisole (BHA), sodium ascorbate, Vitamin E TPGS, ascorbic acid, sorbic acid, and tocopherol.

Immediate-release formulations may be prepared by combining a superdisintegrant such as croscarmellose sodium and different grades of microcrystalline cellulose in different ratios. To aid disintegration, sodium starch glycolate may be added.

In cases where different agents included in a fixed-dose combination are incompatible, cross-contamination can be avoided by incorporation of the agents in different layers in the oral dosage form with the inclusion of barrier layer(s) between the different layers, wherein the barrier layer(s) comprise inert and non-functional material(s).

The above-listed additives should be taken as merely exemplary types of additives that can be included in solid dosage forms. The amounts of such additives can be readily determined by one skilled in the art, according to the particular properties desired.

Tablets of the invention can be prepared by methods well known in the art. Various methods for the preparation of the immediate release, modified release, controlled release, and extended-release dosage forms (e.g., as matrix tablets having one or more modified, controlled, or extended-release layers) and the vehicles therein are well known in the art. For example, a tablet may be made by compression or molding. Compressed tablets may be prepared by compressing, in a suitable machine, an active ingredient in a free-flowing form such as a powder or granules, optionally mixed with a binder, lubricant, inert diluent, preservative, surface-active or dispersing agent. Molded tablets may be produced by molding, in a suitable apparatus, a mixture of powdered compound moistened with an inert liquid diluent. The tablets may optionally be coated or scored and may be formulated so as to provide a slow or controlled release of the active ingredient therein. Generally recognized compendia of methods include: Remington: The Science and Practice of Pharmacy (2005) 21th ed., Mack Publishing Co., Easton, Pa; Sheth et al. (1980), Compressed tablets, in Pharm. dosage forms, Vol. 1, Lieberman & Lachtman, eds., Dekker, NY.

In certain embodiments, solid dosage forms are prepared by mixing the therapeutic compounds with one or more pharmaceutical excipients to form a “bulk blend” composition. The bulk blend composition is homogeneous, i.e., the therapeutic compounds are dispersed evenly throughout so that the bulk blend may be readily subdivided into equally effective unit dosage forms, such as tablets, pills, and capsules. The individual unit dosages may also comprise film coatings, which disintegrate upon oral ingestion or upon contact with diluents. These formulations can be manufactured by conventional pharmaceutical techniques.

Pharmaceutical techniques for preparation of solid dosage forms include the following methods, which may be used alone or in combination: (1) dry mixing, (2) direct compression, (3) milling, (4) dry or non-aqueous granulation, (5) wet granulation, or (6) fusion. See, e.g., Lachman et al., Theory and Practice of Industrial Pharmacy (1986). Other methods include spray drying, pan coating, melt granulation, granulation, fluidized bed spray drying or coating (e.g., Wurster coating), tangential coating, top spraying, tableting, and extruding.

Compressed tablets are solid dosage forms prepared by compacting the bulk blend. In various embodiments, compressed tablets which are designed to dissolve in the mouth will comprise one or more flavoring agents. In other embodiments, the compressed tablets will comprise a film surrounding the final compressed tablet. In some embodiments, the film coating can provide a delayed release of the therapeutic compounds formulation. In other embodiments, the film coating aids in patient compliance (e.g., flavor or sweetener coatings).

Capsules may be prepared by placing the bulk blend inside of a capsule, such as a soft gelatin capsule, a standard gelatin capsule, or a non-gelatin capsule such as a capsule comprising HPMC. The bulk blend also may be placed in a sprinkle capsule, wherein the capsule may be swallowed whole or the capsule may be opened and the contents sprinkled on food prior to eating. In some embodiments, the therapeutic dose is split into multiple capsules. In some embodiments, the entire dose of the therapeutic compounds is delivered in a capsule form. In some embodiments the capsule is a size 000, size 00, or size 0 soft gelatin capsule. In other embodiments, the capsule is a size 1, size 2, size 3, or size 4 soft gelatin capsule. In other embodiments, the capsule is a hard gelatin capsule of equivalent size. Capsules can be capped and packaged using a manual capsule filling machine, or automated.

In certain embodiments, the formulations are fixed-dose pharmaceutical compositions with at least one other pharmacological agent, such as one or more additional active agents as disclosed herein. Fixed-dose combination formulations may contain therapeutically efficacious fixed-dose combinations of formulations of the therapeutic compounds and other pharmacological agents in the form of a single-layer monolithic tablet or multi-layered monolithic tablet or in the form of a core tablet-in-tablet or multi-layered multi-disk tablet or beads inside a capsule or tablets inside a capsule.

Depending on the desired release profile, oral solid dosage forms may be prepared as immediate release formulations, or as modified release formulations, such as controlled release, extended release, sustained release, or delayed release. In some embodiments, oral solid dosage forms are formulated as a delayed release dosage form by utilizing an enteric coating to affect release in the small intestine of the gastrointestinal tract. An enteric-coated oral dosage form may be a compressed or molded or extruded tablet/mold (coated or uncoated) containing granules, powder, pellets, beads or particles of the active ingredient and/or other composition components, which are themselves coated or uncoated. The enteric-coated oral dosage form may also be a capsule (coated or uncoated) containing pellets, beads or granules of the solid carrier or the composition, which are themselves coated or uncoated. Enteric coatings may also be used to prepare other controlled release dosage forms including extended release and pulsatile release dosage forms. Pulsatile release dosage forms may be formulated using techniques known in the art, such as described in U.S. Pat. Nos. 5,011,692, 5,017,381, 5,229,135, and 5,840,329. Other suitable dosage forms are described in U.S. Pat. Nos. 4,871,549, 5,260,068, 5,260,069, 5,508,040, 5,567,441 and 5,837,284. In one embodiment, the controlled release dosage form is pulsatile release solid oral dosage form comprising at least two groups of particles, each containing therapeutic compounds described herein. The first group of particles provides a substantially immediate dose of the therapeutic compound upon ingestion by a subject. The first group of particles can be either uncoated or comprise a coating and/or sealant. The second group of particles comprises coated particles, which may comprise from about 2% to about 75%, preferably from about 2.5% to about 70%, or from about 40% to about 70%, by weight of the total dose of the therapeutic compound, in admixture with one or more binders. Using such means, a single unit dosage form can provide both a first and a second dosage amount in the single form (i.e., the first dosage amount in an immediate release form, and the second dosage amount in a delayed release form).

In an embodiment, gastrorententive sustained release tablets are formulated by using a combination of hydrophilic polymer (e.g., hydroxypropyl methylcellulose), together with swelling agents (e.g., crospovidone, sodium starch glycolate, and croscarmelose sodium), and an effervescent substance (e.g., sodium bicarbonate). Using known methods, gastrorententive tablets can be formulated so as to prolong the gastric emptying time and extend the mean residence time (MRT) in the stomach for optimal drug release and absorption (see, e.g., Arza et al. Formulation and evaluation of swellable and floating gastroretentive ciprofloxacin hydrochloride tablets, AAPS PharmSciTech., 10(1):220-226 (2009)). Coatings for providing a controlled, delayed, or extended release may be applied to the pharmaceutical compositions of the invention or to a core containing the compositions. The coating may comprise a pharmaceutically acceptable ingredient in an amount sufficient, e.g., to provide an extended release from e.g., about 1 hour to about 7 hours following ingestion before release of the compositions. Suitable coatings include one or more differentially degradable coatings including pH-sensitive coatings (enteric coatings), or non-enteric coatings having variable thickness to provide differential release of the therapeutic compounds.

Many other types of modified release systems are known to those of ordinary skill in the art and are suitable for the formulations described herein. Examples of such delivery systems include both polymer- and non-polymer-based systems, silastic systems, peptide-based systems, wax coatings, bioerodible dosage forms, and compressed tablets using conventional binders (see, e.g., Liberman et al. Pharmaceutical Dosage Forms, 2 Ed., Vol. 1, pp. 209-214 (1990); Singh et al. Encyclopedia of Pharmaceutical Technology, 2nd Ed., pp. 751-753 (2002); U.S. Pat. Nos. 4,327,725; 4,624,848; 4,968,509; 5,461,140; 5,456,923; 5,516,527; 5,622,721; 5,686,105; 5,700,410; 5,977,175; 6,465,014; and 6,932,983).

c. Additional Dosage Forms

Pharmaceutical compositions comprising disclosed therapeutic compounds may be prepared as formulations suitable for intramuscular, subcutaneous, intraperitoneal, or intravenous injection, comprising physiologically acceptable sterile aqueous or non-aqueous solutions, dispersions, suspensions or emulsions, liposomes, and sterile powders for reconstitution into sterile injectable solutions or dispersions.

Examples of suitable aqueous and non-aqueous carriers, diluents, solvents, or vehicles include water, ethanol, polyols, suitable mixtures thereof, vegetable oils, and injectable organic esters such as ethyl oleate. Additionally, the compositions of the invention can be dissolved at concentrations of >1 mg/ml using water-soluble beta cyclodextrins (e.g., betasulfobutyl-cyclodextrin and 2-hydroxypropyl-betacyclodextrin. Proper fluidity can be maintained, for example, by the use of a coating such as a lecithin, by the maintenance of the required particle size in the case of dispersions, and by the use of surfactants.

Formulations suitable for subcutaneous injection may contain additives such as preserving, wetting, emulsifying, and dispensing agents. Prevention of the growth of microorganisms can be ensured by various antibacterial and antifungal agents, such as parabens, benzoic acid, benzyl alcohol, chlorobutanol, phenol, and sorbic acid. Isotonic agents, such as sugars and sodium chloride may be used. Prolonged drug absorption of an injectable form can be brought about by use of agents delaying absorption, e.g., aluminum monostearate or gelatin.

Pharmaceutical compositions may be prepared as suspension formulations designed for extended-release via subcutaneous or intramuscular injection. Such formulations avoid first-pass metabolism, and lower dosages of the compounds will be necessary to maintain equivalent plasma levels when compared to oral formulations. In such formulations, the mean particle size of the therapeutic compounds and the range of total particle sizes can be used to control the release of those agents by controlling the rate of dissolution in fat or muscle.

Pharmaceutical compositions may be prepared as effervescent powders containing the therapeutic compounds. Effervescent salts are used to disperse medicines in water for oral administration. Effervescent salts also may be packaged as single dose or multi-dose drink mixes, alone or in combination with other ingredients, such as vitamins or electrolytes. Effervescent salts are granules or coarse powders containing a medicinal agent in a dry mixture, usually composed of sodium bicarbonate and sodium carbonate, citric acid, and/or tartaric acid. When salts of the invention are added to water, the acids and the base react to liberate carbon dioxide gas, thereby causing “effervescence.” Any acid-base combination that results in the liberation of carbon dioxide can be used, as long as the ingredients are suitable for pharmaceutical use and result in a pH of about 6.0 or higher.

Pharmaceutical compositions may be prepared as nanostructured formulations such as nanoemulsions, nanocapsules, nanoparticle conjugates, or nano-encapsulated oral or nasal sprays. Preparations of such nanostructured formulations may be done by reference to the general knowledge of the art. (See, e.g., Jaiswal et al., Nanoemulsion: an advanced mode of drug delivery system, Biotech 3(5):123-27 (2015).)

The prefix “nano” as used in the terms describing various embodiments of a nanostructured formulation denotes a size range in the nanometer (“nm”) scale. Accordingly, sizes of such nanoparticle delivery vehicles include those in the about 1 to about 100 nm, about 100 to about 200 nm, about 200 to about 400 nm, about 400 to about 600 nm, about 600 to about 800 nm, and about 800 to about 1000 nm, as well as “microparticles” in the about 1000 to about 2000 nm (1-2 micrometer (“m”) scale). Particles of certain sizes may be particularly advantageous depending on the method of administration (e.g., for oral liquid emulsion versus for transdermal or topical application). Regardless of method of administration, one will appreciate that smaller particles provide for increased surface area over larger particles such that a higher concentration of therapeutic compound may be applied per volume of particles. A nanoparticle may be metal, lipid, polymer or other materials, or a combination of materials, and nanoparticles may be functionalized such that another moiety also may be attached thereto. Surface functionalization may involve the use of a moiety comprising an anchor group, a spacer and/or a functional group.

Lipid-based nanoparticles (LBNPs) such as liposomes, solid lipid nanoparticles (SLN), and nanostructured lipid carriers (NLC) can be used to transport both hydrophobic and hydrophilic molecules, and can be formulated to display very low or no toxicity, and increase the time of drug action by means of prolonged half-life and controlled release of therapeutic compounds. Lipid nanosystems also can include chemical modifications to avoid immune system detection (e.g., gangliosides or PEG) or to improve solubility of therapeutic compounds. In addition, such nanosystems can be prepared in formulations sensitive to pH so as to promote drug release in an acid environment.

The primary components of nanoparticles are phospholipids, which are organized in a bilayer structure due to their amphipathic properties. In presence of water, they form vesicles, improving the solubility and stability of the therapeutic compounds once they are loaded into their structure. Besides phospholipids, other compounds can be added to the formulations, such as cholesterol, which decreases the fluidity of the nanoparticle and increases the permeability of hydrophobic drugs through the bilayer membrane, improving stability of nanoparticles in blood. Cholesterol-modified liposomes may present a multiple bilayer with sizes from 0.5-10 nm, as multilaminar vesicles (MLVs); a single bilayer with sizes above 100 nm, as large unilamellar vesicles (LUVs); and intermediate sizes (10-100 nm), as small unilamellar vesicles (SUVs).

Pharmaceutical compositions may be prepared as a topical dosage form. Topical dosage forms include transmucosal and transdermal formulations, such as aerosols, emulsions, sprays, ointments, salves, gels, pastes, lotions, liniments, oils, and creams. For such formulations, penetrants and carriers can be included in the pharmaceutical composition. Penetrants are known in the art, and include, for transmucosal administration, detergents, bile salts, and fusidic acid derivatives. For transdermal administration, carriers which may be used include Vaseline®, lanolin, PEG, alcohols, transdermal enhancers, and combinations thereof.

An exemplary topical delivery system is a transdermal delivery device (“patch”) containing the therapeutic compounds. Such transdermal patches may be used to provide continuous or discontinuous infusion of the therapeutic compounds in controlled amounts. Such patches may be constructed for continuous, gradual, pulsatile, or on demand delivery of pharmaceutical agents. A “patch” within the meaning of the invention may be simply a medicated adhesive patch, i.e., a patch impregnated with a composition of the invention for application onto the skin. Thus, a patch may be a single-layer or multi-layer drug-in-adhesive patch, wherein the one or more adhesive layers also contain the therapeutic compounds. A patch may also be a “matrix” (or “monolithic”) patch, wherein the adhesive layer surrounds and overlays the drug layer (wherein a solution or suspension of the therapeutic compounds is in a semisolid matrix). A “reservoir” patch may also be used, comprising a drug layer, typically as a solution or suspension of the therapeutic compounds in a liquid compartment (i.e., the reservoir), separate from an adhesive layer. For example, the reservoir may be totally encapsulated in a shallow compartment molded from a drug-impermeable metallic plastic laminate, with a rate-controlling membrane made of vinyl acetate or a like polymer on one surface. A patch also may be part of a delivery system, for instance used with an electronic device communicatively coupled to the mobile device of a user, and coupled with a mobile application (e.g., to control the delivery rate from the reservoir, and optionally to provide information about delivery back to the application or user).

The following formulations are non-limiting examples of compositions comprising therapeutic compounds, wherein “therapeutic compound” refers to one or more of the disclosed compounds of the invention, such as those having a structure of Formula (I).

### Example 1: Formulation of Tablets

Tablets are prepared as follows:

| Ingredient | Quantity (mg/tablet) |
|---|---|
| Therapeutic Compound | 50.0 |
| Cellulose, microcrystalline | 200.0 |
| Colloidal silicon dioxide | 10.0 |
| Stearic acid | 5.0 |

The ingredients are blended and compressed to form tablets, each weighing 265 mg.

### Example 2: Alternate Formulation of Tablets

Scorable tablets are prepared as follows:

| Ingredient | Quantity (mg/tablet) |
|---|---|
| Therapeutic Compound | 75.0 |
| Starch | 45.0 |
| Microcrystalline cellulose | 35.0 |
| PVP (as 10% solution in water) | 4.0 |
| Sodium carboxymethyl starch | 4.5 |
| Magnesium stearate | 0.5 |
| Talc | 1.0 |

The therapeutic compound, starch and cellulose are passed through a No. 20 mesh U.S. sieve and mixed thoroughly. The solution of polyvinylpyrrolidone (PVP) is mixed with the resultant powders, which are then passed through a 16 mesh U.S. sieve. The granules so produced are dried at 50-60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets. Tablets are scored to provide the ability to create half doses.

### Example 3: Formulation of Capsules

Capsules are prepared as follows:

| Ingredient | Quantity (mg/capsule) |
|---|---|
| Therapeutic Compound | 50.0 |
| Starch | 134.0 |
| Magnesium stearate | 1.0 |

Therapeutic compound, cellulose, starch, and magnesium stearate are blended, passed through a No. 20 mesh U.S. sieve, and filled into hard or soft gelatin capsules.

### Example 4: Formulation of Suspension

Suspensions are prepared as follows:

| Ingredient | Amount | |
|---|---|---|
| Therapeutic Compound | 50.0 | mg |
| Xanthan gum | 4.0 | mg |
| Sodium carboxymethyl cellulose (11%) | 50.0 | mg |
| Microcrystalline cellulose (89%) | 50 | mg |
| Sucrose | 1.75 | g |
| Sodium benzoate | 10.0 | mg |

| | |
|---|---|
| Flavor and Color | q.v. |
| Purified water | To 5.0 ml |

The therapeutic compound, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### Example 5: Formulation of Intravenous Solution

An intravenous formulation is prepared as follows:

| Ingredient | Amount |
|---|---|
| Therapeutic Compound | 500 mg |
| Isotonic saline | 1000 mL |

Therapeutic compound is dissolved in appropriate solvent as will be understood by those of ordinary skill; isotonic saline is used in this Example, but it will be appreciated that other solvents may be used, and additional active or inactive ingredients such as preservatives may be added, as otherwise described above, and within the general knowledge of the art. It will be understood that the amount of therapeutic compound can be adjusted accordingly to reach desired mg/mL. IV fluid solution packagings come in different sizes, such as 50 mL, 100 mL, 250 mL, 500 mL, and 1000 mL.

### Example 6: Formulations of Injectable Solution

Injectable formulations are prepared as follows:

| Ingredient | Amount |
|---|---|
| Therapeutic Compound | 250 mg |
| Isotonic saline | 5 mL |

| Ingredient | Amount |
|---|---|
| Therapeutic Compound | 750 mg |
| Isotonic saline | 10 mL |

Therapeutic compound is dissolved in appropriate solvent as will be understood by those of ordinary skill; isotonic saline is used in this Example, but it will be appreciated that other solvents may be used, and additional active or inactive ingredients such as preservatives may be added, as otherwise described above, and within the general knowledge of the art.

### Example 7: Formulation of Topical Form

A topical formulation is prepared as follows:

| Ingredient | Amount (g) |
|---|---|
| Therapeutic Compound | 50.0 |
| Emulsifying Wax | 30.0 |
| Liquid Paraffin | 20.0 |
| White Soft Paraffin | To 100 |

The white soft paraffin is heated until molten. The liquid paraffin and emulsifying wax are incorporated and stirred until dissolved. Therapeutic compound is added and stirring is continued until dispersed. The mixture is then cooled until solid.

### Example 8: Formulation of Cut Matrix Sublingual or Buccal Tablets

Sublingual or buccal tablets cut into individual forms are prepared as follows:

| Ingredient | Amount (mg/tablet) |
|---|---|
| Therapeutic Compound | 50.0 |
| Glycerol | 210.5 |
| Water | 143.0 |
| Sodium Citrate | 4.5 |
| Polyvinyl Alcohol | 26.5 |
| Polyvinylpyrrolidone | 15.5 |

The glycerol, water, sodium citrate, polyvinyl alcohol, and polyvinylpyrrolidone are admixed together by continuous stirring and maintaining the temperature at about 90° C. When the polymers have gone into solution, the solution is cooled to about 50-55° C. and the medicament is slowly admixed. The homogenous mixture is poured into forms made of an inert material to produce a drug-containing diffusion matrix having a thickness of about 2-4 mm. This diffusion matrix is then cut to form individual tablets having the appropriate size.

### Example 9: Formulation of Individually Formed Sublingual or Buccal Lozenges

Sublingual or buccal lozenges from individual molds are prepared as follows:

| Ingredient | Amount (each lozenge) |
|---|---|
| Therapeutic Compound | 50.0 mg |
| Silica gel powder | 350.0 mg |
| Citric acid powder | 400.0 mg |
| Acacia powder | 600.0 mg |
| Flavoring (optional) | 100.0 mg |
| Polyethylene glycol | 1.0 g |

The inactive ingredients are admixed by continuous stirring and maintaining the temperature at about 90° C. When the PEG has melted and the other ingredients have gone into solution, the solution is cooled to about 50-55° C. and the therapeutic compound are slowly admixed. The homogenous mixture is poured into separate molds and allowed to cool. Reference may also be made to U.S. Pat. No. 10,034,832 and the Examples therein, the entirety of which is incorporated herein.

It should be readily appreciated that the above formulation examples are illustrative only. An “active agent” or “active ingredient” in the above examples will be understood to include the one or more compound(s) of the invention (therapeutic compounds) that comprise the formulation. Accordingly, any of the compounds may be substituted with the same compound in a different dosage amount. It will be understood that reference to particular compounds is merely illustrative, and both active and inactive compounds in any Example may be substituted by other therapeutic compounds.

Moreover, for any of the therapeutic compounds, substitution of the compound by its ion, free base, salt form, polymorph, solvate form, or an isomer or enantiomerically enriched mixture, shall be understood to provide merely an alternative embodiment still within the scope of the invention (with modifications to the formulation and dosage amounts made according to the teachings herein and ordinary skill, if necessary or desired). Further, compositions within the scope of the invention should be understood to be open-ended and may include additional active or inactive compounds and ingredients.

The type of formulation employed for the administration of the therapeutic compounds employed in the methods of the invention generally may be dictated by the compound(s) employed, the type of pharmacokinetic profile desired from the route of administration and the compound(s), and the state of the patient. It will be readily appreciated that any of the above embodiments and classes of embodiments can be combined to form additional embodiments and formulations.

d. Pharmaceutical Kits

In some aspects herein are pharmaceutical kits comprising one or more therapeutic compounds. As used herein, a kit is a packaged combination that optionally includes other elements, such as instructions for use or package insert, i.e., instructions customarily included in commercial packages of therapeutic products, that contain information, for example, prescribing information, about the indications, usage, dosage, administration, combination therapy, contraindications and/or warnings concerning the use of such therapeutic products.

In some embodiments, individual unit dosage forms can be included in multi-dose kits or containers. In some embodiments, contain package inserts and other printed instructions (e.g., on exterior packaging) for administering therapeutic compounds and compositions comprising the same use. In some embodiments, pharmaceutical formulations also can be packaged in single or multiple unit dosage forms for uniformity of dosage and ease of administration. In some embodiments, capsules, tablets, caplets, or other unit dosage forms comprising therapeutic compounds are packaged in blister packs. “Blister pack” refers to any of several types of pre-formed container, especially plastic packaging, that contains separate receptacles (e.g., cavities or pockets) for single unit doses, where such separate receptacles are individually sealed and can be opened individually.

Blister packs thus include such pharmaceutical blister packs known to those of ordinary skill, including Aclar® Rx160, Rx20e, SupRx, and UltRx 2000, 3000, 4000, and 6000 (Honeywell). Within the definition of multi-dose containers, and also often referred to as blister packs, are blister trays, blister cards, strip packs, push-through packs, and the like. In some embodiments, information pertaining to dosing and/or administration will be printed onto a multi-dose kit directly, e.g., on a blister pack or other interior packaging holding the therapeutic compounds or composition comprising the same.

### F. METHODS OF USE

In some aspects herein are methods and uses comprising the disclosed therapeutic compounds. In some embodiments, the therapeutic compounds are administered to a subject to modulate neurotransmission in a subject. In some embodiments, the therapeutic compounds are administered to a subject to treat one or more of a substance use disorder, a behavioral addiction, and a CNS or mental health disorder in a subject. In some embodiments, the therapeutic compounds are administered to a subject in conjunction with psychotherapy, which may include drug-assisted therapy or psychedelic-assisted therapy. In some embodiments, a pharmaceutical composition comprising one or more therapeutic compounds is administered to a subject.

Such methods and uses include therapeutic methods and uses, for example, involving administration of the therapeutic compounds to a subject having a SUD, behavioral addiction, CNS or mental health disorder, or otherwise in need of improving psychological function, thereby resulting in improvement of such disease or disorder, e.g., reduction of symptoms or improvement of functioning. Uses include uses of the therapeutic compounds or compositions thereof in such methods and treatments, and uses of the same in the preparation of a medicament in order to carry out such therapeutic methods. In some embodiments, the methods and uses thereby treat a substance use disorder, a behavioral addiction, a CNS or mental health disorder, or improve psychological functioning, in a subject.

a. Methods for Modulating Neurotransmission

In some aspects herein are methods of modulating neurotransmission in a subject by administering one or more therapeutic compound(s). In some embodiments, modulating neurotransmission comprises regulating one or more of monoamine neuromodulators, monoamine neurotransmitters, and monoamine neurotransmission. In some embodiments, monoamine neuromodulators and neurotransmitters include serotonin, dopamine, adrenaline (epinephrine), and noradrenaline (norepinephrine). In some embodiments, monoamine neurotransmission refers to one or more of serotonergic, dopaminergic, and noradrenergic neurotransmission. Such neuromodulators, neurotransmitters, and neurotransmission are known to one of skill (see, e.g., Hyman, Curr Biol., 2005; 15(5):R154-8).

Modulation of neurotransmission may include any of several mechanisms known to one of skill. Non-limiting examples of such mechanisms include, broadly, stimulating monoamine release (see, e.g., Gudelsky et al., Neurochem. 1996; 66:243-249) and inhibiting monoamine uptake, for example, by acting as a substrate for or blocking a monoamine transporter, such as SERT, DAT, or NET (see, e.g., Sandtner et al., Mol Pharmacol., 2016; 89(1):165-175 and Verrico et al., Psychopharmacology, 2007; 189(4):489-503).

In some embodiments, therapeutic compounds modulate neurotransmission. In some embodiments, modulating neurotransmission comprises regulating levels of monoamines in, for example, the CNS and peripheral tissues. In some embodiments, modulating neurotransmission comprises increasing levels of monoamines in, for example, the CNS and peripheral tissues of a subject to whom a therapeutic compound has been administered. In some embodiments, modulating neurotransmission comprises decreasing levels of monoamines in, for example, the CNS and peripheral tissues of a subject to whom a therapeutic compound has been administered. Detecting a change in monoamine levels in a subject, such as an increase or a decrease, can be achieved according to methods known to one of skill, for example, brain microdialysis (Chefer et al., Curr Protoc Neurosci. 2009; Chapter: Unit 7.1; Darvesh et al., Expert Opin Drug Discov. 2011; 6(2): 109-127) and brain imaging, for example, positron emission tomography (PET) and single photon emission computed tomography (SPECT) (see e.g., Wong & Gjedde, Encyclopedia of Neuroscience, 2009; 939-952 and Takano, Front Psychiatry., 2018; 9:228).

In some embodiments, exemplary disclosed compounds display a comparable or improved pharmacological profile relative to MDMA. Unless otherwise noted, as used herein, MDMA refers to the racemic form of the compound. Herein, “comparable” refers to the absence of a statistically significant difference between measures, as can be determined by one of skill. Representative examples of pharmacological activity include 5-HT and DA transporter (SERT and DAT, respectively) inhibition, such as inhibition of 5-HT and DA re-uptake, and 5-HT and DA releasing activity. Norepinephrine (NE) release and transporter (NET) activity may additionally be evaluated. Entactogen-like properties can be differentiated from stimulants, for example, by determining monoamine release activity. For example, amphetamine shows greater release activity of DA than 5-HT, whereas MDMA and MDDMA promote greater release of 5-HT than DA. See, e.g., DAT/SERT ratios of MDMA (0.8) and methamphetamine (>10) in Simmler et al., Br J Pharmacol. 2013; 168(2): 458-470. Entactogens can also be distinguished from serotonergic drugs. For example, psilocybin may release 5-HT to a comparable extent as MDMA and amphetamine but release only a fraction of DA, e.g., about 10%-20% of monoamine levels released by MDMA and amphetamine.

In some embodiments, therapeutic compounds increase serotonin levels in the CNS. In some embodiments, therapeutic compounds selectively modulate, or modulate to a greater extent, serotonergic neurotransmission over dopamine and noradrenaline neurotransmission. In some embodiments, therapeutic compounds increase serotonin levels in the CNS and/or the peripheral nervous system (PNS). In some embodiments, therapeutic compounds selectively increase, or increase to a greater extent, serotonin levels over dopamine and noradrenaline levels. In some embodiments, the increase is determined relative to baseline levels, such as prior to administration of a therapeutic compound. In some embodiments, following administration of a therapeutic compound, serotonin levels peak prior to peak serotonin levels observed following administration of a reference compound. In some embodiments, a reference compound is administered at the same or at a comparable dose to a therapeutic compound for the purposes of comparison. In some embodiments, peak serotonin levels following administration of a therapeutic compound are observed 5 to 30 minutes prior to peak serotonin levels following administration of a reference compound. In some embodiments, peak levels of serotonin are observed at about 5, 10, 15, 20, 25, or 30 minutes prior to detection of peak serotonin levels following administration of a reference compound.

In embodiments, the reference compound is MDMA. In embodiments, the reference compound is MEAI. In embodiments, both MDMA and MEAI are reference compounds.

In some embodiments, the effects of therapeutic compounds on monoamine levels are short acting. In some embodiments, the effects of therapeutic compounds on monoamine levels are of short duration. In some embodiments, monoamine levels in response to therapeutic compounds return to baseline sooner than reference compounds MEAI and/or MDMA. In some embodiments, monoamine levels in response to therapeutic compounds return to baseline at least about 5 minutes, 10 minutes, 15 minutes, 20 minutes, 25 minutes, 30 minutes, 35 minutes, 40 minutes, 45 minutes 50 minutes, 55 minutes, 60 minutes, 65 minutes, 70 minutes, 80 minutes, 85 minutes, 90 minutes, 95 minutes, 100 minutes, 105 minutes, 110 minutes, 115 minutes, or at least 120 minutes earlier than a return to baseline in response to MEAI and/or MDMA. In some embodiments, the monoamine is serotonin. In some embodiments, the monoamine is dopamine. In some embodiments, the monoamine is noradrenaline (norepinephrine). In some embodiments, levels of two monoamines are measured. In some embodiments, levels of three monoamines are measured.

In some embodiments, therapeutic compounds increase monoamine levels to a lesser extent than MEAI and/or MDMA. In some embodiments, peak monoamine levels in response to therapeutic compounds are about 10%, 20%, 30%, 40%, 50%, 60%, 70%, 80%, 90%, 100%, 125%, 150%, 175%, 200%, 225%, 250%, 275%, 300%, 325%, 350%, 375%, 400%, 425%, 450%, 475%, 500%, 525%, 550%, 575%, 600%, 625%, 650%, 675%, 700%, 725%, 750%, 775%, 800%, 825%, 850%, 875%, 900%, 925%, 950%, 975%, 1000%, 1250%, 1500%, 1750%, 2000%, 2250%, 2500%, 2750%, or about 3000% less than monoamine levels in response to MEAI and/or MDMA. In some embodiments, the monoamine is serotonin. In some embodiments, the monoamine is dopamine. In some embodiments, the monoamine is noradrenaline (norepinephrine).

In embodiments, therapeutic compounds increase 5-HT, DA, and NE levels. In some embodiments, therapeutic compounds increase NE levels to a lesser extent than 5-HT levels in response to therapeutic compounds. In some embodiments, peak NE levels in response to therapeutic compounds are reduced relative to peak 5-HT levels in response to the same. In some embodiments, peak NE levels are reduced relative to peak 5-HT levels by about 25-250%, 50%-225%, 75%-200%, 100%-175%, or 125%-150%. In some embodiments, peak NE levels are reduced relative to peak 5-HT levels by at least 50%, 55%, 60%, 65%, 70%, 75%, 80%, 85%, 90%, 95%, 100%, 105%, 110%, 115%, 120%, 125%, 130%, 135%, 145%, 150%, 155%, 160%, 165%, 170%, 175%, 180%, 185%, 190%, 195%, or 200%.

In some embodiments, therapeutic compounds increase DA levels to a lesser extent than NE levels. In some embodiments, peak DA levels in response to therapeutic compounds are reduced relative to NE levels in response to the same. In some embodiments, peak DA levels are reduced relative to peak NE levels by about 10%-100%, 20%-90%, 30%-80%, 40%-70%, or 50%-60%. In some embodiments, peak DA levels are reduced relative to peak NE levels by at least 10%, 15%, 20%, 25%, 30%, 35%, 40%, 45%, 50%, 55%, 60%, 65%, 70%, 75%, 80%, 85%, 90%, 95%, or 100%.

In some embodiments, therapeutic compounds increase NE levels to a lesser extent than DA levels. In some embodiments, peak NE levels in response to therapeutic compounds are reduced relative to DA levels in response to the same. In some embodiments, peak NE levels are reduced relative to peak DA levels by about 10%-100%, 20%-90%, 30%-80%, 40%-70%, or 50%-60%. In some embodiments, peak NE levels are reduced relative to peak DA levels by at least 10%, 15%, 20%, 25%, 30%, 35%, 40%, 45%, 50%, 55%, 60%, 65%, 70%, 75%, 80%, 85%, 90%, 95%, or 100%.

In certain preferred embodiments, the therapeutic compounds when administered according to the methods of the invention modulate neurotransmission so as to provide the benefits of certain known compounds (e.g., MDMA and/or MEAI) without their side effects or with reduced risks thereof. That the compositions of the invention are useful in methods for modulating neurotransmission, and that such modulation results further results in treatment of a mental health disorder or improvement of psychological functioning, will be apparent from the disclosure herein, and from such knowledge regarding, e.g., how pharmaceutical agents that increase serotonin (for instance, either by inducing its release as with monoamine releasers, or inhibiting its reabsorption as with SSRIs) show benefit for treatment of mental health disorders such as depression and anxiety.

For example, it is shown that 5-HT receptors are implicated in the progression and treatment of psychiatric disorders such as depression and anxiety, and that compositions comprising substances that modulate or effect 5-HT receptors produce augmented therapeutic effects in individuals suffering from psychiatric and neurological disorders, as disclosed herein. Hence, compositions and formulations of the invention that modulate serotonergic neurotransmission, as disclosed in various embodiments herein, can be effective for treatment of such mental health disorders, as well as other disorders that are likewise correlated with dysfunction of serotonergic neurotransmission.

b. Methods of Treatment

In some aspects are provided methods of treatment comprising administering therapeutic compounds to a subject to treat a disease or disorder. In some embodiments, therapeutic compounds are useful to treat substance use disorders, behavioral addictions, and mental health disorders. In some embodiments, therapeutic compounds are administered to a subject in conjunction with psychotherapy.

i. Substance Use Disorders

Broadly, but without being bound by theory, substance use disorders are mediated by the dopamine system (Diana, Front Psychiatry, 2011; 2:64). Additionally, the noradrenergic system has been found to contribute to addiction, including reward and drug seeking behavior (Foster & Weinshenker, Neural Mechanisms of Addiction, 2019; 221-236). In some embodiments, therapeutic compounds or pharmaceutical compositions thereof are used to treat substance use disorders. In some examples, substance use disorders are characterized by excessive use of nicotine, alcohol, and narcotics including prescription drugs and drugs of abuse. Such use may lead to one or more of social, academic, and occupational impairment. Commonly abused substances include, e.g., alcohol, tobacco (nicotine), cannabis, sedatives, hypnotics, anxiolytics, inhalants, opiates, opioids, and stimulants (Jahan & Burgess, “Substance Use Disorder,” Treasure Island (FL): StatPearls Publishing; 2022).

### 1. Alcohol Use Disorder

“Alcohol use disorder” (AUD) refers to the disorder as defined in the Diagnostic and Statistical Manual of Mental Disorders, for example, the DSM-5. Under DSM-5, anyone meeting any two of 11 listed criteria during the same 12 month period receive a diagnosis of AUD. The severity of AUD mild, moderate, or severe is based on the number of criteria met. In some embodiments, therapeutic compounds are useful to treat AUD.

Specifically, the criteria are whether in the past year, a patient has: (1) Had times when you [i. the patient] ended up drinking more, or longer, than you intended? (2) More than once wanted to cut down or stop drinking, or tried to, but couldn't? (3) Spent a lot of time drinking? Or being sick or getting over other aftereffects? (4) Wanted a drink so badly you couldn't think of anything else? (5) Found that drinking or being sick from drinking often interfered with taking care of your home or family? Or caused job troubles? Or school problems? (6) Continued to drink even though it was causing trouble with your family or friends? (7) Given up or cut back on activities that were important or interesting to you, or gave you pleasure, in order to drink? (8) More than once gotten into situations while or after drinking that increased your chances of getting hurt (such as driving, swimming, using machinery, walking in a dangerous area, or having unsafe sex)? (9) Continued to drink even though it was making you feel depressed or anxious or adding to another health problem? Or after having had a memory blackout? (10) Had to drink much more than you once did to get the effect you want? Or found that your usual number of drinks had much less effect than before? (11) Found that when the effects of alcohol were wearing off, you had withdrawal symptoms, such as trouble sleeping, shakiness, restlessness, nausea, sweating, a racing heart, or a seizure? Or sensed things that were not there?

The severity of the AUD is based on the number of criteria met. It is defined as “mild” with the presence of two to three symptoms, “moderate” with the presence of four to five symptoms, or “severe” with the presence of six or more symptoms. In some embodiments, therapeutic compounds are useful to treat mild AUD. In some embodiments, therapeutic compounds are useful to treat moderate AUD. In some embodiments, therapeutic compounds are useful to treat severe AUD.

Although herein the terms “alcohol use disorder” and “AUD” generally refer to the criteria in the DSM-5, or a patient with a diagnosis based thereon, it will be appreciated that the therapeutic compounds, compositions, and methods herein are equally applicable to subjects having the equivalent underlying disorder, whether that disorder is diagnosed based on the criteria in DSM-5 or in DSM-IV (which described two distinct disorders: “alcohol abuse and alcohol dependence with specific criteria for each), whether the diagnosis is based on other clinically acceptable criteria (e.g., as “Alcohol Dependence Syndrome,” see Edwards, The Alcohol Dependence Syndrome: A Concept as Stimulus to Enquiry. Brit. J. Addiction, 81:171-183), or whether the patient has not yet had a formal clinical diagnosis.

“Alcohol dependence” refers to the more severe form of AUD involving physical alcohol dependence. When referring specifically to “physically dependent” alcoholism or alcohol addiction (or “dependent use”) it will be understood to refer to the psychiatric disorder or condition in which an individual has become physically dependent upon or physically addicted to alcohol (e.g. Alcohol Dependence Syndrome). Typically alcohol dependence is characterized by oftentimes serious withdrawal symptoms on cessation of alcohol, or drinking to avoid withdrawal symptoms, tolerance and the persistent desire to drink and continuing drinking despite negative consequences (NICE Guidelines on AUD, 2011). In some embodiments, therapeutic compounds are useful to treat alcohol dependence.

“Harmful use of alcohol or “harmful use” AUD means that defined in the World Health Organization (WHO)'s International Classification of Diseases, 10th Revision (The ICD-10 Classification of Mental and Behavioural Disorders) (ICD-10; WHO, 1992 as: [A] pattern of psychoactive substance use that is causing damage to health. The damage may be physical (e.g., hepatitis) or mental (e.g., depressive episodes secondary to heavy alcohol intake). Harmful use commonly, but not invariably, has adverse social consequences; social consequences in themselves, however, are not sufficient to justify a diagnosis of harmful use. In some embodiments, therapeutic compounds are useful to treat harmful use AUD.

From a clinical perspective, it can be helpful to separate this group of harmful drinkers from those with more severe and enduring physical dependence upon alcohol that would require medical support (e.g., detoxification with benzodiazepine assistance to avoid a severe physical withdrawal syndrome with associated risk of seizures). In some embodiments, therapeutic compounds are useful to treat physical dependence on alcohol.

The distinction between the different severities of AUD may not be immediately clear and may vary over time within a single patient's relationship with alcohol. However, one of ordinary skill will appreciate how to make an appropriate and reasonable clinical determination. Clinical signs and tools used to make the distinction between those AUD patients with non physical “harmful use” and those who are physically dependent on alcohol will be understood; for example, patients who have non-physically dependent “harmful use” of alcohol may (1) drink more than 14 units of alcohol per week, but (2) have no evidence of physical dependence (including no evidence of severe sweating, tremor, vomiting, nausea or confusion on cessation of drinking for >48 hours), and may (3) have a lack of evidence of a strong craving to drink alcohol in order to combat the symptoms of alcohol withdrawal, as well as (4) score 15 or less on the Severity of Alcohol Dependence Questionnaire (SADQ).

The term “alcohol use disorder patient” or “AUD patient” refers to a patient diagnosed with AUD, as defined herein. In one embodiment, the term “AUD patient” refers to a patient diagnosed with AUD who is in abstinence from alcohol, for example, for at least 1 day, such as 3 days or more. The term “AUD patient in abstinence from alcohol” refers to a patient diagnosed with AUD in abstinence from alcohol for a period, for example, for at least 1 day and wherein the period is typically counted from baseline as that term is defined herein.

The term “binge drinking” refers to an abuser of alcohol (i.e., a heavy drinker). Abusers of alcohol may not drink on a consistent basis; for example, they may only drink once a week, but, when drinking, they may drink heavily, which will cause problems, such as suffering from alcohol intoxication. The term “heavy drinker” refers to someone with a heavy alcohol use pattern. According to the National Institute on Alcohol Abuse and Alcoholism (NIAAA) the Substance Abuse and Mental Health Services Administration (SAMHSA) defines “heavy alcohol use” as binge drinking on 5 or more days in the past month. NIAAA defines binge drinking as a pattern of drinking that brings blood alcohol concentration (BAC) levels to 0.08 g/dL. This typically occurs after 4 alcoholic drinks for women and 5 alcoholic drinks for men within a period of about 2 hours. SAMHSA defines “binge drinking” as 5 or more alcoholic drinks for males or 4 or more alcoholic drinks for females on the same occasion (i.e., at the same time or within a couple of hours of each other) on at least 1 day in the past month. In embodiments, therapeutic compounds are useful to treat binge drinking.

The term “alcohol,” for example in relation to “drinks,” “alcoholic drinks,” or “drinking,” refers to ethyl alcohol (ethanol). The term “alcohol craving” refers to a conscious desire or urge to consume alcohol. The term “alcohol use” refers to alcohol consumption.

The term “reducing alcohol use” or “reduction of alcohol use refers to reducing the amount or frequency of alcohol use, for example as assessed by urinalysis e.g., by measuring metabolites of alcohol in urine, such as Ethyl Glucuronide (EtG) or as assessed by using self reported alcohol use with standardized tools like the Timeline Follow Back self report. See, e.g., Robinson et al., Psychol Addict Behav., 2014; 28(1):154-62; Sobell et al., Drug Alcohol Depend., 1996; 42(1):49-5

In some embodiments, reducing alcohol use or reduction of alcohol use refers to a reduction in drinks per day, a reduction in drinks per drinking day, or a reduction in the frequency of drinking, such as a reduction in the percentage of drinking days or percentage of heavy drinking days. In some embodiments, reducing alcohol use or reduction of alcohol use refers to an increase in the time to drinking or the time to the first heavy drinking day. The term “drinking “drinks or “alcoholic drinks” as used herein, is understood in the context of “standard drinks such as spirits or blends that are intended for human consumption, wherein a “standard drink” equals 1 [symbol] g ethanol. The term “heavy drinking day” refers to a day with a total alcohol consumption ≥60 g of ethanol for men and 40 g for women. In some embodiments, administration of therapeutic compounds to a subject results in reduced alcohol use. In some embodiments, the subject is an alcohol use disorder patient.

Alcohol abstinence or abstinence from alcohol refers to not drinking alcohol. The term “promoting alcohol abstinence” or “promotion of alcohol abstinence refers to help maintaining abstinence from alcohol use, in particular after at least 1 day of not drinking alcohol, for example maintaining abstinence from alcohol use for a period of at least 1 week, 2 weeks, 3 weeks, 1 month, 3 months, 6 months 9 months, 1 year, or more, and in particular at least 1 week or more, su ch as 2 weeks and in certain embodiments for the 3, 6 or 9 months of a follow up for longer than 9 months, or for longer than 1 year. In some embodiments, therapeutic compounds promote alcohol abstinence in a subject. In some embodiments, the subject is an alcohol use disorder patient.

Relapse into alcohol use or relapse into alcohol consumption refers to an alcohol intake (i.e., taking alcohol) following a period of alcohol abstinence, for example following a period of alcohol abstinence of at least 1 day or more, such as 3 days, 1 week, 2 weeks, 3 weeks, 1 month, 3 months, 6 months 9 months, 1 year, or more. In some embodiments, therapeutic compounds are used to prevent relapse. In some embodiments, therapeutic compounds are used to delay relapse in a subject. In some embodiments, the subject is an alcohol use disorder patient. Preventing relapse into alcohol use or preventing relapse into alcohol consumption refers to the prevention of alcohol intake by an AUD patient after the patient has stopped the intake of alcohol, in particular after 1 day or more of not taking alcohol. In some embodiments, the term encompasses the permanent stoppage of alcohol intake. In other embodiments, the term encompasses a delay in the resumption of alcohol intake as compared to the time to resumption by a subject that is not administered a compound of the invention The delay in resumption can be, e.g., days (e. 2, 3, 4, 5, 6, 7 days), weeks (e. 1, 2, 3 weeks), months (e. 1, 2, 3, 4, 5, 6 7, 8, 9 months), or longer such as more than 1 year 83. In some embodiments, administration of therapeutic compounds prevents relapse in a subject. In some embodiments, the subject is an alcohol use disorder patient.

“AUD associated with high risk drinking for acute problems” refers to alcohol use disorder associated with daily alcohol consumption 60 g/day of ethanol for men and 40 g/day for women (International Guide for Monitoring Alcohol Consumption and Related Harm, WHO/MSD/MSB/00.4; World Health Organization 2000, p. 51).

[symbol]

“Impulsivity” refers to a predisposition toward rapid, unplanned reactions to internal or external stimuli with diminished regard to the negative consequences of these reactions to the impulsive individual or others” (Moeller et al., Am J Psych, 2001; 158:1783-93).

The cost of excessive alcohol use is high, both to the individual, in terms of physical and mental health, and to society in terms of social and medical care. Physically dependent addiction to alcohol is a significant public health problem affecting up to 4% of the population and new treatments for alcohol addiction also are under investigation by Applicant herein. At the same time, there is evidence that non physically dependent harmful use of alcohol is an even larger (and growing problem with almost 25% of adults in the UK now labeled as problem drinkers and similar statistics elsewhere worldwide. And although recognized as an area of medicine that needs new approaches, none have been forthcoming.

In some embodiments, the therapeutic compounds can reduce a subject's alcohol consumption. In some embodiments, the subject is an alcohol use disorder patient. In some embodiments, such reduction persists for at least 1 month, 2 months, 3 months, 4 months, 5 months, 6 months, 7 months, 8 months, 9 months, 10 months, 11 months, 11 months, or 12 months. In some embodiments, treatment with therapeutic compounds results in reduced rates of relapse, and producing benefits not only to the individual patients treated, but to their friends and family, and to society at large.

### 2. Opioid Use Disorder

Receptors of the opioid system, mu, kappa, delta, and opioid receptor like-1 (ORLI) are G-protein coupled receptors that activate inhibitory G-proteins (Al-Hasani & Bruchas, Anesthesiology. 2011; 115(6):1363-1381. Opioids activate the mesolimbic reward system, which promotes signaling in the ventral tegmental area and results in dopamine release (Kosted & George, Sci Pract Perspect., 2002; 1(1):13-20). Representative examples of opioids include codeine, heroin, hydrocodone, hydromorphone, methadone, meperidine, morphine, and oxycodone. It will be appreciated that “opiates” may be included among “opioids” for purposes of the inventions herein. In some embodiments, a therapeutic compound is used to treat opioid use disorder (which will include opiate use disorder).

Opioid use disorder (OUD) is characterized by an overwhelming desire to use opioids, the development of tolerance to opioids, and withdrawal syndrome once opioids are discontinued. Criteria for opioid use disorder are available to one of skill. In one example, the DSM-5 describes criteria, for example: Opioids are often taken in larger amounts or over a longer period of time than intended; Tolerance, as defined by either of the following: (a) a need for markedly increased amounts of opioids to achieve intoxication or desired effect or (b) markedly diminished effect with continued use of the same amount of an opioid; Withdrawal, as manifested by either of the following: (a) the characteristic opioid withdrawal syndrome or (b) the same (or a closely related) substance is taken to relieve or avoid withdrawal symptoms. In some embodiments, therapeutic compounds alleviate or reduce the signs or symptoms of opioid use disorder. In some embodiments, a therapeutic compound may be used as an adjunct to clinical opioid therapy.

Treatments for OUD include opioid receptor agonists, for example, methadone and buprenorphine, and opioid receptor antagonists, for example, naltrexone (Kampman et al., J. Addict. Med., 2015; 9(5):358-367). However, such treatments are associated with limited success in preventing relapse and, in some cases, carry abuse liability themselves. The dopamine system has been implicated in opioid reward (Fields & Margolis, Trends Neurosci., 2015; 38(4):217-225; Steidl et al., Neurosci. Biobehav. Rev., 2017; 83:72-82). D3 antagonists may be useful to treat opioid addiction and to potentiate the effects of prescribed opiates (Galaj et al., Neurosci. Biobehav. Rev., 2020; 114: 38-52). A compound's effects on opioid-seeking behavior may be evaluated according to methods available to one of skill, including self-administration models, e.g., the IV self-administration reinstatement rodent model described by Fattore et al., Methods Mol Biol. 2021; 2201:231-245.

### 3. Nicotine Dependence and Tobacco Use Disorder

Nicotine addiction is mediated by activation of neuronal nicotinic acetylcholine receptors in the dopamine pathway, and nicotine exposure results in release of dopamine and an increase in extracellular dopamine levels (Laviolette & van der Kooy, Nat. Rev. Neurosci., 2004; 5(1):55-65; Nisell et al., Synapse, 1994; 16(1):36-44; Pierce & Kumaseran, Neurosci. Biobehav. Rev., 2006; 30(2):215-38). Tobacco is a common vehicle for nicotine and so nicotine dependence may be referred to as tobacco use disorder. In some embodiments, a therapeutic compound is used to treat nicotine dependence. In some embodiments, a therapeutic compound is used to treat tobacco use disorder. Criteria for nicotine dependence or tobacco use disorder are available to one of skill. See, e.g., Baker et al., Addiction, 2012; 107(2):263-275. In one example, the DSM-5 describes tobacco use disorder and criteria for the same, for example: Unsuccessful efforts to quit or reduce intake of tobacco; Inordinate amount of time acquiring or using tobacco products; Cravings for tobacco. In some embodiments, therapeutic compounds alleviate or reduce the signs or symptoms of nicotine dependence or tobacco use disorder. A compound's effects on nicotine seeking behavior may be evaluated according to methods available to one of skill, including self-administration, place conditioning, and intracranial self-stimulation paradigms in rodents (O'Dell & Khroyan, Pharmacology Biochemistry and Behavior, 2009; 91(4): 481-488).

### 4. Sedative, Hypnotic, and Anxiolytic Use Disorder

Sedatives, hypnotics, and anxiolytics can cause CNS depression, which may be fatal. Exemplary CNS depressants include benzodiazepines, such as alprazolam, clonazepam, lorazepam, diazepam, chlordiazepoxide, and barbiturates, such as phenobarbital, pentobarbital, butabarbital. Other classes of drugs have properties that share a similar mechanism of action with benzodiazepine and barbiturates, including alcohol. These agents mediate gamma-aminobutyric acid (GABA) effects, producing inhibitory effects within the central nervous system. In some embodiments, a therapeutic compound is used to treat sedative, hypnotic, and anxiolytic use disorder.

Criteria for sedative, hypnotic, and anxiolytic use disorder are available to one of skill. In one example, the DSM-5 describes sedative, hypnotic, and anxiolytic use disorder and criteria for the same, for example: Sedatives, hypnotics, or anxiolytics are often taken in larger amounts or over a longer period than was intended; There is a persistent desire or unsuccessful efforts to cut down or control sedative, hypnotic, or anxiolytic use; A great deal of time is spent in activities necessary to obtain the sedative, hypnotic, or anxiolytic; use the sedative, hypnotic, or anxiolytic; or recover from its effects. In some embodiments, therapeutic compounds alleviate or reduce the signs or symptoms of sedative, hypnotic, and anxiolytic use disorder.

### 5. Stimulant Use Disorder

Stimulants increase synaptic levels of the monoamines dopamine, serotonin, and norepinephrine. Both the dopaminergic and the noradrenergic systems play critical roles in the effects of stimulants, including reward (Sofuoglu & Sewell, Addict Biol., 2009; 14(2): 119-129; Wise, Brain Res., 1978; 152(2):215-47). Non-limiting examples of stimulants include amphetamines, methamphetamine, and cocaine. Nicotine/tobacco may also be considered a stimulant. In some embodiments, a therapeutic compound is used to treat stimulant use disorder. In some embodiments, a therapeutic compound is used to treat cocaine use disorder. In some embodiments, a therapeutic compound is used to treat methamphetamine use disorder.

Criteria for stimulant use disorder are available to one of skill. For example, exemplary DSM-5 criteria include: The stimulant is often taken in larger amounts or over a longer period than was intended; There is a persistent desire or unsuccessful efforts to cut down or control stimulant use; A great deal of time is spent in activities necessary to obtain the stimulant, use the stimulant, or recover from its effects. In some embodiments, therapeutic compounds alleviate or reduce the signs or symptoms of stimulant use disorder.

Treatment options for stimulant use disorder include contingency management, CBT, acupuncture, antidepressants, dopamine agonists, antipsychotics, anticonvulsants, disulfiram, opioid agonists, N-Acetylcysteine, and psychostimulants (Ronsley et al., PLoS One, 2020; 15(6): e0234809). Psychostimulants, for example, bupropion and dexamphetamine, appear to be promising treatment options. In contrast, dopamine modulators, including agonists and antagonists appear to lack efficacy in stimulant use disorders, such as cocaine use disorder (Cochrane Database Syst Rev. 2015 May; 2015(5): CD003352; Cochrane Database Syst Rev. 2016 March; 2016(3): CD006306). The effects of a compound on stimulant addiction may be assessed using methods available to one of skill, including self-administration models, e.g., the hold down procedures described by Zimmer & Roberts, Psychiatric Disorders, 2011; 279-290.

ii. Behavioral Addictions

The concept of “addiction” traditionally referred to use of psychotropic substances and dependence on the same (Pinna et al., Journal of Psychopathology, 2015; 21(4):380-389). However, addiction is frequently also applied to describe a heterogeneous group of syndromes known as behavioral addictions (Konkoly et al., BMC Psychiatry, 2015; 15:4). Addiction is not a unitary construct but rather incorporates a number of features, such as repetitive engagement in behaviors that are rewarding (at least initially), loss of control (increasing engagement over time), persistence despite detrimental consequences, and physical dependence (evidenced by withdrawal symptoms when intake of the substance diminishes) (Chamberlain et al., European Neuropsychopharmacol., 2016; 26(5), 841-855).

Numerous behavioral addictions have been described in the literature: sex addiction, internet gaming addiction, compulsive buying, exercise addiction or overtraining syndrome, love addiction, work addiction or workaholism, and technological addictions (Pinna et al., Journal of Psychopharmacology, 2015; 21(4), 380-9). Akin to the effects of SUDs, behavioral addictions may impair psychological and social functioning. The invention herein is addressed to these behavioral addictions, and to all such others with the same features. In some embodiments, disclosed therapeutic compounds are used to treat behavioral addictions. In some embodiments, therapeutic compounds are used to improve impaired psychological and/or social functioning associated with behavioral addictions.

Certain psychiatric disorders characterized by maladaptive, repetitive behaviors share some at least surface similarities with substance addiction and are referred to as behavioral addictions. This perspective has influenced the DSM-5, which created a new class of disorder, “Substance Related and Addictive Disorders.” At present, there is insufficient evidence to support the categorisation of many behavioral addictions within the substance related and addictive disorders class of the DSM-5, and a need to establish diagnostic criteria in order to classify these behaviors as mental disorders (Pinna et al., Journal of Psychopharmacology, 2015; 21(4), 380-9). Gambling disorder, however, has been recognized as an addictive disorder in the DSM-5 and has established diagnostic criteria.

Behavioral addictions, e.g., gambling, gaming, shopping, sexual behavior, and the like, are defined as an intense desire to repeat some action that is pleasurable, or perceived to improve well-being, or capable of alleviating some personal distress, despite the awareness that such an action may have negative consequences. Substance use disorders generally involve an aspect of impulsivity at their outset, and subsequent substance related changes can then produce organic disruption in the prefrontal cortex that further reduces control over behavior. See, e.g., Goldstein & Volkow, Neuropsychopharmacology, 2011; 36(1), 366-367. In contrast, behavioral addictions whilst also characterized by a loss of control of behavior, often exhibit a greater compulsive than impulsive features. Compulsive, or habitual responding in the absence of reward, is key to behavioral addictions but research has found it difficult to treat. Additional compulsive behaviors that may be treated by therapeutic compounds are described in, e.g., World Health Organization. International Classification of Diseases for Mortality and Morbidity Statistics, 11th Revision, 2018.

Behavioral addictions not only affect the individual, but carry detrimental social, cultural, and economic consequences. There also is extensive evidence for overlapping comorbidity amongst behavioral addictions themselves, and for the co-occurrence of substance use disorders (SUDs) and behavioral addictions. For example, problem gambling has been found to co-occur with Internet addiction and/or alcohol use disorder (Rennert et al., Exp Clin Psychopharmacol. 2014; 22(1): 50-56; Tozzi et al., Swiss Medical Weekly, 2013; 143(wl3768), 1-6). In some embodiments, treatment of a substance use disorder with a therapeutic compound may additionally treat a behavioral addiction.

### 1. Gambling Disorder

Gambling disorder is an addictive disorder in which individuals present with a cluster of symptoms of varying severity that cause psychological and physical harms and social dysfunction due to their gambling practices (American Psychiatric Association, 2013, Substance-Related and Addictive Disorders. In Diagnostic and Statistical Manual of Mental Disorders, 5th ed.). Gambling disorder may also be referred to as compulsive gambling or pathological gambling. Signs of gambling disorder are available to one of skill. In one example, the DSM-5 criteria for gambling disorder include preoccupation with gambling-related thoughts; a need to gamble with larger amounts of money to achieve excitement; repeated unsuccessful attempts to stop or cut back on gambling; restlessness or irritability after attempting to stop or cut back; gambling when experiencing distress; gambling to chase one's losses, deception of family members and loved ones to conceal the extent of one's gambling; jeopardizing jobs, education or significant relationships to pursue gambling; or a reliance on others to provide money to relieve a desperate financial situation caused by gambling. In addition, it must also be confirmed by the therapist that the gambling behavior is not better explained by a manic episode (NPCGambling.org, DSM-5 Diagnostic Criteria: Gambling Disorder). In some embodiments, therapeutic compounds are useful to treat gambling disorder. In some embodiments, therapeutic compounds alleviate or reduce the severity of one or more signs of gambling disorder. In some embodiments, therapeutic compounds may be used in conjunction with psychotherapy to treat gaming disorder.

Individuals diagnosed with gambling disorder are at a heightened risk of developing stress-related conditions such as hypertension, sleep deprivation, cardiovascular disease, and peptic ulcer disease. Common psychiatric sequelae that accompany pathological gambling include the exacerbation and initiation of major depressive episodes, anxiety disorders, or substance use disorders (Fong, Psychiatry (Edgmont). 2005; 2(3): 22-30). Unintended psychological consequences may also include intense levels of guilt and shame, deception of loved ones, increased impulsivity/impaired decision-making, and suicidal ideation, which impact psychological and social functioning. In some embodiments, therapeutic compounds may improve impaired psychological and social aspects associated with gambling disorder.

### 2. Gaming Disorder

Gaming disorder is defined in the 11th Revision of the International Classification of Diseases (ICD-11) as a pattern of gaming behavior, for example, “digital-gaming” or “video-gaming.” Exemplary criteria include impaired control over gaming, increasing priority given to gaming over other activities to the extent that gaming takes precedence over other interests and daily activities, and continuation or escalation of gaming despite the occurrence of negative consequences (World Health Organization. International Classification of Diseases for Mortality and Morbidity Statistics, 11th Revision, 6C51, 2018). For a diagnosis, the behavior must be of sufficient severity to result in significant impairment in personal, family, social, educational, occupational or other important areas of functioning and would normally have been evident for at least 12 months (WHO, “Addictive behaviours: Gaming disorder,” 2020). Methods of identifying gaming disorder are available to one of skill, including ICD-11 criteria and questionnaires, such as the Gaming Disorder Scale for Adolescents (GADIS-A) (Paschke et al., J. Clin. Med., 2020; 9(4), 993). In embodiments, therapeutic compounds are used to treat gaming disorder. In some embodiments, therapeutic compounds are used to alleviate or reduce the severity of one or more signs of gaming disorder. In some embodiments, therapeutic compounds may be used in conjunction with psychotherapy to treat gaming disorder. In some embodiments, therapeutic compounds may improve impaired psychological and social aspects associated with gaming disorder.

### 3. Sexual Addiction

Compulsive sexual behavior disorder is characterised by a persistent pattern of failure to control intense, repetitive sexual impulses or urges resulting in repetitive sexual behavior. Critera for diagnosing sexual addiction are known to one of skill. Exemplary criteria according to the ICD-11 for compulsive sexual behavior disorder include: Engaging in repetitive sexual behaviour has become a central focus of the individual's life to the point of neglecting health and personal care or other interests, activities and responsibilities; The individual has made numerous unsuccessful efforts to control or significantly reduce repetitive sexual behaviour; The individual continues to engage in repetitive sexual behaviour despite adverse consequences (e.g., marital conflict due to sexual behaviour, financial or legal consequences, negative impact on health); The person continues to engage in repetitive sexual behaviour even when the individual derives little or no satisfaction from it (WHO. International Classification of Diseases for Mortality and Morbidity Statistics, 11th Revision, 6C72, 2018). Sexual addiction may also be referred to as compulsive sexual behavior, hypersexuality, or hypersexuality disorder. In some embodiments, therapeutic compounds are used to treat sexual addiction. In some embodiments, therapeutic compounds are used to alleviate or reduce the severity of one or more signs of sexual addiction. In some embodiments, therapeutic compounds may be used in conjunction with psychotherapy to treat sexual addiction. In some embodiments, therapeutic compounds may improve impaired psychological and social aspects associated with sexual addiction.

### 4. Compulsive Buying Behavior

Compulsive buying behavior, also referred to as shopping addiction or compulsive buying disorder, is characterized by excessive or poorly controlled preoccupations, urges or behaviors regarding shopping and spending, which lead to adverse consequences (Black, CNS Drugs, 2001; 15(1):17-27; Granero, Front Psychol., 2016; 7:914). Criteria for shopping addiction are available to one of skill and include, e.g., Do you feel overly preoccupied with shopping and spending?; Do you ever feel that your shopping behavior is excessive, inappropriate or uncontrolled?; Have your shopping desires, urges, fantasies or behaviors ever been overly time consuming, caused you to feel upset or guilty, or led to serious problems in your life (e.g. financial or legal problems, relationship loss)? (Black, CNS Drugs, 2001; 15(1):17-27). In some embodiments, therapeutic compounds are used to treat shopping addiction. In some embodiments, therapeutic compounds are used to alleviate or reduce the severity of one or more signs of shopping addiction. In some embodiments, therapeutic compounds may be used in conjunction with psychotherapy to treat shopping addiction. In some embodiments, therapeutic compounds may improve impaired psychological and social aspects associated with compulsive buying behavior.

### 5. Technology Addiction

In some embodiments, therapeutic compounds are used to treat technology addiction. Technology addiction may encompass internet addiction and/or smartphone addiction. Criteria for such addictions are available to one of skill (Lin et al., PLoS One, 2016; 11(11): e0163010). Exemplary criteria of technology addiction include: Is preoccupied with the internet (thinks about previous online activity or anticipates next online session); Needs to use the internet for increased amounts of time to achieve satisfaction; Has made unsuccessful efforts to control, cut back, or stop internet use. Evidence shows that plasma dopamine levels are positively correlated with aspects of internet addiction (Liu & Luo, Int J Clin Exp Med. 2015; 8(6): 9943-9948). Internet activities, for example, winning a level of a video game or getting “likes” on a picture may enhance dopamine release. In some embodiments, therapeutic compounds are used to alleviate or reduce the severity of one or more signs of technology addiction. In some embodiments, therapeutic compounds may be used in conjunction with psychotherapy to treat technology addiction. In some embodiments, therapeutic compounds may improve impaired psychological and social aspects associated with technology addiction.

iii. CNS and Mental Health Disorders

In some aspects are provided methods of treating CNS disorders with the therapeutic compounds. CNS disorders will be understood to include mental health disorders and neurodegenerative conditions. “Mental health disorder” may be used interchangeably with “psychiatric disorder.” In some examples, a mental health disorder is a condition in a mammal, and preferably in a human, that generally involves negative changes in emotion, mood, thinking, and/or behavior. Examples of mental health disorders include anxiety and stressor related disorders, dissociative disorders, eating disorders, mood disorders, obsessive compulsive and related disorders, personality disorders, schizophrenia and related disorders, sexuality, gender dysphoria, and paraphilias, somatic symptom and related disorders, suicidal behavior and self injury, and substance-related disorders, which includes substance-induced and substance use disorders. See Merck Manual of Diagnosis and Therapy, 20^{th} Ed., 2018. Diagnostic criteria is available to one of skill in the art, including the DSM-5, e.g., chapters Trauma- and Stressor-Related Disorders, Post-Traumatic Stress Disorder, Major Depressive Disorder, Anxiety Disorders, and General Anxiety Disorder, etc.

In some embodiments, mental health disorders include post-traumatic stress disorder (PTSD), adjustment disorder, affective disorder, depression, atypical depression, postpartum depression, catatonic depression, a depressive disorder due to a medical condition, premenstrual dysphoric disorder, seasonal affective disorder, dysthymia, anxiety, phobia disorders, binge disorders, body dysmorphic disorder, alcohol or drug abuse or dependence disorders, substance-related disorders, substance use disorders, alcohol use disorder, gaming disorders, gambling disorder, substance-induced mood disorder, a mood disorder related to another health condition, disruptive behavior disorders, eating disorders, impulse control disorders, obsessive compulsive disorder (OCD), attention deficit hyperactivity disorder (ADHD), personality disorders, attachment disorders, and dissociative disorders.

Included among such mental health disorders are depression including in forms such as treatment resistant depression and major depressive disorder, dysthymia, anxiety and phobia disorders (including generalized anxiety, social anxiety, panic, post traumatic stress and adjustment disorders), feeding and eating disorders (including binge eating, bulimia, and anorexia nervosa), other binge behaviors, body dysmorphic syndromes, disruptive behavior disorders, impulse control disorders, memory loss, dementia of aging, attention deficit hyperactivity disorder, personality disorders (including antisocial, avoidant, borderline, histrionic, narcissistic, obsessive compulsive, paranoid, schizoid and schizotypal personality disorders), attachment disorders, autism, and dissociative disorders.

In some embodiments, mental health disorders of the invention are specifically the “trauma and stressor related disorders,” which include acute stress disorder, adjustment disorders, and PTSD (Merck Manual, 20th Ed.), as well as reactive attachment disorder, disinhibited social engagement disorder, and others (as defined in DSM-5). In other embodiments, the mental health disorder is specifically depression, anxiety, or PTSD.

While the neurophysiology underlying mental health disorders may be distinct, an aspect in common of many is the presence of a deleterious, repetitive, and often “rigid” thought process that negatively impacts an individual's ability to function For someone with PTSD, for instance, symptoms involve re experiencing trauma and the feelings associated with it; for depression it can take the form of a recurrent internal editor that attaches negative connotations to normal life events; and for addiction it is the preoccupation with acquiring and using the substance of choice Thus, in many embodiments, the method of treating a mental health disorder involves the treatment of a disorder related to rigid modes of thinking In different embodiments, the disorder related to rigid modes of thinking can be anxiety, depression, addiction, an alcohol or drug abuse or dependence disorder an eating disorder, obsessive compulsive disorder, or PTSD.

In some embodiments, the pharmaceutical compositions are used to reduce the symptoms of a mental health disorder The symptoms of the mental health disorder to be treated shall be able to be determined by one of skill, by reference to the general understanding of the art regarding symptoms of PTSD, for example, include transient waking dissociative states in which events are relived as if happening (“flashbacks”), nightmares, distressing and intense memories, other intrusive negative memories, distress or physical reactions after being exposed to triggers, blaming self or others for the trauma, decreased interest in things that were once enjoyable and other feelings of emotional numbness, negative feelings about self and the world, inability to remember the trauma clearly, difficulty feeling positive, feelings of isolation, negative affect, difficulty feeling positive, other negative alterations in cognition and mood, avoidance, aggression or irritability, hypervigilance and hyper awareness, difficulty concentrating, difficulty sleeping, heightened startle response, engaging in self destructive, or risky behavior, difficulty sleeping or staying asleep, and suicidal ideation Accordingly, methods of the invention that reduce the symptoms of PTSD would be understood to reduce any such symptoms. Methods that reduce the symptoms of other mental health disorders would be understood to reduce such other symptoms as will be known to those of skill based on the general knowledge in the art.

In embodiments, neurodegenerative conditions include Alzheimer's disease, ataxia, Huntington's disease, Parkinson's disease, motor neuron disease, multiple system atrophy, progressive supranuclear palsy, migraines, cluster headaches, short-lasting unilateral neuralgiform headaches, fibromyalgia, traumatic brain injury (TBI), and mild TBI (mTBI).

### 1. Depressive Disorders

In some embodiments, therapeutic compounds are used to treat depressive disorders. Depression, one of the most common mental illnesses, is characterized by depressed mood and markedly diminished interest or pleasure in activities. Other symptoms include significant weight loss or weight gain, decrease or increase in appetite, insomnia or hypersomnia, psychomotor agitation or retardation, fatigue or loss of energy, feelings of worthlessness or excessive or inappropriate guilt, diminished ability to think or concentrate or indecisiveness, recurrent thoughts of death, suicidal ideation or suicidal attempts. Depression is listed by the WHO as the single largest factor contributing to global disability (Liu et al., J. Psych. Res., 2020; 126:134-140). Major depressive disorder (MDD) is a serious mood disorder characterized by severe and persistent low mood, profound sadness, or sense of despair. Additional depressive disorders include persistent depressive disorder (dysthymia)

### 2. Anxiety Disorders

In some embodiments, therapeutic compounds may be used to treat anxiety disorders. An anxiety disorder may be selected from the any of an adjustment disorder, acute distress disorder, post-traumatic stress disorder (PTSD), generalized anxiety disorder (GAD), panic disorder, panic attacks, agoraphobia, social anxiety disorder, separation anxiety disorder, specific phobia, substance-medication-induced anxiety disorder, anxiety disorder due to another medical condition, and illness anxiety disorder (hypochondriac).

Generalized anxiety disorder (GAD) is a mental disorder characterized by excessive and uncontrollable worry. Patients with GAD may have difficulty controlling their worries or feelings of nervousness, feel restless and have trouble relaxing, have trouble concentrating, have trouble sleeping, and experience headaches, muscle aches, stomach aches, and difficulty sleeping (Generalized Anxiety Disorder: When Worry Gets Out of Control, NIMH, 2016). The effects of a therapeutic compound on a subject's anxiety, such as a reduction of anxiety or alleviation of anxiety, may be determined according to one of skill. Examples of questionnaires related to rating feelings of calming and relaxation include the Relaxation Inventory, the Physiological Tension Scale, the Physical Assessment Scale, the Cognitive Tension Scale, the Taylor Manifest Anxiety Scale (Raskin et al., Archives of General Psychiatry, 1980; 37:93-97), Anxiety Symptoms Questionnaire (Baker et al., Gen. Psychiatry, 2019; 32(6): e100144), Beck Anxiety Inventory, General Anxiety Disorder scales, such as the GAD-7 (Spitzer et al., Arch. Intern. Med., 2006; 166:1092-7), Hamilton Anxiety Scale (HAM-A), State-Trait Anxiety Inventory (Spielberger et al., Manual for the State-Trait anxiety inventory. Consulting Psychologists Press, Inc, 1983), the Depression Anxiety Stress Scales (Lovibond & Lovibond, Behav. Res. Ther., 1995; 33:335-43).

### 3. Improved Mental Health and Psychological Functioning

In addition to treating various mental health disorders, the disclosed methods also can be used to improve overall mental health and psychological functioning in non-disease states, such as by reducing neuroticism and psychological defensiveness, increasing creativity and openness to experience, aiding decision making ability, increasing subjective feelings of wellness and satisfaction, and increasing the ability to fall or stay asleep.

Such improvements may be assessed according to methods known to one of skill. For example, sleep quality may be assessed with use of assessments, such as the Pittsburgh Sleep Quality Index (PSQI), and physiological measures, such as actigraphy data. See, e.g., Foreigner-Cordero et al., Sleep Sci. 2018; 11(3):141-145. Sleep quality may additionally be assessed with sleep diaries, contactless devices, contact devices, and polysomnography (Ibinez et al., PeerJ, 2018; 6:e4849; Rundo & Downey, Handb. Clin. Neurol., 2019; 160:381-392; Smith et al., J. Clin. Sleep Med., 2018; 14(7):1231-1237).

In some embodiments, improving psychological functioning results in improved social functioning. Aspects of social functioning include the quality of interactions with one's environment, including the contexts of work, social activities, and relationships with partners and family (Bosc, Compr. Psychiatry, 2000; 41(1):63-9. Strong social functioning, for example, the ability to forge strong social relationships has been correlated with enhanced mental health and longevity (Blumstein et al., Proc. Biol. Sci., 2018 Jan. 31; 285(1871): 20171934; Holt-Lunstad et al., PLoS Med., 2010; 7(7):e1000316). In some embodiments, the disclosed combinations extend longevity by improving one or more of psychological functioning, social functioning, and quality of life.

Functioning and quality of life may be assessed with use of tools available to one of skill, including, for example, the Adult Behavior Checklist and Social Responsiveness Scale for Adults, Social Functioning Questionnaire, the Global Assessment of Functioning Scale, and Social Adaptation Self-evaluation Scale, the DSM-5 recommended functioning assessment, the World Health Organization Disability Assessment Schedule 2.0 (WHODAS 2.0), and The World Health Organization Quality Of Life Assessment (WHOQoL) (Gold, Journal of the American Academy of Psychiatry and the Law Online, June 2014; 42(2) 173-181; Otter et al., BJPsych Open, 2021 Mar; 7(2): e51; Tyrer et al., Int. J. Soc. Psychiatry, 2005; 51(3):265-275; WHOQoL Group, Soc. Sci. Med., 1995; 1014031409).

c. Methods of Administration

In some aspects are provided methods of administering therapeutic compounds or compositions comprising the same, such as pharmaceutical compositions. In some embodiments, therapeutic compounds and compositions are self-administered. In some embodiments, therapeutic compounds and compositions are administered in combination with therapy or psychotherapy. In some embodiments, therapeutic compounds and compositions are administered in combination with psychological support. In some embodiments, therapeutic compounds and compositions are administered in combination with patient monitoring. In some embodiments, therapeutic compounds and compositions are administered in conjunction with a therapeutically beneficial activity. In any method of administration described herein, therapeutic compounds are administered parenterally. In any method of administration, therapeutic compounds are administered systemically. In any method of administration, therapeutic compounds are administered intravenously. In any method of administration, therapeutic compounds are administered orally.

i. Self-Administration

In some embodiments, psychotherapy is neither necessitated nor desired or no specific type of psychotherapy is necessitated or desired; however, any of the disclosed methods can be used in combination with one or more psychotherapy sessions. The flexibility to participate in specific therapies, as well as to choose between any such therapies (or to decide to forgo any specific therapy), while still receiving clinically significant therapeutic effects, is among the advantages of the invention.

In some embodiments, where the therapeutic compounds or pharmaceutical compositions are administered, such administration may occur without or with reduced risk of side effects that would require physician supervision and therefore allow for treatment at home or otherwise outside of a clinic and without the need for such supervision, and/or additionally without the requirement of adjunctive psychotherapy (although it also may be provided in certain embodiments herein). In some embodiments, a patient receives psychological support. In some embodiments, a patient is monitored. Monitoring may be in person or virtual, such as through telemedicine, or computer- or smartphone-assisted means.

ii. Administration In Conjunction with Therapy or Psychotherapy

In some aspects are provided methods of administering therapeutic compounds in conjunction with therapy or psychotherapy, referred to herein as drug-assisted therapy or psychedelic-assisted therapy. In some embodiments, drug-assisted therapy involves the careful screening of subjects or evaluation of inclusion criteria. In some embodiments, the therapeutic program involves both non-drug and drug-assisted sessions, which may be delivered by trained healthcare professionals. In some embodiments, drug-assisted therapy is conducted in a safe and supportive setting. In some embodiments, subjects are monitored throughout the experience.

Using the compositions and methods of the invention, drug assisted therapy can be safely delivered, is well tolerated and can enhance and intensify the psychotherapeutic processes in the treatment of patients with substance use disorders, behavioral addictions, and mental health disorders. Therapeutic compounds given in an appropriate psychotherapeutic context as described herein can reduce avoidance of emotionally distressing thoughts, images, or memories, while increasing empathy for the self and others.

In some embodiments, drug-assisted therapy also can address symptoms of other conditions that are frequently comorbid with substance use, particularly those symptoms associated with a history of psychological trauma.

In some embodiments, therapeutic compounds are administered in conjunction with psychosocial or behavioral therapy, including any of, as non-limiting examples, 12 step facilitation therapy (e.g., as in NIAAA, Project MATCH Monograph Series, 1995; 1(94):3722) CBT (e.g., as in Arch. Gen. Psychiatry 1999; 56:493-502), interpersonal therapy (e.g., as in Psychol. Addict Behav., 2009; 23(1):168-174), contingency management based therapy (e.g., as in Psychol. Addict Behav., 2009; 23(1):168-174; in J. Consul. Clin. Psychol., 2005; 73(2): 354 59; or in Case Reports in Psychiatry, Vol. 2012, Article ID 731638), community reinforcement approach based therapy (e.g., as in Drug Alcohol Depend, 2004; 74:1 13), motivational interviewing based therapy (e.g., as in J. Consul. Clin. Psychol., 2001; 69(5): 858 62), motivational enhancement based therapy (e.g., as in Drug Alcohol Depend, 2007; 91:97-101) or meditation based therapy, such as transcendental meditation based therapy (e.g., as in Addiction, 2004; 99(7):862 874 or J. Consul. Clin. Psychol., 2000; 68(3):515 52).

In some embodiments, therapeutic compounds are administered in conjunction with standardized psychological treatment or standardized psychological support, which refers to standard counseling sessions, for example once a week, twice a week, or as needed, whether in person or virtual (e.g., over telemedicine or by means of a web program or mobile app), and whether with a human therapist or a virtual or Al “therapist,” and in particular counseling focused on alcohol or other substance consumption, or on behavioral impulses or addictions.

From a practice standpoint, a therapist will be certified in the use of the treatment manual for the drug-assisted psychotherapy administered, and will have completed the appropriate training in delivering that form of drug-assisted psychotherapy. Therapists will, for example, receive targeted and specific training in the field of drug-assisted psychotherapy, such as may be delivered online via a combination of online and video conference meeting training sessions and face-to-face tutorials from established clinical professionals in the field of drug-assisted therapy. Therapists delivering drug-assisted therapy will keep up to date on latest developments and publications in the field of drug-assisted therapy, and may attend national, European, and international conferences and gatherings of drug-assisted therapists and researchers. Therapists delivering drug-assisted therapy will receive regular clinical supervision from established drug-assisted psychedelic therapy practitioners. This may include discussing (anonymized) clinical cases and attending regular peer-group meetings with clinicians from a network of other drug-assisted psychedelic therapy services.

Generally, sufficient training will enable a therapist to respond in a safe and supportive manner during drug-assisted therapy sessions. Recent studies with drug-assisted therapy have shown that most psychiatric emergencies, e.g., acute anxiety associated with the emergence of challenging emotional material during a therapeutic session, can be dealt with through “talking down” an anxious patient, with no necessary requirement for psychiatric medication. However, within the definition of “therapist” include psychiatrists licensed to manage psychiatric emergencies by administering “rescue medications” such as short-acting benzodiazepines (e.g., oral lorazepam) if clinically indicated.

In some embodiments, a patient will participate in a treatment protocol or a method of the invention, or be administered a composition of the invention as part of such a method, if the patient meets certain specified inclusion criteria, does not meet certain specified exclusion criteria, does not meet any specified withdrawal criteria during the course of treatment, and otherwise satisfies the requirements of the particular embodiment of the invention as claimed.

Examples of “inclusion criteria,” “exclusion criteria,” and “withdrawal criteria” within the meaning of the invention include those below.

Exemplary Inclusion Criteria

| | |
|---|---|
| 1 | Informed consent |
| 2 | Primary diagnosis (as defined by DSM-5) of SUD or AUD; for non-physically |
| | dependent AUD, having, e.g., no evidence of physical dependence on alcohol |
| 3 | Between 18 and 65 years old |
| 4 | Able to identify in advance a supportive friend, family member, or significant other: |
| | who could meet with the clinician and receive information about the treatment at |
| | one of the preparation sessions prior to the patient's first drug session |
| | who could accompany the patient to study visits if required |
| | who could act as a “locator” and be contacted by the therapy team to remind the |
| | patient about follow-up appointments or collect outcome data (such as drinking |
| | behavior) in the event that the patient themselves cannot be contacted |
| 5 | Proficient in speaking and reading the language in which the therapy is provided |
| 6 | Agree to comply with requirements of therapy protocol |

Exemplary Exclusion Criteria

| | |
|---|---|
| 1 | Lacking capacity |
| 2 | For non-physically dependent AUD, physical dependence on alcohol; evidenced by |
| | clinical assessment, especially evidence of Alcohol Withdrawal Syndrome symptoms |
| 3 | Personal history of, or a current, primary psychotic disorder, bipolar affective |
| | disorder type 1 or personality disorder |
| 4 | Present a serious suicide risk, e.g., as determined using the clinical judgment of one or |
| | more qualified mental health professionals within a therapy team. Risk may be |
| | determined using information from the Columbia-Suicide Severity Risk Scale (C-SSRS) |
| | which allows classification of severity of suicidal ideation and behavior. The C-SSRS |
| | scale classifies severe risk as (a) current suicidal ideation with intent and/or plan, and (b) |
| | suicidal behavior in the last 3 months. A clinical judgment regarding the level of risk |
| | and subsequent decisions regarding eligibility and care could use a combination of the |
| | information provided by the C-SSRS, the patient's history of previous risk behaviors, |
| | any presenting mental health difficulties, and environmental and clinical factors. A final |
| | decision may include a discussion with the qualified mental health professionals within |
| | the therapy team. |
| 5 | Relevant abnormal clinical findings at the screening visit. These include: |
| | History of cardiac disease or stroke |
| | History of severe liver disease, as evidenced by abnormal liver function test |
| | results, particularly reduction in albumin (normal > to 3.5 gm/dl) |
| | History of epilepsy |
| | History of Malignant Hyperthermia (Central Core Disease) |
| | Current untreated/unstable hypertension (however, as with all exemplary inclusion |
| | and exclusion criteria herein, the criteria are not to be considered a replacement for |
| | clinical judgment; in other words, patients with controlled hypertension, e.g., on |
| | antihypertensives, could be considered eligible, based on clinical judgment) |
| 6 | Regular user of MDMA, MEAI, or entactogens, e.g., more than five times in the last |
| | five years or at least twice in the 6 months prior to the start of the study |
| 7 | Currently taking or unwilling/unable to stop any medications inhibiting CYP2D6 |
| | and/or other metabolic enzyme involved in metabolism of compound administered, |
| | and the following medications: monoamine oxidase inhibitors, Ritonavir, paroxetine, |
| | fluoxetine, citalopram, regular benzodiazepines or any other medications likely to |
| | interact with the compound administered; to be stopped during course of drug-assisted |
| | therapy only (e.g., during exemplary 8 or 13 week courses below) |
| 8 | Regular use of or dependence on other drugs such as benzodiazepines, synthetic |
| | cannabinoids, cocaine, or heroin, unless treatment is for related SUDs |

Exemplary Withdrawal Criteria

| | |
|---|---|
| 1 | Development of concurrent physical illness or deterioration of mental health warranting |
| | additional interventions and support that necessitates removal from therapy |
| 2 | The patient becomes pregnant |
| 3 | Start of medications that may interact with the drug (see exclusion criteria above) |
| 4 | Levels of drinking or substance use that interfere with the patient's ability to engage |
| | with the treatment. If a patient has an isolated lapse to drinking or substance use, |
| | participation may be continued once stable enough to engage in the treatment. |
| 5 | If a patient is using illicit substances that are considered to interact adversely with the |
| | drug sessions or other aspects of the psychotherapy course |

Besides satisfying the criteria above, patients generally will be required to taper and stop any medications with a known or suspected risk of drug interaction with the compound administered. Medication will only be tapered and stopped if deemed appropriate based on clinical decision and discussion with the therapy team. The washout period of medications to be stopped will be dependent on the half-life of that particular medication. A sufficient washout period of 2 weeks (5 weeks for fluoxetine) must be allowed prior to the first drug session and medications should not be restarted until completion of the second drug session (for an embodiment with two sessions total) or third drug session (for an embodiment with three sessions total) unless clinically required. Exemplary psychotropic medication that generally will be excluded include: any drugs inhibiting the liver enzyme CYP2D6 and/or other involved metabolic enzyme, antidepressants of the serotonin and/or noradrenaline re-uptake inhibitor type (SSRI/SNRI), Mirtazapine, Ritonavir and regular use of benzodiazepines. These psychotropic medications may either elevate the risk of serotonin syndrome, or may reduce the therapeutic effects (Hysek et al., PLoS One. 2012; 7(5): e36476). By contrast, exemplary medications that generally will be permitted include: pregabalin, gabapentin, occasional benzodiazepines and Z-hypnotics (e.g., zolpidem, zopiclone, zaleplon, or eszopiclone). Commonly used medications that generally will be permitted include baclofen, acamprosate, naltrexone, and disulfiram. Following completion of the drug-assisted sessions patients will be permitted to re-establish any contraindicated medications if desired and clinically indicated, under the care of their therapy team or other medical provider.

iii. Administration Along with Therapeutically Beneficial Activities

In some embodiments, a subject can participate in one or more therapeutically beneficial activities in conjunction with treatment comprising a therapeutic compound. Such activities include, for example, breathing exercises, meditation and concentration practices, focusing on an object or mantra, listening to music, physical exercise, stretching or bodywork, journaling, grounding techniques, positive self talk, or engaging with a pet or animal. Undertaking such activities can occur with or without the participation or guidance of a therapist or other professional. In some such embodiments, participation in the one or more therapeutically beneficial activities will provide beneficial therapeutic effects greater than taking the therapeutic compound or composition alone, or taking the therapeutic compound or composition as part of drug-assisted therapy. In some such embodiments, participation in the one or more therapeutically beneficial activities will provide beneficial therapeutic effects greater than taking the therapeutic compound or composition alone, or taking the therapeutic compound or composition as part of drug-assisted therapy, in addition to the beneficial therapeutic effects of the therapeutically beneficial activities when participated in alone; that is, the conjunction of the treatment and the activity(ies) will provide synergistic effects.

d. Methods of Assessing Efficacy and Adverse Events

In some aspects are provided methods of assessing efficacy of therapeutic compounds, for example, following administration to a subject. Measures of therapeutic effect includes any outcome measure, endpoint, effect measure, or measure of effect within clinical or medical practice or research which is used to assess the effect, both positive and negative, of an intervention or treatment, whether patient-reported, gathered through laboratory tests such as blood work, urine samples etc., or through medical examination.

In one representative example relating to AUD, measures of therapeutic effect for purposes of the invention will be understood to include: (1) Weekly units of alcohol consumed post-baseline (as defined herein) or other measure of relapse status; (2) Quality of life, including subjective sleep; (3) Psychosocial functioning; (4) Prescribed medication use; and (5) Number and frequency of recreational drugs (if any). Those of skill in the art will appreciate that other equivalent or similar measures of therapeutic effect also may be used, and others are described herein and claimed.

In some embodiments, treatment efficacy is evaluated with use of one or more assessments. “Assessment” refers to any means or method used with a patient, whether before, during, after, or unrelated in time to a specific treatment protocol, to measure, estimate, or evaluate a nature, ability, symptom, disorder, or othercharacteristic ofthe patient, whether qualitatively or quantitatively, and whether performed by the therapist or other clinician (e.g., an interview), by the patient his or herself (e.g., a self-reported questionnaire), by a third-party or by a computer, including a medical device (e.g., as such as definedby the FDA or other regulatory body) or other device (e.g., a medical sensor or biosensor, a watch or fitness tracker, or a “wearable”), and whether graded by a human decision-maker or an artificial intelligence, machine learning, or computer algorithm. Non-limiting examples of assessments include those described in Table 1.

TABLE 1
Exemplary Subject Assessments

| | |
|---|---|
| 1 | The Mini International Neuropsychiatric Interview 5 (MINI 5) (Sheehan et al., J. |
| | Clin. Psychiatry, 1998; 59 Suppl 20: 22-33; quiz 34-57) to screen for comorbid |
| | psychiatric disorders. This generally will be completed in full at screening, once, taking |
| | approximately 20 minutes. |
| 2 | The Columbia Suicide Severity Rating Scale (C-SSRS) (Mundt et al., The Journal of |
| | Clinical Psychiatry, 2013; 74(9): 887-93), to screen for acute and recent suicide and |
| | self-harm thoughts and behaviors, taking approximately five minutes to complete. |
| | Generally to be completed at screening, before each of the drug-assisted sessions, the |
| | days after each drug session and during follow-up. |
| 3 | The Patient Health Questionnaire (PHQ-9) (Kroenke et al., J Gen Intern Med., 2001; |
| | 16(9): 606-13). A brief self-administered screening questionnaire for depressive |
| | symptoms. It will be administered at screening, baseline, the final therapy session and |
| | during follow-up (i.e., here and elsewhere, if follow-up is performed), taking |
| | approximately five minutes. |
| 4 | Generalized Anxiety Disorder 7 (GAD-7) (Spitzer et al., Arch Intern. Med., 2006; |
| | 22; 166(10): 1092-7) is a self-reported questionnaire for screening and severity |
| | measuring of generalized anxiety disorder (GAD). It generally will be administered at |
| | screening, baseline, the final therapy session and during follow-up, taking approximately |
| | five minutes. |
| 5 | Pittsburgh Sleep Quality Index (PSQI) (Buysse et al., Psychiatry Res., 1989; |
| | 28(2): 193-213) is used to assess the level of sleep disturbance, completed at screening, |
| | the baseline assessment and during follow-up. |
| 6 | Interpersonal reactivity Index (IRI) (Davis, JSAS Catalog of Selected Documents in |
| | Psychology, 1980; 10, 85) comprises 28 items answered on a 5 point scale. This scale |
| | measures different aspects of empathy and provides different subscales relating to these. |
| | This generally will be administered at baseline, the final therapy session, and during |
| | follow-up to explore if these core aspects of empathy are modified by drug-assisted |
| | therapy. |
| 7 | The Short Form (36) Health Survey (SF-36) is a gold standard patient-reported |
| | measure of quality of life that can be completed at baseline, and during follow-up. |
| 8 | The Self-Compassion Scale (SCS) (Neff, Self and Identity, 2003; 2, 223-250) |
| | Comprises 26 items answered on a 5 point scale. This scale measures core aspects of |
| | self-compassion including components of mindfulness. This generally will be |
| | administered at baseline, the final therapy session, and during follow-up. |
| 9 | The Trauma History Questionnaire (THQ) (Green 1996) is a self-report measure that |
| | examines experiences with potentially traumatic events using a yes/no format. For each |
| | event endorsed, respondents are asked to provide the frequency of the event as well as |
| | their age at the time of the event. It generally will be administered once, at baseline. |
| 10 | The Alcohol Dependence Syndrome section of the SCID-5-CT (Clinical Trials |
| | Version) (First et al., American Psychiatric Association, 2015), this semi-structured |
| | interview generally will be conducted by a trained member of the therapy team and |
| | completed once, at screening, taking approximately ten minutes. |
| 11 | Short Inventory of Problems for Alcohol (SIP) (Kiluk et al., Psychopharmacology, |
| | 2013; 232(19)) SIP for alcohol is a 15-item instrument that assesses the self-attributed |
| | consequences of drinking taking approximately 5 minutes. This generally will be |
| | completed at screening, baseline, the final therapy session and at 3, 6 and 9 months' |
| | follow-up. |
| 12 | The Alcohol Timeline Follow Back (TLFB) (Sobell et al., Substance Use & Misuse, |
| | 2001; 36, 313-331) is a method of assessing daily drinking and recreational drug use |
| | over time. Patients generally will be given the TLFB diary after screening. Data will be |
| | collected at each therapy session. After completion of therapy, a researcher generally |
| | will call on a monthly basis to collect data throughout the study. Further discussion of |
| | the diary generally will take place formally at 3, 6 and 9 months follow up. |
| 13 | The Clinical Institute Withdrawal Assessment for Alcohol-Revised Version |
| | (CIWA-Ar) (Saitz et al., JAMA, 1994; 272: 519-23). This is a ten-item scale used in |
| | the assessment and management of alcohol withdrawal. Each item on the scale |
| | correlates to the severity of alcohol withdrawal. This generally will be administered at |
| | baseline and, if clinically indicated due to concerns from the therapy team, before each |
| | drug-assisted session, taking approximately 3 minutes (although it will be appreciated |
| | that all exemplary patient assessments may be in some embodiments left to clinical |
| | judgment of the therapy team). |
| 14 | The Penn Alcohol Craving Scale (PACS) (Flannery et al., Alcohol Clin. Exp. Res., |
| | 1999; 23: 1289-1295) is used to assess frequency, intensity and duration of thoughts |
| | about drinking, completed at the screening and baseline assessment and at 3, 6 and 9 |
| | months' follow-up. |
| 15 | Obsessive Compulsive Drinking Scale (OCDS) (Anton et al., Alcohol Clin. Exp. Res., |
| | 1995; 19(1): 92-9) is used to measure obsessive and compulsive thoughts in relation to |
| | alcohol. Completed at the screening and baseline assessment and at 3, 6 and 9 months' |
| | follow-up. |
| 16 | Gambling Attitude and Beliefs 23 Survey (GABS-23) (Bouju et al., Journal of |
| | Gambling Studies, 2014; 30(2): 349-367) The GABS-23 is a short 23-question survey |
| | that explores gambling-related cognitions through five dimensions. All items are coded |
| | on a scale ranging from strongly disagree = 0 to strongly agree = 3. The raw subscale |
| | scores are obtained by adding the scores of the items composing each subscale and range |
| | from 0 to 12 or 15. |
| 17 | The Gambling Timeline Follow Back (G-TLFB) (Sobell et al., Substance Use & |
| | Misuse, 2001; 36: 313-331) is a measure of gambling behavior that uses the timeline |
| | followback methodology to assess the intensity and frequency of gambling behavior |
| | over time. Although not initially designed to assess gambling, the TLFB method has |
| | been used to assess gambling behavior in several treatment studies, and use in these |
| | studies has provided support that a specific treatment leads to positive changes in |
| | gambling behavior (Hodgins et al., Addictive Behaviors, 2001; 26: 121-128). |
| 18 | Problem Gambling Severity Index (PGSI) (Ferris & Wynne, Ottawa, ON: Canadian |
| | Centre on Substance Abuse, The Canadian Problem Gambling Index, 2001; 1-59). This |
| | nine-item screening tool can be self-administered or administered by a clinician. It is a |
| | briefer version of the 31-item Canadian Problem Gambling Index, and uses a four-point |
| | scale (“never” to “almost always”) to describe gambling behavior over the previous 12 |
| | months. |
| 19 | South Oaks Gambling Screen (SOGS) (Lesieur & Bloom, American Journal of |
| | Psychiatry, 1990; 144(9). This 20-item scale is perhaps the most well-known screening |
| | tool for disordered gambling. The SOGS has been shown to accurately identify clients |
| | with problem gambling, but was developed using DSM-III criteria so it does not reflect |
| | the DSM-5 criteria. It has also been revised and validated for use with adolescents. |
| 20 | The “Gambling Attitudes Scale” (GAS) (Kassinove, Journal of Clinical Psychology, |
| | 1998; 54(6), 763-771) assesses attitudes (affective, cognitive and behavior-related |
| | aspects) concerning gambling in general and specifically in casinos, horse betting and |
| | lottery, which can foster the development of pathological gambling. |

In some embodiments, an assessment may be computer-assisted, and other computer-assisted assessments may be performed besides the assessments above. The term “computer-assisted” in “computer-assisted assessment” means an assessment comprising the use of electronic tools such as online tools, smartphones, wireless devices, or health apps (in some such examples, also known as “digital phenotyping”). In some embodiments, computer-assisted assessment will include the use of an electronic psychiatric notes system, where relevant clinical information will be recorded for the duration of the therapy by a therapist interacting face-to-face with a patient, and will also include the use of computer systems where the therapist and patient interact virtually (either synchronously or asynchronously), as well as where a patient only interacts with a computer (“computer” broadly meaning any electronic tool suitable for such purposes, including desktop, laptop, and notebook computers; tablets, smartphones, and other mobile devices; watches, fitness trackers, and personal electronic devices; and the like). In some embodiments, one or more aspects of a psychosocial, behavioral, or drug-assisted therapy may be “computer-assisted,” wherein one or more steps of such therapy involve the use of a computer in addition to or as a replacement for some work which would otherwise be performed by a therapist.

In some embodiments, the methods comprise pharmacovigilance. Pharmacovigilance refers to any efforts to monitor the physiological, psychological, or other effects associated with the drug or any other active agent of the invention, especially in order to identify and evaluate adverse events or adverse reactions. For example, at every visit during a treatment period patients may be asked open-ended questions about how they are feeling and any side effects or adverse events will be documented. Therapists will have access to a checklist of common side effects to aid documentation.

In some embodiments, the methods comprise monitoring adverse events. Adverse event (AE) refers to any untoward medical occurrence in a patient or subject administered a medicinal product and which does not necessarily have a causal relationship with this treatment. An AE can therefore be any unfavorable and unintended sign (including an abnormal laboratory finding), symptom, or disease temporally associated with the use of an investigational medicinal product (IMP), whether or not considered related to the IMP. A moderate rise in pulse and blood pressure is common when taking MDMA. For example, MDMA at doses of 100-125 mg has been associated with increases in heart rate of 26-30 bpm and blood pressure (diastolic 14.4-25 mmg) (Harris et al., Psychopharmacology (Berl); 2002; 162(4): 396-405, Mas et al., J. Pharmacol. Exp. Ther., 1999; 290(1): 136-45, Mithoefer et al., J Psychopharmacol., 2011; 25(4):439-52). These may be monitored following a regime such as that known in the art (see, e.g., Oehen et al., J. Psychopharmacol, 2013; 27(1): 40-52, Chabrol & Oehen, J. Psychopharmacol, 2013; s27(9): 865-6). In embodiments of the invention, blood pressure and pulse are measured throughout administration, for example, a drug-assisted session, starting at baseline before the drug is given, and then at appropriate intervals thereafter (e.g., hourly, every two hours) for six hours. In the event of a patient presenting with acutely elevated BP (e.g., systolic BP [SBP]>200 mm Hg or diastolic BP [DBP]>120 mm Hg) the patient will be referred for immediate emergency hospital care. The upper age range of patients may be limited, e.g., to 65 years, and patients may be screened for cardiac abnormalities. However, therapeutic compounds are expected to show less significant increases in blood pressure and heart rate compared to MDMA. In preferred embodiments, the therapeutic compounds and compositions will have fewer AEs than comparator compounds such as MDMA and MEAI.

In some embodiments, the methods comprise monitoring adverse reactions. Adverse reaction (AR) refers to all untoward and unintended responses to an IMP related to any dose administered. All AR₅ judged as having reasonable causal relationship to a medicinal product qualify as adverse reactions. The expression “reasonable causal relationship” means to convey in general that there is evidence or argument to suggest a causal relationship. In preferred embodiments, the therapeutic compounds and compositions will have fewer AR₅ than comparator compounds such as MDMA, MEAI (5-methoxy-2-aminoidane), and MMAI (5-Methoxy-6-methyl-2-aminoindane).

In some embodiments, the methods comprise monitoring unexpected adverse reactions (UAR). UAR refers to an AR, the nature or severity of which is not consistent with the applicable product information (e.g., investigator's brochure for an unapproved investigational product, summary of product characteristics (SmPC) for an authorized product, or package insert or other product labeling for an approved drug). When the outcome of the adverse reaction is not consistent with the applicable product information this adverse reaction should be considered as unexpected. Side effects documented in the SmPC which occur in a more severe form than anticipated are also considered to be unexpected. In preferred embodiments, the therapeutic compounds and compositions will have fewer UAR₅ than comparator compounds such as MDMA and MEAI.

In some embodiments, the methods comprise monitoring serious adverse events (SAEs) or serious adverse reactions (SARs), which refer to any untoward medical occurrence or effect that at any dose: results in death; is life-threatening (i.e., an event in which the subject was at risk of death at the time of the event, but not an event which hypothetically might have caused death if it were more severe); requires hospitalization, or prolongation of existing inpatients' hospitalization; results in persistent or significant disability or incapacity; or is a congenital anomaly or birth defect. However, this list is non-exclusive, and medical judgment will be exercised in deciding whether an AE or AR is serious in other situations. Important AEs or ARs that are not immediately life-threatening or do not result in death or hospitalization but may jeopardize the subject or may require intervention to prevent one of the other outcomes listed in the definition above, also should be considered serious. In preferred embodiments, the therapeutic compounds and compositions will have fewer SAEs and/or SAR₅ than comparator compounds such as MDMA and MEAI.

In some embodiments, the methods herein comprise monitoring suspected unexpected serious adverse reactions (SUSARs), which refers to any suspected adverse reaction related to an IMP that is both unexpected and serious (i.e., by reference to the definitions for UAR and SAR above). In preferred embodiments, the therapeutic compounds and compositions will have fewer SUSARs than comparator compounds such as MDMA and MEAI.

e. Dosing

In some aspects provided are doses for therapeutic compounds or pharmaceutical compositions thereof. Dosing may be adjusted according to route of administration. In some embodiments, disclosed therapeutic compounds or pharmaceutical compositions thereof are administered parenterally. In some embodiments, disclosed therapeutic compounds or pharmaceutical compositions thereof are administered intravenously. In some embodiments, disclosed therapeutic compounds or pharmaceutical compositions thereof are administered orally. Dosage amounts will be understood by reference to the teachings herein together with the general knowledge in the art, but certain exemplary dosage amounts, known to be useful in the practice of the invention, are listed below for ease of reference.

In some embodiments, where a therapeutic compound is formulated in a liquid solution, a dosage amount may be determined based on the concentration of the therapeutic compound in the solution; for example, where a solution of 250 mg therapeutic compound in 5 mL liquid is used for injection, a volume of liquid for injection may be, e.g., 0.1 mL or less, at least 0.1 mL, at least 0.2 mL, at least 0.3 mL, at least 0.4 mL, at least 0.5 mL, at least 0.6 mL, at least 0.7 mL, at least 0.8 mL, at least 0.9 mL, or at least 1.0 mL, at least 1.2 mL, at least 1.4 mL, at least 1.6 mL, at least 1.8 mL, or at least 2.0 mL as well as amounts within these ranges. In some embodiments, the liquid solution may be formulated for parenteral administration. In some embodiments, the liquid solution may be formulated for IV administration. In some embodiments, the liquid solution may be formulated for intramuscular (IM) administration. In some embodiments, the liquid solution may be formulated for subcutaneous (SC) administration.

In some embodiments, where a therapeutic compound is formulated for parenteral or intravenous administration, it may be present in an amount so that a single dose is (in a milligram dosage amount calculated based on the kilogram weight of the patient), e.g., 0.1 mg/kg or less, at least 0.2 mg/kg, at least 0.3 mg/kg, at least 0.4 mg/kg, at least 0.5 mg/kg, at least 0.6 mg/kg, at least 0.7 mg/kg, at least 0.8 mg/kg, at least 0.9 mg/kg, at least 1.0 mg/kg, at least 1.1 mg/kg, at least 1.2 mg/kg, at least 1.3 mg/kg, or at least 1.4 mg/kg, as well as amounts within these ranges.

In some embodiments, where a therapeutic compound is formulated in a unit dosage form, it may be present in an amount so that a single dose is, e.g., 1 mg or less, at least 1 mg, at least 2 mg, at least 3 mg, at least 4 mg, at least 5 mg, at least 7.5 mg, at least 10 mg, at least 12.5 mg, at least 15 mg, at least 17.5 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 125 mg, at least 150 mg, at least 175 mg, or at least 200 mg. In some embodiments, where a therapeutic compound is formulated in an oral dosage form, it may be present in an amount so that a single dose is (whether or not such dose is present in a unit dosage form), e.g., 1 mg or less, at least 1 mg, at least 2 mg, at least 3 mg, at least 4 mg, at least 5 mg, at least 7.5 mg, at least 10 mg, at least 12.5 mg, at least 15 mg, at least 17.5 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 125 mg, at least 150 mg, at least 175 mg, or at least 200 mg.

In some embodiments, a therapeutic compound may be formulated in a buccal or sublingual dosage form, or another dosage form that avoids first-pass metabolism or otherwise results in higher bioavailability, it may be present in an amount so that a single dose is (whether or not such dose is present in a unit dosage form), e.g., 0.5 mg or less, at least 1.0 mg, at least 1.5 mg, at least 2.0 mg, at least 2.5 mg, at least 3.0 mg, at least 3.5 mg, at least 4.0 mg, at least 5.0 mg, at least 6.0 mg, at least 7.0 mg, at least 8.0 mg, at least 9.0 mg, at least 10 mg, at least 12.5 mg, at least 15 mg, at least 20 mg, at least 25 mg, at least 30 mg, at least 40 mg, or at least 50 mg.

In some embodiments, where a therapeutic compound is formulated in a dosage form not separately discussed above, it may be present in an amount so that a single dose is (whether or not present in a unit dosage form), e.g., 1 mg or less, at least 1 mg, at least 2 mg, at least 3 mg, at least 4 mg, at least 5 mg, at least 7.5 mg, at least 10 mg, at least 12.5 mg, at least 15 mg, at least 17.5 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 125 mg, at least 150 mg, at least 175 mg, or at least 200 mg.

In some embodiments, where a pharmaceutical composition additionally includes an additional active agent, such as a tryptamine or phenethylamine as defined above, it may be present in an amount so that a single dose is (whether or not such dose is present in a unit dosage form), e.g., 50 mg or less, at least 50 mg, at least 60 mg, at least 65 mg, at least 70 mg, at least 75 mg, at least 80 mg, at least 85 mg, at least 90 mg, at least 95 mg, at least 100 mg, at least 125 mg, at least 150 mg, at least 175 mg, or at least 200 mg.

A single dose of an additional active agent may, in some embodiments, be known through the practice of ordinary skill or by general knowledge.

It will be readily appreciated that dosages may vary depending upon whether the treatment is therapeutic or prophylactic, the onset, progression, severity, frequency, duration, probability of or susceptibility of the symptom to which treatment is directed, clinical endpoint desired, previous, simultaneous or subsequent treatments, general health, age, gender, and race of the subject, bioavailability, potential adverse systemic, regional or local side effects, the presence of other disorders or diseases in the subject, and other factors that will be appreciated by the skilled artisan (e.g., medical or familial history).

Dose amount, frequency or duration may be increased or reduced, as indicated by the clinical outcome desired, status of the pathology or symptom, any adverse side effects of the treatment or therapy, or concomitant medications. The skilled artisan with the teaching of this disclosure in hand will appreciate the factors that may influence the dosage, frequency, and timing required to provide an amount sufficient or effective for providing a therapeutic effect or benefit, and to do so depending on the type of therapeutic effect desired, as well as to avoid or minimize adverse effects.

In some embodiments, the dose actually administered will be determined by a physician, in light of the relevant circumstances, including the disorder to be treated, the chosen route of administration, the actual composition or formulation administered, the age, weight, and response of the individual patient, and the severity of the patient's symptoms, and therefore any dosage ranges disclosed herein are not intended to limit the scope of the invention. In some instances, dosage levels below the lower limit of a disclosed range may be more than adequate, while in other cases doses above a range may be employed without causing any harmful side effects, provided for instance that such larger doses also may be divided into several smaller doses for administration, either taken together or separately. In some embodiments, therapeutic compounds are administered parenterally. In some embodiments, therapeutic compounds are administered systemically. In some embodiments, therapeutic compounds are administered intravenously. In some embodiments, therapeutic compounds are administered orally.

In some such embodiments, the therapeutic compounds or pharmaceutical compositions thereof will be administered and dosed in accordance with good medical practice, taking into account the method and scheduling of administration, prior and concomitant medications and medical supplements, the clinical condition of the individual patient and the severity of the underlying disease, the patient's age, sex, body weight, and other such factors relevant to medical practitioners, and knowledge of the particular compound(s) used. Starting and maintenance dosage levels thus may differ from patient to patient, for individual patients across time, and for different pharmaceutical compositions and formulations, but shall be able to be determined with ordinary skill.

It should be appreciated that in other embodiments, e.g., when the compositions of the invention are taken without the direct intervention or guidance of a medical professional, appropriate dosages to achieve a therapeutic effect, including the upper and lower bounds of any dose ranges, can be determined by an individual by reference to available public information and knowledge, and reference to subjective considerations regarding desired outcomes and effects.

Determination of appropriate dosing shall include not only the determination of single dosage amounts, but also the determination of the number and timing of doses, e.g., administration of a particular dosage amount once per day, twice per day, or more than twice per day, and the time(s) of day or time(s) during a psychotherapeutic session preferable for their administration, and any alternating daily dosing regimen.

In some embodiments, especially where a formulation is prepared in unit dosage form, such as a capsule, tablet, or lozenge, suggested dosage amounts may be known by reference to the format of the preparation itself. In other embodiments, where a formulation is prepared in multiple dosage form, for instance liquid suspensions and topical preparations, suggested dosage amounts may be known by reference to the means of administration or by reference to packaging and labeling, package insert(s), marketing materials, training materials, or other information and knowledge available to those of skill or the public.

In some embodiments, a patient will have an option of using online software such as a website, or downloadable software such as a mobile application, to assist with compliance or to provide data relating to treatment. Such software can be used to, e.g., keep track of last dose taken and total doses taken, provide reminders and alerts for upcoming doses, provide feedback to discourage taking doses outside of set schedules, and allow for recording of specific subjective effects, or provide means for unstructured journaling. Data collection can assist with individual patient compliance, can be used to improve or tailor individual patient care plans, and can be anonymized, aggregated, and analyzed (including by Al or natural language processing means) to allow research into effects of various methods of treatment.

f. Therapeutic Effects

As used herein, the terms “subject,” “patient,” and “individual” are used interchangeably, and refer to any mammal but preferably a human. As used herein, the terms “subject,” “patient,” and “individual” include, but are not limited to, one who has a substance use disorder, behavioral addiction, CNS or mental health disorder, or a condition related to any of the aforementioned conditions for which similar treatment may be efficacious, or who seeks improvement in mental health or psychological functioning. When a “subject,” “patient,” or “individual” is participating in a course of therapy as for example in a course of psychedelic-assisted therapy, the term “participant” also may be used. These terms also shall refer to patients in need of treatment, persons predisposed to a disorder, and subjects whether or not diagnosed with a disorder. Moreover, these terms shall likewise refer to persons who have received treatment or therapy for any such conditions, are currently receiving therapy or treatment for a disorder, or who may receive therapy or treatment for a disorder in the future.

In some embodiments, the disclosed methods are used, or are additionally used, to improve mental health and improve psychological functioning in non-disease states, i.e., in an individual without a diagnosed mental disorder, or specific symptoms thereof.

In general, all of the therapeutic compounds, compositions, and methods of the invention will be appreciated to work for all individuals, although individual variation is to be expected, and will be understood. Where there is variation between individuals, modification to the therapeutic compounds, compositions, and methods will be understood based on the teachings herein in combination with the general knowledge of the art.

In some instances, certain personalized approaches (i.e., “personalized” or “precision” medicine) may be utilized, based on individual characteristics, including drug metabolism (e.g., CYP2D6 or CYP3A4) and/or individual genetic variation (e.g., a polymorphism related to an esterase). A “genetic variation” refers to a change in a gene sequence relative to a reference sequence (e.g., a commonly-found or wildtype sequence). Genetic variation may be recombination events or mutations such as substitution/deletion/insertion events like point and splice site mutations. In one embodiment, the genetic variation is a genetic variation in metabotropic glutamate receptor type 5 (mGluR5), which has been implicated in mood and anxiety symptoms in humans. In another embodiment, the genetic variation is one or more single nucleotide polymorphisms (SNPs) in the FKBP5 gene that are associated with elevated levels of FKBP51 protein relative to persons lacking such SNPs. The FKBP5 gene has been implicated in responses to stress and trauma, and such SNPs are correlated with susceptibility to certain depression, PTSD, and anxiety disorders (Yehuda et al., Biological Psychiatry, 2016; 80(5), 372-380; Bierer, American Journal of Psychiatry, 2020; 177:8, 744-753).

As used herein, “therapist” refers to a person who treats a patient using the compositions and methods of the invention, whether that person is a psychiatrist, clinical psychologist, clinical therapist, registered therapist, psychotherapist, or other trained clinician, counselor, facilitator, or guide, although it will be understood that certain requirements will be appropriate to certain aspects of the drug-assisted therapy (e.g., prescribing, dispensing, or administering a drug, offering psychotherapeutic support). In some embodiments, a “person” may also include an Al.

“Treating” or “treatment” of a disorder includes: (i) inhibiting the disorder, i.e., arresting or reducing the development or progression of the disorder or its clinical symptoms; or (ii) relieving the disorder, i.e., causing regression of the disorder or its clinical symptoms. Inhibiting the disorder, for example, would include prophylaxis. Hence, one of skill will understand that a therapeutic amount necessary to effect treatment for purposes of this invention will, for example, be an amount that provides for objective indicia of improvement in patients having clinically-diagnosable symptoms. The effect may be prophylactic in terms of completely or partially preventing a disorder or symptom thereof and/or may be therapeutic in terms of a partial or complete cure for a disorder, an adverse effect attributable to the disorder, and/or a comorbidity simultaneously present with the disorder. In one example, a “comorbidity” present with alcohol use disorder (AUD) includes psychiatric disorders such as antisocial personality disorder, borderline personality disorder, depression, anxiety, schizophrenia, attention deficit hyperactivity disorder, bipolar disorder, obsessive compulsive disorder, binge eating disorder, and PTSD.

“Treatment” as used herein covers any treatment of a disorder in a mammal, and preferably in a human, and includes: (a) preventing a disorder from occurring in a subject who may be predisposed to the disorder but has not yet been diagnosed with it; (b) inhibiting a disorder, i.e., arresting its development; (c) relieving a disorder, i.e., causing regression thereof; (d) protection from or relief of a symptom or pathology caused by or related to a disorder; (e) reduction, decrease, inhibition, amelioration, or prevention of onset, severity, duration, progression, frequency or probability of one or more symptoms or pathologies associated with a disorder; and (f) prevention or inhibition of a worsening or progression of symptoms or pathologies associated with a disorder or comorbid with a disorder. Other such measurements, benefits, and surrogate or clinical endpoints, alone or in combination, will be understood to one of skill based on the teachings herein and the general knowledge in the art.

“Therapeutic effect” or “therapeutic efficacy,” as used herein, means the responses(s) in a mammal, such as a human, after treatment that are judged to be desirable and beneficial. Hence, depending on the disorder to be treated, or improvement in physiological or psychological functioning sought, and depending on the particular constituent(s) in the compositions of the invention under consideration, those responses shall differ, but would be readily understood by those of ordinary skill. Such responses will be understood based on the teachings herein and the general knowledge in the art.

“Therapeutically effective amount,” as used herein, refers to that amount of a compound or combination of therapeutic compounds that is sufficient to effect treatment, when administered to a subject in need of such treatment. The therapeutically effective amount will vary depending upon the subject and disease condition being treated, the weight and age of the subject, the severity of the disease condition, the manner of administration and the like, which can readily be determined by one of ordinary skill in the art.

Administration of a composition in a “therapeutically effective amount” or an “effective amount” to a subject means administration of an amount of a therapeutic compound or a composition thereof in an amount sufficient to achieve a desired effect. In some embodiments, a “therapeutically effective amount” or “effective amount” thus would be the amount sufficient to cause a measurable modulation of a neurotransmitter system, such as one or more of the serotonergic, dopaminergic, and/or noradrenergic systems, where that measurable modulation is known to result in or is shown to result in treatment of a disorder. When an “effective amount” means an amount effective in treating the stated disorder itself, or in the symptoms thereof in a subject, “therapeutic effect” would be understood to mean the responses(s) in the subject after treatment judged to be desirable and beneficial.

### G. WORKING EXAMPLES

The following examples are included for illustrative purposes only and are not intended to limit the scope of the invention. Studies were performed in agreement with procedures to assure data integrity and adherence to GLP standards. Therapeutic compounds were tested in multiple assays as now described.

### Example 10: Functional Dopamine Transporter (DAT) and Serotonin Transporter (SERT) Uptake Assay in Recombinant hDAT-CHO Cells, and Dopamine (DA) and Serotonin Release Assay (5-HT) in Rat Synaptosomes

Purpose: Disclosed compounds were assayed to determine inhibitory activity on monoamine transporters, such as DAT and SERT, and to characterize their effects on dopamine (DA) and serotonin (5-HT) release. Aminoindanes MDMA, MEAI, and MMAI were also assayed for reference. A scintillation proximity assay (SPA) was used to determine SERT and DAT uptake in a recombinant CHO cell line (pIC₅₀). Rat synaptosomes were used to assess the DA and 5-HT release activity of therapeutic compounds (pEC₅₀).

Methods-Evaluation of Monoamine Uptake/Transporter Inhibition: Inhibitory activity on DAT function was measured using an uptake assay in a recombinant CHO cell line stably expressing human DAT (hDAT-CHO). Potency was measured in terms of pIC50 by testing for inhibition of [3H]-dopamine uptake in hDAT-CHO cells in a multiwell format SPA.

Briefly, on experiment days, hDAT-CHO cells were detached using Versene and added at a density of approximately 300,000 cells/mL to the SPA Mixture, which contains the following components in Assay Buffer (20 mM HEPES, 145 mM NaCl, 5 mM KCl, 2 mM CaCl₂), 1 mM MgCl2, 5.5 mM glucose, 0.01% Pluronic F127, pH 7.3): 0.75 mg/mL SPA Imaging beads (RPNQ0001, PerkinElmer), 10 μM pargyline and 80 nM of [3H]-dopamine (NET673, PerkinEImer). The SPA Mixture was added 20 μL/well to 384 well plates containing 0.1 μL/well of test compound in neat DMSO (11 points of 1:5 serial dilution, 0.5% DMSO final) or 0.1 μL of DMSO (total uptake) or 0.1 μL of the standard inhibitor indatraline (at 1 μM final concentration, as nonspecific uptake). Plates were sealed with a Top-seal A and read using a Microbeta counter (PerkinElmer) after 45 minutes of incubation at room temperature. In addition, hSERT-CHO cells were detached using Versene and added at a density of 150,000 cells/mL to the SPA Mixture, which contains the following components in the Assay Buffer described above: 0.5 mg/mL SPA Imaging beads (RPNQ0001, PerkinElmer), 10 μM pargyline and 35 nM of [3H]-serotonin (NET498, PerkinElmer). The SPA Mixture was added 20 μL/well to 384 well plates containing 0.1 μL/well of test compound in neat DMSO (11 points of 1:5 serial dilution, 0.5% DMSO final), 0.1 μL of DMSO (total uptake), or 0.1 μL of the standard inhibitor indatraline (1 μM, nonspecific uptake). Plates were sealed with a Top-seal A and read using a Microbeta counter (PerkinElmer) after 120 minutes of incubation at room temperature.

For both tests, the raw experimental data (cpm) were elaborated for the determination of pIC50/IC50 values as % inhibition of specific uptake. Concentration-response curves of MDMA, MDDMA, citalopram and GBR-12909 were used as internal standards in all experiments, and the signal window was monitored in each plate performing Z′ calculations, as described in Zhang et al., J Biomol Screen. 1999; 4: 67-73.

Methods-Evaluation of Monoamine Release: The potency of therapeutic compounds for stimulating DA release was measured by an endpoint release filtration assay in a 96-well format using rat synaptosomes, essentially as described by Sandtner et al. (Mol Pharmacol., 2016; 89(1):165-75.) and Baumann et al. (Neuropsychopharmacol, 2013; 38:552-562.) with some variations. One male rat was euthanized, and striatum/cortex regions were dissected over ice. The striatum portions were weighed and put in a glass tube containing ice-cold 0.32 M sucrose (dilution 1:24 w/v) for homogenization, while the frontal cortex tissue was diluted in ice-cold 0.32 M sucrose, 1:20 w/v. Tissues were homogenized and centrifuged for 10 mins at 1000×g. The two supernatants from striatum and cortex (crude synaptosomes) were retained on ice until use.

Test compounds were then serially diluted 1:4 in neat DMSO as concentration-response curves (CRC) of 11 points. To each well of a 2-mL 96 deep-well plate (Whatman UNIPLATE 96 well) were added: 2 μL of a concentration-response curve (CRC) of test compounds or 2 μL of DMSO (no-release condition) or 2 μL of MDMA 10 mM solution (final 50 μM, for maximal release), followed by 200 μL/well of complete Krebs-phosphate buffer (126 mM NaCl, 2.4 mM KCl, 0.83 mM CaCl₂), 0.8 mM MgCl2, 0.5 mM KH2PO4, 0.5 mM Na2SO4, 11.1 mM glucose, 0.05 mM pargyline, 1 mg/mL ascorbic acid, freshly added, pH 7.4), containing 1 μM reserpine to block vesicular uptake of substrate. Desipramine (100 nM) and citalopram (100 nM) were also added to the release assay buffer in order to inhibit NET and SERT uptake, respectively. The final compound dilution, then, was 1:200 in 0.5% DMSO.

3H-Dopamine was added to the tube containing the crude striatal synaptosomes preparation, diluted 1:20 (v/v) in complete Krebs-phosphate buffer, and was incubated at RT and gently mixed for 60 minutes. Then, 200 μL/well of 3H-DA loaded synaptosomes were dispensed to the compound plate (final 400 μL/well), and the release reaction proceeded for an additional 15 minutes at RT. The release was terminated by vacuum filtration and washing three times with 1 mL of saline solution (NaCl 0.9%) at RT. The retained radioactivity was counted in a scintillation counter after the addition of 50 μL/well Microscint-20.

The raw data (cpm) from residual radioactivity onto the filter were elaborated for the determination of pEC50/EC50 values. Specifically, the compound effect was expressed as the percent of maximal release obtained with 50 μM MDMA (100% release) in comparison to vehicle (0.5% DMSO, 0% release). Both MDMA and D-amphetamine in CRC were used as internal controls in each release experiment. In each plate, for both 0% and 100% of release, the signal window was monitored and Z′ calculations were determined using the raw data of four replicates. 5-HT release was calculated in an identical fashion, except that 5 nM 3H-serotonin was added to crude cortex synaptosomes preparation diluted 1:13 (v/v) in complete Krebs-phosphate buffer containing 1 μM Reserpine, 100 nM GBR12909 and 100 nM nomifensine to block vesicular, DAT, and NET uptake, respectively. Synaptosomes were then loaded with 3H-5HT for 60 minutes at RT. After dispensing loaded synaptosomes onto a compound plate (200 μL/well), the release reaction proceeded for an additional 15 minutes. Stop by filtration, counting, and data elaboration were performed as described for DA release. Compounds were tested in duplicate (n=2).

Results and Significance: Results are shown in Table 2 and presented as pIC₅₀ for uptake assays and pEC₅₀ for release assays. Higher values indicate greater inhibitory activity or greater release activity. 5-HT release of activity of each exemplary compound was reduced relative to MDMA, MEAI, and MMAI. Additionally, SERT/DAT ratios are presented. SERT/DAT uptake or inhibition ratios have been associated with stimulant and entactogenic effects. In one representative example, the ratio for model entactogen MDMA favors SERT inhibition (DAT/SERT ratio=0.8), whereas stimulants, like methamphetamine, display greater inhibition of DAT (DAT/SERT ratio=10) (Simmler et al., Br J Pharmacol. 2013; 168(2): 458-470). SERT/DAT ratios presented herein were calculated as 1/SERT IC50:1/DAT IC50. The SERT/DAT uptake ratio of exemplary compounds CMP-405, CMP-407, CMP-408 was comparable to or increased relative to MDMA. The SERT/DAT release ratio of CMP-405 exceeded that of MDMA (3.2 vs 2.6).

| | | | | CMP- | CMP- | CMP- | CMP- | CMP- | CMP- |
|---|---|---|---|---|---|---|---|---|---|
| Assay | MDMA | MEAI | MMAI | 401 | 402 | 405 | 406 | 407 | 408 |
| DAT | 5.7 | 4.3 | 4.3 | 4.0 | 4.0 | 4.7 | <4.0 | 4.2 | 4.0 |
| Uptake | | | | | | | | | |
| SPA | | | | | | | | | |
| (pIC₅₀) | | | | | | | | | |
| DA | 6.6 | 5.4 | 4.6 | <4.0 | <4.0 | 5.6 | <4.0 | 4.6 | <4.0 |
| Release | | | | | | | | | |
| (pEC₅₀) | | | | | | | | | |
| SERT | 6.0 | 5.4 | 6.0 | 5.4 | 4.2 | 5.1 | 4.2 | 4.6 | 4.4 |
| Uptake | | | | | | | | | |
| SPA | | | | | | | | | |
| (pIC₅₀) | | | | | | | | | |
| 5-HT | 7.0 | 6.8 | 7.4 | 5.5 | 5.0 | 6.1 | 4.3 | <4.0 | 4.6 |
| Release | | | | | | | | | |
| (pEC₅₀) | | | | | | | | | |
| SERT/ | 2.1 | 9 | 52 | 22 | 1.4 | 1.9 | >1.6 | 2.5 | 2.5 |
| DAT | | | | | | | | | |
| Uptake | | | | | | | | | |
| Ratio | | | | | | | | | |
| SERT/ | 2.6 | 28 | 575 | >35 | >10 | 3.2 | >2.0 | <0.25 | 3.9 |
| DAT | | | | | | | | | |
| Release | | | | | | | | | |
| Ratio | | | | | | | | | |

### Example 11: Measurement of Intracellular Calcium Response in CHO Cells Expressing the Human 5-HT2A Receptor

Purpose: The 5-HT2A selectivities of therapeutic compounds were assessed in agonist and antagonist modes.

Methods: Selectivity for the 5-HT2A receptor was assessed by measuring [ICa2+]i in a CHO recombinant cell line stably expressing the human 5-HT2A receptor, using a Fluorometric Imaging Plate Reader (FLIPR TETRA, Molecular Devices).

The day before the experiment, CHO cells were seeded into black-wells clear-bottom 384-well plates at a density of 10,000 cells per well, in F-12K Medium (ThermoFisher #21127) supplemented with 10% dialyzed fetal bovine serum, and grown overnight at 37° C., 5% C₀₂.

On the day of the experiment, the medium was replaced with assay buffer (20 miM HEPES, 145 mM NaCl, 5 mM KCl, 5.5 mM glucose, 1 mM MgCl2, 2 mM CaCl2 and 2.5 mM probenecid, pH 7.4) containing the cytoplasmic Ca2+ indicator Fluo-4 AM at 2 μM and 0.02% Pluronic F-127. After an incubation of 45-60 minutes at 37° C., 0% C₀₂, cells were washed by Microplate Washer (BIOTEK) 384 with assay buffer, and the plate was transferred to the FLIPR TETRA. Then, intracellular fluorescence was measured in real time by FLIPR TETRA (excitation wavelength at 470-495 nm, emission wavelength at 515-575 nm), using a dual addition protocol.

In the first addition, test compounds (11 points of 1:4 serial dilution, 0.5% DMSO final) were added to the cell plate to evaluate their effect on intracellular calcium levels. In the event of a response, the signal was elaborated for the determination of pEC50/EC50 values. After 10 minutes of incubation, a second addition of a submaximal concentration of serotonin (EC80) was performed. Inhibition of 5-HT response vs. vehicle effect (0.5% DMSO) was elaborated for the determination of pIC50/IC50 values. Concentration-response curves (CRC) of 5-HT and LSD were used as internal controls in the agonist format, full agonist and partial agonist, respectively. A CRC of ketanserin was used as the internal antagonist control. The activity of MDMA was also assessed and used as a reference.

Results and Significance: The 5-HT_{2A} selectivities of test compounds (n=2) were calculated and compared to MDMA. Agonist and antagonist activity of MEAI and CMP-405 was comparable to MDMA, which displayed selectivities of less than 4.3 (represented as pEC₅₀ and pIC₅₀, respectively) in each mode. For reference, the mean pEC₅₀ values of full 5-HT_{2A} receptor agonist serotonin and partial agonist LSD were approximately 8.4 and 7.8, respectively, and the mean pEC₅₀ value of 5-HT_{2A} receptor antagonist ketanserin was 5.5. Regarding antagonist mode, the pIC₅₀ values for serotonin, LSD, and ketanserin were 5.5, 5.0, and 9.0, respectively.

### Example 12: Assessment of Solubility in PBS (pH 7.4)

Purpose: The solubility of therapeutic compounds in PBS was evaluated using an HPLC-UV-based method.

Methods: Assays were conducted with ibuprofen as a reference for high solubility and progesterone as a reference for low solubility. Stock solutions of therapeutic compounds (20 mM) were prepared in DMSO, on the same day of the experiment when feasible. These solutions were compared with an external standard solution of known concentration via HPLC-UV. To prepare the standard, 4 μL from a 20 mM compound stock solution was added to DMSO in a vial/well, and mixed with shaking at room temperature for 10 minutes. The standard buffer used for the assessment was PBS buffer (pH 7.4), but alternative buffers may also be used, such as biologically relevant Fluids, including Simulated Gastric fluid (SGF) at pH 1.2, Fasted State Simulated Intestinal Fluid (FaSSIF) at pH 6.5, and Fed State Simulated Intestinal Fluid (FeSSIF) at pH 5.0.

An isotonic phosphate buffer (iPBS) was prepared by dissolving a phosphate buffered saline tab (Sigma-Aldrich P4417-50Tab) in 200 mL of deionized water, the final composition of the solution being 10 mM PBS, 2.7 mM kCl, and 137 mM NaCl, yielding a final pH of 7.4 at 25° C. Samples were prepared by dispensing 8 μL from a 20 mM compound stock solution in DMSO in a vial/well in duplicate, adding 392 μL of buffer in each vial/well, and mixed by shaking at room temperature for 120 minutes. The final compound concentration was approximately 400 μM, while the final concentration of DMSO was about 2%. The solutions were then filtered prior to analysis.

Concentrations were determined by comparing UV absorbance of the test solution and of the known standard solution following HPLC separation using a generic fast gradient method employing a DAD detector and UV detection at 230 and 254 nm. Concerning the HPLC Mobile phase, Phase A was a 50 mM ammonium acetate aqueous solution (pH adjusted to 7.4 with ammonia), and Phase B was acetonitrile. For data analysis, chromatographic peaks were automatically integrated, and the parameters were optimized to enable consistent integration of all chromatograms in the entire analytical run. The solubility of each compound was expressed as the ratio of compound amount in the sample test solution to the amount of compound in the standard solution, calculated from the equation “solubility of sample=peak area of sample/peak area of standard×concentration of standard.”

Results and Significance: The solubility of tested compounds was classified as follows:
Compounds with a solubility above 250 M were classified as Highly Soluble;
Compounds with a solubility between 100 μM to 250 μM were classified as Medium Soluble; and
Compounds with solubility below 100 M were classified as Low Soluble

All tested compounds were determined to be highly soluble, and the solubility of MEAI and CMP-405 was comparable to MDMA. Solubility is a key physicochemical property of a chemical entity that can be considered in pharmacological and pharmaceutical contexts, such as when considering absorption and ease of formulation.

| Compound | Solubility (μM) | Characterization |
|---|---|---|
| MDMA | 386 | High solubility |
| MEAI | 374 | High solubility |
| CMP-405 | 392 | High solubility |

### Example 13: In Vitro Study of Clearance in Rat and Human Liver Microsomes

Purpose: The in vitro metabolic stability of therapeutic compounds was evaluated by determining clearance in liver microsomes of rat and human origin. For comparative purposes, MDMA and aminoindanes MEAI and MMAI were also assayed.

Methods: The general procedure involved incubating test compounds with liver microsomes and subsequently detecting the compounds to determine intrinsic clearance. Clearance was estimated from the rate of depletion k (min-1), the volume of the incubation V (mL), and the amount of microsomal proteins in the incubation M (mg). Intrinsic clearance is defined by the following equation: Clᵢₙₜ=k*V/M.

Values for CLint are expressed as μL/min/mg liver microsomes. Verapamil and dextromethorphan were used as positive controls for CYP3A4 and CYP2D6 isoforms, respectively, to confirm metabolic activity. Stock solutions were prepared in DMSO for therapeutic compounds and positive controls verapamil and dextromethorphan (10 mM for all). Working solutions were prepared by diluting stock in MeOH to reach 50 mM. However, this concentration can be varied, e.g., when the test item is not soluble in the selected solvent.

Prior to experimentation, frozen liver microsomes were thawed in a water bath at 37° C. and kept on ice until use. The microsomes were then diluted with 50 mM potassium phosphate buffer pH 7.4 to a protein concentration of 0.56 mg/mL. To prepare test and control items in final incubation conditions, 5 μL of test compound and positive control working solutions were added to 445 μL of microsomes incubation mixture at 0.56 mg/mL and 50 μL of regenerating system.

As it relates to the automated incubation procedure for microsomes incubation, the following procedure was followed. Briefly, 150 μL of quenching solution (ACN containing an appropriate Internal Standard, i.e. Rolipram and Diclofenac for positive and negative ion mode, respectively) was manually dispensed or dispensed by using the Hamilton system in each well of 96 deep well 1 mL plates. Next, 800-1000 μL-aliquots of NADPH regenerating system were pre-warmed at 37° C. for 5 minutes. 5 μL of 50 M test items and control were added to 445 μL of the 0.56 mg/mL microsomes solution, and the incubation mixture was pre-warmed in a 96 deep well 2 mL plate (incubation plate) at 37° C. for 5 minutes. The incubation reactions were initiated by adding 50 μL of pre-warmed NADPH regenerating system to the incubation mixtures.

50 μL-aliquots were sampled from incubation mixtures at: 0, 3, 10, 15, 30, and 45 minutes. The samples were centrifuged at 3,000 rpm for 10 minutes and further diluted in order to optimize the analytical condition prior to the LC MS/MS or Rapid Fire-MS/MS analysis. Incubations of test items were run in duplicate (n=2) with a single positive control (n=1). For sample analysis, an LC-MS/MS system was used to monitor the test samples/control to internal standard peak area ratios as representative of the test or control item's concentration. Data from test item incubation was processed to give mean values where applicable. Metabolic stability was calculated from the ratio of peak area of the remaining test or control item with internal standard versus time.

Peak areas for test and control items were integrated using Integrator Software from Agilent or Analyst or MultiQuant Software from AB SciexTM and were exported to XLFit or Morphit (The Edge), which were designed to calculate in vitro metabolic stability parameters. The integrated peak areas of the test and control items at the selected time points were divided by the respective peak areas of the IS, and the percent of parent remaining was calculated by normalizing the peak area ratio of parent to IS at 0 min. Observed rate constant (kobs) for parent degradation was calculated by determining the slope of the line of the graph of the natural log of percentage parent remaining versus time of incubation. This was scaled for the protein in the incubation relative to that in the liver.

Results and Significance: Intrinsic clearance (L/min/mg protein) of MDMA, MEAI, MMAI, and exemplary disclosed compounds is shown in Table 3. Each of CMP-401, CMP-402, and CMP-405 exhibited greater clearance in human microsomes relative to MDMA. CMP-402 exhibited the greatest clearance in both human and rat microsomes. Generally, compounds with greater intrinsic clearance are likely to be cleared rapidly in vivo, thereby minimizing their duration of action. In contrast, metabolically stable molecules with low clearance may exhibit a relatively prolonged duration of action.

TABLE 3
Clearance of MDMA, MEAI, MMAI, and CMP-405

| | Cl (μL/min/mg protein) - | Cl (μL/min/mg protein) - Rat |
|---|---|---|
| Compound | Human Microsomes | Microsomes |
| MDMA | 41 | 395 |
| MEAI | 137 | 156 |
| MMAI | 46.7 | 200 |
| CMP-401 | 87.2 | 121 |
| CMP-402 | 758 | 2050 |
| CMP-405 | 137 | 143 |

### Example 14: In Vitro Evaluation of Membrane Permeability and Interactions with P-Glycoprotein (P-Gp) in MDCKII MDR1 Cells

Purpose: Compounds were screened to assess their rate of transport across a cell membrane, i.e., apparent permeability (Pₐₚₚ) at pH 7.4. Whether the compounds act as substrates for P-glycoprotein (P-gp), an efflux transporter was also determined.

Methods: A bidirectional permeability study was conducted to evaluate the apparent permeability (Pₐₚₚ) of therapeutic compounds. Additionally, the MDCKII-MDR1 cell line (Madin-Darby Canine Kidney clone II cell line heterologously expressing the human P-glycoprotein transporter) and mock cell line MDCKII were used to determine whether a compound acted as a P-gp substrate, in the absence and in the presence of a potent P-gp inhibitor. Test control items (digoxin, metoprolol, and atenolol) and control P-gp inhibitor (GF120918) stock solution were prepared in DMSO. Bidirectional assays (Apical to Basolateral [AB] and Basolateral to Apical [BA]) were run, in both the absence and presence of P-gp inhibitor GF120918 in MDCKII-MDR1 and mock cell line MDCKII, using as transport buffer HBSSH at a pH of 7.4 (n=3).

Compounds were tested at a single concentration (i.e. at 3 or 10 M) at one time point (i.e. 60 min). Alternative concentrations or time points may also be investigated. As reference compounds, digoxin (P-gp substrate), atenolol (low permeability) and metoprolol (high permeability) were included at a single concentration (e.g., 10 or 25 PM) and at single time point (e.g., 60 min). Digoxin transport was evaluated in two directions (apical-to-basolateral [AB] and basolateral-to-apical [BA]) in the absence and presence of GF120918 or in mock cells, MDCKII (n=3). Atenolol and metoprolol were only tested in the AB direction in absence of P-gp inhibitor GF120918 or in mock cells, MDCKII (n=3). The integrity of the cell monolayer was evaluated after the permeability experiment using the paracellular permeability marker Lucifer yellow (LY) in the apical to basolateral direction in each well.

MDCKII or MDCKII-MDR1 cells were cultivated in DMEM (high glucose, GlutaMAXTM supplemented with pyruvate, 10% FBS HI and Pen/Strep). Cells were seeded onto microporous PET (Polyethylene Terephthalate) membranes in HTS 96-Multiwell Insert plates at a density of ˜175,000-245,000 cells/cm² (25000-35000 cells/well; 50 μL/well) in DMEM medium and incubated for 3-4 days at 37° C.-5% CO₂. Medium was changed the day before the experiment.

Stock solutions of test items were prepared in DMSO at a concentration of 5 or 10 mM and stored at −20° C. until use. The potent P-gp inhibitor, GF120918 (stock solution 10 mM) and the reference controls digoxin, atenolol, metoprolol (stock solutions 3 or 10 mM) were prepared in DMSO and stored at −20° C. until use.

Donor working solutions of test items and reference controls were prepared at a concentration of 10 or 25 M, diluting the stock solutions in HBSSH. Receiver working solutions contained transport buffer only. All working solutions were prepared such that the final concentration of DMSO was 1% (v/v). A donor working solution containing LY was also prepared in transport buffer at 100 M.

MDCKII and MDCKII-MDR1 cells were preincubated (37° C., 15 to 30 minutes) in receiver working solutions containing transport buffer on apical (A) and basolateral (B) sides. Following pre-incubation, test items and control items (digoxin) transport were measured in two directions (apical to basolateral [AB]) and basolateral to apical [BA]), and these directions were performed in triplicate sets of wells, in both the absence and presence of 10 M GF120918 or mock MDCKII cells. For [AB] directional transport, 75 μL of donor working solution was added to the A (apical) compartment and 235 μL of receiver working solution was added to the B (basolateral) compartment. For [BA] directional transport, 235 μL donor working solution with test items or reference controls was added to the B compartment and 75 μL receiver working solution was added to the A compartment. Transport of permeability reference controls, atenolol and metoprolol, were measured in one direction (apical to basolateral [AB]) (n=3).

The cells were incubated (at 37° C., with shaking) for 60 minutes. Samples were removed from donor solutions and transport buffer (blank samples) for t=0 samples (Cₒ) and at the end of the incubation period, from the receptor site (basolateral compartment for A→B direction and apical compartment for B→A direction) and from donor side (Cₒ fin). Samples were extracted by protein precipitation with acetonitrile containing rolipram (for positive ion mode) or diclofenac (for negative ion mode) as generic internal standard compounds and centrifuged for 10 minutes at 3,000 rpm.

Additionally, to evaluate the integrity of the cell monolayer, LY permeability was measured in one direction, A→B, at the end of incubation. Residual solutions in the apical compartment were gently removed, and 75 μL of donor working solution containing LY at 100 M was then added to the A compartment and 235 μL of receiver working solution to the B compartment. The cells were incubated (at 37° C.) for 60 minutes.

100 μL from each sample receiver well, 100 μL/well of donor solution containing 100 M LY and 100 μL/well of Transport buffer were transferred to a 96-well clear bottom black plate. Fluorescence was measured using a fluorescence plate reader, such as the Tecan Spectrafluor plus, at Ex=485 nm, Em=535 nm.

The samples themselves were analyzed using a LC MS/MS system via discrete or cassette analysis to monitor the test item or positive control to internal standard peak area ratios as representative of the test or control item's concentrations. The rate of transport (Papp) of test items and digoxin were determined in the apical to basolateral ([AB]) and basolateral to apical ([BA]) directions in the absence and presence of GF120918, where feasible. The rate of transport (Papp) of metoprolol and atenolol will be determined in the apical to basolateral ([AB]) direction in the absence of GF120918 or mock MDCKII cells.

The efflux ratio of test items and of digoxin in the absence and presence of GF120918 or in mock MDCKII cells was then calculated. Comparing the efflux ratios generated in the presence and absence of GF120918 or in mock MDCKII cells indicated whether test items were P-gp substrates. A test item was considered to be a P-gp substrate when the efflux ratio in the absence of inhibitor was >2 and if the ratio was significantly reduced in the presence of inhibitor or in mock MDCKII cells.

Mass balance as a percentage (%) was calculated using the following equation:

% Recovery=100×(CD(t)+CR(t))/C₀

Where CD(t) is the measured concentration in the donor well at time t (expressed as IS ratio), CR(t) is the measured concentration in the receiver well at time t (expressed as IS ratio), Co is the initial concentration in the donor solution (expressed as IS ratio). The amount of compound associated with the cells or plastic was not determined.

The percentage of cell integrity was calculated using the following equation: % Integrity=100×[1-RFUbasolateral/RFUapical]

LY RFU values were normalized by background mean values. Wells were considered fully acceptable if the % Integrity was >98%, acceptable with caution for values included between 98% and 95%, and not acceptable for values<95%.

Results and Significance: Compounds were classified as follows (Cambridge MedChem Consulting, ADME, 2019):

| Papp (nm/s) | Classification |
|---|---|
| >150 | High Permeability |
| 50-150 | Medium Permeability |
| <50 | Low Permeability |

CMP-405 and MEAI were classified as highly permeable, whereas MDMA was less permeable. None of the tested compounds were determined to act as P-gp substrates. Results for MDMA, MEAI, and exemplary compound CMP-405 are shown in Table 4.

TABLE 4
Permeability of MDMA, MEAI, and CMP-405

| | MDCKII | | |
|---|---|---|---|
| | Permeability app | MDCKII-MDR1 | |
| Compound | A > B (nm/s) | B-A ratio | P-gp Substrate? |
| MDMA | 140 | 1.5 | No |
| MEAI | 174 | 1.0 | No |
| CMP-405 | 162 | 1.1 | No |

Each exemplary compound showed greater apparent permeability than MDMA. Measures of permeability are widely used to understand absorption of a compound into cells. P-gp is an efflux transporter found in different tissues in the body, such as the brain and liver. P-gp influences drug transport in various ways. Thus, permeability and P-gp substrate screening provide insight into the movement of a compound, such as in a biological system.

### Example 15: In Vitro Assessment of CYP45 Inhibition

Purpose: CYP45 enzymes mediate variability in drug pharmacokinetics and, consequently, responses to treatment. Herein, the inhibitory activity oftherapeutic compounds on such enzymes was determined to evaluate the potential for drug-drug interactions. CYP450 enzyme isoforms CYP1A2, CYP2C8, CYP2B6, CYP2C9, CYP2C19, CYP2D6, and CYP3A4 were examined in this study. MDMA, MEAT, and MMAI were tested for comparative purposes.

Methods: Recombinant human CYP450 isoenzymes were used to metabolize pro-fluorescent probe substrates to fluorescent products. IC₅₀ values determined from the effect of test compounds on the metabolism of these probe substrates were then used to determine inhibitory potency against CYP450 isoforms. The chemical reagents included:

| | Supplier/Cat. | |
|---|---|---|
| Component | Number | Storage |
| Trizma | Sigma T2663 | Store at RT |
| EDTA | Sigma E6635 | Store at RT |
| Sodium Bicarbonate | Sigma S5761 | Store at RT |
| Potassium Phosphate | Sigma P8709 | Store at 4° C. |
| monobasic solution | | |
| Potassium Phosphate dibasic | Sigma P8584 | Store at 4° C. |
| solution | | |
| NAPD monosodium salt | Sigma N3886 | Store at −20° C. |
| Glucose-6-phosphate | Sigma G7250 | Store at −20° C. |
| disodium | | |
| salt hydrate | | |
| Glucose-6-phosphate | Sigma G6378 | Store at −20° C. |
| dehydrogenase | | |
| Miconazole | Sigma M3512 | Store at RT |
| DMSO | Sigma 527963 | Store at RT |

The substrates included the following:

| Component | Supplier/Cat. Number | MW |
|---|---|---|
| Ethoxyresorufin (ER) | Sigma E3763 | 249.249 |
| 7-Methoxy-4- | SB-355907 | 302.203 |
| trifluoromethylcoumarin-3- | | |
| acetic acid (FCA) | | |
| 3-Butyryl-7-methoxycoumarin | SB-363399 | 249.241 |
| (BMC) | | |
| 4-Methylaminomethyl-7- | SB-285033 | 219.239 |
| methoxycoumarin (MMMC) | | |
| 7-Benzyloxyquinoline (7-BQ) | Sigma B5182 | 235.288 |
| Vivid DBOMF substrate | Life Technologies P2974 | 572.6 |
| Vivid BOMCC substrate | Life Technologies P2974 | 307.3 |

The test system was based on a three-step procedure with a “mix and read” format, where reaction and reading were performed at 37° C. The assays described herein measure in vitro inhibitory effects (pIC₅₀) of compounds on the human P450 isoforms (1A2, 2B6, 2C8, 2C9, 2C19, 2D6, 3A4) expressed in recombinant microsomes. Pro-fluorescent probe substrates were metabolized to fluorescent products by the enzymes. Fluorescence was measured in kinetic mode (1 read/minutes for 10 minutes), and the fluorescence rate was calculated. Data were normalized to controls: DMSO represents 7. effect (i.e., no inhibition), while 10 M of the CYP inhibitor miconazole demonstrates 100% effect (i.e., complete inhibition).

Inhibition of the fluorescent signal resulting from P450 activation indicated inhibitory activity and facilitated calculation of the compound pIC₅₀. A quality check was included in each compound plate, wherein the signal window was monitored by performing Z′ calculations (Z′≥0. [symbol] ) and the antagonist potency of internal standards was within the pIC₅₀±2SD range. Concentration response curves (CRCs) of compounds were run on two different test occasions (n 2) by preparing serial dilutions from the same compound stock solution.

| Solutions | Components | Guidelines |
|---|---|---|
| Assay buffer | 100 mM Trizma HCl + 0.5 mM EDTA | Store 4° C., shelf life: 3 mos. |
| Vivid Assay Buffer | Potassium Phosphate Buffer | Store 4° C., |
| | 100 mM, pH 8.0 | shelf life: 3 months |
| Cofactor buffer | 2% (w/v) sodium bicarbonate | Store 4° C., |
| | | shelf life: 3 months |
| Cofactor | 7.8 mg glucose-6-phosphate, 1.7 mg | Prepare fresh each time on |
| | NADP, 6 Units glucose-6-phosphate | the day of the experiment |
| | dehydrogenase/mL of 2% sodium | |
| | bicarbonate | |
| Vivid Regeneration | 333 mM Glucose-6-phosphate, 30 U/ml | Store at −80° C. |
| System | Glucose-6-phosphate dehydrogenase in | |
| | Vivid Assay Buffer | |
| NADP | 10 mM in Vivid Assay Buffer | Store at −80° C. |
| 1A2 Substrate | 50 μM ER | Dissolve 12 μg/ml in |
| | | acetonitrile |
| 2C9 Substrate | 12.5 mM FCA | Dissolve 3.67 mg/mL in |
| | | acetonitrile |
| 2C19 Substrate | 2.5 mM BMC | Dissolve 0.615 mg/mL in |
| | | DMSO |
| 2D6 Substrate | 2.5 mM MMMC | Dissolve 0.547 mg/mL in |
| | | methanol |
| 3A4 Substrate | 2.5 mM 7-BQ | Dissolve 0.588 mg/mL in |
| | | acetonitrile |

To prepare the compound plate, test compounds serial dilutions 1 to 3 were performed from a 10 mM stock solution in DMSO by Biomek FX to generate 10 point CRC with the highest concentrations in columns 3 and 13.

100% DMSO was placed in columns 1 and 2 and used for assay low controls. CYP inhibitor (10 mM miconazole solution in DMSO) was placed in columns 23 and 24 and served as an assay high controls. 1 μL copy plates were then stamped into V-bottom drug plates at a concentration that is 200 fold the final assay concentration. The copy plates were diluted prior to the experiment with assay buffer to reach 4 times the final assay concentration (2% w/v DMSO).

A reference compound (e.g., miconazole) was included in each compound plate in row A, column 3 and 13. The reference compound was diluted and stamped together with test compounds. The final concentrations of the 10 point CRCs of test compounds in the assay plate were 5.00E-05; 1.67E-05; 5.56E-06; 1.85E-06; 6.17E-07; 2.06E-07; 6.86E-08; 2.29E-08; 7.62E-09 and 2.54E-09, respectively. All solutions were prepared immediately before the assay.

As for assay plate preparation, microsomes and substrate were mixed together according to the following table:

| Mix | 1A2/ER | 2C9/FCA | 2C19/BMC | 2D6/MMMC | 3A4HR/7BQ |
|---|---|---|---|---|---|
| Assay buffer (mL) | 14 | 14 | 14 | 14 | 14 |
| Microsomes (μL) | 330 | 330 | 330 | 330 | 330 |
| (0.1 mg/mL protein) | | | | | |
| Substrate (μL) | 224 | 90 | 90 | 90 | 224 |

Microsomes were added to the buffer prior to the substrate addition. The solution was carefully mixed without vortexing. 30 μL/well of the mixture was then transferred to the assay plate (384-well black plate) using a 16-channel pipette. For each P450 isoformn, a separate plate was prepared and labeled with a barcode. For fluorescence measurement, 10 μL/well of the test compounds were transferred from the compound plate to the assay plate using Biomek FX and the appropriate protocol. The assay plate was then incubated at 37° C. for 10 minutes on a shaker to allow for the interaction between compounds and enzymes.

10 μL/well of the cofactor was then added to the assay plate using the Multidrop before placing the assay plate in the plate reader EnVision to measure fluorescence. The overall final DMSO concentration for the assay was 0.5%. Plates were read every minute for 10 minutes, according to a P450 isoform specific protocol, as shown below.

| | | | | 2D6/ | 3A4HR/ |
|---|---|---|---|---|---|
| Assay | 1A2/ER | 2C9/FCA | 2C19/BMC | MMMC | 7BQ |
| Excitation | 535 | 405 | 405 | 405 | 405 |
| Emission | 590 | 515 | 515 | 515 | 535 |
| Mirror | 50/50 | 505 FITC | 505 FITC | 505 FITC | 505 FITC |

The CYP3A4 and CYP2C8 were assayed using the Vivid CYP3A4 Baculosomes, CYP2C8 Baculosomes and Vivid DBOMF Substrate (Life Technologies, cat. P2377, PV6138 and P2974 respectively), while 2B6 is tested by Vivid CYP2B6 Baculosomes and Vivid BOMCC Substrate (Life Technologies, cat. P3028 and P2975). Test compounds were prepared as specified above.

The assay plate was prepared by preparing pre-mix by diluting P450 Baculosomes Plus Reagent and Vivid Regeneration System in 1× Vivid CYP450 Reaction Buffer (Potassium Phosphate Buffer, 100 mM, pH 8.0). Briefly, 8 μL/ml of P450 Baculosomes Plus Reagent and 16 μL/ml of Vivid Regeneration System were added to achieve the appropriate final concentrations and then mixed by inversion. 30 μL of pre-mix were dispensed into each well of a black, 384-well plate.

Next, 10 μL/well from the compound plate was added and incubated for 10 minutes at 37° C. on a plate shaker, while a mixture of Vivid Substrate and Vivid NADP+ in Vivid CYP450 Reaction Buffer was prepared. The reaction was initiated by adding 10 μL/well of the Vivid Substrate and NADP+ mixture, before transferring the plate into the fluorescent plate reader (immediately, within 2 minutes), and fluorescence was monitored every minute for 10 minutes according to the specific protocol.

Regarding data analysis, inhibition is expressed as a percentage. Normalization was based on the inhibition rate in the presence of 10 M miconazole (100% effect=C₂) and inhibition rate in presence of 0.5% DMSO (0% effect=C1), according to the following formula: Y=100×[(data-C1)/(C2−C1)]. Curve fitting and pIC₅₀ estimations are carried out using a four-parameter logistic model in Morphit.

Results and Significance: The inhibitory activity, represented as the IC₅₀ (μM), of test compounds are presented in Table 5. Like MMAI, CMP-401, CMP-402, and CMP-405 displayed inhibitory activity at CYP1A2. All tested compounds, with the exception of CMP-401 and CMP-402, inhibited CYP2D6. MMAI alone was additionally found to inhibit CYP2C9. All compounds were tested in duplicate (n=2). Overall, therapeutic compounds showed limited activity at the majority of tested isoforms, indicating a low likelihood of facilitating drug-drug interactions.

TABLE 5
Inhibition of CYP450 isoforms

| CYP450 | | | | CMP- | CMP- | CMP- |
|---|---|---|---|---|---|---|
| Isoform | MDMA | MEAI | MMAI | 401 | 402 | 405 |
| CYP1A2 | >50 | >50 | 2.11 | 1.21 | 1.07 | 0.61 |
| CYP2B6 | >50 | >50 | >50 | >50 | >50 | >50 |
| CYP2C19 | >50 | >50 | >50 | >50 | >50 | >50 |
| CYP2C8 | >50 | >50 | >50 | >50 | >50 | >50 |
| CYP2C9 | >50 | >50 | 35.7 | >50 | >50 | >50 |
| CYP2D6 | 1.6 | 37 | 33.6 | >50 | >50 | 23 |
| CYP3A4 | >50 | >50 | >50 | >50 | >50 | >50 |

### Example 16: In Vitro Determination of Plasma Protein Binding and Tissue Protein Binding Using an Equilibrium Dialysis Assay

Purpose: The plasma protein binding of therapeutic compounds in rat and human specimens, including blood, brain, and lung samples, was evaluated.

Methods: Plasma protein binding and blood tissue binding of test items (0.5 μM) was determined using biological samples from male Sprague Dawley (SD) rats and humans. Clozapine was used as a control compound for plasma protein binding and blood and brain tissue binding, as it exhibits extensive binding to plasma proteins (˜97%). Dexamethasone was used as a control compound for lung tissue binding. Various biological samples were spiked with test compounds according to the methods that follow.

Preparation of plasma or blood samples: An appropriate amount of test compound was dissolved in DMSO to yield a 10 mM solution. Further dilutions, to obtain 50 μM working solutions (WS), were then prepared using 50% acetonitrile in Milli-Q water. These 100×WS were used to spike plasma or blood to obtain a final concentration of 0.5 μM. From this initial spiking solution, control samples (n=3), referred to as T=0, were immediately extracted and used to calculate the recovery of the test item.

Preparation of brain homogenate samples: Test items were dissolved in DMSO (or an appropriate solvent) to give a 10 mM solution. Further dilutions, to obtain 166.7 μM working solutions (WS), were then prepared using 50% acetonitrile in Milli Q water. These 100×WS were used to spike the brain homogenate to obtain a final concentration of 5 μM. From this initial spiking solution, control samples (n=3) referred to as T=0, were immediately extracted by protein precipitation.

Preparation of lung homogenate samples: Test compounds were dissolved in DMSO (or an appropriate solvent) to give a 10 mM solution. Further dilutions, to obtain 71 μM working solutions (WS), were then prepared using 50% acetonitrile in MilliQ water. 71 μM WS was used to spike the lung homogenate to obtain a final concentration of 5 μM. From this initial spiking solution control samples (n=3), named T=0, were immediately extracted and used to calculate the recovery of the test item.

Preparation of tissue homogenate samples: Test compounds were dissolved in DMSO (or an appropriate solvent) to give a 10 mM solution. Further dilutions, to obtain working solutions (WS), were then prepared using 50% acetonitrile in MilliQ water. WS were used to spike the tissue homogenate to obtain a final concentration of 5 μM in neat tissue. From this initial spiking, solution control samples (n=3), named T=0, were immediately extracted and used to calculate the recovery of the test item.

An equilibrium dialysis method was performed to determine protein binding in the various biological samples described herein. Materials were prepared and methods were in accordance with the following procedures.

Membranes were prepared for use in the equilibrium dialysis experiment. by soaking in deionized water for at least 60 minutes. After this period, 20% of pure ethanol was added, and the membranes were left in this solution for at least 20 minutes. The membranes were then rinsed in Milli-Q water prior to use.

To initiate the experiment, 150 μL of test item-free buffer (isotonic phosphate buffer for plasma and blood, artificial CSF buffer for brain or HBSS buffer for lung) was dispensed on one half-well of a 96-well dialyzer apparatus (HTDialysis LLC), and 150 μL of spiked matrix (plasma, blood, brain or lung) was loaded on the other half-well. Alternative volumes may be used according to the operating instructions of HTDialysis.

The artificial cerebrospinal fluid (CSF) buffer was prepared as follows, NaCl 3.652 g, KCl 93.2 mg, MgCl2 119.96 mg, CaCl₂ 92.61 mg, Na₂HPO₄*H₂O 268.0 mg. These were dissolved in 0.5 μL of MilliQ water, and the pH was adjusted to a pH of 7.4 with H₃PO₄.

Dialysis buffer was prepared by dissolving Na₂HPO₄ 8.69 g, KH₂PO₄ 1.90 g, and NaCl 4.11 g in 1 μL of MilliQ water, and adjusting the pH to 7.4 with H₃PO₄. At the end of the equilibration period (device is sealed and incubated under shaking for 5 hours at 37° C.), 50 μL of dialysed matrix will be added to 50 μL of corresponding test item free dialysed buffer, and vice versa for buffer, such that the volume of buffer to matrix will be the same. Samples were extracted by protein precipitation with 300 μL of acetonitrile containing rolipram (for positive ionization mode) or diclofenac (for negative ionization mode) as internal standard and centrifuged for 10 minutes at 2800 rpm. Supernatants were collected (100 μL), diluted with 18% ACN in Milli Q water (200 μL).

Samples were then injected onto an HPLC MS/MS or UPLC MS/MS system. In order to control the integrity of the membrane after the dialysis process, the presence of protein was assessed in the buffer compartment. 5 μL of dialysed buffer will be added with 250 μL of Bio-Rad protein assay reagent (Bio-Rad) diluted 1:5 with Milli-Q water. A change in color from brown to a brilliant blue indicates protein contamination. Contaminated samples were excluded from all calculations.

Samples were analyzed in a LC-MS/MS system using an analytical method to monitor the compound to internal standard peak area ratios as representative of the compound concentrations. Alternatively, samples were analyzed by RapidFire.

Protein binding in plasma, blood, brain, and lung tissue samples were determined with the following formula: Afu=Buffer/x, wherein “x” may be plasma, blood, brain, or lung, and “Afu” is the apparent fraction unbound, and the other variables refer to the analyte/internal standard ratio determined in their specific compartment (buffer, plasma, blood, brain homogenate, and lung homogenate); Fucr-(1/D)/[(1/Afu)-1+(1/D)], wherein fucr is the fraction unbound corrected, D is the matrix dilution factor (D=1 for plasma, 2 for blood, 3 for brain, 7 for lung). % Binding=(1−fucr)×100 and % Unbound=100—% Bound. See, e.g., Kalvass et al., Drug Metab Dispos., 2018; 46(4):458-469.

Each test item and control compound (clozapine or dexamethasone) was processed in triplicate, and the average and standard deviation (SD) were calculated. If the SD was greater than 20%, the median (with range) was calculated. For result validation, the percent binding values for clozapine and dexamethasone must have been within Ave±2SD (where Ave is calculated from historical data).

Recovery was determined by comparing the sum of area ratio (analyte/internal standard) determined in buffer and plasma, blood, brain, or lung compartments with area ratio of the T=0 samples, a recovery value will obtained, as described in the following formula: Recovery=[(Buffer+Matrix)/T=0]×100, wherein “Buffer” is the analyte/internal standard ratio determined in buffer compartment, “Matrix” is the analyte/internal standard ratio determined in plasma, blood, brain, or lung compartment, and T=0 are the control samples.

Regarding recovery, a value of > [symbol] 00 or <50 indicates that the experimental data is not valid; 50≤recovery <80 or 1 [symbol] 0<recovery ≤ [symbol] 00 indicates that the experimental data should be met with caution, and 80≤recovery ≤1 [symbol] 0 indicate the data is valid.

Results and Significance: Protein binding of MDMA, aminoindanes MEAI and MMAI, and exemplary compounds in various biological samples is shown in the table below. While CMP-401 and CMP-402 showed low binding (>50% fraction unbound) in human plasma, comparable to MDMA, MEAI, and MMAI, CMP-405 showed high binding in plasma specimens. Unbound fractions of CMP-401 (>50%) and CMP-405 (24%) in rat brain were detected, exceeding that of MMAI (12%).

| Specimen | MDMA | MEAI | MMAI | CMP-401 | CMP-402 | CMP-405 |
|---|---|---|---|---|---|---|
| Human Plasma | >50 | >50 | >50 | >50 | >50 | Low |
| (Fu %) | | | | | | Recovery |
| Rat Plasma | >50 | >50 | >50 | Low | Not run | Low |
| (Fu %) | | | | Recovery | | Recovery |
| Rat brain | 40 | 33 | 12 | >50 | Low | 24 |
| tissue binding | | | | | Recovery | |
| (Fu %) | | | | | | |

Broadly, the affinity of a compound to bind to plasma proteins and tissue influences its pharmacological efficacy. Generally, the free (unbound) form of a drug is available to reach a target site and exert pharmacological activity. In some examples, the extent of plasma protein binding may affect the volume of distribution, half-life, and clearance of a compound.

### Example 17: In Vivo Pharmacokinetics of Therapeutic Compounds Following IV Administration

Purpose: The pharmacokinetics of therapeutic compounds, including penetration into the brain, were determined in rats following intravenous (IV) administration.

Methods: Three experimental groups, as outlined below, were included in the study:

Group 1: Therapeutic compounds were administered via the IV route to three rats at a target dose level of 0.5 mg/kg. Blood and brain samples were collected at 2 h after IV administration.
Group 2: Therapeutic compounds were administered via the IV route to three rats (brain penetration group) at a target dose level of 0.5 mg/kg. Blood and brain samples were collected at 2 h after IV administration.
Group 3: Therapeutic compounds were orally administered to three rats at a target dose level of 1 mg/kg.

All test formulations were prepared on the day of administration prior to dosing, stored at room temperature, and used as soon as possible. Formulations were prepared and administered by volume IV dosing. Actual body weights were determined on the day of dose administration, and the dose volumes were adjusted to the weight of the animal at the time of dose administration.

Blood was collected into K3 EDTA tubes at each of the following time points post-administration:

Group 1 (IV): 0.083, 0.33, 1, 2, 4, 6, 8, and 24 hours after dosing
Group 2 (IV): 2 hours after dosing for terminal brain analysis

All blood samples were thoroughly and gently mixed following collection and placed on wet ice. Within 0.5 h of collection, blood was centrifuged (3000 g for 10 minutes at approximately 4° C.). Within 0.5 hours, 20 μL of each plasma sample was transferred into micronic tubes containing 80 μL of 0.1N Hepes buffer (pH 7.0-7.5). Residual plasma was transferred in micronic tubes as a second aliquot.

Brain specimens were diluted with 4 volumes of 0.1 N Hepes buffer and homogenized using the Precellys system. Blood and brain samples were assayed using an optimized method based on protein precipitation with acetonitrile followed by HPLC/MS-MS analysis. Given the unknown stability of the analytes in blood and brain, calibration standards (CS) and quality control samples (QC) were prepared in blood and brain samples on the day of dosing and stored together with study samples. It is assumed that this procedure accounted for any possible analyte degradation.

Study samples, CS, QC, and blanks were spiked with an internal standard (IS), to improve the precision of the assay. Study samples were analyzed together with CS, QC and blank samples (including double blanks). From the calibration curve, the linear range of the analytical method was determined and the lower/upper limits of quantitation were specified.

The results of blood and brain specimens were subjected to non-compartmental pharmacokinetic analysis using Phoenix WinNonlin 6.3, with the following parameters reported: Calculated concentration of test items in the corresponding formulation (expressed as mg/mL), dose levels (expressed as mg/kg) for each compound, actual collection times (expressed as hh: mM), blood concentrations (expressed as ng/ml) for each compound, brain concentrations (expressed as ng/g) for each compound, PK parameters (iv: Cₘₐₓ, AUC, CLp, Vss, t_{1/2}, if applicable; oral (PO): Cmax, tₘₐₓ, AUC, F %, Fa %, Eh, AUC BB ratio, if applicable) for each compound, and abnormal clinical signs and relevant findings, if any.

Results and Significance: The tested compounds showed a range of values for both highest measured concentration (Cmax) and exposure. Half-life was less than 2 hours for all tested compounds after IV administration, while clearance was classified as low-moderate for all compounds except for MDMA. Comparative pharmacokinetic properties are shown in Table 6. Regarding brain penetration, MEAI and CMP-405 showed a comparable brain to plasma ratio at 2 hours post-administration, 2.21 and 2.12, respectively. However, while the kp,uu of MEAI was calculated at 0.71, assuming free plasma concentration equals total plasma concentration, the unbound fraction of CMP-405 was not detected, precluding calculation of the compound's kp,uu.

TABLE 6
Pharmacokinetics of IV MDMA, MEAI, and CMP-405

| PK | | | |
|---|---|---|---|
| Parameters | MDMA (1 mg/kg) | MEAI (0.5 mg/kg) | CMP-405 (0.5 mg/kg) |
| Cₘₐₓ (ng/mL) | 625 | 1400 | 808 |
| Cₗₐₛₜ (ng/mL) | 12.1 | 24.7 | 8.08 |
| Tₗₐₛₜ (h) | 4 | 3 | 2 |
| AUCₗₐₛₜ | 367 | 773 | 330 |
| (h*ng/mL) | | | |
| AUC_{inf} | 384 | 788 | 334 |
| (h*ng/mL) | | | |
| Clearance | 45.6 | 10.6 | 25.5 |
| (mL/min/kg) | | | |
| T_{1/2} (h) | 0.953 | 0.494 | 0.33 |
| Vₛₛ (L/kg) | 3.02 | 0.376 | 0.602 |

### Example 18: In Vivo Analysis of Monoamines in Rat Brains in a Microdialysis Assay

Purpose: The effects of IP administration of therapeutic compounds, including exemplary compound CMP-405, on extracellular dopamine (DA), serotonin (5-HT), and noradrenaline (NE) levels over time in the nucleus accumbens (NAcc) were assessed in a microdialysis assay.

Methods and Procedure: This study was conducted in compliance with previously described procedures to assure data integrity, adherence to GLP regulations, and standard preclinical study and surgery procedures. The in vivo microdialysis technique was applied to measure the over-time variations of DA, 5-HT, and NE extracellular (EC) concentrations in conscious, freely-moving Sprague Dawley rats. Levels of the neurotransmitters in microdialysis samples were determined using LC-MS/MS analysis. The experiment was also conducted with MDMA for comparative purposes.

As it relates to cannula implantation, the rats were anesthetized with isoflurane 4% in oxygen (2-2.5 L/min), then the isoflurane concentration 2-2.5% was maintained via a nose cone adapted to the stereotaxic apparatus. The rats were treated with meloxicam (0.2 mg/kg, subcutaneously, sc; Meloxidolor) and amoxicilline (150 mg/kg, sc, Betamox LA). The skull was then shaved and disinfected. Rats were then placed in a stereotaxic apparatus for small animals. One vertical guide cannula was inserted through a 1 mm hole drilled on the exposed skull. Coordinates, with respect to the bregma to target specific brain areas, followed indications from stereotaxic atlas of the rat brain. Specifically a guide cannula (MAB 4.12.IC, Agn Tho's AB, Lidinga, Sweden) was inserted in the NAcc (anteroposterior+1.6; lateral −1.6; dorsoventral −6.0). The guide cannula was then secured with dental cement and 2-4 screws. At the end of the surgery the animal was isolated in a single cage during recovery, for a period of at least one week, during which animal health conditions were checked twice daily and the weight recorded twice a week. Animals continued to be housed singly throughout the experiment. One day before the experiment the cannula stylet was removed and a microdialysis probe with a dialyzing length of 2 mm (MAB 4.12.2 Cu, Agn Tho's AB, Lidingo, Sweden) was inserted. Probes can be re-used 2 times for 2 experimental sessions after appropriate cleaning with fresh milliQ water.

Post-surgery, the rats were maintained in a heating chamber prior to waking up in order to avoid hypothermia that could be caused by anaesthesia. 5 mL of physiologic solution was subcutaneously administered immediately after surgery, and once/day for 2-3 days, if signs of dehydration are noticed. Meloxicam (Meloxidolor) 0.2 mg/kg was again administered subcutaneously the day after surgery as well as after 48 h after surgery together with Amoxicilline Long Acting (Betamox LA) 150 mg/kg. Health status and body weight was checked every day during the first week.

The animals were treated with the therapeutic compounds, and MDMA, or their vehicle (saline) according to a Latin square design over 5 separate experimental days (one day/week). The treatments were administered in a volume of 1 mL/kg intraperitoneally.

On the day of the experiment, each animal was taken into the laboratory in its home cage. Inlet tubing of the probe was attached to a dual quartz-lined two channel liquid swivel mounted on a low mass spring counterbalance arm which, in turn, was connected to a gas-tight syringe on a microinfusion pump. Artificial cerebrospinal fluid (aCSF; containing KCl 2.5 mM, NaCl 125 mM, CaCl₂) 1.3 mM, MgCl 2 1.18 mM, Na2HPO4 2 mM, pH 7.4) was perfused through the probe (Univentor 864 Syringe Pump, Agn Tho's, Sweden) at a steady flow rate of 1 μL/min. An equilibration period of about two hours of perfusion was allowed before collecting samples. Following the equilibration period, 4 basal samples were collected for each experimental animal followed by further 10 post-treatment samples. Samples were collected every 20 minutes (up to 3 hours and 20 minutes post-treatment) by means of a refrigerated auto-sampler (Univentor 820 Microsampler, AgnTho's, Sweden) maintained at +8 “C. At the end of all experimental sessions, 2 μL of 0.1% methylene blue solution was injected through the microdialysis probe, animals were euthanized, and their brains removed to confirm the correct probe-position.

As mentioned, the concentration of DA, 5-HT and NE in CSF samples were determined using an optimized method based on LC-MS/MS analysis. Study samples were spiked with appropriate Internal Standard (IS) to improve the precision of the assay. Liquid chromatography separations were performed using Agilent HP1100 system (Agilent Techs.) equipped with a binary pump, a column oven and a PAL CTC Autosampler. The LC system was coupled with an API4000 Triple Quadrupole System (ABSciex) equipped with a TIS ion source. Analyst software 1.6.2 (ABSciex) was used to control the instruments.

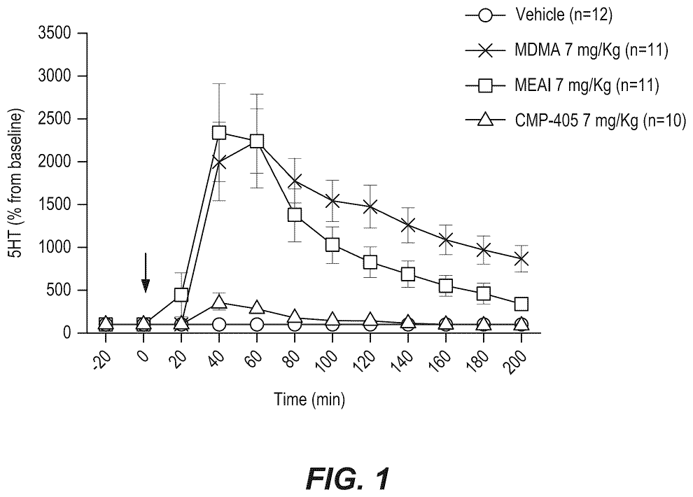

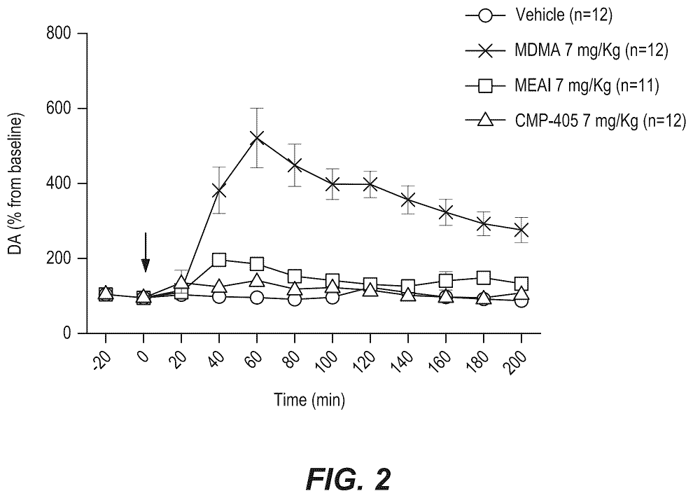

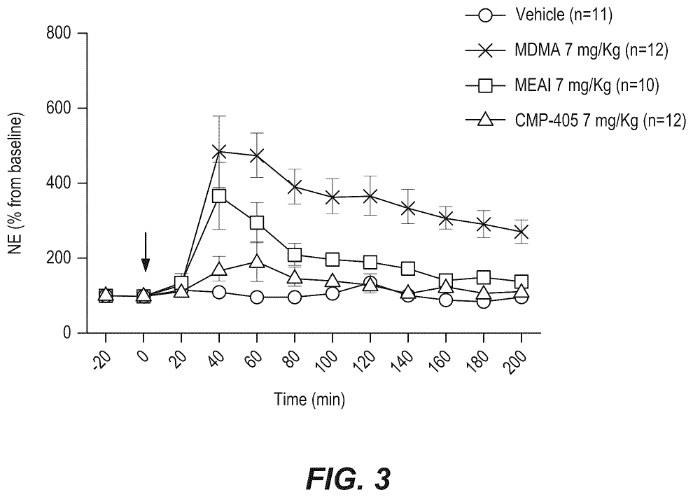

Results and Significance: FIGS. 1-3 show the effects of exemplary compound CMP-405 and reference compounds MDMA and MEAI on extracellular concentrations of 5-HT (FIG. 1), DA (FIG. 2), and NE (FIG. 3) in the rat brain. Overall, treatment with CMP-405 resulted in reduced monoamine release and a more rapid return to baseline (100%) compared to MDMA and MEAI. Regarding 5-HT, CMP-405 treatment resulted in a peak 5-HT level of 289% at 60 min, whereas 5-HT peaked at 2232% at 60 min in response to MDMA and 2365% at 40 min following administration of MEAI. Notably, MEAI caused a greater increase in 5HT levels than MDMA. 5-HT levels in response to CMP-405 returned to baseline at 180 min, whereas levels in response to MDMA (980%) and MEAI (470%) far exceeded baseline at this same time point. Regarding NE levels, CMP-405 administration resulted in a peak NE level of 188% (60 min), whereas NE levels in response to MDMA peaked at 473% (40 min) and at 366% (40 min) in response to MEAI.

TABLE 7
Monoamine Levels Following I.P. Administration
of 7 mg/kg MDMA, MEAI, and CMP-405

| Time | Serotonin (5-HT) | | | Dopamine (DA) | | | Norepinephrine (NE) | | |
|---|---|---|---|---|---|---|---|---|---|
| Post- | | | CMP- | | | CMP- | | | CMP- |
| Admin | MDMA | MEAI | 405 | MDMA | MEAI | 405 | MDMA | MEAI | 405 |
| −20 min | 102% | 102% | 103% | 107% | 104% | 101% | 100% | 103% | 99% |
| 0 min | 98% | 98% | 97% | 93% | 96% | 99% | 100% | 97% | 101% |
| 20 min | 135% | 446% | 114% | 113% | 95% | 134% | 113% | 132% | 106% |
| 40 min | 2000% | 2365% | 353% | 381% | 197% | 122% | 473% | 366% | 167% |
| 60 min | 2232% | 2248% | 289% | 520% | 187% | 139% | 457% | 295% | 188% |
| 80 min | 1780% | 1391% | 182% | 448% | 154% | 117% | 375% | 208% | 147% |
| 100 min | 1545% | 1036% | 157% | 399% | 141% | 123% | 348% | 194% | 136% |
| 120 min | 1472% | 830% | 140% | 396% | 130% | 113% | 350% | 187% | 125% |
| 140 min | 1255% | 688% | 115% | 357% | 131% | 99% | 320% | 167% | 107% |
| 160 min | 1083% | 564% | 113% | 324% | 137% | 98% | 295% | 140% | 122% |
| 180 min | 972% | 470% | 100% | 293% | 144% | 96% | 275% | 149% | 105% |
| 200 min | 871% | 340% | 105% | 278% | 127% | 107% | 255% | 133% | 106% |

### Example 19: The Effects of Compound Administration on Rat Body Temperature

Purpose: The effects of IP administration of therapeutic compounds, including exemplary compound CMP-405, on body temperature were compared to MDMA and MEAT.

Methods and Procedure: This study was conducted in compliance with previously described procedures to assure data integrity, adherence to GLP regulations, and standard preclinical study and surgery procedures. The in vivo microdialysis technique was applied to measure the over-time variations of DA, 5-HT, and NE extracellular (EC) concentrations in conscious, freely-moving Sprague Dawley rats. At the end of each experimental session (200 minutes after injection), body temperature (° C.) of Sprague Dawley rats was monitored using a digital thermometer with a rectal probe.

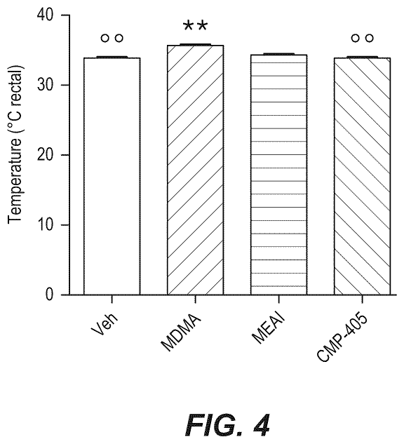

Results and Significance: FIG. 4 shows the effects of test compounds on body temperature. The body temperature of rats treated with MDMA was 1.6° C. higher than those treated with vehicle. The observed change in rat body temperature following administration of either MEAI or CMP-405 was lower than the change observed following administration of MDMA. The change in temperature caused by CMP-405 was significantly lower than the change in temperature caused by MDMA (p<0.01). Of all compounds tested, MEAI showed the highest average increase in body temperature. However the difference between MEAI and CMP-405 was not significant.

### H. PROPHETIC EXAMPLES

### Example 20: Exemplary Drug-Assisted Psychotherapy Protocol for the Treatment of a Substance Use Disorder, a Behavioral Addiction, a CNS or Mental Health Disorder, or the Improvement of Psychological Functioning

In certain exemplary treatment methods wherein the pharmaceutical compositions of the invention are shown to be useful in methods for treating substance use disorder, behavioral disorders, CNS and mental health disorders, or improving mental functioning, the treatment flow can be conceived in phases as follows: (1) screening and initial eligibility check; (2) pre-dose course preparatory group therapy course, e.g., a four-week course of weekly sessions; (3) baseline visit and eligibility confirmation; (4) a drug-assisted psychotherapy course, e.g., over eight or 13 weeks; (5) follow-up and data collection, e.g., at 3 months, 6 months, and 9 months.

It will be readily appreciated however that this exemplary treatment flow is merely illustrative of one use of the therapeutic compounds in a method of treating any condition described herein, that a use need not include every illustrated aspect (e.g., the follow-up or data collection of step five), or be in the exact order below, and moreover, that the steps, stages, or phases described (and their delineation as such) are simply for purposes of assisting a reader with the conceptualization of a use of the invention, whereas the full breadth and scope of the invention shall be known by reference to the claims.

TABLE 8
Exemplary Treatment Flow

(1) Screening and Eligibility Check
(2) Pre-Drug Course Preparatory Group Therapy Course
(3) Baseline Assessment Visit and Eligibility Check
(4) Drug-Assisted Psychotherapy Course
(5) Follow-Up and Data Collection

### 1) Screening and Initial Eligibility Check

Patients who meet the diagnostic criteria for a substance use disorder, behavioral addiction, or CNS or mental health disorder will be screened prior to entering the pre-drug course group therapy alcohol reduction program. Alternatively, a subject in need of improving psychological functioning, whether self-assessed or as determined by an observer, may take part in such treatment protocol. Eligible subjects will attend a baseline visit after the pre-course program to ensure eligibility. Potential patients will be invited to a screening visit where their eligibility for drug-assisted psychotherapy using the methods of the invention will be determined. Diagnostic criteria is available in any of the APA's DSM-5, the WHO's ICD-10, or another reference available to one of skill in the art. The screening visit will include informed consent, medical, psychiatric and substance use history, prescribed medication use, basic physiological observations and alcohol breath test, urine drugs, and pregnancy screen as clinically indicated.

During screening the therapeutic process will be discussed and explained. The patients' understanding of the procedure, requirements, and commitments will be confirmed and any questions answered before informed consent is obtained. If video and/or audio recordings will be made during therapy sessions patients will be asked to consent. If patients do not wish to have sessions recorded (or at any point decide to withdraw consent for the whole or part of the session) recordings will be stopped and deleted if requested. Potential patients also may be contacted and partake in a short telephone screen before invitation to a full screening visit, to minimize inconvenience to “obviously” ineligible.

If a psychiatric assessment is sought, it will be performed by a trained psychiatrist. Assessments may include: (1) the Mini International Neuropsychiatric Interview 5 (MINI 5) (Sheehan et al., J. Clin. Psych., 1998; 59 Suppl 20:22-33; quiz 34-57) to screen for comorbid psychiatric disorders; (2) diagnostic evaluation of the presence of a substance use disorder, e.g., Alcohol Use Disorder according to DSM-5 (assessed using SCID-5-CT (Clinical Trials Version) (First et al., American Psychiatric Association, 2015), a behavioral addiction, e.g., Gambling Disorder according to the DSM-5 (see, e.g., Grant et al., Compr Psychiatry. 2017 May; 75: 1-5), or mental health disorder, e.g., Major Depressive Disorder according to the DSM-5 (see, e.g., Uher et al., Depress Anxiety., 2014; 31(6):459-7) or the Primary Care PTSD Screen for DSM-5 (PC-PTSD-5); (3) clinical risk assessment for suicidality using the Colombia Suicide Severity Rating Scale (CSSRS) (Mundt, The Journal of Clinical Psychiatry, 2013; 74(9): 887-93). Clinical data collection may include years of substance use, compulsive behavior, or CNS or mental health disorders. Other questionnaires, e.g., relating to psychosocial functioning, quality of life, or sleep quality, may be completed as desired.

Patients also may be given an electronic journal to help track their substance use behavior, compulsive behavior, CNS or mental health symptoms, or psychological functioning for the duration of the therapy.
2) Pre-Drug Course Preparatory Group Therapy Course

After screening, participants will attend weekly sessions of group therapy, which will be run by a trained professional with expertise in substance use disorders, behavioral addictions, CNS and mental health disorders, or improvement in psychological functioning. In one embodiment, participants will attend weekly sessions of group therapy for four weeks, although shorter or longer courses may also be used, and in some embodiments the preparatory group therapy course may be skipped entirely. The purpose of the group will be to provide psycho-social support prior to starting the course of drug assisted therapy.

Adequate preparation also benefits from consideration of a person's support network, motivation and after-care planning to improve post-treatment outcomes (Kouimtsidis, J. Addict. Res. Ther., 2017; 8:326). Prior to being considered ready for the drug-assisted course, patients thus may attend weekly sessions of group therapy. Groups of generally between 5 and 10 participants can be facilitated by one or two trained staff with appropriate experience.

In some embodiments, each weekly group therapy session will last for 60 minutes. Groups will be closed and confidential. Patients will be enrolled into the groups on a rolling basis and will leave after a minimum (e.g., of four) weeks attendance to be enrolled into the drug-assisted psychotherapy course when deemed appropriate by facilitators.

If a patient is deemed not sufficiently engaged in the preparation phase (e.g., as deemed by the group facilitators) and/or has not managed to reduce their use of substances, compulsive behaviors, or improve mental health and/or psychological functioning, their enrollment into the drug-assisted psychotherapy course can be delayed until they are considered to be adequately prepared or otherwise ready to progress (e.g., by attending further group sessions, or by repeating the entire course or one or more weeks of the course).

### 3) Baseline Assessment Visit and Eligibility

After the participant has successfully completed the pre-treatment preparation group therapy course, they will be assessed for eligibility to start the drug-assisted psychotherapy course. This will be the “baseline” point. During this visit, continued consent will be confirmed and vital signs reassessed (pulse, blood pressure, etc.). A brief physical examination and ECG will be performed, and blood samples for routine laboratory tests (urea and electrolytes, full blood count, liver function tests and Gamma-GT) will be completed. A blood test is not required if the patient has not had one in, e.g., the last three months.

### 4a) Drug-Assisted Psychotherapy Course-Two Drug Sessions

In this exemplary embodiment of a drug-assisted psychotherapy treatment regimen, a patient will receive ten total therapy sessions during the treatment phase: eight 60-minute psychotherapy sessions, and two drug-assisted psychotherapy sessions. However, it will be readily appreciated that the total time of the drug-assisted sessions will differ depending on the duration of the therapeutic compound administered, and such total time will be determined by the practice of ordinary skill based on general knowledge and the teachings herein. In preferred embodiments, the totaltime ofthe drug-assistedpsychotherapy sessions is reduced, due to the short-acting nature of the therapeutic compound. In certain preferred embodiments, the average total time of a drug-assisted session is therefore reduced to less than 6 hours, less than 5 hours, less than 4 hours, less than 3 hours, less than 2 hours, and about 1 hour or less. Sessions will take place approximately 1-2 weeks apart and start as soon as possible after baseline.

Exemplary Eight-Week Drug-Assisted Psychotherapy Course

| Week | Session | Activity |
|---|---|---|
| 1 | 1 | Non-Drug Prep Session |
| | | (60-minute therapy session) |
| 2 | 2 | Non-Drug Prep Session |
| | | (60-minute therapy session) |
| 3 | 3 | Drug-Assisted Therapy Session |
| | | 1 (less than 6 hours) |
| | 4 | Next-Day Non-Drug Integration/ |
| | | Follow-up Session (60 mins) |
| 4 | 5 | Non-Drug Integration Session |
| | | (60-minute therapy session) |
| 5 | 6 | Non-Drug Integration Session |
| | | (60-minute therapy session) |
| 6 | 7 | Drug-Assisted Therapy Session |
| | | 2 (less than 6 hours) |
| | 8 | Next-Day Non-Drug Integration/ |
| | | Follow-up Session (60 mins) |
| 7 | 9 | Non-Drug Integration Session |
| | | (60-minute therapy session) |
| 8 | 10 | Non-Drug Integration & Final |
| | | Evaluation Session (~90 mins) |

Drug-assisted therapy sessions may be run by a therapist pair, preferably one male and one female, although they also may be run by two male or two female therapists, a single therapist, or more than two therapists. In some embodiments, psychological support during each drug session preferably will be provided by two members comprising a “therapy team.” Although the term therapy team will be used herein, it will be appreciated that modifications can be made to accommodate a single therapist, and in such embodiments “team” will refer to that therapist alone (or where appropriate in context, to that therapist and another therapist or clinician not necessarily present with the patient during a drug-assisted or other therapy session, but nevertheless a member of the patient's overall care team). A therapy team for example may comprise a psychiatrist and a co-therapist who is a clinical psychologist or psychotherapist, but either member may be a “therapist” as the term is defined above.

A dyadic male-female pair is used in certain embodiments herein. A dyadic male-female pair is a feature of certain MDMA-assisted therapy (Greer & Tolbert, Journal of Psychoactive Drugs, 1998; 30: 371-379); however, the co-therapist pair herein need not necessarily be male-female; same-sex pairs are also appropriate. Where a dyadic therapy pair is used, a patient will get to know both therapists in the preparatory sessions before their first drug session, thereby developing a facilitative relationship, which will aim to reduce discomfort or difficulties that might arise during the course of therapy. Guidelines for each therapeutic session in exemplary embodiments follow. It will be appreciated that the nature of psychotherapy means that the client often drives the sessions according to the client's individual needs. The therapeutic approach also may be guided in part with reference to the Manual for MDMA-Assisted Psychotherapy in the Treatment of PTSD (Mithoefer, A Manual for MDMA-Assisted Psychotherapy in the Treatment of PTSD, 2017; Ver. 8.1).

In an embodiment, at the start of all psychotherapy sessions, SUD patients will be asked about substance use to confirm sobriety during therapy. For such patients, on all drug-assisted psychotherapy sessions, a drug test and urinalysis (dipstick) for recreational drugs and pregnancy (if applicable) will be performed. A positive result for recreational drugs other than cannabis (THC) on any of these tests would terminate the session.

Sessions 1 and 2: Therapists will discuss the compound to be administered, how it works, the expected effects, risks and review of the historical evidence of drugs such as MDMA as a tool for psychotherapy. The patient will be invited to share aspects of their life history that they feel important for the therapists to know, given that thoughts, feelings, images and memories about past experiences may come to the patient's mind during the drug session. Discussion will include curious questions about the patient's internal experiences (e.g., thoughts, emotions, imagery) and their understanding of this. The patient will be invited to discuss their current coping responses and any goals/hopes for participating, alongside intentions for what they would like to explore during the drug session.

Session 3 (Drug-assisted session): Patients will arrive at an agreed time in the morning, for instance between approximately 8-9 am. Continued consent will be registered and eligibility will be checked, including assessment of physical health, alcohol breath test and urine drugs screen for recreational drugs and pregnancy (if applicable). Vital sign measurements such as heart rate, blood pressure, and temperature will be taken at baseline before the drug is administered and before discharge (or more frequently if clinically indicated, including by use of medical sensors or biosensors that provide continuous, near-continuous, or periodic measurements). Consent to video and audio record the session also will be confirmed, and if withdrawn recording will not take place (or only audio recording may take place, if such specific consent is confirmed).

In an embodiment, patients will receive an initial dose of drug, as in the formulations of the Examples or an equivalent. Patients will be invited to relax for 90 minutes, lying down with eyeshades on, if they wish. The patient will have the option to listen to music, either through headphones or into the room, and may continue to do so throughout the therapy session. During this initial phase of the drug effects most patients will prefer to have little communication but rather to acclimatize to the psychological and physical effects of the drug.

After 90 minutes the patient will be invited to sit and for psychotherapy, and therefore such prolongation can be optimized or determined either by reference to the subjective (psychological) effects, or the pharmacokinetic (pharmacological) effects. Patients are not encouraged to get up and move around during the session, but rather to remain sitting or lying throughout. However, if a patient wishes to get up this can be facilitated if the therapy team judge it safe to do so. Physical touch from the therapists toward the patient is often employed as a therapeutic tool in MDMA therapy, and can be employed in the drug-assisted therapy of the methods herein. This technique can be used as a supportive grounding technique and to allow some patients to externalize their psychological pain. This technique will be carefully managed with professional boundaries by the therapists. The patient will be required to remain in the facility until they are deemed fit to return home. They will be assessed by the attendant doctor and signed off as medically fit for discharge. If not deemed ready to leave they should be required to remain in the therapy facility until deemed medically fit for discharge. The patient will then be met at the facility by their pre-arranged significant other, who must accompany them home. The patient should not be permitted to leave on their own.

Session 4: The day following session 3, the patient will undertake a follow-up session, with the therapy team. This generally will involve a 60-minute face-to-face follow-up session discussing aspects of drug-assisted psychotherapy undertaken the day before. Patients will be given the contact details of a clinical member of the therapy team whom they can contact if required for further telephone support during the week after each drug session. In some embodiments, patients will receive one or more remote or virtual follow-up after session 4, for example by daily phone calls or using another follow-up means as described further below.

Session 5 and 6: The patient has the opportunity to reflect upon the drug session and to explore the material that emerged. There are opportunities to reflect on new insights or perspectives gained and how this may impact on the issues unpinning their addictive behaviors, mental health disorders, and other areas of difficulty. Any intentions for the next drug session will be discussed.

Session 7 (Drug-assisted session): Same as for session 3 above.

Session 8: Same as session 4, above.

Session 9 and 10: Same as sessions 5 and 6, above. In addition, patients will be invited to consider any meaningful goals and/or values that they would like to focus on and move toward beyond the treatment phase. In session 10, the final session in this exemplary embodiment, the patient may complete outcome measures after the therapy session has ended. For example, this will include the Trauma History Questionnaire (THQ) (Green, Measurement of Stress, Trauma, and Adaptation. Lutherville, MD: Sidran Press; 1996. pp. 366-369) with the therapist. This questionnaire will give the therapy team information about the patient's past history of trauma. The visit including the therapy session will take approximately 1.5 hours. In this embodiment, after a patient finishes the tenth visit, treatment is either considered complete, or the patient progresses to the follow-up phase, further described below.

### 4b) Dru-Assisted Psychotherapy Course-Three Drug Sessions

In another exemplary embodiment of a drug-assisted psychotherapy treatment regime, a patient will receive 16 total therapy sessions during the treatment phase: thirteen 60-minute psychotherapy sessions, and three drug-assisted psychotherapy sessions (or another appropriately chosen length of time, and preferably a reduced period oftime under six hours, under five hours, under four hours, or about three hours or less, as discussed above and in view of the teachings herein). Sessions will take place approximately 1-2 weeks apart and start as soon as possible after baseline (generally, within approximately one to three weeks).

Exemplary Thirteen-Week Drug-Assisted Psychotherapy Course

| Week | Session | Activity |
|---|---|---|
| 1 | 1 | Non-Drug Prep Session |
| | | (60-minute therapy session) |
| 2 | 2 | Non-Drug Prep Session |
| | | (60-minute therapy session) |
| 3 | 3 | Drug-Assisted Therapy Session |
| | | 1 (less than 6 hours) |
| | 4 | Next-Day Non-Drug Integration/ |
| | | Follow-up Session (60 mins) |
| 4 | 5 | Non-Drug Integration Session |
| | | (60-minute therapy session) |
| 5 | 6 | Non-Drug Integration Session |
| | | (60-minute therapy session) |
| 6 | 7 | Drug-Assisted Therapy Session |
| | | 2 (less than 6 hours) |
| | 8 | Next-Day Non-Drug Integration/ |
| | | Follow-up Session (60 mins) |
| 7 | 9 | Non-Drug Integration Session |
| | | (60-minute therapy session) |
| 8 | 10 | Non-Drug Integration Session |
| | | (60-minute therapy session) |
| 9 | 11 | Non-Drug Integration Session |
| | | (60-minute therapy session) |
| 10 | 12 | Non-Drug Integration Session |
| | | (60-minute therapy session) |
| | 13 | Drug-Assisted Therapy Session |
| | | 3 (less than 6 hours) |
| 11 | 14 | Next-Day Non-Drug Integration/ |
| | | Follow-up Session (60 mins) |
| 12 | 15 | Non-Drug Integration Session |
| | | (60-minute therapy session) |
| 13 | 16 | Non-Drug Integration & Final |
| | | Evaluation Session (~90 mins) |

Phases one through four preceding this 13-week drug-assisted psychotherapy course generally are the same as above. Also as above, the drug-assisted psychotherapy sessions are run by a therapy team, with guidelines for each therapeutic session generally as described above, with modifications such as those in response to or accommodation of the overall longer course of treatment and the additional drug session, as will be appreciated by those of ordinary skill from the teachings herein and the general knowledge in the art.

Sessions 1-9: Generally, as described above for the eight-week course.

Session 10-12: Generally, the same as sessions 5 and 6.

Sessions 13-14: Generally, the same as sessions 3 and 4, and 7 and 8.

Session 15 and 16: Generally, the same as sessions 9 and 10 in the eight-week course above. Patients will continue to consider any meaningful goals and/or values that they would like to focus on and move toward beyond the treatment phase. In session 16, the final session in this exemplary embodiment, the patient will complete outcome measures after the therapy session has ended, such as the Trauma History Questionnaire (THQ). The visit including the therapy session will take approximately 1.5 hours. In this embodiment, after patients finish the sixteenth visit, treatment is either considered complete, or the patient progresses to the follow-up phase below. In alternative embodiments, one or more of sessions 10-12 for example may be eliminated or made optional, as may be one or more other sessions, or one or more sessions in the eight week treatment protocol above (e.g., session 6 or 9).

### 5) Follow-Up and Data Collection

“Follow-up” phase refers to the period following the last session of the drug-assisted psychotherapy course (e.g., session 10 or session 16 above), or if a patient discontinued treatment, from two weeks after the final psychotherapy session to the final follow-up visit. In an exemplary embodiment of the treatment protocol, outcomes will be assessed at 3, 6 and 9 months after the end of baseline. In such embodiments, the final follow-up visit is thus at 9 months post baseline completion. In other embodiments, a final follow-up visit may be 12 months or longer post baseline.

Means of performing follow-up visits and gathering follow-up data (e.g., to determine therapeutic efficacy and measure therapeutic effect) include in-person visits, telemedicine and other virtual visits, phone calls, automated inquiries and check-ins (e.g., email questionnaires, mobile apps, etc.), and the like. Accordingly, it will be appreciated that a “visit” need not be an in-person or face-to-face visit with a member of the therapy team, or another clinician.

Audit data from clinical notes indicates that formal assessment data for patients seeking treatment for substance use disorders and others is typically sparse and difficult to collect. Accordingly, in preferred embodiments herein, means are provided to not only collect high quality data about patient outcomes, but to do so in a way that increases compliance and successful data collection. Such means will ease the burdens of collecting follow-up data (to patients, caregivers, and others), minimize attrition rates (previously shown to be in the range of 10-35% by Hallgren & Witkiewitz, Alcohol Clin. Exp. Res., 2013; 37(12): 2152-60), and avoid the pitfalls of using statistical analysis to predict outcomes for those lost to follow-up by collecting good quality long term outcome data in these studies from the outset, e.g., by advantageously enhancing contact with patients or by using optimized means.

For example, when in person visits are used, patients will be invited to attend visits (estimated duration, 1.5 hours) at 3, 6 and 9 months post baseline. All patients will receive a reminder before the visit. At such follow-up visits, patients will be asked to complete, e.g., a urine test for recreational drugs, breathalyzer test for alcohol, and questionnaires, e.g., the DSM-5 criteria for substance use disorders, behavioral addictions, and CNS or mental health disorders, the Montgomery-Asberg Depression Rating Scale (MADRS), the Generalized Anxiety Disorder questionnaire (GAD-7), the Posttraumatic Stress Disorder Checklist (PCL-5), or other appropriate follow-up assessment. In some examples, improved psychological functioning may be determined by one or more of the absence of psychological dysfunction, the absence of psychological distress, and the presence of positive psychological functioning, e.g., with use of the Subjective Happiness Happiness Scale (SHS-4), The Scales of Psychological Wellbeing and other assessments and tools available to one of skill in the art. Aspects of psychological wellbeing that may be evaluated include autonomy, environmental mastery, personal growth, purpose in life, positive relations with others, and self-acceptance (Ryff & Keyes, J. Personality & Social Psych., 1995; 69(4), 719).

Follow-up telephone calls (estimated duration: 5-10 mins) also can be utilized to gather follow-up data to determine therapeutic efficacy and measure therapeutic effect. Systematic efforts will be made to contact patients to collect follow-up data; however, because some patients may be difficult to contact, patients can be asked (as an eligibility criterion) to identify a significant other who can be contacted in the event that the therapy team is unable to contact the patient to collect outcome data. When a patient is unreachable, and if the patient has consented, a significant other also may be asked to give information about outcome, e.g., related to substance use, compulsive behaviors related to behavioral addiction, mental health, and psychological functioning, etc.

### Example 21: Open Label within-Subject Safety and Tolerability Feasibility Study of Drug-Assisted Psychotherapy in Patients with Substance Use Disorder (SUD)

In this Example is described a study to investigate the use of the therapeutic compounds to treat AUD in patients with a substance use disorder (SUD). Exemplary SUDs include alcohol use disorder, nicotine dependency, opioid use disorder, stimulant use disorder, sedative, hypnotic, or anxiolytic use disorder, and tobacco use disorder. Subject may or may not complete a detoxification or similar course before their drug-assisted psychotherapy course. In the study of this Example, participants will receive two sessions with the drug. Psychological support will be provided before, during, and after each session. Safety and tolerability will be assessed alongside psychological and physiological outcome measures. Substance use behavior, mental well-being, and functioning data will be collected for nine months after baseline.

The study of this Example will demonstrate that the disclosed drug treatment of the methods of the invention is well-tolerated by all participants. No unexpected adverse events will be observed. Psychosocial functioning will improve across the cohort. Regarding substance use, at nine months post baseline the average substance consumption by participants will compare favorably to other observational studies with similar populations of people with the SUD. Such results, in view of the further description and disclosures provided herein, additionally demonstrate the therapeutic potential of drug-assisted psychotherapy for SUDs using the therapeutic compounds.

### 1) Methods

More specifically, the study of this Example is an open label within-subject safety and tolerability feasibility study, in patients with SUD. All patients will receive drug-assisted therapy. The main outcome measures will be the number of patients completing the eight week psychotherapy course, the number accepting the second booster dose of drug on drug-assisted days and adverse events. Secondary outcome measures will include changes in substance use behavior, e.g., drinking behavior, nicotine use, opioid use, stimulant use, sedative, hypnotic, or anxiolytic use, tobacco use (measured by consumption at 3, 6 and 9 months since baseline), measures of mental well-being, psycho-social functioning, quality of life and concomitant drug use.

Patients with a diagnosis of any SUD will be recruited. These patients may be recruited from healthcare and partner organizations, from primary care, by word of mouth, or by answering advertisements placed in appropriate locations. The sample size will be based on a power calculation of required group size to adequately test the hypothesis.

After completing a group therapy preparation course, patients will receive an eight week course of abstinence-based therapy comprising 10 psychotherapy sessions (such as described in further detail above). On two of these sessions (session 3 and 7) patients will be dosed with open-label drug during a drug-assisted therapy session. In certain preferred embodiments, the average total time of a drug-assisted session is therefore reduced to less than 6 hours, less than 5 hours, less than 4 hours, less than 3 hours, less than 2 hours, and about 1 hour or less. The eight week therapeutic course will start as soon as possible after a patient has completed the preparation course (with a delay up to two weeks).

In an alternative aspect, reflecting an alternative embodiment such as described above, patients will receive 16 therapy sessions, comprising of 13 60-minute non-drug psychotherapy sessions and three, 6-8 hour drug-assisted therapy sessions. Drug-assisted sessions will take place between three and five weeks apart (the interval between drug sessions 1 and 2 is three weeks, and the interval between drug sessions 2 and 3 is five weeks). This 13 week therapeutic course also will start as soon as possible after a patient has completed the four week group preparation course (with a delay up to two weeks).

Good Manufacturing Practice (GMP) drug (i.e., substantially pure compound) will be synthesized or commercially obtained and formulated into the investigational medicinal product. On each dosing session, patients will receive an initial oral dose of the drug (e.g., as in the formulation Examples above) followed two-hours later by a booster dose of one-half the initial dose. The booster dose will serve to prolong the experience, allowing for greater time for psychotherapy under the influence of the drug.

Other sessions (i.e., sessions 1, 2, 4, 5, 6, and 8-10) will comprise one hour psychotherapy sessions, and employ aspects of Motivational Interviewing and “third wave” cognitive-behavioral approaches. Patients will remain in the study for a duration of approximately 10 months.

### 2) Trial Procedures

SUD will be identified using available criteria, e.g. with reference to the DSM-5. Screening will comprise of written informed consent, an evaluation of the patient's physical and mental health background, a psychiatric interview (MINI), assessments of depression and anxiety severity will be assessed using the PHQ-9 and GAD-7 questionnaires. In some cases, the severity of substance used will be established using questionnaires, e.g., the Severity of Alcohol Dependence Questionnaire (SADQ) and the Short Inventory of Problems Alcohol and Drugs (SIP-AD). Patients will receive a thorough physical health check comprising of an electrocardiogram (ECG), routine blood tests, blood pressure, heart rate, and physical examination. Following screening, eligible patients will first receive a four-week course of supportive group therapy (motivational group meetings) in order to gradually reduce their intake of a the substance of abuse at issue. It may be necessary to confirm the absence of physical withdrawal. In one representative example, the Clinical Institute Withdrawal Assessment for Alcohol—Revised Version (CIWA-Ar) (Saitz et al., Medical Clinics of North America. 1997; 81(4):881-907) may be used to confirm the absence of alcohol withdrawal. Dip-stick urinalysis for recreational drugs can be completed at the research team's discretion. If female, dip-stick urinalysis for pregnancy will be completed. Patients will also be asked to complete post-preparation questionnaires. Subsequently, eligible patients enter the eight week (or 13 week) course of psychotherapy. This will entail weekly 60-minute outpatient non-drug psychotherapy sessions, delivered by two clinicians trained in delivering MDMA-assisted psychotherapy, with additional training as necessary to provide drug-assisted psychotherapy using the therapeutic compounds.

Dosing with the drug will occur twice during the eight week course, on weeks 3 and 6. Physiological changes, observer and subject ratings of distress and the intensity of the acute psychoactive effects of the drug will be measured throughout the drug-assisted session. Acute anxiety will be managed primarily psychologically, but sedative medication (oral lorazepam) will be available.

Patients will optionally remain overnight in the treatment center after each drug-assisted session, overseen by medically-trained “night sitters,” on hand to support patients as required but instructed to avoid delivering any psychotherapeutic interventions.

Patients will be seen the morning after each drug-assisted session for an integration psychotherapy session, and then telephoned daily for six days to assess changes to mood, suicidal risk factors (using C-SSRS) and quality of sleep (using the Leeds Sleep questionnaire). Following the end of the eight week (or alternatively, 13 week) therapeutic course participants will carry out additional follow-up questionnaires. They will then be seen again at 3, 6 and 9 months (since baseline) for longer-term follow-up data collection.

### 3) Data Analysis

All data will be recorded on paper case report forms (CRFs) and then digitized into MS Excel spreadsheets. Analysis and graphing will be performed using GraphPad (Prism) or MS Excel. When calculating Timeline Follow Back results, alcohol consumption levels at last observation will be used in the case of drop-outs or when participants undergo the group therapy preparation course more than once (Hamer & Simpson, American J. Psychiatry, 2009; 166(6):639-41).

### 4) Results

Severity of AUD criteria at screening and baseline: as per the inclusion criteria, all eligible patients will score above the diagnostic threshold on the DSM-5 SCID questionnaire for AUD. AUD severity will also be measured using the SIP questionnaire and the SADQ questionnaire, and no patients found to be physically dependent on alcohol will be included.

Physiological and tolerability effects during drug sessions: Drug-assisted psychotherapy sessions with the therapeutic compound will be administered during the trial to the patients. Temperature, blood pressure and heart rate will be measured at t=0, before taking the drug, then half-hourly up to t=2 hours, then hourly thereafter for a minimum of six hours from the time of dosing.

All of these physiological parameters are expected to remain within normal limits for all these sessions. A mild, transient rise on blood pressure, temperature and heart rate over the course of the drug session is expected to be seen. No patients are expected to experience sustained abnormal physiological disturbance, symptomatic experiences of raised blood pressure, heart rate or temperature or any other adverse events during drug sessions. No medical interventions are expected to be required in respect of these or any other physiological events during drug sessions.

Subjective Units of Distress and Participant report of Drug Effects will also be measured, hourly, throughout the drug sessions. Most subjects are predicted to report mildly raised Distress scores at the beginning of the sessions before taking the drug-consistent with expected anxiety ahead of dosing-which subsequently is predicted to be reduced during the course of the session as the positive effects of the drug emerge. Participants will give their own subjective score (0-10) of whether they felt Drug Effects, and the therapists also will record their own objective score of how “altered” the participant appeared. There is expected to be no significant difference between Observers' and Participants' Drug Effects scores. Drug Effects will rise expectedly over the first two hours, with a further increase after the booster dose is given at t=2 hours, and a subsequent plateau then decline over the following six hours. By the end of the drug session day all drug effects will have returned to baseline.

Changes in drinking behavior: Data will be collected in respect of Units of Alcohol Consumed per Week in the month before participants' baseline, immediately after baseline, throughout the eight week drug therapy course and for up to nine months after baseline, and patients are expected to reduce their drinking behavior in significant amounts.

Comparisons will be made for the prevalence of average number of units consumed per week between each respective time point of 3, 6, and 9 months following the final therapy session. Appropriate statistical analyses will be executed on the resulting figures via Pearsons correlation coefficients to identify the associated change between each point of TLFB data collection and baseline. The differences will then be assessed via Fisher r-z transformation (Lowry, 2001-2003, Significance of the difference between two correlation coefficients, Retrieved from http://vassarstats.net/rdiff.html) to test the hypothesis that drug-assisted psychotherapy compared to placebo-assisted psychotherapy is effective in this population of participants with AUD.

Seven-day follow-up after drug sessions: Considerable medical and popular press literature reports the anecdotal observation of recreational ecstasy users experiencing an acute “come down” effect and a drop in mood in the days after using the drug recreationally. In order to measure this prospectively with the therapeutic compound of the invention, participants' mood states will be measured by daily Profile Of Mood States (POMS) measurements for seven days after each drug session. Positive scores represent depressed affect, zero represents no change in mood or affect and results below zero represent a positively felt mood. Average scores across both drug sessions for all participants are expected to reveal no evidence of any mood disturbance during the week after taking each session of the therapeutic compound. Participants instead are expected to sustain a positive mood for seven days, contrasting with anecdotal reports from recreational ecstasy users, and additionally allowing comparison with results seen with clinical MDMA use.

Other mental health measures and quality of life measures: Brief assessments of mood and anxiety will be made at screening, baseline, after the eight week drug therapy course and at 3,6 and 9-month follow-ups, using the PHQ-9 and GAD-7 rating scales respectively. Scores are expected to demonstrate a reduction in both anxiety and depression after screening and baseline timepoints, followed by a transient rise in anxiety and depression scores 3 months after baseline and a further reduction at 6 months and a moderate rise again at 9 months post baseline. A variety of further data will be collected, including changes to the quality of sleep, quality of life measures and changes to compassion and empathy scales.

Suicidality: Participants will undergo the C-SSRS rating scale at screening, baseline, throughout the eight week therapy course, in the week after each drug session and at 3, 6 and 9 month follow-up visits. No participants are expected to report current suicidal ideation, intent or plans or self-harm behavior during the course of the study.

Adverse Events: The acute effects of drug-assisted psychotherapy are expected to be well-tolerated by participants. No unexpected adverse events are expected to occur. No psychotic symptoms will be observed in any patients.

### 5) Analysis

In view of such examples, and by modifications as may be made by those of ordinary skill in view of the disclosure herein and general knowledge in the art, the drug-assisted therapy of the invention will also be demonstrated to be useful in the treatment of other substance use disorders, CNS and mental health disorders, and in the improvement of mental health and psychological functioning

### Example 22: Randomized Double-Blind Between-Subject Controlled Study of Drug-Assisted Psychotherapy in Patients with Alcohol Use Disorder (AUD)

This Example describes a randomized, double-blind controlled study of efficacy of drug-assisted therapy for treatment of patients with AUD. AUD can refer to either harmful use, such as by a non-physically dependent subject, or to physical dependence. It will be understood that the methods provided herein are applicable to other substance use disorders, behavioral addictions, or CNS or mental health disorders, as would be known to one of skill. Such methods may also be applied to assess improvement in psychological functioning.

This is a randomized between-subject controlled study, in patients with non-physically dependent alcohol use disorder. Half of the patients will receive drug-assisted psychotherapy and the other half will receive placebo-assisted psychotherapy. In this study will be shown that drug-assisted psychotherapy is an effective treatment for patients with AUD. In other words, the study of this Example will show that drug-assisted psychotherapy is superior to placebo dose drug-assisted psychotherapy and can reduce use of alcohol and improve quality of life in patients with AUD.

Outcomes regarding abstinence from alcohol, concomitant drug use (the number and frequency of both prescribed medication use and recreational drug use), quality of life, and psychosocial functioning will be evaluated. The main outcome measure will be efficacy, which will be measured in weekly units of alcohol consumed at 3, 6 and 9 months post baseline. Here, as also in the above Example, the study will adhere to the principles outlined in the Medicines for Human Use (Clinical Trials) Regulations 2004 (SI 2004/1031), amended regulations (SI 2006/1928) and the International Conference on Harmonisation Good Clinical Practice (ICH GCP) guidelines, the Data Protection Act, and such other regulatory requirements as appropriate and as would be understood to those of ordinary skill in the art.

A population of adult patients with a primary diagnosis of AUD, aged between 18 and 65, who do not have physical dependence (and therefore do not require medical detoxification) and who meet the inclusion and exclusion criteria, will be recruited (in a number sufficient to generate statistically significant results). Patients will remain in the study for a duration of approximately 14 months.

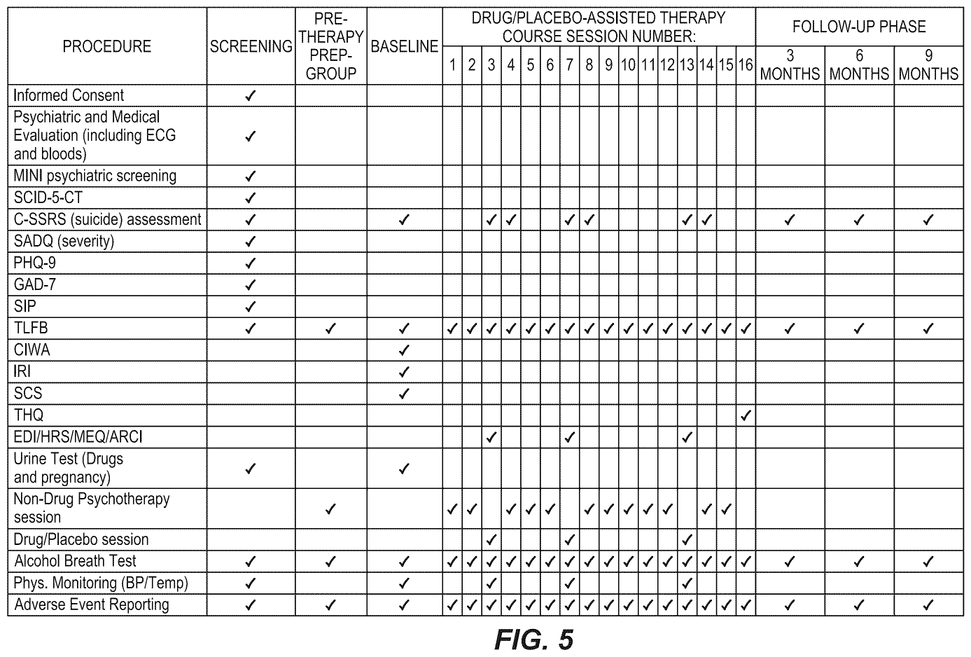

FIG. 5 shows an exemplary drug-assisted psychotherapy protocol. As depicted, and as also further described in the other Examples herein, patients will first receive a four-week course of supportive group therapy (motivational group meetings) in order to gradually reduce their intake of alcohol. Once abstinent, they will enter a 13-week course of drug-assisted therapy, comprising fifteen psychotherapy sessions (or, in an alternate aspect, an eight-week course as in the embodiment described above). Patients are randomized to either subtherapeutic/active control (placebo) or therapeutic dose.

On three psychotherapy sessions (session 3, session 7 and session 13) patients will be given either active control (subtherapeutic) dose of drug or active therapeutic (initial+booster) dose of drug during the 6-8 hour (or as otherwise appropriate, as discussed above) drug-assisted therapy sessions. Patients may optionally stay overnight, or go home after their drug session. Other sessions (1, 2, 4, 5, 6, 8, 9, 10, 11, 13, 12, 14 and 15) will comprise non-drug-assisted 1-hour psychotherapy sessions.

The sessions in general will be as described in other prophetic examples herein. From Session 15 or if discontinued treatment, from 2 weeks post the final psychotherapy session to the final follow-up visit (9 months post baseline). Patients will be followed up at 3, 6 and 9 months from the date of baseline.

Results are obtained as above, and through this Example, drug-assisted psychotherapy is further demonstrated to be useful in treating AUD and to have such other benefits as claimed. Accordingly, in embodiments, the pharmaceutical compositions of the invention (and their use in the methods of the invention) are used to improve the symptoms of a mental health disorder, wherein the mental health disorder is a substance abuse disorder. In some embodiments, that substance use disorder is AUD. The symptoms of the substance use disorders, behavioral addictions, or CNS or mental health disorders to be treated shall be able to be determined by one of skill, by reference to the general understanding of the art regarding that disorder, for example by reference to the DSM-5). Accordingly, methods of the invention, and the compositions of the invention when used in those methods, will be understood as improving any such symptoms.

In some embodiments, and as shown by examples as above, the therapeutic compounds and pharmaceutical compositions thereof (and their use in the methods of the invention) will produce a therapeutic effect, and such effect may be (1) an improvement in a symptom of a comorbid psychiatric disorder; (2) an increase in quality of life; (3) an increase in psychosocial functioning; (4) a decrease in use or frequency of prescription medication; (5) a decrease in use or frequency of recreational drugs; (6) a decrease in units of alcohol (or substance of abuse) consumed; (7) a reduction of cravings relating to alcohol (or other substance of abuse); (8) a prevention of relapse into drinking or substance use.

### Example 23: Effects of a Therapeutic Compound on Gambling Disorder

The goal of the proposed study is to evaluate the efficacy and safety of a disclosed therapeutic compound in individuals with gambling disorder, as determined by DSM-5 criteria. The hypothesis to be tested is that such compound will be more effective in reducing gambling severity and improving quality of life in subjects with gambling disorder compared to placebo.

Subjects are instructed to self-administer a therapeutic compound or placebo approximately one hour before a gambling session or when feeling urges to gamble. Alternatively, a subject may schedule a drop-in visit with a treatment provider to administer a therapeutic compound or placebo.

The study progresses for a period of time during which assessments are administered at predetermined time points. Outcomes of such assessments are evaluated at each timepoint.

Responses to the Yale Brown Obsessive Compulsive Scale—Pathological Gambling (PG-YBOCS), which assesses gambling severity, are a primary measure of the study.

Secondary measures of efficacy include:
Responses to the Clinical Global Impression-Improvement and Severity scales (CGI), which assesses overall disorder severity.
Responses to the Gambling Symptom Assessment Scale (G-SAS), which assesses severity of gambling symptoms.
Responses to the Hamilton Anxiety Rating Scale (HAM-A), which assesses levels of anxiety.
Responses to the Sheehan Disability Scale (SDS), which assesses level of disability resulting from gambling (or target disorder).
Responses to the Hamilton Depression Rating Scale (HAM-D), which assesses level of depression.
Responses to the Perceived Stress Scale (PSS), which assesses the level of perceived stress the individual experiences.
Responses to the Quality of Life Inventory (QOLI), which assesses the subject's overall perceived quality of life.

In an exemplary study, subjects administered therapeutic compounds report a substantial reduction in gambling severity, reductions in depression, anxiety, and stress, and an improvement in quality of life.

In view of such examples, and by modifications as may be made by those of ordinary skill in view of the disclosure herein and general knowledge in the art, the drug-assisted therapy of the invention will also be demonstrated to be useful in the treatment of other substance use disorders, behavioral addictions, and CNS and mental health disorders, and in the improvement of mental health and psychological functioning

The foregoing description, for purposes of explanation, uses specific nomenclature to provide a thorough understanding of the invention. However, it will be apparent to one skilled in the art that specific details are not required in order to practice the invention. Thus, the foregoing description of specific embodiments of the invention is presented for purposes of illustration and description. It is not intended to be exhaustive or to limit the invention to the precise forms disclosed; obviously, many modifications and variations are possible in view of the above teachings. The embodiments were chosen and described in order to best explain the principles of the invention and its practical applications, through the elucidation of specific examples, and to thereby enable others skilled in the art to best utilize the invention and various embodiments with various modifications as are suited to the particular use contemplated, when such uses are beyond the specific examples disclosed.

Accordingly, the scope of the invention shall be defined solely by the following claims and their equivalents, through which the numerous and varied above benefits, advantages, and improvements of the invention will be further appreciated and known.

1-78. (canceled)

79. A method of treating a medical condition in a mammal in need of such treatment, the method comprising administering a compound having the structure of any of:

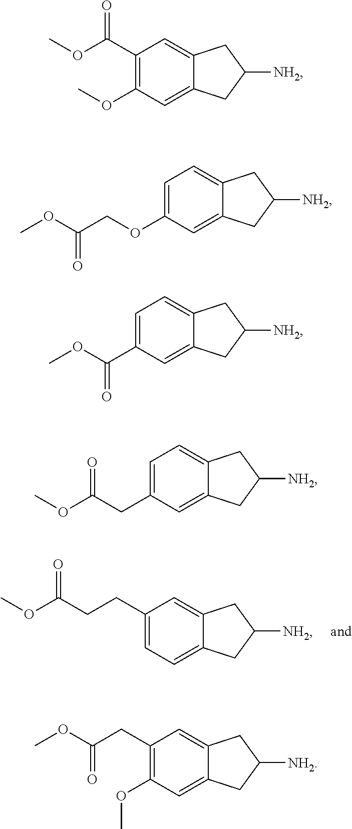 [SMILES: COC(=O)c1cc2c(cc1OC)CC(N)C2 ; COC(=O)COc1ccc2c(c1)CC(N)C2 ; COC(=O)c1ccc2c(c1)CC(N)C2 ; COC(=O)Cc1ccc2c(c1)CC(N)C2 ; COC(=O)CCc1ccc2c(c1)CC(N)C2 ; COC(=O)Cc1cc2c(cc1OC)CC(N)C2]

80. The method of claim 79, comprising administering the compound having the structure

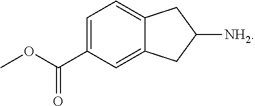

81-82. (canceled)

83. The method of claim 79, wherein the medical condition is a substance abuse disorder, a behavioral addiction, or a mental health disorder.

84-87. (canceled)

88. The method of claim 83, wherein the substance abuse disorder, behavioral addiction, or mental health disorder is selected from the group consisting of: alcohol use disorder, nicotine dependency, opioid use disorder, stimulant use disorder, sedative, hypnotic, or anxiolytic use disorder, tobacco use disorder, gambling disorder, compulsive sexual behavior, sexual addiction, gaming addiction, shopping addiction, internet addiction, kleptomania, pyromania, compulsive buying, pornography addiction, binge eating disorder, internet gaming addiction, exercise addiction or overtraining syndrome, love addiction, work addiction or workaholism, and technological addictions, post-traumatic stress disorder (PTSD), an anxiety disorder, a depressive disorder, adjustment disorder, affective disorder, depression, atypical depression, postpartum depression, catatonic depression, a depressive disorder due to a medical condition, premenstrual dysphoric disorder, seasonal affective disorder, dysthymia, anxiety, phobia disorders, binge disorders, body dysmorphic disorder, alcohol or drug abuse or dependence disorders, substance-related disorders, substance-induced mood disorder, a mood disorder related to another health condition, disruptive behavior disorders, eating disorders, impulse control disorders, obsessive compulsive disorder (OCD), attention deficit hyperactivity disorder (ADLID), personality disorders, attachment disorders, and dissociative disorders.

89. The method of claim 83, wherein the substance abuse disorder is any of alcohol use disorder, nicotine dependence, sedative, hypnotic, or anxiolytic use disorder, opioid abuse disorder, stimulant use disorder, and tobacco use disorder.

90. The method of claim 83, wherein the behavioral addiction is any of gambling disorder, compulsive sexual behavior, sexual addiction, gaming addiction, shopping addiction, internet addiction, kleptomania, pyromania, compulsive buying, pornography addiction, binge eating disorder, internet gaming addiction, exercise addiction or overtraining syndrome, love addiction, work addiction or workaholism, and technology addiction.

91. The method of claim 83, wherein the mental health disorder is any of an anxiety disorder, a depressive disorder, OCD, or PTSD.

92. The method of claim 91, wherein the anxiety disorder is generalized anxiety disorder (GAD).

93. The method of claim 91, wherein the depressive disorder is major depressive disorder (MDD) or treatment-resistant depression (TRD).

94-96. (canceled)

97. The method of claim 79, wherein the compound is administered in combination with one or more psychotherapy sessions.

98. The method of claim 97, wherein the patient has successfully completed a group therapy preparation course prior to the one or more psychotherapy sessions.

99. (canceled)

100. The method of claim 97, wherein the one or more psychotherapy sessions is between 5 and 20 psychotherapy sessions, during which at least two of said psychotherapy sessions the patient is administered the compound.

101. The method of claim 97, wherein the method results in one or more of: (a) a reduction of substance use, (b) a reduction of substance cravings, (c) a promotion of substance abstinence, (d) a prevention of relapse, or (e) an improvement of at least one symptom of the substance use disorder.

102. The method of claim 97, wherein the method results in one or more of: (a) an increase in quality of life, (b) an increase in psychosocial functioning, (c) a decrease in use or frequency of a prescription medication, (d) a decrease in use or frequency of a recreational drug, (e) a decrease in obsessive compulsive thoughts, (f) a decrease in suicidality, (g) an increase in feelings of empathy, or (h) an increase in self-compassion.

103. The method of claim 97, wherein the method results in an improvement of at least one symptom of a comorbid psychiatric disorder, such as antisocial personality disorder, borderline personality disorder, depression, anxiety, schizophrenia, attention deficit hyperactivity disorder, bipolar disorder, obsessive compulsive disorder, binge eating disorder, or PTSD.

104. The method of claim 103, wherein the results are measured at least 1 month, at least 3 months, at least 6 months, at least 9 months, or at least 1 year from baseline, or from the first psychotherapy session.

105. The method of claim 104, wherein the patient is also administered a therapeutically effective amount of an additional active agent selected from the group consisting of: an opioid antagonist (e.g., nalmefene, naltrexone), a CB-1 antagonist (e.g., rimonabant), a CRH1 receptor antagonist (e.g., verucerfont, pexacerfont), a NK1R antagonist (e.g., tradipitant), an OTR agonist (e.g., oxytocin), a GABA agent (e.g., topiramate, baclofen, a benzodiazepine, such as alprazolam, diazepam or lorazepam), a voltage-gated sodium channel inhibitor (e.g., oxacarbazepine, valproic acid, zonisamide), a voltage-dependent calcium channel agonist (e.g., gabapentin, pregabalin), an α7 nicotinic acetylcholine receptor agonist (e.g., varenicline), a 5-HT3 antagonist (e.g., ondansetron), a 5-HT1A receptor partial agonist (e.g., aripiprazole), a 5-HT2A receptor antagonist (e.g., quetiapine, olanzapine, mirtazapine), a 5-HT reuptake inhibitor (e.g., trazodone), a SERT inhibitor (e.g., duloxetine), an al adrenoreceptor antagonist (e.g., doxazosin, prazosin), a glucocorticoid receptor antagonist (e.g., mifepristone), an al adrenoreceptor agonist (e.g., guanfacine), an AChE inhibitor (e.g., citicoline), a dopamine D2 receptor antagonist (e.g., tiapride), an α2 adrenoreceptor agonist (e.g., clonidine), an NMDA receptor antagonist (e.g., acamprosate), an aldehyde dehydrogenase inhibitor (e.g., disulfiram), and pharmaceutically acceptable salts thereof.
